SEQUENCE LISTING (152 sequences)

SEQ ID NO: 1 - protein, 117 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 2 - protein, 8 aa, synthetic construct
GFSLNDYW

SEQ ID NO: 3 - protein, 7 aa, synthetic construct
IDVGGSL

SEQ ID NO: 4 - protein, 11 aa, synthetic construct
ARGGLTYGFDL

SEQ ID NO: 5 - protein, 110 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 6 - protein, 6 aa, synthetic construct
EDISSY

SEQ ID NO: 7 - skipped

SEQ ID NO: 8 - protein, 12 aa, synthetic construct
HYYATISGLGVA

SEQ ID NO: 9 - protein, 114 aa, synthetic construct
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 10 - protein, 110 aa, synthetic construct
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 11 - protein, 121 aa, synthetic construct
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSS

SEQ ID NO: 12 - protein, 8 aa, synthetic construct
GSTFSTYA

SEQ ID NO: 13 - protein, 8 aa, synthetic construct
FSGSGGFT

SEQ ID NO: 14 - protein, 14 aa, synthetic construct
AIPARGYNYGSFQH

SEQ ID NO: 15 - protein, 108 aa, synthetic construct
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVL

SEQ ID NO: 16 - protein, 6 aa, synthetic construct
NIGSKS

SEQ ID NO: 17 - skipped

SEQ ID NO: 18 - protein, 11 aa, synthetic construct
QVWDSSSDHVV

SEQ ID NO: 19 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 21 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 22 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 24 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 25 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 27 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 28 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 30 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 31 - protein, 446 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 32 - protein, 217 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 450 aa, synthetic construct
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 34 - protein, 214 aa, synthetic construct
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 107 aa, synthetic construct [sequence omitted]

SEQ ID NO: 36 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 38 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 39 - protein, 289 aa, Homo sapiens
MRIFAVFIFMTYWHLLNAFTVTVPKDLYVVEYGSNMTIECFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 40 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 41 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 42 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 43 - protein, 118 aa, synthetic construct
QVQLVESGGGLVQPGTSLRLSCSVSGFSLYSYNMGWVRQAPGKGLEYIGIISGGTIGHYASWAKGRFTISRDTSKTTLYLQMNSLTTEDTATYFCARAFYDDYDYNVWGPGTLVTVSS

SEQ ID NO: 44 - protein, 111 aa, synthetic construct
AIQLTQSPSSLSASVGGTVTITCQSSQSVYGNNQLSWYQQKPGQPPKLLIYQASKLETGVPSRFRGSGSGTQFTLTISSLQSEDFATYYCAGGYSSSSDTTFGGGTEVVVK

SEQ ID NO: 45 - protein, 8 aa, synthetic construct
GFSLYSYN

SEQ ID NO: 46 - protein, 8 aa, synthetic construct
IISGGTIG

SEQ ID NO: 47 - protein, 10 aa, synthetic construct
AFYDDYDYNV

SEQ ID NO: 48 - protein, 8 aa, synthetic construct
QSVYGNNQ

SEQ ID NO: 49 - skipped

SEQ ID NO: 50 - protein, 11 aa, synthetic construct
AGGYSSSSDTT

SEQ ID NO: 51 - protein, 8 aa, synthetic construct
GITFSNSG

SEQ ID NO: 52 - protein, 8 aa, synthetic construct
IWYDGSKR

SEQ ID NO: 53 - protein, 6 aa, synthetic construct
ATNDDY

SEQ ID NO: 54 - protein, 6 aa, synthetic construct
QSVSSY

SEQ ID NO: 55 - skipped

SEQ ID NO: 56 - protein, 9 aa, synthetic construct
QQSSNWPRT

SEQ ID NO: 57 - protein, 113 aa, synthetic construct [sequence omitted]

SEQ ID NO: 58 - protein, 107 aa, synthetic construct [sequence omitted]

SEQ ID NO: 59 - protein, 8 aa, synthetic construct
GYTFTNYY

SEQ ID NO: 60 - protein, 8 aa, synthetic construct
INPSNGGT

SEQ ID NO: 61 - protein, 13 aa, synthetic construct
ARRDYRFDMGFDY

SEQ ID NO: 62 - protein, 10 aa, synthetic construct
KGVSTSGYSY

SEQ ID NO: 63 - skipped

SEQ ID NO: 64 - protein, 9 aa, synthetic construct
QHSRDLPLT

SEQ ID NO: 65 - protein, 120 aa, synthetic construct [sequence omitted]

SEQ ID NO: 66 - protein, 111 aa, synthetic construct [sequence omitted]

SEQ ID NO: 67 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 8 aa, synthetic construct
GITFSNSG

SEQ ID NO: 70 - protein, 8 aa, synthetic construct
IWYDGSKR

SEQ ID NO: 71 - protein, 6 aa, synthetic construct
ATNDDY

SEQ ID NO: 72 - protein, 6 aa, synthetic construct
QSVSSY

SEQ ID NO: 73 - skipped

SEQ ID NO: 74 - protein, 9 aa, synthetic construct
QQSSNWPRT

SEQ ID NO: 75 - protein, 113 aa, synthetic construct [sequence omitted]

SEQ ID NO: 76 - protein, 107 aa, synthetic construct [sequence omitted]

SEQ ID NO: 77 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 127 aa, synthetic construct
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 80 - protein, 108 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 81 - protein, 124 aa, synthetic construct
EMQLVESGGGLVQPGRSMKLSCAGSGFTLSDYGVAWVRQAPKKGLEWVAYISYAGGTTYYRESVKGRFTISRDNAKSTLYLQMDSLRSEDTATYYCTIDGYGGYSGSHWYFDFWGPGTMVTVSS

SEQ ID NO: 82 - protein, 106 aa, synthetic construct
DIQMTQSPSLLSASVGDRVTLNCRTSQNVYKNLAWYQQKLGEAPKLLIYNANSLQAGIPSRFSGSGSGTDFTLTISSLQPEDVATYFCQQYYSGNTFGAGTNLELK

SEQ ID NO: 83 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYLMYSKLTVEKKNWVERNSYSCSVVHEGLHNHHTTKSFSRTPGK

SEQ ID NO: 84 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVKDFMPEDIYVEWTNNGKTELNYKNTEPVLDSDGSYFMYSRLRVEKKNWVERNSYSCSVVHEGLHNHHTTKSFSRTPGK

SEQ ID NO: 85 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 86 - protein, 118 aa, synthetic construct
EVQLVESGGGLVQPGGSLRLSCAASGFTFSDSWIHWYRQAPGKGLEWYAWISPYGGSTYYADSVKGRFTISADTSKNTAYLQMNSLRAEDTAVYYCARRHWPGGFDYWGQGTLVTVSS

SEQ ID NO: 87 - protein, 107 aa, synthetic construct [sequence omitted]

SEQ ID NO: 88 - protein, 118 aa, synthetic construct; identical to SEQ ID NO: 43

SEQ ID NO: 89 - protein, 111 aa, synthetic construct; identical to SEQ ID NO: 44

SEQ ID NO: 90 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFERGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 92 - protein, 329 aa, synthetic construct; identical to SEQ ID NO: 25

SEQ ID NO: 93 - protein, 329 aa, synthetic construct
  VARIANT 117: note = X is A, V, L, I, P, F, M or W, preferably I, P, F, M or W, more preferably F
  VARIANT 118: note = X is L, D or E, preferably D or E, more preferably E
  VARIANT 119: note = X is not G, preferably K, R or H, more preferably R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEXXXGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 329 aa, synthetic construct; identical to SEQ ID NO: 91

SEQ ID NO: 95 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 96 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 107 aa, synthetic construct [sequence omitted]

SEQ ID NO: 98 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 10 aa, synthetic construct
AFYDDYDYNV

SEQ ID NO: 100 - protein, 12 aa, synthetic construct
ARAFYDDYDYNV

SEQ ID NO: 101 - protein, 7 aa, synthetic construct
ISGGTIG

SEQ ID NO: 102 - protein, 16 aa, synthetic construct
IISGGTIGHYASWAKG

SEQ ID NO: 103 - protein, 5 aa, synthetic construct
SYNMG

SEQ ID NO: 104 - protein, 8 aa, synthetic construct
GFSLYSYN

SEQ ID NO: 105 - protein, 11 aa, synthetic construct
AGGYSSSSDTT

SEQ ID NO: 106 - protein, 7 aa, synthetic construct
QASKLET

SEQ ID NO: 107 - protein, 8 aa, synthetic construct
QSVYGNNQ

SEQ ID NO: 108 - protein, 13 aa, synthetic construct
QSSQSVYGNNQLS

SEQ ID NO: 109 - protein, 116 aa, synthetic construct
QSVEESGGRLVTPGTPLTLTCTVSGFSLYSYNMGWVRQAPGKGLEYIGIISGGTIGHYASWAKGRFTISKTSSTTVDLKMTSLTTEDTATYFCARAFYDDYDYNVWGPGTLVTVSS

SEQ ID NO: 110 - protein, 111 aa, synthetic construct
AAVLTQTPSPVSAAVGGTVTISCQSSQSVYGNNQLSWYQQKPGQPPKLLIYQASKLETGVPSRFKGSGSGTQFTLTISDLESDDAATYYCAGGYSSSSDTTFGGGTEVVVK

SEQ ID NO: 111 - protein, 118 aa, synthetic construct; identical to SEQ ID NO: 43

SEQ ID NO: 112 - protein, 111 aa, synthetic construct; identical to SEQ ID NO: 44

SEQ ID NO: 113 - protein, 288 aa, Homo sapiens; identical to SEQ ID NO: 41

SEQ ID NO: 114 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 115 - DNA, 2115 bp, Homo sapiens
  source 1..2115: mol_type = other DNA
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 116 - protein, 121 aa, synthetic construct
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 117 - protein, 8 aa, synthetic construct
GFTFTDFY

SEQ ID NO: 118 - protein, 10 aa, synthetic construct
IRDKAKGYTT

SEQ ID NO: 119 - protein, 12 aa, synthetic construct
AREGHTAAPFDY

SEQ ID NO: 120 - protein, 107 aa, synthetic construct
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIK

SEQ ID NO: 121 - protein, 6 aa, synthetic construct
QNIDKY

SEQ ID NO: 122 - protein, 9 aa, synthetic construct
LQHISRPRT

SEQ ID NO: 123 - protein, 127 aa, synthetic construct; identical to SEQ ID NO: 79

SEQ ID NO: 124 - protein, 8 aa, synthetic construct
GYRFSNFV

SEQ ID NO: 125 - protein, 8 aa, synthetic construct
INPYNGNK

SEQ ID NO: 126 - protein, 20 aa, synthetic construct
ARVGPYSWDDSPQDNYYMDV

SEQ ID NO: 127 - protein, 108 aa, synthetic construct; identical to SEQ ID NO: 80

SEQ ID NO: 128 - protein, 7 aa, synthetic construct
HSIRSRR

SEQ ID NO: 129 - protein, 9 aa, synthetic construct
QVYGASSYT

SEQ ID NO: 130 - protein, 29 aa, synthetic construct
QSVEESGGRLVTPGTPLTLTCTVSGFSLY

SEQ ID NO: 131 - protein, 14 aa, synthetic construct
WVRQAPGKGLEYIG

SEQ ID NO: 132 - protein, 31 aa, synthetic construct
RFTISKTSSTTVDLKMTSLTTEDTATYFCAR

SEQ ID NO: 133 - protein, 11 aa, synthetic construct
WGPGTLVTVSS

SEQ ID NO: 134 - protein, 23 aa, synthetic construct
AAVLTQTPSPVSAAVGGTVTISC

SEQ ID NO: 135 - protein, 15 aa, synthetic construct
WYQQKPGQPPKLLIY

SEQ ID NO: 136 - protein, 32 aa, synthetic construct
GVPSRFKGSGSGTQFTLTISDLESDDAATYYC

SEQ ID NO: 137 - protein, 10 aa, synthetic construct
FGGGTEVVVK

SEQ ID NO: 138 - protein, 30 aa, synthetic construct
QVQLVESGGGLVQPGTSLRLSCSVSGFSLY

SEQ ID NO: 139 - protein, 14 aa, synthetic construct
WVRQAPGKGLEYIG

SEQ ID NO: 140 - protein, 32 aa, synthetic construct
RFTISRDTSKTTLYLQMNSLTTEDTATYFCAR

SEQ ID NO: 141 - protein, 11 aa, synthetic construct
WGPGTLVTVSS

SEQ ID NO: 142 - protein, 23 aa, synthetic construct
AIQLTQSPSSLSASVGGTVTITC

SEQ ID NO: 143 - protein, 15 aa, synthetic construct
WYQQKPGQPPKLLIY

SEQ ID NO: 144 - protein, 32 aa, synthetic construct
GVPSRFRGSGSGTQFTLTISSLQSEDFATYYC

SEQ ID NO: 145 - protein, 10 aa, synthetic construct
FGGGTEVVVK

SEQ ID NO: 146 - protein, 5 aa, synthetic construct
NYYMY

SEQ ID NO: 147 - protein, 17 aa, synthetic construct
GINPSNGGTNFNEKFKN

SEQ ID NO: 148 - protein, 11 aa, synthetic construct
RDYRFDMGFDY

SEQ ID NO: 149 - protein, 15 aa, synthetic construct
RASKGVSTSGYSYLH

SEQ ID NO: 150 - protein, 7 aa, synthetic construct
LASYLES

SEQ ID NO: 151 - protein, 9 aa, synthetic construct
QHSRDLPLT

SEQ ID NO: 152 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0002600 A1
(43) Pub. Date: Jan. 2, 2025

(54) MULTISPECIFIC BINDING AGENTS AGAINST PD-L1 AND CD137 IN COMBINATION THERAPY

(71) Applicants: Genmab A/S, Valby (DK); BioNTech SE, Mainz (DE)

(72) Inventors: Alexander MUIK, Mainz (DE); Kristina SCHÖDEL, Mainz (DE); Nora PENCHEVA, Utrecht (NL); Maria N. JURE-KUNKEL, Plainsboro, NJ (US); Ugur SAHIN, Mainz (DE); Sina FELLERMEIER-KOPF, Mainz (DE); Patricia GARRIDO CASTRO, Utrecht (NL); Jordan BLUM, Plainsboro, NJ (US); Friederike GIESEKE, Mainz (DE); Esther C. W. BREIJ, Utrecht (NL); Lars GUELEN, Utrecht (NL); Joost NEIJSSEN, Utrecht (NL); Karsten BECKMANN, Mainz (DE); Claudia PAULMANN, Mainz (DE); Bart-Jan DE KREUK, Utrecht (NL); Richard G. HIBBERT, Utrecht (NL); Janine SCHUURMAN, Utrecht (NL); Aran LABRIJN, Utrecht (NL); Ivan KUZMANOV, Mainz (DE)

(21) Appl. No.: 18/696,689

(22) PCT Filed: Oct. 5, 2022
(86) PCT No.: PCT/EP2022/077748

§ 371 Date: Mar. 28, 2024

Related U.S. Application Data
(60) Provisional application No. 63/341,408, filed on May 12, 2022.
(60) Provisional application No. 63/257,889, filed on Oct. 20, 2021.
(60) Provisional application No. 63/253,103, filed on Oct. 6, 2021.

(51) Int. Cl. C07K 16/28; A61K 9/00; A61K 39/00; A61P 35/00

(52) U.S. Cl. CPC C07K 16/2878; A61K 9/0019; A61P 35/00; C07K 16/2818; C07K 16/2827; A61K 2039/507; C07K 2317/31; C07K 2317/524; C07K 2317/526; C07K 2317/53; C07K 2317/565; C07K 2317/567; C07K 2317/71

(57) ABSTRACT

The present invention provides combination therapy using a binding agent that binds to human CD137 and to human PD-L1 in combination with a PD-1 inhibitor to reduce or prevent progression of a tumor or treating cancer.

### RELATED APPLICATIONS

This application is a 35 U.S.C. § 371 filing of International Patent Application No. PCT/EP2022/077748, filed Oct. 5, 2022, which claims priority to U.S. Provisional Patent Application Serial Nos. 63/341,408, filed May 12, 2022; 63/257,889, filed Oct. 20, 2021; and 63/253,103, filed Oct. 6, 2021, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Mar. 28, 2024, is named 753023_GMB9-011US_ST26.xml and is 147,684 bytes in size.

### TECHNICAL FIELD

The present invention relates to combination therapy using a binding agent that binds to human CD137 and to human PD-L1 in combination with a PD-1 inhibitor to reduce or prevent progression of a tumor or treating cancer.

### BACKGROUND

CD137 (4-1BB) is a member of the TNFR family and is a co-stimulatory molecule on CD8⁺ and CD4+ T cells, regulatory T cells (Tregs), Natural Killer T cells (NK(T) cells), B cells and neutrophils. On T cells, CD137 is not constitutively expressed, but induced upon T-cell receptor (TCR) activation (for example, on tumor infiltrating lymphocytes (TILs) (Gros et al., J. Clin Invest 2014; 124(5):2246-59)). Stimulation via its natural ligand 4-1BBL or agonist antibodies leads to signaling using TRAF-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD8⁺ T-cell survival. Agonistic antibodies against CD137 have been shown to promote anti-tumor control by T cells in various pre-clinical models (Murillo et al., Clin Cancer Res 2008; 14(21):6895-906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (AU 2004279877) and utomilumab, a human IgG2 antibody (Fisher et al., 2012, Cancer Immunol. Immunother. 61: 1721-1733).

Programmed death ligand 1 (PD-L1, PDL1, CD274, B7H1) is a 33 kDa, single-pass type I membrane protein. Three isoforms of PD-L1 have been described, based on alternative splicing. PD-L1 belongs to the immunoglobulin (Ig) superfamily and contains one Ig-like C2-type domain and one Ig-like V-type domain. Freshly isolated T and B cells express negligible amounts of PD-L1 and a fraction (about 16%) of CD14⁺ monocytes constitutively express PD-L1. However, interferon-γ (IFNγ) is known to upregulate PD-L1 on tumor cells.

PD-L1 obstructs anti-tumor immunity by 1) tolerizing tumor-reactive T cells by binding to its receptor, programmed cell death protein 1 (PD-1) (CD279) on activated T cells; 2) rendering tumor cells resistant to CD8⁺ T cell and Fas ligand-mediated lysis by PD-1 signaling through tumor cell-expressed PD-L1; 3) tolerizing T cells by reverse signaling through T cell-expressed CD80 (B7.1); and 4) promoting the development and maintenance of induced T regulatory cells. PD-L1 is expressed in many human cancers, including melanoma, ovarian, lung and colon cancer (Latchman et al., 2004 Proc Natl Acad Sci USA 101, 10691-6).

PD-L1 blocking antibodies have shown clinical activity in several cancers known to overexpress PD-L1 (incl. melanoma, NSCLC). For example, atezolizumab is a humanized IgG1 monoclonal antibody against PD-L1. It is currently in clinical trials as an immunotherapy for several indications including various types of solid tumors (see e.g. Rittmeyer et al., 2017 Lancet 389:255-265) and is approved for non-small-cell lung cancer and bladder cancer indications. Avelumab, a PD-L1 antibody, (Kaufman et al Lancet Oncol. 2016; 17(10):1374-1385) has been approved by the FDA for the treatment of adults and pediatric patients 12 years and older with metastatic Merkel cell carcinoma, and is currently in clinical trials in several cancer indiciations, including bladder cancer, gastric cancer, head and neck cancer, mesothelioma, NSCLC, ovarian cancer and renal cancer. Durvalumab, a PD-L1 antibody, is approved for locally advanced or metastatic urothelial carcinoma indications, and is in clinical development in multiple solid tumors and blood cancers (see e.g. Massard et al., 2016 J Clin Oncol. 34(26):3119-25). Further anti-PD-L1 antibodies have been described e.g. in WO2004004771.

Horton et al (J Immunother Cancer. 2015; 3(Suppl 2): 010) discloses combination of an agonistic 4-1BB antibody with a neutralizing PD-L1 antibody. WO 2019/025545 provides binding agents, such as bispecific antibodies, binding human PD-L1 and binding human CD137.

However, despite these advances in the art there is a considerable need for improved therapies to prevent progression of a tumor or treating cancer.

### SUMMARY

The present inventors have surprisingly found that a combination of (i) stimulation with a binding agent binding human CD137 and binding human PD-L1 and (ii) a PD-1 inhibitor (in particular a PD-1 antibody) amplifies the immune response.

Thus, in a first aspect, the present disclosure provides a binding agent for use in a method for reducing or preventing progression of a tumor or treating cancer in a subject, said method comprising administering to said subject the binding agent prior to, simultaneously with, or after administration of a PD-1 inhibitor, wherein the binding agent comprises a first binding region binding to CD137 and a second binding region binding to PD-L1; and wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively, then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

In a second aspect, the present disclosure provides a kit comprising (i) a binding agent comprising a first binding region binding CD137 and a second region binding to PD-L1, and (ii) a PD-1 inhibitor, wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,
   then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

In a third aspect, the present disclosure provides a kit of the second aspect for use in a method for reducing or preventing progression of a tumor or treating cancer in a subject.

In a fourth aspect, the present disclosure provides a method for reducing or preventing progression of a tumor or treating cancer in a subject, said method comprising administering to said subject a binding agent prior to, simultaneously with, or after administration of a PD-1 inhibitor, wherein the binding agent comprises a first binding region binding to CD137 and a second binding region binding to PD-L1, and wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,
   then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

### BRIEF DESCRIPTION OF THE FIGURES

FIG. 1 shows a schematic representation of the anticipated mode of action of CD137xPD-L1 bispecific antibodies. (A) PD-L1 is expressed on antigen-presenting cells (APCs) as well as on tumor cells. PD-L1 binding to T cells expressing the negative regulatory molecule PD-1 effectively overrides T cell activation signals and eventually leads to T cell inhibition. (B) Upon addition of a CD137xPD-L1 bispecific antibody, the inhibitory PD-1:PD-L1 interaction is blocked via the PD-L1-specific arm and at the same time, the bispecific antibody, through the cell-cell interaction provides agonistic signaling to CD137 expressed on the T cells resulting in strong T cell costimulation.

FIG. 2 shows the MC38 syngeneic tumor model that was established by subcutaneous inoculation of 1×10⁶ MC38 cells into C57BL/6 mice. When tumors reached an average volume of 64 mm³, mice were randomized and treated with mbsIgG2a-PD-L1x4-1BB (5 mg/kg), an anti-mouse PD-1 antibody (anti-mPD-1; 10 mg/kg), either alone or in combination, or PBS (all 2QWX3). A. Data shown are the median tumor volume per treatment group (n=10) with data carried forward for animals that reached termination criteria. Growth curves were discontinued when<50% of the animals within a treatment group remained alive (PBS, mbsIgG2a-PD-L1x4-1BB, anti-mPD-1) or until Day 35 (combination of mbsIgG2a-PD-L1x4-1BB with anti-mPD-1). Arrows indicate days of treatment. B. Progression-free survival, defined as the percentage of mice with tumor volume smaller than 500 mm³, is shown as Kaplan Meier curve. Mantel Cox analysis was used to compare survival between treatment groups on Day 45 (Table 8).

FIG. 3 shows analysis of the proliferation dose-response of GEN1046 and anti-PD-1 antibody Nivolumab in an antigen-specific T cell assay with active PD1/PD-L1 axis. CFSE-labeled T cells electroporated with a claudin-6-specific TCR- and PD-I-IVT-RNA were incubated with claudin-6-IVT-RNA-electroporated immature dendritic cells in the presence of (A) GEN1046 (at 3-fold serial dilutions from 1 to 0.00015 μg/mL) or (B) Nivolumab (at 4-fold serial dilutions from 0.8 to 0.00005 μg/mL) for five days. CD8⁺ T cell proliferation was measured by flow cytometry. Data shown are expansion indices as a function of the antibody concentration. Error bars (SD) indicate variation within the experiment (n=3 replicates in (A); n=2 duplicates in (B), using cells from one representative donor). Curves were fitted by 4-parameter logarithmic fit and EC₅₀ values and Hill-Slopes (shown in Table 9 and 10) were determined using GraphPad Prism software v9.0.

FIG. 4 shows release of the PD-1/PD-L1-mediated T cell inhibition and additional co-stimulation of CD8⁺ T cell proliferation by GEN1046 in the absence or presence of anti-PD-1 antibody Nivolumab. CFSE-labelled T cells electroporated with a claudin-6-specific TCR- and PD-1-in vitro translated (IVT)-RNA were incubated with claudin-6-IVT-RNA-electroporated immature dendritic cells in the presence of 0.2 μg/mL, 0.0067 μg/mL or 0.0022 μg/mL GEN1046 in combination with a fixed concentration of 1.6. μg/mL Nivolumab or 0.8 μg/mL non-binding control antibody IgG1-ctrl for five days (n=2 technical replicates per condition, using cells from n=3 individual donors). Medium only, 0.8 μg/mL IgG1-ctrl only and 1.6 μg/mL Nivolumab only were used to determine baseline proliferation in the absence of GEN1046. CD8⁺ T cell proliferation was measured by flow cytometry. Bar graphs represent the mean±SD of expansion indices per indicated condition calculated using FlowJo software v10.7.1. The dashed line represents baseline proliferation in the presence of the anti-PD-1 antibody Nivolumab.

FIG. 5 is a schematic representation of a first-in-human, open-label, dose-escalation trial with expansion cohorts to evaluate safety of GEN1046 in subjects with malignant solid tumors.

FIG. 6 is a waterfall plot showing progression-free survival in subjects having received prior therapy with a checkpoint inhibitor (gray line) and checkpoint inhibitor naiive patients (black line).

FIG. 7 compares time since last prior anti-PD-(L)1 in subjects across CPI-experienced expansion cohorts (GEN1046 monotherapy) with clinical response (PR), compared to those with stable disease (SD) or progressive disease (PD). Response groups were compared using a Wilcoxon test. PR vs. PD: p=0.0017; PR vs. SD: p=0.034.

FIG. 8 shows binding of IgG1-PD1 to PD-1 of different species. CHO-S cells transiently transfected with PD-1 of different species were incubated with IgG1-PD1, pembrolizumab, or non-binding control antibodies IgG1-ctrl-FERR and IgG4-ctrl and binding analyzed using flow cytometry. Non-transfected CHO-S cells incubated with IgG1-PD1 were included as a negative control. A-B. Data shown are the geometric mean fluorescence intensities (gMFI)±SD of duplicate wells from one representative experiment out of four experiments. C-D. Data shown are the gMFI±SD of duplicate wells from one representative experiment out of two experiments. E. Data shown are the geometric mean fluorescence intensities (gMFI)±SD of duplicate wells from one representative experiment out of four experiments. Abbreviations: gMFI=geometric mean fluorescence intensity; PD-1=programmed cell death protein 1; PE=R-Phycoerythrin.

FIG. 9 shows competitive binding of IgG1-PD1 with PD-L1 and PD-L2 to human PD-1. CHO-S cells transiently transfected with human PD-1 were incubated with 1 μg/mL biotinylated recombinant human PD-L1 (A) or PD-L2 (B) in the presence of IgG1-PD1 or pembrolizumab. IgG1-ctrl-FERR was included as a negative control. Cells were stained with streptavidin-allophycocyanin, and the percentage of cells binding biotinylated PD-L1 or PD-L2 was determined by measuring the percentage of streptavidin-allophycocyanin+cells using flow cytometry. The percentage of streptavidin-allophycocyanin+cells in the no antibody control and in a non-transfected sample are indicated with dashed lines. Data shown are from single replicates from one representative experiment out of three separate experiments. Abbreviations: Ab=antibody; CHO-S=Chinese hamster ovary, suspension; ctrl=control; FERR=L234F/L235E/G236R-K409R; PD-1=programmed cell death protein 1; PD-L1=programmed cell death 1 ligand 1; PD-L2=programmed cell death 1 ligand 2.

FIG. 10 shows functional inhibition of the PD-1/PD-L1 checkpoint by IgG1-PD1. Blockade of the PD-1/PD-L1 axis was tested using a cell-based bioluminescent PD-1/PD-L1 blockade reporter assay. Data shown are mean luminescence±SD of duplicate wells in one representative experiment out of five (pembrolizumab and IgG1-PD1), three (IgG1-ctrl-FERR) or two (nivolumab) experiments. Abbreviations: FERR=L234F/L235E/G236R-K409R; PD1=programmed cell death protein 1; PD-L1=programmed cell death 1 ligand 1; RLU=relative light units; SD=standard deviation.

FIG. 11 shows the enhancement of CD8⁺ T-cell proliferation by IgG1-PD1 in an antigen-specific T-cell proliferation assay. Human CD8⁺ T cells were electroporated with RNA encoding a CLDN6-specific TCR and RNA encoding PD-1 and labeled with CFSE. The T cells were then co-cultured with iDCs electroporated with CLDN6-encoding RNA, in the presence of IgG1-PD1, pembrolizumab, nivolumab, or IgG1-ctrl-FERR. CFSE dilution in T cells was analyzed by flow cytometry after 4 d and used to calculate the expansion index. Data from one representative donor (26268_B) out of four donors evaluated in three independent experiments are shown. Error bars represent SD of duplicate wells. Curves were fitted by 4-parameter logarithmic fit using GraphPad Prism. Abbreviations: CFSE=carboxyfluorescein succinimidyl ester; FERR=L234F/L235E/G236R-K409R; PD1=programmed cell death protein 1; SD=standard deviation.

FIG. 12 shows IgG1-PD1-induced IFNγ secretion in an allogeneic MLR assay. Three unique donor pairs of allogeneic human mDCs and CD8⁺ T cells were cocultured in the presence of IgG1-PD1 or pembrolizumab for 5 d. IgG1-ctrl-FERR and an IgG4 isotype control were included as negative controls. IFNγ secretion was analyzed in the supernatant using an IFNγ-specific immunoassay. Data shown are mean ±standard error of the mean (SEM) concentration for three unique allogeneic donor pairs. Abbreviations: FERR=L234F/L235E/G236R-K409R; IFN=interferon; IgG=immunoglobulin G; mDC=mature dendritic cell; MLR=mixed lymphocyte reaction; SEM=standard error of the mean.

FIG. 13 shows IgG1-PD1-induced cytokine secretion in an allogeneic MLR assay. Three unique donor pairs of allogeneic human mDCs and CD8⁺ T cells were cocultured in the presence of 1 μg/mL IgG1-PD1 or pembrolizumab for 5 d. IgG1-ctrl-FERR was included as a negative control. Cytokine secretion was analyzed in the supernatant using Luminex. (A) Cytokine levels are represented as the average fold change over the cytokine levels measured in untreated cocultures. (B) Shown are the levels of cytokine production of three unique allogeneic donor pairs, with horizontal lines indicating the mean, upper, and lower limits. Abbreviations: FC=fold change; FERR=L234F/L235E/G236R-K409R; GM-CSF=granulocyte macrophage colony-stimulating factor; IgG=immunoglobulin G; IL=interleukin; MCP-1=monocyte chemoattractant protein 1; mDC=mature dendritic cell; MLR=mixed lymphocyte reaction; TNF=tumor necrosis factor.

FIG. 14 shows C1q binding to membrane-bound IgG1-PD1. Binding of C1q to IgG1-PD1 was analyzed using stimulated human CD8⁺ T cells. After incubation with IgG1-PD1, IgG1-ctrl-FERR, IgG1-ctrl, or positive control antibody IgG1-CD52-E430G (without inertness mutations and with a hexamerization-enhancing mutation), cells were incubated with human serum as a source of C1q. Binding of C1q was detected with a FITC-conjugated rabbit anti-C1q antibody. Data shown are the geometric mean fluorescence intensities (gMFI)+standard deviation (SD) from duplicate wells from one representative donor out of seven donors across three comparable experiments. Abbreviations: FITC=fluorescein isothiocyanate; gMFI=geometric mean fluorescence intensity; PE=R-phycoerythrocyanin.

FIG. 15 shows FcγR binding of IgG1-PD1. The binding of IgG1-PD1 to immobilized human recombinant FcγR constructs was analyzed by SPR in a qualified assay (n=1). FcγRIa (A), FcγRIIa-H131 (B), FcγRIIa-R131 (C), FcγRIIb (D), FcγRIIIa-F158 (E), and FcγRIIIa-V158 (F) binding of IgG1-PD1. The antibody IgG1-ctrl (without the FER inertness mutations) was included as a positive control for binding. Abbreviations: ctrl=control; FcγR=Fe gamma receptor; IgG=immunoglobulin G; PD-1=programmed cell death protein 1; RU=resonance units.

FIG. 16 shows FcγR binding of IgG1-PD1 and several other anti-PD-1 antibodies. The binding of IgG1-PD1, nivolumab, pembrolizumab, dostarlimab, and cemiplimab to immobilized human recombinant FcγR constructs was analyzed by SPR (n=3). FcγRla (A), FcγRIIa-H131 (B), FcγRIIa-R131 (C), FcγRIIb (D), FcγRIIIa-F158 (E), and FcγRIIIa-V158 (F) binding of the test antibodies. The IgG1-ctrl and IgG4-ctrl antibodies were included as positive controls for FcγR binding of IgG1 and IgG4 molecules with wild-type Fc regions. Shown is the binding response±SD of three separate experiments. Abbreviations: ctrl=control; FcγR=Fe gamma receptor; IgG=immunoglobulin G; PD-1=programmed cell death protein 1; RU=resonance units.

FIG. 17 shows FcγRla binding of IgG1-PD1 and several other anti-PD-1 antibodies. The binding of IgG1-PD1, nivolumab, pembrolizumab, dostarlimab, and cemiplimab to CHO-S cells transiently expressing human FcγRIla was analyzed by flow cytometry. IgG1-ctrl and IgG1-ctrl-FERR were included as a positive and negative control, respectively. Abbreviations: ctrl=control; FcγR=Fe gamma receptor; FERR=L234F/L235E/G236R-K409R; hulgG=human immunoglobulin G; PD-1=programmed cell death protein 1; PE=R-phycoerythrin.

FIG. 18 shows total human IgG in mouse plasma samples. Mice were injected intravenously with 1 or 10 mg/kg IgG1-PD1 at t=0 and serial plasma samples were taken at 10 min, 4 h, 1 d, 2 d, 8 d, 14 d, and 21 d after injection. Total hulgG in plasma samples was determined by ECLIA for each mouse. Data are represented as mean hulgG concentration ±SD of three individual mice. Dashed lines indicate the plasma concentrations of wild-type (wt) hulgG predicted by a two-compartment model based on IgG clearance in humans (Bleeker et al., 2001, Blood. 98(10):3136-42). Dotted lines indicate the LLOQ and ULOQ. Abbreviations: hulgG=human IgG; IgG=immunoglobulin G; LLOQ=lower limit of quantitation; PD-1=programmed cell death protein 1; SD=standard deviation; ULOQ=upper limit of quantitation.

FIG. 19 shows antitumor activity of IgG1-PD1 in human PD-1 knock-in mice. The MC38 colon cancer syngeneic tumor model was established by SC implantation in hPD-1 KI mice. Mice were administered 0.5, 2, or 10 mg/kg IgG1-PD1 or pembrolizumab or 10 mg/kg IgG1-ctrl-FERR 2QWx3 (9 mice per group). (A) Average tumor volume ±SEM in each group, until the last time point the group was complete. (B) Tumor volumes of the different groups on the last day all groups were complete (Day 11). Data shown are the tumor volumes in individual mice in each treatment group, as well as mean tumor volume ±SEM per treatment group. Mann-Whitney analysis was used to compare tumor volumes of the treatment groups to the IgG1-ctrl-FERR-treated group, with *p<0.05, **p<0.01, and ***p<0.001. C. Progression-free survival, defined as the percentage of mice with tumor volume smaller than 500 mm³, is shown as a Kaplan-Meier curve. Analysis excluded one mouse from the 2 mg/kg IgG1-PD1 group that was found dead due to undetermined cause on day 16, before the tumor volume had exceeded 500 mm³. Abbreviations: 2QWx3=twice per week for three weeks; ctrl=control; FERR=L234F/L235E/G236R/K409R mutations; IgG=immunoglobulin G; KI=knock-in; PD-1=programmed cell death protein 1; SC=subcutaneous; SEM=standard error of the mean.

FIG. 20 shows IL-2 secretion induced by IgG1-PD1 in combination with GEN1046 in an allogeneic MLR assay. Two unique donor pairs of allogeneic human mDCs and CD8⁺ T cells were co-cultured for 5 days in the presence of IgG1-PD1 (1 μg/mL), pembrolizumab (research grade, 1 μg/mL), GEN1046 (0.001 to 30 μg/mL), or the combination of either pembrolizumab or IgG1-PD1 and GEN1046. IgG1-ctrl-FERR (100 μg/mL), IgG4 (100 μg/mL), bsIgG1-PD-L1xctrl (30 μg/mL), bsIgG1-ctrlx4-iBB (30 μg/mL) and IgG1-ctrl-FEAL (30 μg/mL) were included as control antibodies. IL-2 secretion was analyzed in the supernatant by Luminex. Data shown are the mean IL-2 levels ±SEM of 2 unique allogeneic donor pairs. Abbreviations: bsIgG1=bispecific immunoglobulin Gi; ctrl=control; FERR=mutations L234F/L235E/G236R, K409R; FEAL=mutations L234F/L235E/D265A, F405L; IL=interleukin; IgG=immunoglobulin G; mDCs=mature dendritic cells; MLR=mixed lymphocyte reaction; PD1=programmed cell death protein 1; PD-L1=programmed cell death 1 ligand 1; SEM=standard error of the mean.

FIG. 21 shows enhancement of CD8^{m} T-cell proliferation by IgG1-PD1 in combination with GEN1046 in an antigen-specific T-cell stimulation assay. Human CD8⁺ T cells were electroporated with RNA encoding a CLDN6-specific TCR and RNA encoding PD1 and labeled with CFSE. The T cells were then co-cultured with iDCs electroporated with CLDN6, in the presence of 0.8 μg/mL IgG1-PD1, pembrolizumab, or IgG1-ctrl-FERR, either alone or in combination with the indicated concentrations of GEN1046. CFSE dilution in T cells was analyzed by flow cytometry after 4 days and used to calculate the expansion index. Data from one representative donor out of four donors evaluated in two independent experiments are shown. Error bars represent SD of duplicate wells. Dotted line indicates expansion index of CD8′ T cells co-cultured with mock-electroporated (i.e. not expressing CLDN6) iDCs. Abbreviations: CFSE=carboxyfluorescein succinimidyl ester; CLDN6=claudin 6; ctrl=control; FERR=mutations L234F/L235E/G236R, K409R; iDCs=immature dendritic cells; IgG1=immunoglobulin Gi; PD1=programmed cell death protein 1; PD-L1=programmed cell death 1 ligand 1; RNA=ribonucleic acid; SD=standard deviation; TCR=T-cell receptor.

FIG. 22 shows enhancement of cytokine secretion by IgG1-PD1 in combination with GEN1046 after antigen-specific CD8⁺ T-cell stimulation. Human CD8^{t} T cells expressing a CLDN6-specific TCR and PD1 were co-cultured with CLDN6-expressing iDCs as in FIG. 21, in the presence of 0.8 μg/mL IgG1-PD1, pembrolizumab, or IgG1-ctrl-FERR, either alone or in combination with the indicated concentrations of GEN1046. Cytokine concentrations in culture supernatants were determined after 4 days by multiplexed electrochemiluminescence immunoassay. Data from one representative donor out of four donors evaluated in two independent experiments are shown. Error bars represent SD of duplicate wells. Abbreviations: CLDN6=claudin 6; ctrl=control; FERR=mutations L234F/L235E/G236R, K409R; GM-CSF=granulocyte/macrophage colony-stimulating factor; iDCs=immature dendritic cells; IgG1=immunoglobulin Gi; IFN=interferon; IL=interleukin; PD1=programmed cell death protein 1; PD-L1=programmed cell death 1 ligand 1; RNA=ribonucleic acid; SD=standard deviation; TCR=T-cell receptor.

FIG. 23 shows the MC38 colon cancer model that was established by SC inoculation of 1×10⁶ MC38 cells into C57BL/6 mice. When tumors reached an average volume of 60 mm³, mice were randomized and treated with the indicated antibodies or combinations thereof (all 2QWX3). A. Data shown are the median tumor volume per treatment group (n=10) with data carried forward for animals that reached termination criteria. Growth curves were discontinued when <50% of the animals within a treatment group remained alive (mIgG2a-ctrl-AAKR, mbsIgG2a-PD-L1×4-1BB, anti-mouse PD-1 antibody [anti-mPD-1]) or until Day 69 (combination of mbsIgG2a-PD-L1×4-1BB with anti-mPD-1). Downward facing triangles indicate days of treatment. B. Progression-free survival, defined as the percentage of mice with tumor volume smaller than 500 mm³, is shown as Kaplan Meier curve.

FIG. 24 shows the (re)challenge of mice with complete tumor regression upon treatment and a control group of tumor-naive mice. Mice were (re)challenged with 1×10⁶ MC38 tumor cells that were SC injected on Day 121 after the treatment with antibodies was initiated. Data shown are mean tumor volumes ±SEM.

FIG. 25 shows the cytokine levels in peripheral blood of MC38-tumor bearing C57BL/6 mice treated with mbsIgG2a-PD-L1×4-1BB, an anti-mPD-1 antibody either as single agents or in combination, or nonbinding control antibody IgG2a-ctrl-AAKR. Peripheral blood samples were taken at baseline (one day before treatment [Day −1], dotted line) and two days after each treatment (Day 2 and Day 5). Cytokine analysis was performed by ECLIA.

FIG. 26 shows quantitative IHC and ISH data on cellular immune and tumor markers expressed in resected tumor tissues from the MC38 colon cancer model. C57BL/6 mice were inoculated with 1×10⁶ MC38 cells. When tumors reached an average volume of 50-70 mm³, mice were randomized and treated with mbsIgG2a-PD-L1×4-1BB, anti-mPD-1 or the combination thereof. Tumors were resected on Day 7 (n=5 per treatment group) or Day 14 (n=5 per treatment group) after treatment initiation. Some of the resected tumor samples were too small to perform IHC analysis, resulting in analysis of 4-5 tumors per treatment group. Sections of resected tumors (4 m) were stained using anti-CD3, anti-CD4, anti-CD8 or anti-PD-L1 antibodies by immunohistochemistry (IHC), or were stained for 4-1BB or PD-L2 by in situ hybridization (ISH). Data from IHC are depicted as % marker postive cells of the total cells counted in the slide as well as mean ±SEM per treatment group. Data from ISH are depicted as RNAscope H-score per slide as well as mean ±SEM per treatment group.

FIG. 27 shows GzmB and Ki67 expression in CD8 T-cell subsets from dissociated tumor tissue from the MC38 colon cancer model. C57BL/6 mice were inoculated with 1×10⁶ MC38 cells. When tumors reached an average volume of 50-70 mm³, mice were randomized and treated with mbsIgG2a-PD-L1×4-1BB, anti-mPD-1 or the combination thereof. Tumors were resected on Day 7 (n=5 per treatment group) after treatment initiation, dissociated to single cells suspensions and analyzed by flow cytometry. Data shown are the percentage of GzmB+(A) or Ki67T cells (B) within the CD8⁺ T-cell population of individual mice and the mean ±SEM per treatment group. Mann-Whitney statistical analysis was performed to compare the percentage of GzmB+or Ki67T cells within the CD8⁺ T-cell population between treatment groups, with * p<0.05 and **p<0.01.

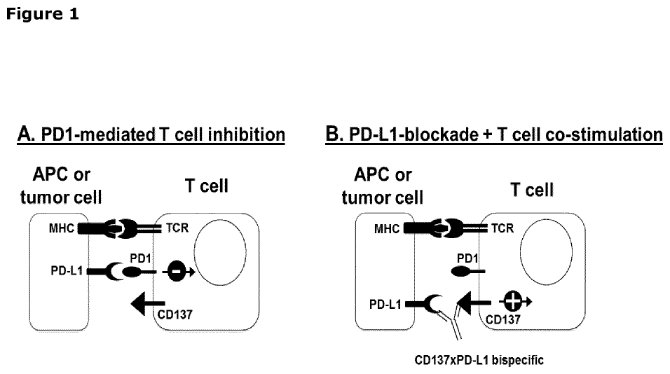

TABLE 1
Sequences: In the following reference is given to sequences and SEQ ID NOs
which are shown inter alia in the sequence listing. Also, reference is given
to specific examples of antibodies of the invention described herein, but
without limiting the present invention thereto. These exemplary, but not
limiting antibodies of the invention are designated herein by referring
to the designation of the antibody. Bold and underlined are F; E; G; A;
L; R and G, corresponding with positions 234; 235; 236; 265; 405; 409
and 430, respectively, said positions being in accordance with
EU-numbering. IN SEQ ID Nos.: 83 and 84 bold amino acids represent
the-AAKR or-AALT mutations required for controlled Fab-arm exchange.
In variable regions, said CDR regions that were annotated in
accordance with IMGT definitions (unless otherwise stated or
contradicted by context), are underlined.

| SEQ | | | |
|---|---|---|---|
| ID | NAME | SEQUENCE | Organism |
| 1 | VH_CD137-009-H7 | EVQLVESGGGLVQPGRSLRLSCTASGFSLNDYWMSWVRQ | synthetic |
| | | APGKGLEWVGYIDVGGSLYYAASVKGRFTISRDDSKSIAYLQ | construct |
| | | MNSLKTEDTAVYYCARGGLTYGFDLWGQGTLVTVSS | |
| 2 | VH_CD137-009- | GFSLNDYW | synthetic |
| | H7_CDR1 | | construct |
| 3 | VH_CD137-009- | IDVGGSL | synthetic |
| | H7_CDR2 | | construct |
| 4 | VH_CD137-009- | ARGGLTYGFDL | synthetic |
| | H7_CDR3 | | construct |
| 5 | VL_CD137-009-L2 | DIVMTQSPSSLSASVGDRVTITCQASEDISSYLAWYQQKPGKA | synthetic |
| | | PKRLIYGASDLASGVPSRFSASGSGTDYTFTISSLQPEDIATY | construct |
| | | YCHYYATISGLGVAFGGGTKVEIK | |
| 6 | VL_CD137-009- | EDISSY | synthetic |
| | L2_CDR1 | | construct |
| 7 | VL_CD137-009- | GAS | synthetic |
| | L2_CDR2 | | construct |
| 8 | VL_CD137-009- | HYYATISGLGVA | synthetic |
| | L2_CDR3 | | construct |
| 9 | VH_CD137-009 | QSLEESGGRLVTPGTPLTLTCTVSGFSLNDYWMSWVRQAPG | synthetic |
| | | KGLEWIGYIDVGGSLYYASWAKGRFTISRTSTTVDLKMTSL | construct |
| | | TTEDTATYFCARGGLTYGFDLWGPGTLVTVSS | |
| 10 | VL_CD137-009 | DIVMTQTPASVSEPVGGTVTINCQASEDISSYLAWYQQKPG | synthetic |
| | | QRPKRLIYGASDLASGVPSRFSASGSGTEYALTISDLESAD | construct |
| | | AATYYCHYYATISGLGVAFGGGTEVVVK | |
| 11 | VH-PD-L1-547 | EVQLLEPGGGLVQPGGSLRLSCEASGSTFSTYAMSWVRQA | synthetic |
| | | PGKGLEWVSGFSGSGGFTFYADSVRGRFTISRDSSKNTLF | construct |
| | | LQMSSLRAEDTAVYYCAIPARGYNYGSFQHWGQGTLVTVSS | |
| 12 | VH-PD-L1-547- | GSTFSTYA | synthetic |
| | CDR1 | | construct |
| 13 | VH-PD-L1-547- | FSGSGGFT | synthetic |
| | CDR2 | | construct |
| 14 | VH-PD-L1-547- | AIPARGYNYGSFQH | synthetic |
| | CDR3 | | construct |
| 15 | VL-PD-L1-547 | SYVLTQPPSVSVAPGQTARITCGGNNIGSKSVHWYQQKPG | synthetic |
| | | QAPVLVVYDDNDRPSGLPERFSGSNSGNTATLTISRVEAG | construct |
| | | DEADYYCQVWDSSSDHVVFGGGTKLTVL | |
| 16 | VL-PD-L1-547- | NIGSKS | synthetic |
| | CDR1 | | construct |
| 17 | VL-PD-L1-547- | DDN | synthetic |
| | CDR2 | | construct |
| 18 | VL-PD-L1-547- | QVWDSSSDHVV | synthetic |
| | CDR3 | | construct |
| 19 | IgG1-Fc | ASTKGPSVFPLAPSSKSTSGGTAALGCLVKDYFPEPVTVSWN | synthetic |
| | | SGALTSGVHTFPAVLQSSGLYSLSSVVTVPSSSLGTQTYICN | construct |
| | | VNHKPSNTKVDKRVEPKSCDKTHTCPPCPAPELLGGPSVFLF | |
| | | PPKPKDTLMISRTPEVTCVVVDVSHEDPEVKFNWYVDGVEV | |
| | | HNAKTKPREEQYNSTYRVVSVLTVLHQDWLNGKEYKCKVSN | |
| | | KALPAPIEKTISKAKGQPREPQVYTLPPSREEMTKNQVSLT | |
| | | CLVKGFYPSDIAVEWESNGQPENNYKTTPPVLDSDGSFFLYS | |
| | | KLTVDKSRWQQGNVFSCSVMHEALHNHYTQKSLSLSPGK | |
| 20 | IgG1-Fc_F405L | ASTKGPSVFPLAPSSKSTSGGTAALGCLVKDYFPEPVTVSWN | synthetic |
| | | SGALTSGVHTFPAVLQSSGLYSLSSVVTVPSSSLGTQTYICN | construct |
| | | VNHKPSNTKVDKRVEPKSCDKTHTCPPCPAPELLGGPSVFLF | |
| | | PPKPKDTLMISRTPEVTCVVVDVSHEDPEVKFNWYVDGVEV | |
| | | HNAKTKPREEQYNSTYRVVSVLTVLHQDWLNGKEYKCKVSN | |
| | | KALPAPIEKTISKAKGQPREPQVYTLPPSREEMTKNQVSLT | |
| | | CLVKGFYPSDIAVEWESNGQPENNYKTTPPVLDSDGSFLLYS | |
| | | KLTVDKSRWQQGNVFSCSVMHEALHNHYTQKSLSLSPGK | |
| 21 | IgG1-Fc_K409R | ASTKGPSVFPLAPSSKSTSGGTAALGCLVKDYFPEPVTVSWN | synthetic |
| | | SGALTSGVHTFPAVLQSSGLYSLSSVVTVPSSSLGTQTYICNV | construct |
| | | NHKPSNTKVDKRVEPKSCDKTHTCPPCPAPELLGGPSVFLFP | |
| | | PKPKDTLMISRTPEVTCVVVDVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | RLTVDKSRWQQGNVFSCSVMHEALHNHYTQKSLSLSPGK | |
| 22 | IgG1-Fc_FEA | ASTKGPSVFPLAPSSKSTSGGTAALGCLVKDYFPEPVTVSWN | synthetic |
| | | SGALTSGVHTFPAVLQSSGLYSLSSVVTVPSSSLGTQTYICNV | construct |
| | | NHKPSNTKVDKRVEPKSCDKTHTCPPCPAPEFEGGPSVFLFP | |
| | | PKPKDTLMISRTPEVTCVVVAVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | KLTVDKSRWQQGNVFSCSVMHEALHNHYTQKSLSLSPGK | |
| 23 | IgG1-FEAR-Fc | ASTKGPSVFPLAPSSKSTSGGTAALGCLVKDYFPEPVTVSWN | synthetic |
| | | SGALTSGVHTFPAVLQSSGLYSLSSVVTVPSSSLGTQTYICNV | construct |
| | | NHKPSNTKVDKRVEPKSCDKTHTCPPCPAPEFEGGPSVFLFP | |
| | | PKPKDTLMISRTPEVTCVVVAVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | RLTVDKSRWQQGNVFSCSVMHEALHNHYTQKSLSLSPGK | |
| 24 | IgG1-FEAL-Fc | ASTKGPSVFPLAPSSKSTSGGTAALGCLVKDYFPEPVTVSWN | synthetic |
| | | SGALTSGVHTFPAVLQSSGLYSLSSVVTVPSSSLGTQTYICNV | construct |
| | | NHKPSNTKVDKRVEPKSCDKTHTCPPCPAPEFEGGPSVFLFP | |
| | | PKPKDTLMISRTPEVTCVVVAVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFLLYS | |
| | | KLTVDKSRWQQGNVFSCSVMHEALHNHYTQKSLSLSPGK | |
| 25 | IgG1-Fc without C- | ASTKGPSVFPLAPSSKSTSGGTAALGCLVKDYFPEPVTVSWN | synthetic |
| | terminal Lysine | SGALTSGVHTFPAVLQSSGLYSLSSVVTVPSSSLGTQTYICNV | construct |
| | | NHKPSNTKVDKRVEPKSCDKTHTCPPCPAPELLGGPSVFLFP | |
| | | PKPKDTLMISRTPEVTCVVVDVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | KLTVDKSRWQQGNVFSCSVMHEALHNHYTQKSLSLSPG | |
| 26 | IgG1-Fc_F405L | ASTKGPSVFPLAPSSKSTSGGTAALGCLVKDYFPEPVTVSWN | synthetic |
| | without C-terminal | SGALTSGVHTFPAVLQSSGLYSLSSVVTVPSSSLGTQTYICNV | construct |
| | Lysine | NHKPSNTKVDKRVEPKSCDKTHTCPPCPAPELLGGPSVFLFP | |
| | | PKPKDTLMISRTPEVTCVVVDVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFLLYS | |
| | | KLTVDKSRWQQGNVFSCSVMHEALHNHYTQKSLSLSPG | |
| 27 | IgG1-Fc_K409R | ASTKGPSVFPLAPSSKSTSGGTAALGCLVKDYFPEPVTVSWN | synthetic |
| | without C-terminal | SGALTSGVHTFPAVLQSSGLYSLSSVVTVPSSSLGTQTYICNV | construct |
| | Lysine | NHKPSNTKVDKRVEPKSCDKTHTCPPCPAPELLGGPSVFLFP | |
| | | PKPKDTLMISRTPEVTCVVVDVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | RLTVDKSRWQQGNVFSCSVMHEALHNHYTQKSLSLSPG | |
| 28 | IgG1-Fc_FEA | ASTKGPSVFPLAPSSKSTSGGTAALGCLVKDYFPEPVTVSWN | synthetic |
| | without C-terminal | SGALTSGVHTFPAVLQSSGLYSLSSVVTVPSSSLGTQTYICNV | construct |
| | Lysine | NHKPSNTKVDKRVEPKSCDKTHTCPPCPAPEFEGGPSVFLFP | |
| | | PKPKDTLMISRTPEVTCVVVAVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | KLTVDKSRWQQGNVFSCSVMHEALHNHYTQKSLSLSPG | |
| 29 | IgG1-FEAR-Fc | ASTKGPSVFPLAPSSKSTSGGTAALGCLVKDYFPEPVTVSWN | synthetic |
| | without C-terminal | SGALTSGVHTFPAVLQSSGLYSLSSVVTVPSSSLGTQTYICNV | construct |
| | Lysine | NHKPSNTKVDKRVEPKSCDKTHTCPPCPAPEFEGGPSVFLFP | |
| | | PKPKDTLMISRTPEVTCVVVAVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | RLTVDKSRWQQGNVFSCSVMHEALHNHYTQKSLSLSPG | |
| 30 | IgG1-FEAL-Fc | ASTKGPSVFPLAPSSKSTSGGTAALGCLVKDYFPEPVTVSWN | synthetic |
| | without C-terminal | SGALTSGVHTFPAVLQSSGLYSLSSVVTVPSSSLGTQTYICNV | construct |
| | Lysine | NHKPSNTKVDKRVEPKSCDKTHTCPPCPAPEFEGGPSVFLFP | |
| | | PKPKDTLMISRTPEVTCVVVAVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFLLYS | |
| | | KLTVDKSRWQQGNVFSCSVMHEALHNHYTQKSLSLSPG | |
| 31 | CD137-009 heavy | EVQLVESGGGLVQPGRSLRLSCTASGFSLNDYWMSWVRQ | synthetic |
| | chain | APGKGLEWVGYIDVGGSLYYAASVKGRFTISRDDSKSIAYLQ | construct |
| | | MNSLKTEDTAVYYCARGGLTYGFDLWGQGTLVTVSSASTK | |
| | | GPSVFPLAPSSKSTSGGTAALGCLVKDYFPEPVTVSWNSGAL | |
| | | TSGVHTFPAVLQSSGLYSLSSVVTVPSSSLGTQTYICNVNHKP | |
| | | SNTKVDKRVEPKSCDKTHTCPPCPAPEFEGGPSVFLFPPKPK | |
| | | DTLMISRTPEVTCVVVAVSHEDPEVKFNWYVDGVEVHNAK | |
| | | TKPREEQYNSTYRVVSVLTVLHQDWLNGKEYKCKVSNKALP | |
| | | APIEKTISKAKGQPREPQVYTLPPSREEMTKNQVSLTCLVKG | |
| | | FYPSDIAVEWESNGQPENNYKTTPPVLDSDGSFFLYSRLTVD | |
| | | KSRWQQGNVFSCSVMHEALHNHYTQKSLSLSPG | |
| 32 | CD137-009 light | DIVMTQSPSSLSASVGDRVTITCQASEDISSYLAWYQQKPGK | synthetic |
| | chain | APKRLIYGASDLASGVPSRFSASGSGTDYTFTISSLQPEDIATY | construct |
| | | YCHYYATISGLGVAFGGGTKVEIKRTVAAPSVFIFPPSDEQLK | |
| | | SGTASVVCLLNNFYPREAKVQWKVDNALQSGNSQESVTEQ | |
| | | DSKDSTYSLSSTLTLSKADYEKHKVYACEVTHQGLSSPVTKSF | |
| | | NRGEC | |
| 33 | PD-L1-547 heavy | EVQLLEPGGGLVQPGGSLRLSCEASGSTFSTYAMSWVRQA | synthetic |
| | chain | PGKGLEWVSGFSGSGGFTFYADSVRGRFTISRDSSKNTLFLQ | construct |
| | | MSSLRAED | |
| | | TAVYYCAIPARGYNYGSFQHWGQGTLVTVSSASTKGPSVFP | |
| | | LAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKRVEPKSCDKTHTCPPCPAPEFEGGPSVFLFPPKPKDTLMI | |
| | | SRTPEVTCVVVAVSHEDPEVKFNWYVDGVEVHNAKTKPRE | |
| | | EQYNSTYRVVSVLTVLHQDWLNGKEYKCKVSNKALPAPIEK | |
| | | TISKAKGQPREPQVYTLPPSREEMTKNQVSLTCLVKGFYPSD | |
| | | IAVEWESNGQPENNYKTTPPVLDSDGSFLLYSKLTVDKSRW | |
| | | QQGNVFSCSVMHEALHNHYTQKSLSLSPG | |
| 34 | PD-L1-547 light | SYVLTQPPSVSVAPGQTARITCGGNNIGSKSVHWYQQKPG | synthetic |
| | chain | QAPVLVVYDDNDRPSGLPERFSGSNSGNTATLTISRVEAGD | construct |
| | | EADYYCQVWDSSSDHVVFGGGTKLTVLGQPKAAPSVTLFP | |
| | | PSSEELQANKATLVCLISDFYPGAVTVAWKADSSPVKAGVET | |
| | | TTPSKQSNNKYAASSYLSLTPEQWKSHRSYSCQVTHEGSTV | |
| | | EKTVAPTECS | |
| 35 | Kappa-C | RTVAAPSVFIFPPSDEQLKSGTASVVCLLNNFYPREAKVQWK | synthetic |
| | | VDNALQSGNSQESVTEQDSKDSTYSLSSTLTLSKADYEKHKV | construct |
| | | YACEVTHQGLSSPVTKSFNRGEC | |
| 36 | Lambda-C | GQPKAAPSVTLFPPSSEELQANKATLVCLISDFYPGAVTVAW | synthetic |
| | | KADSSPVKAGVETTTPSKQSNNKYAASSYLSLTPEQWKSHR | construct |
| | | SYSCQVTHEGSTVEKTVAPTECS | |
| 37 | Human CD137 | MGNSCYNIVATLLLVLNFERTRSLQDPCSNCPAGTFCDNNR | Homo |
| | (UniProtKB- | NQICSPCPPNSFSSAGGQRTCDICRQCKGVFRTRKECSSTSN | sapiens |
| | Q07011; | AECDCTPGFHCLGAGCSMCEQDCKQGQELTKKGCKDCCF | |
| | incl. signal | GTFNDQKRGICRPWTNCSLDGKSVLVNGTKERDVVCGPSP | |
| | peptide sequence: | ADLSPGASSVTPPAPAREPGHSPQIISFFLALTSTALLFLL | |
| | aa 1-23) | FFLTLRFSVVKRGRKKLLYIFKQPFMRPVQTTQEEDGCSCR | |
| | | FPEEEEGGCEL | |
| 38 | Human CD137 | LQDPCSNCPAGTFCDNNRNQICSPCPPNSFSSAGGQRTCDI | Homo |
| | (UniProtKB- | CRQCKGVFRTRKECSSTSNAECDCTPGFHCLGAGCSMCEQ | sapiens |
| | Q07011; mature | DCKQGQELTKKGCKDCCFGTFNDQKRGICRPWTNCSLDGK | |
| | sequence) | SVLVNGTKERDVVCGPSPADLSPGASSVTPPAPAREPGHSP | |
| | | QIISFFLALTSTALLFLLFFLTLRFSVVKRGRKKLLYIFKQPFMR | |
| | | PVQTTQEEDGCSCRFPEEEEGGCEL | |
| 39 | Human PD-L1 | MRIFAVFIFMTYWHLLNAFTVTVPKDLYVVEYGSNMTIECF | Homo |
| | (UniProtKB- | PVEKQLDLAALIVYWEMEDKNIIQFVHGEEDLKVQHSSYRQ | sapiens |
| | Q9NZQ7; incl. | RARLLKDQLSLGNAALQITDVKLQDAGVYRCMISYGGADYK | |
| | signal peptide | RITVKVNAPYNKINQRILVVDPVTSEHELTCQAEGYPKAEVI | |
| | sequence: aa 1-18) | WTSSDHQVLSGKTTTTNSKREEKLFNVTSTLRINTTTNEIFYC | |
| | | TFRRLDPEENHTAELVIPELPLAHPPNERTHLVILGAILLCLGV | |
| | | ALTFIFRLRKGRMMDVKKCGIQDTNSKKQSDTHLEET | |
| 40 | Human PD-L1 | FTVTVPKDLYVVEYGSNMTIECKFPVEKQLDLAALIVYWEM | Homo |
| | (UniProtKB- | EDKNIIQFVHGEEDLKVQHSSYRQRARLLKDQLSLGNAALQI | sapiens |
| | Q9NZQ7; mature | TDVKLQDAGVYRCMISYGGADYKRITVKVNAPYNKINQRIL | |
| | sequence) | VVDPVTSEHELTCQAEGYPKAEVIWTSSDHQVLSGKTTTTN | |
| | | SKREEKLFNVTSTLRINTTTNEIFYCTFRRLDPEENHTAELVIP | |
| | | ELPLAHPPNERTHLVILGAILLCLGVALTFIFRLRKGRMMDVK | |
| | | KCGIQDTNSKKQSDTHLEET | |
| 41 | Human PD-1 | MQIPQAPWPVVWAVLQLGWRPGWFLDSPDRPWNPPTF | Homo |
| | | SPALLVVTEGDNATFTCSFSNTSESFVLNWYRMSPSNQTDK | sapiens |
| | | LAAFPEDRSQPGQDCRFRVTQLPNGRDFHMSVVRARRND | |
| | | SGTYLCGAISLAPKAQIKESLRAELRVTERRAEVPTAHPSPSP | |
| | | RPAGQFQTLVVGVVGGLLGSLVLLVWVLAVICSRAARGTIG | |
| | | ARRTGQPLKEDPSAVPVFSVDYGELDFQWREKTPEPPVPCV | |
| | | PEQTEYATIVFPSGMGTSSPARRGSADGPRSAQPLRPEDGH | |
| | | CSWPL | |
| 42 | CTLA-4 | MACLGFQRHKAQLNLATRTWPCTLLFFLLFIPVFCKAMHVA | Homo |
| | | QPAVVLASSRGIASFVCEYASPGKATEVRVTVLRQADSQVTE | sapiens |
| | | VCAATYMMGNELTFLDDSICTGTSSGNQVNLTIQGLRAMD | |
| | | TGLYICKVELMYPPPYYLGIGNGTQIYVIDPEPCPDSDFLLWI | |
| | | LAAVSSGLFFYSFLLTAVSLSKMLKKRSPLTTGVYVKMPPTEP | |
| | | ECEKQFQPYFIPIN | |
| 43 | VH_MAB-19-0618 | QVQLVESGGGLVQPGTSLRLSCSVSGFSLYSYNMGWVRQA | synthetic |
| | | PGKGLEYIGIISGGTIGHYASWAKGRFTISRDTSKTTLYLQMN | construct |
| | | SLTTEDTATYFCARAFYDDYDYNVWGPGTLVTVSS | |
| 44 | VL_MAB-19-0618 | AIQLTQSPSSLSASVGGTVTITCQSSQSVYGNNQLSWYQQK | synthetic |
| | | PGQPPKLLIYQASKLETGVPSRFRGSGSGTQFTLTISSLQSED | construct |
| | | FATYYCAGGYSSSSDTTFGGGTEVVVK | |
| 45 | VH_MAB-19-0618 | GFSLYSYN | synthetic |
| | CDR1 | | construct |
| 46 | VH_MAB-19-0618 | IISGGTIG | synthetic |
| | CDR2 | | construct |
| 47 | VH_MAB-19-0618 | AFYDDYDYNV | synthetic |
| | CDR3 | | construct |
| 48 | VL_MAB-19-0618 | QSVYGNNQ | synthetic |
| | CDR1 | | construct |
| 49 | VL_MAB-19-0618 | QAS | synthetic |
| | CDR2 | | construct |
| 50 | VL_MAB-19-0618 | AGGYSSSSDTT | synthetic |
| | CDR3 | | construct |
| 51 | VH_IgG1-PD1- | GITFSNSG | synthetic |
| | MDX1106-FEAL | | construct |
| | CDR1 | | |
| 52 | VH_IgG1-PD1- | IWYDGSKR | synthetic |
| | MDX1106-FEAL | | construct |
| | CDR2 | | |
| 53 | VH_IgG1-PD1- | ATNDDY | synthetic |
| | MDX1106-FEAL | | construct |
| | CDR3 | | |
| 54 | VL_IgG1-PD1- | QSVSSY | synthetic |
| | MDX1106-FEAL | | construct |
| | CDR1 | | |
| 55 | VL_IgG1-PD1- | DAS | synthetic |
| | MDX1106-FEAL | | construct |
| | CDR2 | | |
| 56 | VL_IgG1-PD1- | QQSSNWPRT | synthetic |
| | MDX1106-FEAL | | construct |
| | CDR3 | | |
| 57 | IgG1-PD1- | QVQLVESGGGVVQPGRSLRLDCKASGITFSNSG | synthetic |
| | MDX1106- | MHWVRQAPGKGLEWVAVIWYDGSKRYYADSV | construct |
| | FEAL VH | KGRFTISRDNSKNTLFLQMNSLRAEDTAVYYCA | |
| | | TNDDYWGQGTLVTVSS | |
| 58 | IgG1-PD1- | EIVLTQSPATLSLSPGERATLSCRASQSVSSYLAW | synthetic |
| | MDX1106- | YQQKPGQAPRLLIYDASNRATGIPARFSGSGSGT | construct |
| | FEAL VL | DFTLTISSLEPEDFAVYYCQQSSNWPRTFGQGTK | |
| | | VEIK | |
| 59 | Pembrolizumab VH | GYTFTNYY | synthetic |
| | CDR1 | | construct |
| 60 | Pembrolizumab VH | INPSNGGT | synthetic |
| | CDR2 | | construct |
| 61 | Pembrolizumab VH | ARRDYRFDMGFDY | synthetic |
| | CDR3 | | construct |
| 62 | Pembrolizumab VL | KGVSTSGYSY | synthetic |
| | CDR1 | | construct |
| 63 | Pembrolizumab VL | LAS | synthetic |
| | CDR2 | | construct |
| 64 | Pembrolizumab VL | QHSRDLPLT | synthetic |
| | CDR3 | | construct |
| 65 | Pembrolizumab VH | QVQLVQSGVEVKKPGASVKVSCKASGYTFTNYY | synthetic |
| | | MYWVRQAPGQGLEWMGGINPSNGGTNFNEKFK | construct |
| | | NRVTLTTDSSTTTAYMELKSLQFDDTAVYYCAR | |
| | | RDYRFDMGFDYWGQGTTVTVSS | |
| 66 | Pembrolizumab VL | EIVLTQSPATLSLSPGERATLSCRASKGVSTSGYS | synthetic |
| | | YLHWYQQKPGQAPRLLIYLASYLESGVPARFSGS | construct |
| | | GSGTDFTLTISSLEPEDFAVYYCQHSRDLPLTFGG | |
| | | GTKVEIK | |
| 67 | Pembrolizumab | QVQLVQSGVEVKKPGASVKVSCKASGYTFTNYYMYWVRQ | synthetic |
| | Heavy chain | APGQGLEWMGGINPSNGGTNFNEKFKNRVTLTTDSSTTTA | construct |
| | | YMELKSLQFDDTAVYYCARRDYRFDMGFDYWGQGTTVTV | |
| | | SSASTKGPSVFPLAPCSRSTSESTAALGCLVKDYFPEPVTVSW | |
| | | NSGALTSGVHTFPAVLQSSGLYSLSSVVTVPSSSLGTKTYTCN | |
| | | VDHKPSNTKVDKRVESKYGPPCPPCPAPEFLGGPSVFLFPPK | |
| | | PKDTLMISRTPEVTCVVVDVSQEDPEVQFNWYVDGVEVHN | |
| | | AKTKPREEQFNSTYRVVSVLTVLHQDWLNGKEYKCKVSNK | |
| | | GLPSSIEKTISKAKGQPREPQVYTLPPSQEEMTKNQVSLTCL | |
| | | VKGFYPSDIAVEWESNGQPENNYKTTPPVLDSDGSFFLYSRL | |
| | | TVDKSRWQEGNVFSCSVMHEALHNHYTQKSLSLSLGK | |
| 68 | Pembrolizumab | EIVLTQSPATLSLSPGERATLSCRASKGVSTSGYSYLHWYQQ | synthetic |
| | Light chain | KPGQAPRLLIYLASYLESGVPARFSGSGSGTDFTLTISSLEPED | construct |
| | | FAVYYCQHSRDLPLTFGGGTKVEIKRTVAAPSVFIFPPSDEQL | |
| | | KSGTASVVCLLNNFYPREAKVQWKVDNALQSGNSQESVTE | |
| | | QDSKDSTYSLSSTLTLSKADYEKHKVYACEVTHQGLSSPVTKS | |
| | | FNRGEC | |
| 69 | Nivolumab VH | GITFSNSG | synthetic |
| | CDR1 | | construct |
| 70 | Nivolumab VH | IWYDGSKR | synthetic |
| | CDR2 | | construct |
| 71 | Nivolumab VH | ATNDDY | synthetic |
| | CDR3 | | construct |
| 72 | Nivolumab VL | QSVSSY | synthetic |
| | CDR1 | | construct |
| 73 | Nivolumab VL | DAS | synthetic |
| | CDR2 | | construct |
| 74 | Nivolumab VL | QQSSNWPRT | synthetic |
| | CDR3 | | construct |
| 75 | Nivolumab VH | QVQLVESGGGVVQPGRSLRLDCKASGITFSNSGMHWVRQ | synthetic |
| | | APGKGLEWVAVIWYDGSKRYYADSVKGRFTISRDNSKNTLF | construct |
| | | LQMNSLRAEDTAVYYCATNDDYWGQGTLVTVSS | |
| 76 | Nivolumab VL | EIVLTQSPATLSLSPGERATLSCRASQSVSSYLAWYQQKPGQ | synthetic |
| | | APRLLIYDASNRATGIPARFSGSGSGTDFTLTISSLEPEDFAVY | construct |
| | | YCQQSSNWPRTFGQGTKVEIK | |
| 77 | Nivolumab Heavy | QVQLVESGGGVVQPGRSLRLDCKASGITFSNSGMHWVRQ | synthetic |
| | chain | APGKGLEWVAVIWYDGSKRYYADSVKGRFTISRDNSKNTLF | construct |
| | | LQMNSLRAEDTAVYYCATNDDYWGQGTLVTVSSASTKGPS | |
| | | VFPLAPCSRSTSESTAALGCLVKDYFPEPVTVSWNSGALTSG | |
| | | VHTFPAVLQSSGLYSLSSVVTVPSSSLGTKTYTCNVDHKPSN | |
| | | TKVDKRVESKYGPPCPPCPAPEFLGGPSVFLFPPKPKDTLMI | |
| | | SRTPEVTCVVVDVSQEDPEVQFNWYVDGVEVHNAKTKPRE | |
| | | EQFNSTYRVVSVLTVLHQDWLNGKEYKCKVSNKGLPSSIEKT | |
| | | ISKAKGQPREPQVYTLPPSQEEMTKNQVSLTCLVKGFYPSDI | |
| | | AVEWESNGQPENNYKTTPPVLDSDGSFFLYSRLTVDKSRW | |
| | | QEGNVFSCSVMHEALHNHYTQKSLSLSLGK | |
| 78 | Nivolumab Light | EIVLTQSPATLSLSPGERATLSCRASQSVSSYLAWYQQKPGQ | synthetic |
| | chain | APRLLIYDASNRATGIPARFSGSGSGTDFTLTISSLEPEDFAVY | construct |
| | | YCQQSSNWPRTFGQGTKVEIKRTVAAPSVFIFPPSDEQLKS | |
| | | GTASVVCLLNNFYPREAKVQWKVDNALQSGNSQESVTEQ | |
| | | DSKDSTYSLSSTLTLSKADYEKHKVYACEVTHQGLSSPVTKSF | |
| | | NRGEC | |
| 79 | VH_IgG1-b12 | QVQLVQSGAEVKKPGASVKVSCQASGYRFSNFV | synthetic |
| | | IHWVRQAPGQRFEWMGWINPYNGNKEFSAKFQ | construct |
| | | DRVTFTADTSANTAYMELRSLRSADTAVYYCAR | |
| | | VGPYSWDDSPQDNYYMDVWGKGTTVIVSS | |
| 80 | VL_IgG1-b12 | EIVLTQSPGTLSLSPGERATFSCRSSHSIRSRRVA | synthetic |
| | | WYQHKPGQAPRLVIHGVSNRASGISDRFSGSGSG | construct |
| | | TDFTLTITRVEPEDFALYYCQVYGASSYTFGQGT | |
| | | KLERK | |
| 81 | m4-1BB-3H3 VH | EMQLVESGGGLVQPGRSMKLSCAGSGFTLSDYG | synthetic |
| | | VAWVRQAPKKGLEWVAYISYAGGTTYYRESVK | construct |
| | | GRFTISRDNAKSTLYLQMDSLRSEDTATYYCTID | |
| | | GYGGYSGSHWYFDFWGPGTMVTVSS | |
| 82 | m4-1BB-3H3 VL | DIQMTQSPSLLSASVGDRVTLNCRTSQNVYKNL | synthetic |
| | | AWYQQKLGEAPKLLIYNANSLQAGIPSRFSGSGS | construct |
| | | GTDFTLTISSLQPEDVATYFCQQYYSGNTFGAGT | |
| | | NLELK | |
| 83 | AALT | AKTTAPSVYPLAPVCGDTTGSSVTLGCLVKGYFP | synthetic |
| | | EPVTLTWNSGSLSSGVHTFPAVLQSDLYTLSSSV | construct |
| | | TVTSSTWPSQSITCNVAHPASSTKVDKKIEPRGPT | |
| | | IKPCPPCKCPAPNAAGGPSVFIFPPKIKDVLMISLS | |
| | | PMVTCVVVDVSEDDPDVQISWFVNNVEVLTAQT | |
| | | QTHREDYNSTLRVVSALPIQHQDWMSGKEFKCK | |
| | | VNNKALPAPIERTISKPKGSVRAPQVYVLPPPEEE | |
| | | MTKKQVTLTCMVTDFMPEDIYVEWTNNGKTEL | |
| | | NYKNTEPVLDSDGSYLMYSKLTVEKKNWVERN | |
| | | SYSCSVVHEGLHNHHTTKSFSRTPGK | |
| 84 | AAKR | AKTTAPSVYPLAPVCGDTTGSSVTLGCLVKGYFP | synthetic |
| | | EPVTLTWNSGSLSSGVHTFPAVLQSDLYTLSSSV | construct |
| | | TVTSSTWPSQSITCNVAHPASSTKVDKKIEPRGPT | |
| | | IKPCPPCKCPAPNAAGGPSVFIFPPKIKDVLMISLS | |
| | | PMVTCVVVDVSEDDPDVQISWFVNNVEVLTAQT | |
| | | QTHREDYNSTLRVVSALPIQHQDWMSGKEFKCK | |
| | | VNNKALPAPIERTISKPKGSVRAPQVYVLPPPEEE | |
| | | MTKKQVTLTCMVKDFMPEDIYVEWTNNGKTEL | |
| | | NYKNTEPVLDSDGSYFMYSRLRVEKKNWVERN | |
| | | SYSCSVVHEGLHNHHTTKSFSRTPGK | |
| 85 | constant region | RADAAPTVSIFPPSSEQLTSGGASVVCFLNNFYPK | synthetic |
| | mouse kappa LC | DINVKWKIDGSERQNGVLNSWTDQDSKDSTYSM | construct |
| | | SSTLTLTKDEYERHNSYTCEATHKTSTSPIVKSFN | |
| | | RNEC | |
| 86 | MPDL3280A VH | EVQLVESGGGLVQPGGSLRLSCAASGFTFSDSWI | synthetic |
| | | HWYRQAPGKGLEWYAWISPYGGSTYYADSVKG | construct |
| | | RFTISADTSKNTAYLQMNSLRAEDTAVYYCARR | |
| | | HWPGGFDYWGQGTLVTVSS | |
| 87 | MPDL3280A VL | DIQMTQSPSSLSASVGDRVTITCRASQDVSTAVA | synthetic |
| | | WYQQKPGKAPKLLIYSASFLYSGVPSRFSGSGSG | construct |
| | | TDFTLTISSLQPEDFATYYCQQYLYHPATFGQGT | |
| | | KVEIK | |
| 88 | VH IgG1-PD1 | QVQLVESGGGLVQPGTSLRLSCSVSGFSLYSYN | synthetic |
| | | MGWVRQAPGKGLEYIGIISGGTIGHYASWAKGR | construct |
| | | FTISRDTSKTTLYLQMNSLTTEDTATYFCARAFY | |
| | | DDYDYNVWGPGTLVTVSS | |
| 89 | VL IgG1-PD1 | AIQLTQSPSSLSASVGGTVTITCQSSQSVYGNNQL | synthetic |
| | | SWYQQKPGQPPKLLIYQASKLETGVPSRFRGSGS | construct |
| | | GTQFTLTISSLQSEDFATYYCAGGYSSSSDTTFGG | |
| | | GTEVVVK | |
| 90 | constant region | ASTKGPSVFPLAPSSKSTSGGTAALGCLVKDYFP | synthetic |
| | human HC | EPVTVSWNSGALTSGVHTFPAVLQSSGLYSLSSV | construct |
| | IgGlm(f)-L234F- | VTVPSSSLGTQTYICNVNHKPSNTKVDKRVEPKS | |
| | L235E-G236R | CDKTHTCPPCPAPEFERGPSVFLFPPKPKDTLMIS | |
| | | RTPEVTCVVVDVSHEDPEVKFNWYVDGVEVHN | |
| | | AKTKPREEQYNSTYRVVSVLTVLHQDWLNGKE | |
| | | YKCKVSNKALPAPIEKTISKAKGQPREPQVYTLP | |
| | | PSREEMTKNQVSLTCLVKGFYPSDIAVEWESNG | |
| | | QPENNYKTTPPVLDSDGSFFLYSKLTVDKSRWQ | |
| | | QGNVFSCSVMHEALHNHYTQKSLSLSPG | |
| 91 | constant region | ASTKGPSVFPLAPSSKSTSGGTAALGCLVKDYFP | synthetic |
| | human HC | EPVTVSWNSGALTSGVHTFPAVLQSSGLYSLSSV | construct |
| | IgGlm(f)-L234F- | VTVPSSSLGTQTYICNVNHKPSNTKVDKRVEPKS | |
| | L235E-G236R- | CDKTHTCPPCPAPEFERGPSVFLFPPKPKDTLMIS | |
| | K409R | RTPEVTCVVVDVSHEDPEVKFNWYVDGVEVHN | |
| | | AKTKPREEQYNSTYRVVSVLTVLHQDWLNGKE | |
| | | YKCKVSNKALPAPIEKTISKAKGQPREPQVYTLP | |
| | | PSREEMTKNQVSLTCLVKGFYPSDIAVEWESNG | |
| | | QPENNYKTTPPVLDSDGSFFLYSRLTVDKSRWQ | |
| | | QGNVFSCSVMHEALHNHYTQKSLSLSPG | |
| 92 | constant region | ASTKGPSVFPLAPSSKSTSGGTAALGCLVKDYFPEPVTVSWN | synthetic |
| | human HC | SGALTSGVHTFPAVLQSSGLYSLSSVVTVPSSSLGTQTYICNV | construct |
| | IgGlm(f) | NHKPSNTKVDKRVEPKSCDKTHTCPPCPAPELLGGPSVFLFP | |
| | | PKPKDTLMISRTPEVTCVVVDVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | KLTVDKSRWQQGNVFSCSVMHEALHNHYTQKSLSLSPG | |
| 93 | constant region | ASTKGPSVFPLAPSSKSTSGGTAALGCLVKDYFPEPVTVSWN | synthetic |
| | human HC | SGALTSGVHTFPAVLQSSGLYSLSSVVTVPSSSLGTQTYICNV | construct |
| | IgGlm(f)-L234F- | NHKPSNTKVDKRVEPKSCDKTHTCPPCPAPEX₁X₂X₃GPSVF | |
| | L235E-G236R and | LFPPKPKDTLMISRTPEVTCVVVDVSHEDPEVKFNWYVDGV | |
| | variants | EVHNAKTKPREEQYNSTYRVVSVLTVLHQDWLNGKEYKCK | |
| | | VSNKALPAPIEKTISKAKGQPREPQVYTLPPSREEMTKNQVS | |
| | | LTCLVKGFYPSDIAVEWESNGQPENNYKTTPPVLDSDGSFFL | |
| | | YSKLTVDKSRWQQGNVFSCSVMHEALHNHYTQKSLSLSPG | |
| | | wherein: | |
| | | X₁ = A, V, L, I, P, F, M or W, | |
| | | preferably I, P, F, M or W, | |
| | | more preferably F; | |
| | | X₂ = L, D or E, preferably D or E, | |
| | | more preferably E; | |
| | | X₃ = not G, preferably K, R or | |
| | | H, more preferably R; | |
| | | most preferably X₁X₂X₃ = FER | |
| 94 | constant region | ASTKGPSVFPLAPSSKSTSGGTAALGCLVKDYFP | synthetic |
| | human HC | EPVTVSWNSGALTSGVHTFPAVLQSSGLYSLSSV | construct |
| | IgGlm(f)-L234F- | VTVPSSSLGTQTYICNVNHKPSNTKVDKRVEPKS | |
| | L235E-G236R- | CDKTHTCPPCPAPEFERGPSVFLFPPKPKDTLMIS | |
| | K409R | RTPEVTCVVVDVSHEDPEVKFNWYVDGVEVHN | |
| | | AKTKPREEQYNSTYRVVSVLTVLHQDWLNGKE | |
| | | YKCKVSNKALPAPIEKTISKAKGQPREPQVYTLP | |
| | | PSREEMTKNQVSLTCLVKGFYPSDIAVEWESNG | |
| | | QPENNYKTTPPVLDSDGSFFLYSRLTVDKSRWQ | |
| | | QGNVFSCSVMHEALHNHYTQKSLSLSPG | |
| 95 | constant region | ASTKGPSVFPLAPSSKSTSGGTAALGCLVKDYFPEPVTVSWN | synthetic |
| | human HC | SGALTSGVHTFPAVLQSSGLYSLSSVVTVPSSSLGTQTYICNV | construct |
| | IgGlm(f)-E430G | NHKPSNTKVDKRVEPKSCDKTHTCPPCPAPELLGGPSVFLFP | |
| | | PKPKDTLMISRTPEVTCVVVDVSHEDPEVKFNWYVDGVEV | |
| | | HNAKTKPREEQYNSTYRVVSVLTVLHQDWLNGKEYKCKVS | |
| | | NKALPAPIEKTISKAKGQPREPQVYTLPPSREEMTKNQVSLT | |
| | | CLVKGFYPSDIAVEWESNGQPENNYKTTPPVLDSDGSFFLYS | |
| | | KLTVDKSRWQQGNVFSCSVMHGALHNHYTQKSLSLSPG | |
| 96 | constant region | ASTKGPSVFPLAPCSRSTSESTAALGCLVKDYFPEPVTVSWN | synthetic |
| | human HC IgG4 | SGALTSGVHTFPAVLQSSGLYSLSSVVTVPSSSLGTKTYTCNV | construct |
| | | DHKPSNTKVDKRVESKYGPPCPSCPAPEFLGGPSVFLFPPKP | |
| | | KDTLMISRTPEVTCVVVDVSQEDPEVQFNWYVDGVEVHN | |
| | | AKTKPREEQFNSTYRVVSVLTVLHQDWLNGKEYKCKVSNK | |
| | | GLPSSIEKTISKAKGQPREPQVYTLPPSQEEMTKNQVSLTCL | |
| | | VKGFYPSDIAVEWESNGQPENNYKTTPPVLDSDGSFFLYSRL | |
| | | TVDKSRWQEGNVFSCSVMHEALHNHYTQKSLSLSLGK | |
| 97 | constant region | RTVAAPSVFIFPPSDEQLKSGTASVVCLLNNFYPR | synthetic |
| | human kappa LC | EAKVQWKVDNALQSGNSQESVTEQDSKDSTYSL | construct |
| | | SSTLTLSKADYEKHKVYACEVTHQGLSSPVTKSF | |
| | | NRGEC | |
| 98 | Heavy chain human | ASTKGPSVFPLAPSSKSTSGGTAALGCLVKDYFPEPVTVSWN | synthetic |
| | IgG1-LALA | SGALTSGVHTFPAVLQSSGLYSLSSVVTVPSSSLGTQTYICNV | construct |
| | | NHKPSNTKVDKKVEPKSCDKTHTCPPCPAPEAAGGPSVFLF | |
| | | PPKPKDTLMISRTPEVTCVVVDVSHEDPEVKFNWYVDGVE | |
| | | VHNAKTKPREEQYNSTYRVVSVLTVLHQDWLNGKEYKCKV | |
| | | SNKALPAPIEKTISKAKGQPREPQVYTLPPSREEMTKNQVSL | |
| | | TCLVKGFYPSDIAVEWESNGQPENNYKTTPPVLDSDGSFFLY | |
| | | SKLTVDKSRWQQGNVFSCSVMHEALHNHYTQKSLSLSPG | |
| 99 | HCDR 3 inter- | AFYDDYDYNV | synthetic |
| | section of Kabat | | construct |
| | and IMGT | | |
| | (= Kabat) | | |
| 100 | HCDR 3 IMGT | ARAFYDDYDYNV | synthetic |
| | | | construct |
| 101 | HCDR 2 inter- | ISGGTIG | synthetic |
| | section of Kabat | | construct |
| | and IMGT | | |
| | (= IMGT) | | |
| 102 | HCDR 2 Kabat | IISGGTIGHYASWAKG | synthetic |
| | | | construct |
| | HCDR 1 inter- | SYN | synthetic |
| | section of Kabat | | construct |
| | and IMGT | | |
| 103 | HCDR 1 Kabat | SYNMG | synthetic |
| | | | construct |
| 104 | HCDR 1 IMGT | GFSLYSYN | synthetic |
| | | | construct |
| 105 | LCDR 3 inter- | AGGYSSSSDTT | synthetic |
| | section Kabat | | construct |
| | IMGT | | |
| | LCDR2 inter-section | QAS | synthetic |
| | of Kabat and IMGT | | construct |
| | (= IMGT) | | |
| 106 | LCDR2 Kabat | QASKLET | synthetic |
| | | | construct |
| 107 | LCDR1 inter-section | QSVYGNNQ | synthetic |
| | of Kabat and IMGT | | construct |
| | (= IMGT) | | |
| 108 | LCDR1 Kabat | QSSQSVYGNNQLS | synthetic |
| | | | construct |
| 109 | VH MAB-19-0202 | QSVEESGGRLVTPGTPLTLTCTVSGFSLYSYNMGWVRQAP | synthetic |
| | | GKGLEYIGIISGGTIGHYASWAKGRFTISKTSSTTVDLKM | construct |
| | | TSLTTEDTATYFCARAFYDDYDYNVWGPGTLVTVSS | |
| 110 | VL MAB-19-0202 | AAVLTQTPSPVSAAVGGTVTISCQSSQSVYGNNQLSWYQQ | synthetic |
| | | KPGQPPKLLIYQASKLETGVPSRFKGSGSGTQFTLTISDL | construct |
| | | ESDDAATYYCAGGYSSSSDTTFGGGTEVVVK | |
| 111 | VH IgG1-PD1 | QVQLVESGGGLVQPGTSLRLSCSVSGFSLYSYNMGWVRQA | synthetic |
| | (H5 derived from | PGKGLEYIGIISGGTIGHYASWAKGRFTISRDTSKTTLYL | construct |
| | MAB-19-0202) | QMNSLTTEDTATYFCARAFYDDYDYNVWGPGTLVTVSS | |
| 112 | VL IgG1-PD1 | AIQLTQSPSSLSASVGGTVTITCQSSQSVYGNNQL | synthetic |
| | (L4 derived from | SWYQQKPGQPPKLLIYQASKLETGVPSRFRGSGS | construct |
| | MAB-19-0202) | GTQFTLTISSLQSEDFATYYCAGGYSSSSDTTFGG | |
| | | GTEVVVK | |
| 113 | Human PD-1 | MQIPQAPWPVVWAVLQLGWRPGWFLDSPDRPWNPPTFSP | Homo |
| | complete | ALLVVTEGDNATFTCSFSNTSESFVLNWYRMSPSNQTDK | sapiens |
| | | LAAFPEDRSQPGQDCRFRVTQLPNGRDFHMSVVRARRND | |
| | | SGTYLCGAISLAPKAQIKESLRAELRVTERRAEVPTAHP | |
| | | SPSPRPAGQFQTLVVGVVGGLLGSLVLLVWVLAVICSRA | |
| | | ARGTIGARRTGQPLKEDPSAVPVFSVDYGELDFQWREKT | |
| | | PEPPVPCVPEQTEYATIVFPSGMGTSSPARRGSADGPRS | |
| | | AQPLRPEDGHCSWPL | |
| 114 | Human PD-1 | FLDSPDRPWNPPTFSPALLVVTEGDNATFTCSFS | Homo |
| | complete | NTSESFVLNWYRMSPSNQTDKLAAFPEDRSQPG | sapiens |
| | extracellular | QDCRFRVTQLPNGRDFHMSVVRARRNDSGTYLC | |
| | domain | GAISLAPKAQIKESLRAELRVTERRAEVPTAHP | |
| | | SPSPRPAGQFQTLV | |
| 115 | Nucleic acid human | agtttcccttccgctcacctccgcctgagcagtggagaaggcggc | Homo |
| | PD-1 | actctggtggggctgctccaggcatgcagatcccacaggcgccct | sapiens |
| | | ggccagtcgtctgggcggtgctacaactgggctggcggccaggat | |
| | | ggttcttagactccccagacaggccctggaacccccccaccttct | |
| | | ccccagccctgctcgtggtgaccgaaggggacaacgccaccttca | |
| | | cctgcagcttctccaacacatcggagagcttcgtgctaaactggt | |
| | | accgcatgagccccagcaaccagacggacaagctggccgccttcc | |
| | | ccgaggaccgcagccagcccggccaggactgccgcttccgtgtca | |
| | | cacaactgcccaacgggcgtgacttccacatgagcgtggtcaggg | |
| | | cccggcgcaatgacagcggcacctacctctgtggggccatctccc | |
| | | tggcccccaaggcgcagatcaaagagagcctgcgggcagagctca | |
| | | gggtgacagagagaagggcagaagtgcccacagcccaccccagcc | |
| | | cctcacccaggccagccggccagttccaaaccctggtggttggtg | |
| | | tcgtgggcggcctgctgggcagcctggtgctgctagtctgggtcc | |
| | | tggccgtcatctgctcccgggccgcacgagggacaataggagcca | |
| | | ggcgcaccggccagcccctgaaggaggacccctcagccgtgcctg | |
| | | tgttctctgtggactatggggagctggatttccagtggcgagaga | |
| | | agaccccggagccccccgtgccctgtgtccctgagcagacggagt | |
| | | atgccaccattgtctttcctagcggaatgggcacctcatcccccg | |
| | | cccgcaggggctcagctgacggccctcggagtgcccagccactga | |
| | | ggcctgaggatggacactgctcttggcccctctgaccggcttcct | |
| | | tggccaccagtgttctgcagaccctccaccatgagcccgggtcag | |
| | | cgcatttcctcaggagaagcaggcagggtgcaggccattgcaggc | |
| | | cgtccaggggctgagctgcctgggggcgaccggggctccagcctg | |
| | | cacctgcaccaggcacagccccaccacaggactcatgtctcaatg | |
| | | cccacagtgagcccaggcagcaggtgtcaccgtcccctacaggga | |
| | | gggccagatgcagtcactgcttcaggtcctgccagcacagagctg | |
| | | cctgcgtccagctccctgaatctctgctgctgctgctgctgctgc | |
| | | tgctgctgcctgcggcccggggctgaaggcgccgtggccctgcct | |
| | | gacgccccggagcctcctgcctgaacttgggggctggttggagat | |
| | | ggccttggagcagccaaggtgcccctggcagtggcatcccgaaac | |
| | | gccctggacgcagggcccaagactgggcacaggagtgggaggtac | |
| | | atggggctggggactccccaggagttatctgctccctgcaggcct | |
| | | agagaagtttcagggaaggtcagaagagctcctggctgtggtggg | |
| | | cagggcaggaaacccctccacctttacacatgcccaggcagcacc | |
| | | tcaggccctttgtggggcagggaagctgaggcagtaagcgggcag | |
| | | gcagagctggaggcctttcaggcccagccagcactctggcctcct | |
| | | gccgccgcattccaccccagcccctcacaccactcgggagaggga | |
| | | catcctacggtcccaaggtcaggagggcagggctggggttgactc | |
| | | aggcccctcccagctgtggccacctgggtgttgggagggcagaag | |
| | | tgcaggcacctagggccccccatgtgcccaccctgggagctctcc | |
| | | ttggaacccattcctgaaattatttaaaggggttggccgggctcc | |
| | | caccagggcctgggtgggaaggtacaggcgttcccccggggccta | |
| | | gtacccccgccgtggcctatccactcctcacatccacacactgca | |
| | | cccccactcctggggcagggccaccagcatccaggcggccagcag | |
| | | gcacctgagtggctgggacaagggatcccccttccctgtggttct | |
| | | attatattataattataattaaatatgagagcatgctaaggaaaa | |
| 116 | VH CD52- | QVQLQESGPGLVRPSQTLSLTCTVSGFTFTDFYMNWVRQP | synthetic |
| | CAMPATH-1H | PGRGLEWIGFIRDKAKGYTTEYNPSVKGRVTMLVDTSKNQF | construct |
| | | SLRLSSVTAADTAVYYCAREGHTAAPFDYWGQGSLVTVSS | |
| 117 | VH CD52- | GFTFTDFY | synthetic |
| | CAMPATH-1H | | construct |
| | CDR1 | | |
| 118 | VH CD52- | IRDKAKGYTT | synthetic |
| | CAMPATH-1H | | construct |
| | CDR2 | | |
| 119 | VH CD52- | AREGHTAAPFDY | synthetic |
| | CAMPATH-1H | | construct |
| | CDR3 | | |
| 120 | VL CD52- | DIQMTQSPSSLSASVGDRVTITCKASQNIDKYLNWYQQKPGK | synthetic |
| | CAMPATH-1H | APKLLIYNTNNLQTGVPSRFSGSGSGTDFTFTISSLQPEDIA | construct |
| | | TYYCLQHISRPRTFGQGTKVEIK | |
| 121 | VL CD52- | QNIDKY | synthetic |
| | CAMPATH-1H | | construct |
| | CDR1 | | |
| | VL CD52- | NTN | synthetic |
| | CAMPATH-1H | | construct |
| | CDR2 | | |
| 122 | VL CD52- | LQHISRPRT | synthetic |
| | CAMPATH-1H | | construct |
| | CDR3 | | |
| 123 | VH gp 120-b12 | QVQLVQSGAEVKKPGASVKVSCQASGYRFSNFVIHWVRQA | synthetic |
| | | PGQRFEWMGWINPYNGNKEFSAKFQDRVTFTADTSANTA | construct |
| | | YMELRSLRSADTAVYYCARVGPYSWDDSPQDNYYMDVW | |
| | | GKGTTVIVSS | |
| 124 | VH gp120-b12 | GYRFSNFV | synthetic |
| | CDR1 | | construct |
| 125 | VH gp120-b12 | INPYNGNK | synthetic |
| | CDR2 | | construct |
| 126 | VH gp120-b12 | ARVGPYSWDDSPQDNYYMDV | synthetic |
| | CDR3 | | construct |
| 127 | VL gp120-b12 | EIVLTQSPGTLSLSPGERATFSCRSSHSIRSRRVAWYQHKPG | synthetic |
| | | QAPRLVIHGVSNRASGISDRFSGSGSGTDFTLTITRVEPEDF | construct |
| | | ALYYCQVYGASSYTFGQGTKLERK | |
| 128 | VL gp120-b12 | HSIRSRR | synthetic |
| | CDR1 | | construct |
| | VL gp120-b12 | GVS | synthetic |
| | CDR2 | | construct |
| 129 | VL gp120-b12- | QVYGASSYT | synthetic |
| | CDR3 | | construct |
| 130 | MAB-19-0202-HC | QSVEESGGRLVTPGTPLTLTCTVSGFSLY | synthetic |
| | FR1 | | construct |
| 131 | MAB-19-0202-HC | WVRQAPGKGLEYIG | synthetic |
| | FR2 | | construct |
| 132 | MAB-19-0202-HC | RFTISKTSSTTVDLKMTSLTTEDTATYFCAR | synthetic |
| | FR3 | | construct |
| 133 | MAB-19-0202-HC | WGPGTLVTVSS | synthetic |
| | FR4 | | construct |
| 134 | MAB-19-0202-LC | AAVLTQTPSPVSAAVGGTVTISC | synthetic |
| | FR1 | | construct |
| 135 | MAB-19-0202-LC | WYQQKPGQPPKLLIY | synthetic |
| | FR2 | | construct |
| 136 | MAB-19-0202-LC | GVPSRFKGSGSGTQFTLTISDLESDDAATYYC | synthetic |
| | FR3 | | construct |
| 137 | MAB-19-0202-LC | FGGGTEVVVK | synthetic |
| | FR4 | | construct |
| 138 | MAB-19-0202-H5 | QVQLVESGGGLVQPGTSLRLSCSVSGFSLY | synthetic |
| | FR1 | | construct |
| 139 | MAB-19-0202-H5 | WVRQAPGKGLEYIG | synthetic |
| | FR2 | | construct |
| 140 | MAB-19-0202-H5 | RFTISRDTSKTTLYLQMNSLTTEDTATYFCAR | synthetic |
| | FR3 | | construct |
| 141 | MAB-19-0202-H5 | WGPGTLVTVSS | synthetic |
| | FR4 | | construct |
| 142 | MAB-19-0202-L4 | AIQLTQSPSSLSASVGGTVTITC | synthetic |
| | FR1 | | construct |
| 143 | MAB-19-0202-L4 | WYQQKPGQPPKLLIY | synthetic |
| | FR2 | | construct |
| 144 | MAB-19-0202-L4 | GVPSRFRGSGSGTQFTLTISSLQSEDFATYYC | synthetic |
| | FR3 | | construct |
| 145 | MAB-19-0202-L4 | FGGGTEVVVK | synthetic |
| | FR4 | | construct |
| 146 | Pembrolizumab VH | NYYMY | synthetic |
| | CDR1 (Kabat | | construct |
| | numbering) | | |
| 147 | Pembrolizumab VH | GINPSNGGTNFNEKFKN | synthetic |
| | CDR2 (Kabat | | construct |
| | numbering) | | |
| 148 | Pembrolizumab VH | RDYRFDMGFDY | synthetic |
| | CDR3 (Kabat | | construct |
| | numbering) | | |
| 149 | Pembrolizumab VL | RASKGVSTSGYSYLH | synthetic |
| | CDR1 (Kabat | | construct |
| | numbering) | | |
| 150 | Pembrolizumab VL | LASYLES | synthetic |
| | CDR2 (Kabat | | construct |
| | numbering) | | |
| 151 | Pembrolizumab VL | QHSRDLPLT | synthetic |
| | CDR3 (Kabat | | construct |
| | numbering) | | |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in a preferred embodiment of the binding agent used herein the first heavy chain comprises or consists essentially of or consists of an amino acid sequence set forth in SEQ ID NO: 23 or 29 [IgG1-Fc_FEAR] and in another preferred embodiment of the binding agent used herein the second heavy chain comprises or consists essentially of or consists of an amino acid sequence set forth in SEQ ID NO: 24 or 30 [IgG1-Fc_FEAL], then in a further preferred embodiment of the binding agent used herein the first heavy chain comprises or consists essentially of or consists of an amino acid sequence set forth in SEQ ID NO: 23 or 29 [IgG1-Fc_FEAR] and the second heavy chain comprises or consists essentially of or consists of an amino acid sequence set forth in SEQ ID NO: 24 or 30 [IgG1-Fc_FEAL].

Preferably, the terms used herein are defined as described in “A multilingual glossary of biotechnological terms: (IUPAC Recommendations)”, H.G.W. Leuenberger, B. Nagel, and H. Kolbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; R6mpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, 4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The term “binding agent” in the context of the present disclosure refers to any agent capable of binding to desired antigens. In certain embodiments of the present disclosure, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

As used herein, “immune checkpoint” refers to regulators of the immune system, and, in particular, co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2. In certain embodiments, the inhibitory signal is the interaction between CTLA-4 and CD80 or CD86 to displace CD28 binding. In certain embodiments the inhibitory signal is the interaction between LAG-3 and MHC class II molecules. In certain embodiments, the inhibitory signal is the interaction between TIM-3 and one or more of its ligands, such as galectin 9, PtdSer, HMGB1 and CEACAM1. In certain embodiments, the inhibitory signal is the interaction between one or several KIRs and their ligands. In certain embodiments, the inhibitory signal is the interaction between TIGIT and one or more of its ligands, PVR, PVRL2 and PVRL3. In certain embodiments, the inhibitory signal is the interaction between CD94/NKG2A and HLA-E. In certain embodiments, the inhibitory signal is the interaction between VISTA and its binding partner(s). In certain embodiments, the inhibitory signal is the interaction between one or more Siglecs and their ligands. In certain embodiments, the inhibitory signal is the interaction between GARP and one or more of its ligands. In certain embodiments, the inhibitory signal is the interaction between CD47 and SIRPa. In certain embodiments, the inhibitory signal is the interaction between PVRIG and PVRL2. In certain embodiments, the inhibitory signal is the interaction between CSF1R and CSF1. In certain embodiments, the inhibitory signal is the interaction between BTLA and HVEM. In certain embodiments, the inhibitory signal is part of the adenosinergic pathway, e.g., the interaction between A2AR and/or A2BR and adenosine, produced by CD39 and CD73. In certain embodiments, the inhibitory signal is the interaction between B7-H3 and its receptor and/or B7-H4 and its receptor. In certain embodiments, the inhibitory signal is mediated by IDO, CD20, NOX or TDO.

The terms “checkpoint inhibitor” (CPI) and “immune checkpoint (ICP) inhibitor” are used herein synonymously. The terms refer to molecules, such as binding agents, which totally or partially reduce, inhibit, interfere with or negatively modulate one or more checkpoint proteins or that totally or partially reduce, inhibit, interfere with or negatively modulate expression of one or more checkpoint proteins, like molecules, such as binding agents, which inhibit an immune checkpoint, in particular, which inhibit the inhibitory signal of an immune checkpoint. In one embodiment, the immune checkpoint inhibitor binds to one or more checkpoint proteins. In one embodiment, the immune checkpoint inhibitor binds to one or more molecules regulating checkpoint proteins. In one embodiment, the immune checkpoint inhibitor binds to precursors of one or more checkpoint proteins e.g., on DNA- or RNA-level. Any agent that functions as a checkpoint inhibitor according to the present disclosure can be used. The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In one embodiment, the checkpoint inhibitor can be any compound, such as any binding agent, which inhibits the inhibitory signal of an immune checkpoint, wherein the inhibitory signal is selected from the group consisting of: the interaction between PD-1 and PD-L1 and/or PD-L2; the interaction between CTLA-4 and CD80 or CD86 to displace CD28 binding; the interaction between LAG-3 and MHC class II molecules; the interaction between TIM-3 and one or more of its ligands, such as galectin 9, PtdSer, HMGB1 and CEACAM1; the interaction between one or several KIRs and their ligands; the interaction between TIGIT and one or more of its ligands, PVR, PVRL2 and PVRL3; the interaction between CD94/NKG2A and HLA-E; the interaction between VISTA and its binding partner(s); the interaction between one or more Siglecs and their ligands; the interaction between GARP and one or more of its ligands; the interaction between CD47 and SIRPa; the interaction between PVRIG and PVRL2; the interaction between CSF1R and CSF1; the interaction between BTLA and HVEM; part of the adenosinergic pathway, e.g., the interaction between A2AR and/or A2BR and adenosine, produced by CD39 and CD73; the interaction between B7-H3 and its receptor and/or B7-H4 and its receptor; an inhibitory signal mediated by IDO, CD20, NOX or TDO. In one embodiment, the checkpoint inhibitor is at least one selected from the group consisting of PD-1 inhibitors, PD-L1 inhibitors, PD-L2 inhibitors, CTLA-4 inhibitors, TIM-3 inhibitors, KIR inhibitors, LAG-3 inhibitors, TIGIT inhibitors, VISTA inhibitors, and GARP inhibitors. In one embodiment, the checkpoint inhibitor may be a blocking antibody, such as a PD-1 blocking antibody, a CTLA4 blocking antibody, a PD-L1 blocking antibody, a PD-L2 blocking antibody, a TIM-3 blocking antibody, a KIR blocking antibody, a LAG-3 blocking antibody, a TIGIT blocking antibody, a VISTA blocking antibody, or a GARP blocking antibody. Examples of a PD-1 blocking antibody include pembrolizumab, nivolumab, cemiplimab, and spartalizumab. Examples of a CTLA4 blocking antibody include ipilimumab and tremelimumab. Examples of a PD-L1 blocking antibody include atezolizumab, durvalumab, and avelumab.

In one embodiment, the anti-PD-1 antibody or antigen-binding fragment thereof comprises a heavy chain variable region (VH) comprising the CDR1, CDR2, and CDR3 sequences of SEQ ID NO: 43, and a light chain variable region (VL) comprising the CDR1, CDR2, and CDR3 sequences of SEQ ID NO: 44.

In one embodiment, the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO: 45;
(ii) a CDR-H2 comprising the amino acid sequence of SEQ ID NO: 46; and
(iii) a CDR-H3 comprising the amino acid sequence of SEQ ID NO: 47; and

wherein the light chain variable region comprises:
(i) a CDR-L1 comprising the amino acid sequence of SEQ ID NO: 48;
(ii) a CDR-L2 comprising the amino acid sequence of SEQ ID NO: 49; and
(iii) a CDR-L3 comprising the amino acid sequence of SEQ ID NO: 50.

In one embodiment of the anti-PD-1 antibodies described herein, the heavy chain variable domain comprises the amino acid sequence of SEQ ID NO: 43 and the light chain variable domain comprises the amino acid sequence of SEQ ID NO: 44.

The term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies.

The structure of immunoglobulins has been well characterized. See, e.g., Fundamental Immunology Ch. 7 (Paul, W., ed., 2^{nd} ed. Raven Press, N.Y. (1989)). Briefly, immunoglobulins generally comprise several chains, typically two identical heavy chains and two identical light chains which are linked via disulfide bonds. These chains are primarily composed of immunoglobulin domains or regions, such as the V_{L} or VL (variable light chain) domain/region, C_{L} or CL (constant light chain) domain/region, VH or VH (variable heavy chain) domain/region, and the C_{H} or CH (constant heavy chain) domains/regions C_{H}1 (CH1), C_{H}2 (CH2), C_{H}3 (CH3), and C_{H}4 (CH4). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a VL and a CL. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules using DomainGapAlign (Lefranc MP., Nucleic Acids Research 1999; 27:209-212 and Ehrenmann F., Kaas Q. and Lefranc M.-P. Nucleic Acids Res., 38, D301-307 (2010); see also internet http address www.imgt.org. Unless otherwise stated or contradicted by context, reference to amino acid positions in the constant regions in the present disclosure is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242).

There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics. Thus, classes of amino acids may be reflected in one or both of the following tables:

TABLE 2
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Alternative Physical and Functional Classifications of
Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, M, R, |
| | T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in | A, C, D, E, G, H, K, N, Q, R, S, |
| turn formation | P, and T |
| Flexible residues | Q, T, K, S, G, P, D, E, and R |

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Substitution of one amino acid for another may be classified as a conservative or non-conservative substitution. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. In the context of the present disclosure, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues. Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

The term “amino acid corresponding to position . . . ” and similar expressions as used herein refer to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present disclosure.

The term “antibody” (Ab) in the context of the present disclosure refers to an immunoglobulin molecule, a fragment of an immunoglobulin molecule, or a derivative of either thereof, which has the ability to specifically bind to an antigen (in particular an epitope on an antigen) under typical physiological conditions, preferably with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3 (or CDR-H1, CDR-H2 and CDR-H3), the CDRs of a VL are termed LCDR1, LCDR2 and LCDR3 (or CDR-L1, CDR-L2 and CDR-L3). The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and components of the complement system such as C1q. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The variable regions of the heavy and light chains of the immunoglobulin molecule contain a binding domain that interacts with an antigen. The terms “binding region” and “antigen-binding region” are used herein interchangeably and refer to the region which interacts with the antigen and comprises both a VH region and a VL region. An antibody as used herein comprises not only monospecific antibodies, but also multispecific antibodies which comprise multiple, such as two or more, e.g., three or more, different antigen-binding regions.

As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO 2007/059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody; (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January;5(1):111-24); and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

An antibody as generated can possess any isotype. As used herein, the term “isotype” refers to the immunoglobulin class (for instance IgG (such as IgG1, IgG2, IgG3, IgG4), IgD, IgA (such as IgA1, IgA2), IgE, IgM, or IgY) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody disclosed herein may be a sequence variant of a naturally-occurring IgG1 antibody, including variations in the constant regions.

IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The term “multispecific antibody” in the context of the present disclosure refers to an antibody having at least two different antigen-binding regions defined by different antibody sequences. In some embodiments, said different antigen-binding regions bind different epitopes on the same antigen.

However, in preferred embodiments, said different antigen-binding regions bind different target antigens. In one embodiment, the multispecific antibody is a “bispecific antibody” or “bs”. A multispecific antibody, such as a bispecific antibody, can be of any format, including any of the bispecific or multispecific antibody formats described herein below.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

The term “human antibody”, as used herein, is intended to include antibodies having variable and framework regions derived from human germline immunoglobulin sequences and a human immunoglobulin constant domain. The human antibodies disclosed herein may include amino acid residues not encoded by human germline immunoglobulin sequences (e.g., mutations, insertions or deletions introduced by random or site-specific mutagenesis in vitro or by somatic mutation in vivo).

However, the term “human antibody”, as used herein, is not intended to include antibodies in which CDR sequences derived from the germline of another non-human species, such as a mouse, have been grafted onto human framework sequences.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generically for different kinds of modifications of antibodies, and processes for antibody engineering are well-known for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody may be performed by other methods than those described herein. Chimeric monoclonal antibodies for therapeutic applications in humans are developed to reduce anticipated antibody immunogenicity of non-human antibodies, e.g. rodent antibodies. They may typically contain non-human (e.g. murine or rabbit) variable regions, which are specific for the antigen of interest, and human constant antibody heavy and light chain domains. The terms “variable region” or “variable domain” as used in the context of chimeric antibodies, refer to a region which comprises the CDRs and framework regions of both the heavy and light chains of an immunoglobulin, as described below.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO 92/22653 and EP 0 629 240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

As used herein, a protein which is “derived from” another protein, e.g., a parent protein, means that one or more amino acid sequences of the protein are identical or similar to one or more amino acid sequences in the other or parent protein. For example, in an antibody, binding arm, antigen-binding region, constant region, or the like which is derived from another or a parent antibody, binding arm, antigen-binding region, or constant region, one or more amino acid sequences are identical or similar to those of the other or parent antibody, binding arm, antigen-binding region, or constant region. Examples of such one or more amino acid sequences include, but are not limited to, those of the VH and VL CDRs and/or one or more or all of the framework regions, VH, VL, CL, hinge, or CH regions. For example, a humanized antibody can be described herein as “derived from” a non-human parent antibody, meaning that at least the VL and VH CDR sequences are identical or similar to the VH and VL CDR sequences of said non-human parent antibody. A chimeric antibody can be described herein as being “derived from” a non-human parent antibody, meaning that typically the VH and VL sequences may be identical or similar to those of the non-human parent antibody. Another example is a binding arm or an antigen-binding region which may be described herein as being “derived from” a particular parent antibody, meaning that said binding arm or antigen-binding region typically comprises identical or similar VH and/or VL CDRs, or VH and/or VL sequences to the binding arm or antigen-binding region of said parent antibody. As described elsewhere herein, however, amino acid modifications such as mutations can be made in the CDRs, constant regions or elsewhere in the antibody, binding arm, antigen-binding region or the like, to introduce desired characteristics. When used in the context of one or more sequences derived from a first or parent protein, a “similar” amino acid sequence preferably has a sequence identity of at least about 50%, such as at least about 60%, at least about 70%, at least about 80%, at least about 90%, at least about 95%, or at least about 97%, 98% or 99%.

Non-human antibodies can be generated in a number of different species, such as mouse, rabbit, chicken, guinea pig, llama and goat.

Monoclonal antibodies can be produced by a variety of techniques, including conventional monoclonal antibody methodology, e.g., the standard somatic cell hybridization technique of Kohler and Milstein, Nature 256:495 (1975). Other techniques for producing monoclonal antibodies can be employed, e.g., viral or oncogenic transformation of B-lymphocytes or phage display techniques using libraries of antibody genes, and such methods are well known to a person skilled in the art.

Hybridoma production in such non-human species is a very well-established procedure. Immunization protocols and techniques for isolation of splenocytes of immunized animals/non-human species for fusion are known in the art. Fusion partners (e.g., murine myeloma cells) and fusion procedures are also known.

When used herein, unless contradicted by context, the term “Fab-arm” or “arm” refers to one heavy chain-light chain pair and is used interchangeably with “half molecules” herein.

The term “binding arm comprising an antigen-binding region” means an antibody molecule or fragment that comprises an antigen-binding region. Thus, a binding arm can comprise, e.g., the six VH and VL CDR sequences, the VH and VL sequences, a Fab or Fab′ fragment, or a Fab-arm.

When used herein, unless contradicted by context, the term “Fe region” refers to an antibody region consisting of the two Fc sequences of the heavy chains of an immunoglobulin, wherein said Fe sequences comprise at least a hinge region, a CH2 domain, and a CH3 domain. In one embodiment, the term “Fe region”, as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

In the context of the present disclosure, the term “induce Fc-mediated effector function to a lesser extent” used in relation to an antibody, including a multispecific antibody, means that the antibody induces Fc-mediated effector functions, such function in particular being selected from the list of IgG Fc receptor (FcgammaR, FcγR) binding, C1q binding, ADCC or CDC, to a lesser extent compared to a human IgG1 antibody comprising (i) the same CDR sequences, in particular comprising the same first and second antigen-binding regions, as said antibody and (ii) two heavy chains comprising human IgG1 hinge, CH2 and CH3 regions.

Fc-mediated effector function may be measured by binding to FcγRs, binding to C1q, or induction of Fc-mediated cross-linking via FcγRs.

The term “hinge region” as used herein refers to the hinge region of an immunoglobulin heavy chain.

Thus, for example, the hinge region of a human IgG1 antibody corresponds to amino acids 216-230 according to the EU numbering as set forth in Kabat (Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example, the CH1 region of a human IgG1 antibody corresponds to amino acids 118-215 according to the EU numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example, the CH2 region of a human IgG1 antibody corresponds to amino acids 231-340 according to the EU numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example, the CH3 region of a human IgG1 antibody corresponds to amino acids 341-447 according to the EU numbering as set forth in Kabat (ibid). However, the CH3 region may also be any of the other subtypes as described herein.

The term “monovalent antibody” means in the context of the present disclosure that an antibody molecule is capable of binding a single molecule of the antigen, and thus is not capable of antigen cross-linking.

A “CD137 antibody” or “anti-CD137 antibody” is an antibody as described above, which binds specifically to the antigen CD137.

A “CD137xPD-L1 antibody” or “anti-CD137xPD-L1 antibody” is a bispecific antibody, which comprises two different antigen-binding regions, one of which binds specifically to the antigen CD137 and one of which binds specifically to the antigen PD-L1.

The term “biosimilar” (e.g., of an approved reference product/biological drug) as used herein refers to a biologic product that is similar to the reference product based on data from (a) analytical studies demonstrating that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product). In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. A biosimilar can be, e.g., a presently known antibody having the same primary amino acid sequence as a marketed antibody, but may be made in different cell types or by different production, purification, or formulation methods.

As used herein, the terms “binding” or “capable of binding” in the context of the binding of an antibody to a predetermined antigen or epitope typically is a binding with an affinity corresponding to a K_{D} of about 10⁻⁷ M or less, such as about 10^{×8} M or less, such as about 10^{×9} M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “ka” (sec⁻¹), as used herein, refers to the dissociation rate constant of a particular antibody-antigen interaction. Said value is also referred to as the k_{ff} value.

The term “K_{D}” (M), as used herein, refers to the dissociation equilibrium constant of a particular antibody-antigen interaction.

Two antibodies have the “same specificity” if they bind to the same antigen and to the same epitope.

Whether an antibody to be tested recognizes the same epitope as a certain antigen-binding antibody, i.e., the antibodies bind to the same epitope, may be tested by different methods well known to a person skilled in the art.

The competition between the antibodies can be detected by a cross-blocking assay. For example, a competitive ELISA assay may be used as a cross-blocking assay. E.g., target antigen may be coated on the wells of a microtiter plate and antigen-binding antibody and candidate competing test antibody may be added. The amount of the antigen-binding antibody bound to the antigen in the well indirectly correlates with the binding ability of the candidate competing test antibody that competes therewith for binding to the same epitope. Specifically, the larger the affinity of the candidate competing test antibody is for the same epitope, the smaller the amount of the antigen-binding antibody bound to the antigen-coated well. The amount of the antigen-binding antibody bound to the well can be measured by labeling the antibody with detectable or measurable labeling substances.

An antibody competing for binding to an antigen with another antibody, e.g., an antibody comprising heavy and light chain variable regions as described herein, or an antibody having the specificity for an antigen of another antibody, e.g., an antibody comprising heavy and light chain variable regions as described herein, may be an antibody comprising variants of said heavy and/or light chain variable regions as described herein, e.g. modifications in the CDRs and/or a certain degree of identity as described herein.

An “isolated multispecific antibody” as used herein is intended to refer to a multispecific antibody which is substantially free of other antibodies having different antigenic specificities (for instance an isolated bispecific antibody that specifically binds to CD137 and PD-L1 is substantially free of monospecific antibodies that specifically bind to CD137 or PD-L1).

The term “monoclonal antibody” as used herein refers to a preparation of antibody molecules of single molecular composition. A monoclonal antibody composition displays a single binding specificity and affinity for a particular epitope.

When used herein the term “heterodimeric interaction between the first and second CH3 regions” refers to the interaction between the first CH3 region and the second CH3 region in a first-CH3/second-CH3 heterodimeric antibody.

When used herein the term “homodimeric interactions of the first and second CH3 regions” refers to the interaction between a first CH3 region and another first CH3 region in a first-CH3/first-CH3 homodimeric antibody and the interaction between a second CH3 region and another second CH3 region in a second-CH3/second-CH3 homodimeric antibody.

When used herein the term “homodimeric antibody” refers to an antibody comprising two first Fab-arms or half-molecules, wherein the amino acid sequence of said Fab-arms or half-molecules is the same.

When used herein the term “heterodimeric antibody” refers to an antibody comprising a first and a second Fab-arm or half-molecule, wherein the amino acid sequence of said first and second Fab-arms or half-molecules are different. In particular, the CH3 region, or the antigen-binding region, or the CH3 region and the antigen-binding region of said first and second Fab-arms/half-molecules are different.

The term “reducing conditions” or “reducing environment” refers to a condition or an environment in which a substrate, such as a cysteine residue in the hinge region of an antibody, is more likely to become reduced than oxidized.

The present disclosure also describes multispecific antibodies, such as bispecific antibodies, comprising functional variants of the VL regions, VH regions, or one or more CDRs of the bispecific antibodies of the examples. A functional variant of a VL, VH, or CDR used in the context of a bispecific antibody still allows each antigen-binding region of the bispecific antibody to retain at least a substantial proportion (at least about 50%, 60%, 70%, 80%, 90%, 95% or more) of the affinity and/or the specificity/selectivity of the parent bispecific antibody and in some cases such a bispecific antibody may be associated with greater affinity, selectivity and/or specificity than the parent bispecific antibody.

Such functional variants typically retain significant sequence identity to the parent bispecific antibody. The percent identity between two sequences is a function of the number of identical positions shared by the sequences (i.e., % homology=#of identical positions/total #of positions x 100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

In the context of the present disclosure, unless otherwise indicated, the following notations are used to describe a mutation: i) substitution of an amino acid in a given position is written as e.g. K409R which means a substitution of a lysine in position 409 of the protein with an arginine; and ii) for specific variants the specific three or one letter codes are used, including the codes Xaa and X to indicate any amino acid residue. Thus, the substitution of lysine with arginine in position 409 is designated as: K409R, and the substitution of lysine with any amino acid residue in position 409 is designated as K409X. In case of deletion of lysine in position 409 it is indicated by K409*.

Exemplary variants include those which differ from the VH and/or VL and/or CDRs of the parent sequences mainly by conservative substitutions; for example, 12, such as 11, 10, 9, 8, 7, 6, 5, 4, 3, 2 or 1 of the substitutions in the variant are conservative amino acid residue replacements.

In the context of the present disclosure, conservative substitutions may be defined by substitutions within the classes of amino acids as defined in tables 2 and 3.

The term “CD137” as used herein, refers to CD137 (4-1BB), also referred to as tumor necrosis factor receptor superfamily member 9 (TNFRSF9), which is the receptor for the ligand TNFSF9/4-1BBL.

CD137 (4-1BB) is believed to be involved in T-cell activation. Other synonyms for CD137 include, but are not limited to, 4-1BB ligand receptor, CDwi37, T-cell antigen 4-1BB homolog and T-cell antigen ILA. In one embodiment, CD137 (4-1BB) is human CD137 (4-1BB), having UniProt accession number Q07011. The sequence of human CD137 is also shown in SEQ ID NO: 37. Amino acids 1-23 of SEQ ID NO: 37 correspond to the signal peptide of human CD137; while amino acids 24-186 of SEQ ID NO: 37 correspond to the extracellular domain of human CD137; and the remainder of the protein, i.e. from amino acids 187-213 and 214-255 of SEQ ID NO: 37 are transmembrane and cytoplasmic domain, respectively.

The “Programmed Death-1 (PD-1)” receptor refers to an immuno-inhibitory receptor belonging to the CD28 family. PD-1 (also known as CD279 or SLEB2) is expressed predominantly on previously activated T cells in vivo, and binds to two ligands, PD-L1 (also known as B7-H1 or CD274) and PD-L2 (also known as B7-DC or CD273). The term “PD-1” as used herein includes human PD-1 (hPD-1), variants, isoforms, and species homologs of hPD-1, and analogs having at least one common epitope with hPD-1, in particular a protein having the amino acid sequence (NCBI Reference Sequence: NP_005009.2) as set forth in SEQ ID NO: 113 of the sequence listing, or a protein being preferably encoded by a nucleic acid sequence (NCBI Reference Sequence: NM_005018.2) as set forth in SEQ ID NO: 115. “Programmed Death Ligand-1 (PD-L1)” is one of two cell surface glycoprotein ligands for PD-1 (the other being PD-L2) that downregulates T cell activation and cytokine secretion upon binding to PD-1.

The term “PD-L1” as used herein includes human PD-L1 (hPD-L1), variants, isoforms, and species homologs of hPD-L1, such as macaque (cynomolgus monkey), African elephant, wild boar and mouse PD-L1 (cf., e.g., Genbank accession no. NP_054862.1, XP_005581836, XP_003413533, XP_005665023 and NP_068693, respectively), and analogs having at least one common epitope with hPD-L1. The sequence of human PD-L1 is also shown in SEQ ID NO: 40 (mature sequence), and in SEQ ID NO: 39, wherein amino acids 1-18 are predicted to be a signal peptide. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

The term “dysfunctional”, as used herein, refers to an immune cell that is in a state of reduced immune responsiveness to antigen stimulation. Dysfunctional includes unresponsive to antigen recognition and impaired capacity to translate antigen recognition into downstream T cell effector functions, such as proliferation, cytokine production (e.g., IL-2) and/or target cell killing.

The term “anergy”, as used herein, refers to the state of unresponsiveness to antigen stimulation resulting from incomplete or insufficient signals delivered through the T cell receptor (TCR). T cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T cells do not undergo clonal expansion and/or acquire effector functions.

The term “exhaustion”, as used herein, refers to immune cell exhaustion, such as T cell exhaustion as a state of T cell dysfunction that arises from sustained TCR signaling that occurs during many chronic infections and cancer. It is distinguished from anergy in that it arises not through incomplete or deficient signaling, but from sustained signaling. Exhaustion is defined by poor effector function, sustained expression of inhibitory receptors and a transcriptional state distinct from that of functional effector or memory T cells. Exhaustion prevents optimal control of diseases (e.g., infection and tumors).

Exhaustion can result from both extrinsic negative regulatory pathways (e.g., immunoregulatory cytokines) as well as cell intrinsic negative regulatory pathways (inhibitory immune checkpoint pathways, such as described herein).

“Enhancing T cell function” means to induce, cause or stimulate a T cell to have a sustained or amplified biological function, or renew or reactivate exhausted or inactive T cells. Examples of enhancing T cell function include increased secretion of y-interferon from CD8⁺ T cells, increased proliferation, increased antigen responsiveness (e.g., tumor clearance) relative to such levels before the intervention.

In one embodiment, the level of enhancement is as least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 200%, or more. Manners of measuring this enhancement are known to one of ordinary skill in the art.

The term “inhibitory nucleic acid” or “inhibitory nucleic acid molecule” as used herein refers to a nucleic acid molecule, e.g., DNA or RNA, that totally or partially reduces, inhibits, interferes with or negatively modulates one or more PD-1 proteins. Inhibitory nucleic acid molecules include, without limitation, oligonucleotides, siRNA, shRNA, antisense DNA or RNA molecules, and aptamers (e.g., DNA or RNA aptamers).

The term “oligonucleotide” as used herein refers to a nucleic acid molecule that is able to decrease protein expression, in particular expression of a PD-1 protein, such as the PD-1 proteins described herein. Oligonucleotides are short DNA or RNA molecules, typically comprising from 2 to 50 nucleotides. Oligonucleotides maybe single-stranded or double-stranded. A PD-1 inhibitor oligonucleotide may be an antisense-oligonucleotide.

Antisense-oligonucleotides are single-stranded DNA or RNA molecules that are complementary to a given sequence, in particular to a sequence of the nucleic acid sequence (or a fragment thereof) of a PD-1 protein. Antisense RNA is typically used to prevent protein translation of mRNA, e.g., of mRNA encoding a PD-1 protein, by binding to said mRNA. Antisense DNA is typically used to target a specific, complementary (coding or non-coding) RNA. If binding takes place, such a DNA/RNA hybrid can be degraded by the enzyme RNase H. Moreover, morpholino antisense oligonucleotides can be used for gene knockdowns in vertebrates. For example, Kryczek et al., 2006 (J Exp Med, 203:871-81) designed B7-H4-specific morpholinos that specifically blocked B7-H4 expression in macrophages, resulting in increased T cell proliferation and reduced tumor volumes in mice with tumor associated antigen (TAA)-specific T cells.

The terms “siRNA” or “small interfering RNA” or “small inhibitory RNA” are used interchangeably herein and refer to a double-stranded RNA molecule with a typical length of 20-25 base pairs that interferes with expression of a specific gene, such as a gene coding for a PD-1 protein, with a complementary nucleotide sequence. In one embodiment, siRNA interferes with mRNA therefore blocking translation, e.g., translation of a PD-1 protein. Transfection of exogenous siRNA may be used for gene knockdown, however, the effect maybe only transient, especially in rapidly dividing cells.

Stable transfection may be achieved, e.g., by RNA modification or by using an expression vector. Useful modifications and vectors for stable transfection of cells with siRNA are known in the art. siRNA sequences may also be modified to introduce a short loop between the two strands resulting in a “small hairpin RNA” or “shRNA”. shRNA can be processed into a functional siRNA by Dicer. shRNA has a relatively low rate of degradation and turnover. Accordingly, the PD-1 inhibitor may be a shRNA.

The term “aptamer” as used herein refers to a single-stranded nucleic acid molecule, such as DNA or RNA, typically in a length of 25-70 nucleotides that is capable of binding to a target molecule, such as a polypeptide. In one embodiment, the aptamer binds to an immune PD-1 protein such as the PD-1 checkpoint proteins described herein. For example, an aptamer according to the disclosure can specifically bind to a PD-1 protein or polypeptide, or to a molecule in a signaling pathway that modulates the expression of a PD-1 protein or polypeptide. The generation and therapeutic use of aptamers is well known in the art (see, e.g., U.S. Pat. No. 5,475,096).

The terms “small molecule inhibitor” or “small molecule” are used interchangeably herein and refer to a low molecular weight organic compound, usually up to 1000 daltons, that totally or partially reduces, inhibits, interferes with, or negatively modulates one or more PD-1 proteins as described above. Such small molecular inhibitors are usually synthesized by organic chemistry, but may also be isolated from natural sources, such as plants, fungi, and microbes. The small molecular weight allows a small molecule inhibitor to rapidly diffuse across cell membranes. For example, various A2AR antagonists known in the art are organic compounds having a molecular weight below 500 daltons.

The term “cell based therapy” refers to the transplantation of cells (e.g., T lymphocytes, dendritic cells, or stem cells) expressing an immune PD-1 inhibitor into a subject for the purpose of treating a disease or disorder (e.g., a cancer disease).

The term “oncolytic virus” as used herein, refers to a virus capable of selectively replicating in and slowing the growth or inducing the death of a cancerous or hyperproliferative cell, either in vitro or in vivo, while having no or minimal effect on normal cells. An oncolytic virus for the delivery of a PD-1 inhibitor comprises an expression cassette that may encode a PD-1 inhibitor that is an inhibitory nucleic acid molecule, such as a siRNA, shRNA, an oligonucleotide, antisense DNA or RNA, an aptamer, an antibody or a fragment thereof or a soluble PD-1 protein or fusion. The oncolytic virus preferably is replication competent and the expression cassette is under the control of a viral promoter, e.g., synthetic early/late poxvirus promoter. Exemplary oncolytic viruses include vesicular stomatitis virus (VSV), rhabdoviruses (e.g., picornaviruses such as Seneca Valley virus; SVV-001), coxsackievirus, parvovirus, Newcastle disease virus (NDV), herpes simplex virus (HSV; OncoVEX GMCSF), retroviruses (e.g., influenza viruses), measles virus, reovirus, Sinbis virus, vaccinia virus, as exemplarily described in WO 2017/209053 (including Copenhagen, Western Reserve, Wyeth strains), and adenovirus (e.g., Delta-24, Delta-24-RGD, ICOVIR-5, ICOVIR-7, Onyx-015, ColoAdl, H101, AD5/3-D24-GMCSF). Generation of recombinant oncolytic viruses comprising a soluble form of a PD-1 inhibitor and methods for their use are disclosed in WO 2018/022831, herein incorporated by reference in its entirety. Oncolytic viruses can be used as attenuated viruses.

“Treatment cycle” is herein defined as the time period, within the effects of separate dosages of the binding agent add on due to the pharmacodynamics of the binding agent, or in other words the time period after the subject's body is essentially cleared from the administrated biding agent. Multiple small doses in a small time window, e.g. within 2-24 few hours, such as 2-12 hours or on the same day, might be equal to a larger single dose.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications. In one embodiment, “treatment” refers to the administration of an effective amount of a therapeutically active binding agent, such as of a therapeutically active antibody, of the present disclosure with the purpose of easing, ameliorating, arresting or eradicating (curing) symptoms or disease states.

The response to treatment as well as the resistance to, failure to respond to and/or relapse from treatment with a binding agent of the present disclosure may be determined according to the Response Evaluation Criteria in Solid Tumors; version 1.1 (RECIST Criteria v1.1). The RECIST Criteria are set forth in the table below (LD: longest dimension).

TABLE 4
Definition of Response (RECIST Criteria vl.1)

| | Category | Criteria |
|---|---|---|
| Based on | Complete Response | Disappearance of all target lesions. Any pathological lymph |
| target | (CR) | nodes must have reduction in short axis to <10 mm. |
| lesions | Partial Response | ≥30% decrease in the sum of the LD of target lesions, |
| | (PR) | taking as reference the baseline sum LD. |
| | Stable Disease | Neither sufficient shrinkage to qualify for PR nor sufficient |
| | (SD) | increase to qualify for PD, taking as reference the smallest |
| | | sum of LDs since the treatment started. |
| | Progressive Disease | ≥20% increase in the sum of the LDs of target lesions, |
| | (PD) | taking as reference the smallest sum of the LDs recorded |
| | | since the treatment started or the appearance of one or more |
| | | new lesions. |
| Based on | CR | Disappearance of all non-target lesions and normalization of |
| non-target | | tumor marker level. All lymph nodes must be non- |
| lesions | | pathological in size (<10 mm short axis). |
| | SD | Persistence of one or more non-target lesion(s) or/and |
| | | maintenance of tumor marker level above the normal limits. |
| | PD | Appearance of one or more new lesions and/or unequivocal |
| | | progression of existing non-target lesions. |

The “best overall response” is the best response recorded from the start of the treatment until disease progression/recurrence (the smallest measurements recorded since the treatment started will be used as the reference for PD). Subjects with CR or PR are considered to be objective response. Subjects with CR, PR or SD are considered to be in disease control. Subjects with NE are counted as non-responders.

The best overall response is the best response recorded from the start of the treatment until disease progression/recurrence (the smallest measurements recorded since the treatment started will be used as the reference for PD). Subjects with CR, PR or SD are considered to be in disease control. Subjects with NE are counted as non-responders.

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from Day 1 in Cycle 1 to the first documented progression or death due to any cause.

“Overall survival (OS)” is defined as the number of days from Day 1 in Cycle 1 to death due to any cause. If a subject is not known to have died, then OS will be censored at the latest date the subject was known to be alive (on or before the cut-off date).

In the context of the present disclosure, the term “treatment regimen” refers to a structured treatment plan designed to improve and maintain health.

The term “effective amount” or “therapeutically effective amount” refers to an amount effective, at dosages and for periods of time necessary, to achieve a desired therapeutic result. A therapeutically effective amount of a binding agent, such as an antibody, like a multispecific antibody or monoclonal antibody, may vary according to factors such as the disease state, age, sex, and weight of the individual, and the ability of the binding agent to elicit a desired response in the individual. A therapeutically effective amount is also one in which any toxic or detrimental effects of the binding agent or a fragment thereof, are outweighed by the therapeutically beneficial effects. In the case that a reaction in a patient is insufficient with an initial dose, higher doses (or effectively higher doses achieved by a different, more localized route of administration) may be used. In case that unwanted side effects occur in a patient with a dose, lower doses (or effectively lower doses achieved by a different, more localized route of administration) may be used.

As used herein, the term “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease.

The term “cancer” according to the present disclosure also comprises cancer metastases. By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs.

Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

Terms such as “reduce”, “inhibit”, “interfere”, and “negatively modulate” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in one embodiment relate to an increase or enhancement by at least about 10%, at least about 20%, at least about 30%, at least about 40%, at least about 50%, at least about 80%, or at least about 100%.

“Physiological pH” as used herein refers to a pH of 7.5 or about 7.5.

As used in the present disclosure, “% by weight” refers to weight percent, which is a unit of concentration measuring the amount of a substance in grams (g) expressed as a percent of the total weight of the total composition in grams (g).

The term “freezing” relates to the solidification of a liquid, usually with the removal of heat.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides and can also refer to therapeutically active fragments thereof. It can also include therapeutically active variants of a protein. Examples of therapeutically active proteins include, but are not limited to, antigens for vaccination and immunostimulants such as cytokines.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

According to the present disclosure, a part or fragment of a peptide or protein preferably has at least one functional property of the peptide or protein from which it has been derived. Such functional properties comprise a pharmacological activity, the interaction with other peptides or proteins, an enzymatic activity, the interaction with antibodies, and the selective binding of nucleic acids. E.g., a pharmacological active fragment of a peptide or protein has at least one of the pharmacological activities of the peptide or protein from which the fragment has been derived. A part or fragment of a peptide or protein preferably comprises a sequence of at least 6, in particular at least 8, at least 10, at least 12, at least 15, at least 20, at least 30 or at least 50, consecutive amino acids of the peptide or protein. A part or fragment of a peptide or protein preferably comprises a sequence of up to 8, in particular up to 10, up to 12, up to 15, up to 20, up to 30 or up to 55, consecutive amino acids of the peptide or protein.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference amino acid sequence consists of 200 amino acid residues, the degree of identity is given for at least about 100, at least about 120, at least about 140, at least about 160, at least about 180, or about 200 amino acid residues, in some embodiments continuous amino acid residues. In some embodiments, the degree of similarity or identity is given for the entire length of the reference sequence.

Homologous amino acid sequences exhibit according to the present disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence.

In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In a preferred embodiment, the binding agent used in the present disclosure is in substantially purified form.

The term “genetic modification” or simply “modification” includes the transfection of cells with nucleic acid. The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the present disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded protein.

According to the present disclosure, an analog of a peptide or protein is a modified form of said peptide or protein from which it has been derived and has at least one functional property of said peptide or protein. E.g., a pharmacological active analog of a peptide or protein has at least one of the pharmacological activities of the peptide or protein from which the analog has been derived. Such modifications include any chemical modification and comprise single or multiple substitutions, deletions and/or additions of any molecules associated with the protein or peptide, such as carbohydrates, lipids and/or proteins or peptides. In one embodiment, “analogs” of proteins or peptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said proteins and peptides.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In one embodiment, an antigen is a disease-associated antigen, such as a tumor antigen, a viral antigen, or a bacterial antigen, or an epitope derived from such antigen.

The term “epitope” refers to an antigenic determinant in a molecule such as an antigen, i.e., to a part in or fragment of the molecule that is recognized by the immune system, for example, that is recognized by antibodies T cells or B cells, in particular when presented in the context of MHC molecules. In one embodiment, “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen-binding peptide (in other words, the amino acid residue is within the footprint of the specifically antigen-binding peptide).

An epitope of a protein preferably comprises a continuous or discontinuous portion of said protein and is preferably between about 5 and about 100, preferably between about 5 and about 50, more preferably between about 8 and about 0, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present disclosure is a T cell epitope.

The term “optional” or “optionally” as used herein means that the subsequently described event, circumstance or condition may or may not occur, and that the description includes instances where said event, circumstance, or condition occurs and instances in which it does not occur.

As used herein, the terms “linked”, “fused”, or “fusion” are used interchangeably. These terms refer to the joining together of two or more elements or components or domains.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably. Similarly, the term “method for preventing” in the context of progression of a disease, such as progression of a tumor or cancer, relates to any method that is intended to prevent the disease from progressing in an individual.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer),. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

### Aspects and Embodiments of the Present Disclosure

In a first aspect, the present disclosure provides a binding agent for use in a method for reducing or preventing progression of a tumor or treating cancer in a subject, said method comprising administering to said subject the binding agent prior to, simultaneously with, or after administration of a PD-1 inhibitor, wherein the binding agent comprises a first binding region binding to CD137 and a second binding region binding to PD-L1; and

wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,

then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof. It is to be understood that neither combinations nor combined use of a binding agent comprising a first binding region binding to CD137 and a second binding region binding to PD-L1 with an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, are part of the invention provided herein. It is also to be understood that neither combinations nor combined use of a binding agent comprising a first binding region binding to CD137 and a second binding region binding to PD-L1 with an antibody comprising a heavy chain variable region (VH) comprising the CDR1, CDR2 and CDR3 sequences set forth in SEQ ID NO: 146, 147 and 148, respectively, and a light chain variable region (VL) comprising the CDR1, CDR2, and CDR3 sequences set forth in SEQ ID NO: 149, 150, and 151, respectively, are part of the invention provided herein (CDRs defined by Kabat numbering). While references are made to pembrolizumab in the present application and experimental data relating to pembrolizumab are presented herein, combinations or combined use with pembrolizumab is not intended to be included in any aspect or embodiment of the present invention.

Binding agent binding to CD137 and PD-L1

In one embodiment, CD137 is human CD137, in particular human CD137 comprising the sequence set forth in SEQ ID NO: 38. In one embodiment, PD-L1 is human PD-L1, in particular human PD-L1 comprising the sequence set forth in SEQ ID NO: 40. In one embodiment, CD137 is human CD137 and PD-L1 is human PD-L1. In one embodiment, CD137 is human CD137 comprising the sequence setforth in SEQ ID NO: 38, and PD-L1 is human PD-L comprising the sequence set forth in SEQ ID NO: 40.

In one embodiment of the binding agent according to the first aspect,
a) the first binding region binding to human CD137 comprises a heavy chain variable region (VH) comprising the CDR1, CDR2, and CDR3 sequences of SEQ ID NO: 1 or 9, and a light chain variable region (VL) comprising the CDR1, CDR2, and CDR3 sequences of SEQ ID NO: 5 or 10; and
b) the second antigen-binding region binding to human PD-L1 comprises a heavy chain variable region (VH) comprising the CDR1, CDR2, and CDR3 sequences of SEQ ID NO: 11, and a light chain variable region (VL) comprising the CDR1, CDR2, and CDR3 sequences of SEQ ID NO: 15.

In one embodiment of the binding agent according to the first aspect,
a) the first binding region binding to human CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively;
and
b) the second antigen-binding region binding to human PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively.

In one embodiment of the binding agent according to the first aspect, the first binding region binding to human CD137 comprises a heavy chain variable region (VH) comprising an amino acid sequence having at least 90%, at least 95%, at least 97%, at least 99%, or 100% sequence identity to SEQ ID NO: 1 or 9 and a light chain variable region (VL) region and comprising an amino acid sequence having at least 90%, at least 95%, at least 97%, at least 99%, or 100% sequence identity to SEQ ID NO: 5 or 10.

In further embodiment of the binding agent according to the first aspect, the second binding region binding to human PD-L1 comprises a heavy chain variable region (VH) comprising an amino acid sequence having at least 90%, at least 95%, at least 97%, at least 99%, or 25 100% sequence identity to SEQ ID NO: 11 and a light chain variable region (VL) region comprising an amino acid sequence having at least 90%, at least 95%, at least 97%, at least 99%, or 100% sequence identity to SEQ ID NO: 15.

In one embodiment of the binding agent according to the first aspect,
a) the first binding region binding to human CD137 comprises a heavy chain variable region (VH) comprising an amino acid sequence having at least 90%, at least 95%, at least 97%, at least 99%, or 100% sequence identity to SEQ ID NO: 1 or 9 and a light chain variable region (VL) region and comprising an amino acid sequence having at least 90%, at least 95%, at least 97%, at least 99%, or 100% sequence identity to SEQ ID NO: 5 or 10; and
b) the second binding region binding to human PD-L1 comprises a heavy chain variable region (VH) comprising an amino acid sequence having at least 90%, at least 95%, at least 97%, at least 99%, or 25 100% sequence identity to SEQ ID NO: 11 and a light chain variable region (VL) region comprising an amino acid sequence having at least 90%, at least 95%, at least 97%, at least 99%, or 100% sequence identity to SEQ ID NO: 15.

In one embodiment of the binding agent according to the first aspect, the first binding region binding to human CD137 comprises a heavy chain variable region (VH) comprising the amino acid sequence set forth in SEQ ID NO: 1 or 9 and a light chain variable region (VL) region comprising the amino acid sequence set forth in SEQ ID NO: 5 or 10.

In a further embodiment of the binding agent according to the first aspect, the second binding region binding to human PD-L1 comprises a heavy chain variable region (VH) comprising the amino acid sequence set forth in SEQ ID NO: 11 and a light chain variable region (VL) region comprising the amino acid sequence set forth in SEQ ID NO: 15.

In one embodiment of the binding agent according to the first aspect,
a) the first binding region binding to human CD137 comprises a heavy chain variable region (VH) comprising the amino acid sequence set forth in SEQ ID NO: 1 or 9 and a light chain variable region (VL) region comprising the amino acid sequence set forth in SEQ ID NO: 5 or 10;
and
b) the second binding region binding to human PD-L1 comprises a heavy chain variable region (VH) comprising the amino acid sequence set forth in SEQ ID NO: 11 and a light chain variable region (VL) region comprising the amino acid sequence set forth in SEQ ID NO: 15.

In one embodiment of the binding agent according to the first aspect,
a) the first binding region binding to human CD137 comprises a heavy chain variable region (VH) comprising the amino acid sequence set forth in SEQ ID NO: 1 and a light chain variable region (VL) region comprising the amino acid sequence set forth in SEQ ID NO: 5;
and
b) the second binding region binding to human PD-L1 comprises a heavy chain variable region (VH) comprising the amino acid sequence set forth in SEQ ID NO: 11 and a light chain variable region (VL) region comprising the amino acid sequence set forth in SEQ ID NO: 15.

The binding agent may in particular be an antibody, such as a multispecific antibody, e.g., a bispecific antibody. Also, the binding agent may be in the format of a full-length antibody or an antibody fragment.

It is further preferred that the binding agent is a human antibody or a humanized antibody.

Each variable region may comprise three complementarity determining regions (CDR1, CDR2, and CDR3) and four framework regions (FR1, FR2, FR3, and FR4).

The complementarity determining regions (CDRs) and the framework regions (FRs) may be arranged from amino-terminus to carboxy-terminus in the following order: FR1, CDR1, FR2, CDR2, FR3, CDR3, FR4.

In one embodiment of the first aspect, the binding agent comprises
i) a polypeptide comprising said first heavy chain variable region (VH) and a first heavy chain constant region (CH), and
ii) a polypeptide comprising said second heavy chain variable region (VH) and a second heavy chain constant region (CH).

In one embodiment of the first aspect, the binding agent comprises
i) a polypeptide comprising said first light chain variable region (VL) and further comprising a first light chain constant region (CL), and
ii) a polypeptide comprising said second light chain variable region (VL) and further comprising a second light chain constant region (CL).

In one embodiment of the first aspect, the binding agent is an antibody comprising a first binding arm and a second binding arm, wherein the first binding arm comprises
i) a polypeptide comprising said first heavy chain variable region (VH) and said first heavy chain constant region (CH), and
ii) a polypeptide comprising said first light chain variable region (VL) and said first light chain constant region (CL);
and the second binding arm comprises
iii) a polypeptide comprising said second heavy chain variable region (VH) and said second heavy chain constant region (CH), and
iv) a polypeptide comprising said second light chain variable region (VL) and said second light chain constant region (CL).

In one embodiment of the first aspect, the binding agent comprises i) a first heavy chain and light chain comprising said antigen-binding region capable of binding to CD137, the first heavy chain comprising a first heavy chain constant region and the first light chain comprising a first light chain constant region; and ii) a second heavy chain and light chain comprising said antigen-binding region capable of binding PD-L1, the second heavy chain comprising a second heavy chain constant region and the second light chain comprising a second light chain constant region.

Each of the first and second heavy chain constant regions (CH) may comprise one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

Each of the first and second heavy chain constant regions (CHs) may comprise a CH3 region, wherein the two CH3 regions comprise asymmetrical mutations. Asymmetrical mutations mean that the sequences of said first and second CH3 regions contain amino acid substitutions at non-identical positions. For example, one of said first and second CH3 regions contains a mutation at the position corresponding to position 405 in a human IgG1 heavy chain according to EU numbering, and the other of said first and second CH3 regions contains a mutation at the position corresponding to position 409 in a human IgG1 heavy chain according to EU numbering.

In said first heavy chain constant region (CH) at least one of the amino acids in a position corresponding to a position selected from the group consisting of T366, L368, K370, D399, F405, Y407, and K409 in a human IgG1 heavy chain according to EU numbering may have been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering may have been substituted. In particular embodiments, the first and the second heavy chains are not substituted in the same positions (i.e., the first and the second heavy chains contain asymmetrical mutations).

In one embodiment of the binding agent according to the first aspect, (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In one embodiment of the first aspect, the binding agent induces Fc-mediated effector function to a lesser extent compared to another antibody comprising the same first and second antigen binding regions and two heavy chain constant regions (CHs) comprising human IgG1 hinge, CH2 and CH3 regions.

In one particular embodiment of the binding agent according to the first aspect, said first and second heavy chain constant regions (CHs) are modified so that the antibody induces Fc-mediated effector function to a lesser extent compared to an antibody which is identical except for comprising non-modified first and second heavy chain constant regions (CHs). In particular, each or both of said non-modified first and second heavy chain constant regions (CHs) may comprise, consists of or consist essentially of the amino acid sequence set forth in SEQ ID NO: 19 or 25.

The Fc-mediated effector function may be determined by measuring binding of the binding agent to Fcγ receptors, binding to C1q, or induction of Fc-mediated cross-linking of Fcγ receptors. In particular, the Fc-mediated effector function may be determined by measuring binding of the binding agent to C1q.

The first and second heavy chain constant regions of the binding agent may have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

In one embodiment of the binding agent according to the first aspect, in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

In one embodiment of the binding agent according to the first aspect, the positions corresponding to positions L234 and L235 in a human IgG1 heavy chain according to EU numbering may be F and E, respectively, in said first and second heavy chains.

In particular, the positions corresponding to positions L234, L235, and D265 in a human IgG1 heavy chain according to EU numbering may be F, E, and A, respectively, in said first and second heavy chain constant regions.

In one embodiment of the binding agent according to the first aspect, the positions corresponding to positions L234 and L235 in a human IgG1 heavy chain according to EU numbering of both the first and second heavy chain constant regions are F and E, respectively, wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

In one embodiment of the binding agent according to the first aspect, the positions corresponding to positions L234, L235, and D265 in a human IgG1 heavy chain according to EU numbering of both the first and second heavy chain constant regions are F, E, and A, respectively, wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

In one embodiment of the binding agent according to the first aspect, the constant region of said first and/or second heavy chain comprises an amino acid sequence selected from the group consisting of
a) the sequence set forth in SEQ ID NO: 19 or SEQ ID NO: 25 [IgG1-FC];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

In one embodiment of the binding agent according to the first aspect, the constant region of said first or second heavy chain, such as the second heavy chain, comprises or consists essentially of or consists of an amino acid sequence selected from the group consisting of
a) the sequence set forth in SEQ ID NO: 20 or SEQ ID NO: 26 [IgG1-F405L];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

In one embodiment of the binding agent according to the first aspect, the constant region of said first or second heavy chain, such as the first heavy chain comprises or consists essentially of or consists of an amino acid sequence selected from the group consisting of
a) the sequence set forth in SEQ ID NO: 21 or 27 [IgG1-F409R];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

In one embodiment of the binding agent according to the first aspect, the constant region of said first and/or second heavy chain comprises or consists essentially of or consists of an amino acid sequence selected from the group consisting of
a) the sequence set forth in SEQ ID NO: 22 or SEQ ID NO: 28 [IgG1-Fc_FEA];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

In one embodiment of the binding agent according to the first aspect, the constant region of said first and/or second heavy chain, such as the second heavy chain, comprises or consists essentially of or consists of an amino acid sequence selected from the group consisting of
a) the sequence set forth in SEQ ID NO: 24 or SEQ ID NO: 30 [IgG1-Fc_FEAL];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

In one embodiment of the binding agent according to the first aspect, the constant region of said first and/or second heavy chain, such as the first heavy chain, comprises or consists essentially of or consists of an amino acid sequence selected from the group consisting of
a) the sequence set forth in SEQ ID NO: 23 or SEQ ID NO: 29 [IgG1-Fc_FEAR];
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

In one embodiment of the first aspect, the binding agent comprises a kappa (x) light chain constant region.

In one embodiment of the first aspect, the binding agent comprises a lambda (a) light chain constant region.

In one embodiment of the binding agent according to the first aspect, the first light chain constant region is a kappa (x) light chain constant region or a lambda (Q) light chain constant region.

In one embodiment of the binding agent according to the first aspect, the second light chain constant region is a lambda (Q) light chain constant region or a kappa (x) light chain constant region.

In one embodiment of the binding agent according to the first aspect, the first light chain constant region is a kappa (x) light chain constant region and the second light chain constant region is a lambda (a) light chain constant region or the first light chain constant region is a lambda (Q) light chain constant region and the second light chain constant region is a kappa (x) light chain constant region.

In one embodiment of the binding agent according to the first aspect, the kappa (x) light chain comprises an amino acid sequence selected from the group consisting of
a) the sequence set forth in SEQ ID NO: 35;
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

In one embodiment of the binding agent according to the first aspect, the lambda (a) light chain comprises an amino acid sequence selected from the group consisting of
a) the sequence set forth in SEQ ID NO: 36;
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The binding agent (in particular, antibody) according to the first aspect is of an isotype selected from the group consisting of IgG1, IgG2, IgG3, and IgG4. In particular, the binding agent may be a full-length IgG1 antibody. In preferred embodiments of the first aspect, the binding agent (in particular, antibody) is of the IgG1m(f) allotype.

In a preferred embodiment of the binding agent according to the first aspect, the binding agent comprises
i) a first heavy chain and light chain comprising said antigen-binding region capable of binding to CD137, wherein the first heavy chain comprising the sequence set forth in SEQ ID NO: 31, and the first light chain comprising the sequence set forth in SEQ ID NO: 32;
ii) a second heavy chain and light chain comprising said antigen-binding region capable of binding PD-L1, wherein the second heavy chain comprising the sequence set forth in SEQ ID NO: 33, and the second light chain comprising the sequence set forth in SEQ ID NO: 34.

The binding agent for use according to the first aspect may in particular be acasunlimab or a biosimilar thereof.

In currently preferred embodiments, the amount of binding agent administered in each dose and/or in each treatment cycle is
a) about 0.3-5 mg/kg body weight or about 25-400 mg in total; and/or
b) about 2.1×10⁻⁹-3.4×10⁻⁸ mol/kg body weight or about 1.7×10⁻⁷-2.7×10⁻⁶ mol in total.

According to these embodiments, the dose defined in mg/kg may be converted to flat dose, and vice versa, based on the median body weight of the subjects to whom the binding agent is administered being 80 kg

The amount of binding agent administered in each dose and/or in each treatment cycle may in particular be about 0.3-4.0 mg/kg body weight or about 25-320 mg in total; and/or about 2.1×10⁻⁹-2.7×10⁻⁸ mol/kg body weight or about 1.7×10⁻⁷-2.2×10⁻⁶ mol in total.

The amount of binding agent administered in each dose and/or in each treatment cycle may in particular be about 0.38-4.0 mg/kg body weight or about 30-320 mg in total; and/or about 2.6×10⁻⁹-2.7×10⁻⁸ mol/kg body weight or about 2.4×10⁻⁷-2.2×10⁻⁶ mol in total.

The amount of binding agent administered in each dose and/or in each treatment cycle may in particular be about 0.5-3.3 mg/kg body weight or about 40-260 mg in total; and/or about 3.4×10⁻⁹-2.2×10⁻⁸ mol/kg body weight or about 2.7×10⁻⁷-1.8×10⁻⁶ mol in total.

The amount of binding agent administered in each dose and/or in each treatment cycle may in particular be about 0.6-2.5 mg/kg body weight or about 50-200 mg in total; and/or about 4.3×10⁻⁹-1.7×10⁻⁸ mol/kg body weight or about 3.4×10⁻⁷-1.4×10⁻⁶ mol in total.

The amount of binding agent administered in each dose and/or in each treatment cycle may in particular be about 0.8-1.8 mg/kg body weight or about 60-140 mg in total; and/or about 5.1×10⁻⁹-1.2×10⁻⁸ mol/kg body weight or about 4.1×10⁻⁷-9.5×10⁻⁷ mol in total.

The amount of binding agent administered in each dose and/or in each treatment cycle may in particular be about 0.9-1.8 mg/kg body weight or about 70-140 mg in total; and/or about 6.0×10⁻⁹-1.2×10⁻⁸ mol/kg body weight or about 4.8×10⁻⁷-9.5×10⁻⁷ mol in total.

The amount of binding agent administered in each dose and/or in each treatment cycle may in particular be about 1-1.5 mg/kg body weight or about 80-120 mg in total; and/or about 6.8×10⁻⁹-1.0×10⁻⁸ mol/kg body weight or about 5.5×10⁻⁷-8.2×10⁻⁷ mol in total.

The amount of binding agent administered in each dose and/or in each treatment cycle may in particular be about 1.1-1.4 mg/kg body weight or about 90-110 mg in total; and/or about 7.7×10⁻⁹-9.4×10⁻⁹ mol/kg body weight or about 6.1×10-?-7.5×10⁻⁷ mol in total.

The amount of binding agent administered in each dose and/or in each treatment cycle may in particular be about 1.2-1.3 mg/kg body weight or about 95-105 mg in total; and/or about 6.8×10⁻⁹-8.9×10⁻⁹ mol/kg body weight or about 6.5×10⁻⁷-7.2×10⁻⁷ mol in total.

The amount of binding agent administered in each dose and/or in each treatment cycle may in particular be about 0,8-1.5 mg/kg body weight or about 65-120 mg in total; and/or about 5.5×10⁻⁹-1.0×10⁻⁸ mol/kg body weight or about 4.4×10⁻⁷-8.2×10⁻⁷ mol in total.

The amount of binding agent administered in each dose and/or in each treatment cycle may in particular be about 0.9-1.3 mg/kg body weight or about 70-100 mg in total; and/or about 6.0×10⁻⁹-8.5×10⁻⁹ mol/kg body weight or about 4.8×10⁻⁷-6.8×10⁻⁷ mol in total. about 0.9-1.1 mg/kg body weight or about 75-90 mg in total; and/or about 6.4×10⁻⁹-7.7×10⁻⁹ mol/kg body weight or about 5.1×10⁻⁷-6.1×10⁻⁷ mol in total.

Further, the amount of binding agent administered in each dose and/or in each treatment cycle may in particular be 0.3-4.0 mg/kg body weight or 25-320 mg in total; and/or 2.1×10⁻⁹-2.7×10⁻⁸ mol/kg body weight or 1.7×10⁻⁷-2.2×10⁻⁶ mol in total.

The amount of binding agent administered in each dose and/or in each treatment cycle may in particular be 0.38-4.0 mg/kg body weight or 30-320 mg in total; and/or 2.6×10⁻⁹-2.7×10⁻⁸ mol/kg body weight or 2.4×10⁻⁷-2.2×10⁻⁶ mol in total.

The amount of binding agent administered in each dose and/or in each treatment cycle may in particular be 0.5-3.3 mg/kg body weight or 40-260 mg in total; and/or 3.4×10⁻⁹-2.2×10⁻⁸ mol/kg body weight or 2.7×10⁻⁷-1.8×10⁻⁶ mol in total.

The amount of binding agent administered in each dose and/or in each treatment cycle may in particular be 0.6-2.5 mg/kg body weight or 50-200 mg in total; and/or 4.3×10⁻⁹-1.7×10⁻⁸ mol/kg body weight or 3.4×10-?-1.4×10⁻⁶ mol in total.

The amount of binding agent administered in each dose and/or in each treatment cycle may in particular be 0.8-1.8 mg/kg body weight or 60-140 mg in total; and/or 5.1×10⁻⁹-1.2×10⁻⁸ mol/kg body weight or 4.1×10-?-9.5×10⁻⁷ mol in total.

The amount of binding agent administered in each dose and/or in each treatment cycle may in particular be 0.9-1.8 mg/kg body weight or 70-140 mg in total; and/or 6.0×10⁻⁹-1.2×10⁻⁸ mol/kg body weight or 4.8×10-?-9.5×10⁻⁷ mol in total.

The amount of binding agent administered in each dose and/or in each treatment cycle may in particular be 1-1.5 mg/kg body weight or 80-120 mg in total; and/or 6.8×10⁻⁹-1.0×10⁻⁸ mol/kg body weight or 5.5×10⁻⁷-8.2×10⁻⁷ mol in total.

The amount of binding agent administered in each dose and/or in each treatment cycle may in particular be 1.1-1.4 mg/kg body weight or 90-110 mg in total; and/or 7.7×10⁻⁹-9.4×10⁻⁹ mol/kg body weight or 6.1×10⁻⁷-7.5×10⁻⁷ mol in total.

The amount of binding agent administered in each dose and/or in each treatment cycle may in particular be 1.2-1.3 mg/kg body weight or 95-105 mg in total; and/or 6.8×10⁻⁹-8.9×10⁻⁹ mol/kg body weight or 6.5×10⁻⁷-7.2×10⁻⁷ mol in total.

The amount of binding agent administered in each dose and/or in each treatment cycle may in particular be 0,8-1.5 mg/kg body weight or 65-120 mg in total; and/or 5.5×10⁻⁹-1.0×10⁻⁸ mol/kg body weight or 4.4×10⁻⁷-8.2×10⁻⁷ mol in total.

The amount of binding agent administered in each dose and/or in each treatment cycle may in particular be 0.9-1.3 mg/kg body weight or 70-100 mg in total; and/or 6.0×10⁻⁹-8.5×10⁻⁹ mol/kg body weight or 4.8×10⁻⁷-6.8×10⁻⁷ mol in total.

The amount of binding agent administered in each dose and/or in each treatment cycle may in particular be 0.9-1.1 mg/kg body weight or 75-90 mg in total; and/or 6.4×10⁻⁹-7.7×10⁻⁹ mol/kg body weight or 5.1×10⁻⁷-6.1×10⁻⁷ mol in total.

The amount of binding agent administered in each dose and/or in each treatment cycle may be
a) about 1.1 mg/kg body weight or about 80 mg in total; and/or
b) about 6.8×10⁻⁹ mol/kg body weight or about 5.5×10-? mol in total.

The amount of binding agent administered in each dose and/or in each treatment cycle may be
a) 1.1 mg/kg body weight or 80 mg in total; and/or
b) 6.8×10⁻⁹ mol/kg body weight or 5.5×10⁻⁷ mol in total.

It is currently preferred that the amount of binding agent administered in each dose and/or in each treatment cycle is
a) about 1.25 mg/kg body weight or about 100 mg in total; and/or
b) about 8.5×10⁻⁹ mol/kg body weight or about 6.8×10⁻⁷ mol in total.

It is equally preferred that the amount of binding agent administered in each dose and/or in each treatment cycle is
a) 1.25 mg/kg body weight or 100 mg in total; and/or
b) 8.5×10⁻⁹ mol/kg body weight or 6.8×10-? mol in total.

The binding agent may be administered in any manner and by any route known in the art. In a preferred embodiment, the binding agent is administered systemically, such as parenterally, in particular intravenously.

The binding agent may be administered in the form of any suitable pharmaceutical composition as described herein. In a preferred embodiment, the binding agent is administered in the form of an infusion.

The binding agent for use according to the invention may be administered by using intravenous (IV) infusion, such as by intravenous infusion over a minimum of 30 minutes, such as over a minimum of 60 minutes e.g., by using intravenous infusion over 30 to 120 minutes. Preferably, the binding agent for use according to the invention is administered by using intravenous (IV) infusion over 30 minutes.

The binding agent can be administered prior to, simultaneously with, or after administration of the PD-1 inhibitor.

In one embodiment, the binding agent is administered prior to the administration of the PD-1 inhibitor.

For example, the gap between the end of the administration of the binding agent and the beginning of the administration of the PD-1 inhibitor can be at least about 10 min, such as at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min, and up to about 14 days (up to about 2 weeks), such as up to about 13 days, up to about 12 days, up to about 11 days, up to about 10 days, up to about 9 days, up to about 8 days, up to about 7 days (up to about 1 week), up to about 6 days, up to about 5 days, up to about 4 days, up to about 3 days, up to about 2 days, up to about 1 day (up to about 24 h), up to about 18 h, up to about 12 h, up to about 6 h, up to about 5 h, up to about 4 h, up to about 3 h, up to about 2.5 h, or up to about 2 h.

In one embodiment, the binding agent is administered after the administration of the PD-1 inhibitor. For example, the gap between the end of the administration of the PD-1 inhibitor and the beginning of the administration of the binding agent can be at least about 10 min, such as at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min, and up to about 14 days (up to about 2 weeks), such as up to about 13 days, up to about 12 days, up to about 11 days, up to about 10 days, up to about 9 days, up to about 8 days, up to about 7 days (up to about 1 week), up to about 6 days, up to about 5 days, up to about 4 days, up to about 3 days, up to about 2 days, up to about 1 day (up to about 24 h), up to about 18 h, up to about 12 h, up to about 6 h, up to about 5 h, up to about 4 h, up to about 3 h, up to about 2.5 h, or up to about 2 h.

In one embodiment, the binding agent is administered simultaneously with the PD-1 inhibitor. For example, the binding agent and the PD-1 inhibitor may be administered using a composition comprising both drugs. Alternatively, the binding agent may be administered into one extremity of the subject, and the PD-1 inhibitor may be administered into another extremity of the subject.

### PD-1 Inhibitor

In one embodiment, the PD-1 inhibitor prevents inhibitory signals associated with PD-1. In one embodiment, the PD-1 inhibitor is an antibody, or fragment thereof that disrupts or inhibits inhibitory signaling associated with PD-1. In one embodiment, the PD-1 inhibitor is a small molecule inhibitor that disrupts or inhibits inhibitory signaling. In one embodiment, the PD-1 inhibitor is a peptide-based inhibitor that disrupts or inhibits inhibitory signaling. In one embodiment, the PD-1 inhibitor is an inhibitory nucleic acid molecule that disrupts or inhibits inhibitory signaling.

Inhibiting or blocking of PD-1 signaling, as described herein, results in preventing or reversing immune-suppression and establishment or enhancement of T cell immunity against cancer cells. In one embodiment, inhibition of PD-1 signaling, as described herein, reduces or inhibits dysfunction of the immune system. In one embodiment, inhibition of PD-1 signaling, as described herein, renders dysfunctional immune cells less dysfunctional. In one embodiment, inhibition of PD-1 signaling, as described herein, renders a dysfunctional T cell less dysfunctional.

In one embodiment, the PD-1 inhibitor prevents the interaction between PD-1 and PD-L1.

The PD-1 inhibitor may be an antibody, an antigen-binding fragment thereof, or a construct thereof comprising an antibody portion with an antigen-binding fragment of the required specificity. Antibodies or antigen-binding fragments thereof are as described herein. Antibodies or antigen-binding fragments thereof that are PD-1 inhibitors include in particular antibodies or antigen-binding fragments thereof that bind to PD-1. Antibodies or antigen-binding fragments may also be conjugated to further moieties, as described herein. In particular, antibodies or antigen-binding fragments thereof are chimerized, humanized or human antibodies.

In a preferred embodiment, an antibody that is a PD-1 inhibitor is an isolated antibody.

In one embodiment, the PD-1 inhibitor is an antibody, a fragment or construct thereof that prevents the interaction between PD-1 and PD-L1.

The PD-1 inhibitor may be an inhibitory nucleic acid molecule, such as an oligonucleotide, siRNA, shRNA, an antisense DNA or RNA molecule, and an aptamer (e.g., DNA or RNA aptamer), in particular an antisense-oligonucleotide. In one embodiment, the PD-1 checkpoint inhibitor being siRNA interferes with mRNA therefore blocking translation, e.g., translation of a PD-1 protein.

In one embodiment, the PD-1 inhibitor is an antibody, an antigen-binding portion thereof or a construct thereof that disrupts or inhibits the interaction between the PD-1 receptor and one or more of its ligands, PD-L1 and/or PD-L2. Antibodies which bind to PD-1 and disrupt or inhibit the interaction between PD-1 and one or more of its ligands are known in the art. In certain embodiments, the antibody, antigen-binding portion thereof or a construct thereof binds specifically to PD-1 In further preferred embodiments, the PD-1 inhibitor is an antibody that binds to PD-1, such as a PD-1 blocking antibody. Without being bound by theory the combination of a binding agent comprising a first binding region binding to CD137 and a second binding region binding to PD-L1 with an antibody binding to PD-1 is believed to increase the response rate and lead to improved duration of response in subjects receiving the combination therapy because the combination therapy leads to complete blockade of the PD-1 pathway with concurrent conditional activation of 4-1BB. A PD-1 blocking antibody blocks interaction with both PD-L1 and PD-L2. It is further believed that the combination therapy with an antibody binding to PD-1 makes increased amounts of PD-L1 available to be bound by the binding agent.

Exemplary PD-1 inhibitors include, without limitation, anti-PD-1 antibodies such as BGB-A317 (BeiGene; see U.S. Pat. No. 8,735,553, WO 2015/35606 and US 2015/0079109), lambrolizumab (e.g., disclosed as hPD109A and its humanized derivatives h409A1, h409A16 and h409A17 in WO2008/156712), AB137132 (Abcam), EH12.2H7 and RMP1-14 (#BE0146; Bioxcell Lifesciences Pvt. LTD.), MIH4 (Affymetrix eBioscience), nivolumab (OPD1VO, BMS-936558; Bristol Myers Squibb; see U.S. Pat. No. 8,008,449; WO 2013/173223; WO 2006/121168), pembrolizumab (KEYTRUDA; MK-3475; Merck; see WO 2008/156712), pidilizumab (CT-011; CureTech; see Hardy et al., 1994, Cancer Res., 54(22):5793-6 and WO 2009/101611), PDR001 (Novartis; see WO 2015/112900), MEDI0680 (AMP-514; AstraZeneca; see WO 2012/145493), TSR-042 (see WO 2014/179664), cemiplimab (REGN-2810; Regeneron; H4H7798N; cf. US 2015/0203579 and WO 2015/112800), JS001 (TAIZHOU JUNSHI PHARMA; see Si-Yang Liu et al., 2007, J. Hematol. Oncol. 70:136), AMP-224 (GSK-2661380; cf. Li et al., 2016, Int J Mol Sci 17(7):1151 and WO 2010/027827 and WO 2011/066342), PF-06801591 (Pfizer), tislelizumab (BGB-A317; BeiGene; see WO 2015/35606, U.S. Pat. No. 9,834,606, and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANBO11; see WO2014/179664), GLS-010 (Wuxi/Harbin Gloria Pharmaceuticals; also known as WBP3055; see Si-Yang et al., 2017, J. Hematol. Oncol. 70:136), STI-1110 (Sorrento Therapeutics; see WO 2014/194302), AGEN2034 (Agenus; see WO 2017/040790), MGA012 (Macrogenics; see WO 2017/19846), IBI308 (Innovent; see WO 2017/024465, WO 2017/025016, WO 2017/132825, and WO 2017/133540), cetrelimab (JNJ-63723283; JNJ-3283; see Calvo et al., J. Clin. Oncol. 36, no. 5_suppl (2018) 58), genolimzumab (CBT-501; see Patel et al., J. ImmunoTher. Cancer, 2017, 5(Suppl 2):P242), sasanlimab (PF-06801591; see Youssef et al., Proc. Am. Assoc. Cancer Res. Ann. Meeting 2017; Cancer Res 2017;77(13 Suppl):Abstract), toripalimab (JS-001; see US 2016/0272708), camrelizumab (SHR-1210; INCSHR-1210; see US 2016/376367; Huang et al., Clin. Cancer Res. 2018; 24(6):1296-1304), spartalizumab (PDR001; see WO 2017/106656; Naing et al., J. Clin. Oncol. 34, no. 15_suppl (2016) 3060-3060), BCD-100 (JSC BIOCAD, Russia; see WO 2018/103017), balstilimab (AGEN2034; see WO 2017/040790), sintilimab (IBI-308; see WO 2017/024465 and WO 2017/133540), ezabenlimab (BI-754091; see US 2017/334995; Johnson et al., J. Clin. Oncol. 36, no. 5_suppl (2018) 212-212), zimberelimab (GLS-010; see WO 2017/025051), LZM-009 (see US 2017/210806), AK-103 (see WO 2017/071625, WO 2017/166804, and WO 2018/036472), retifanlimab (MGA-012; see WO 2017/019846), Sym-021 (see WO 2017/055547), CS1003 (see CN107840887), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105, 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28(9):665-678 and Sasikumar and Ramachandra, 2018, BioDrugs, 32(5):481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831. In a certain embodiment, the PD-1 inhibitor is nivolumab (OPD1VO; BMS-936558) or a biosimilar thereof, pembrolizumab (KEYTRUDA; MK-3475) or a biosimilar thereof, pidilizumab (CT-011), PDR001, MEDI0680 (AMP-514) or a biosimilar thereof, TSR-042, REGN2810, JS001, AMP-224 (GSK-2661380), PF-06801591, BGB-A317, BI 754091, or SHR-1210.

The PD-1 inhibitor may in particular be pembrolizumab or a biosimilar thereof. Alternatively, the antibody may be nivolumab or a biosimilar thereof.

In certain embodiments, the PD-1 inhibitor immunoregulator is an anti-PD-1 antibody or antigen-binding fragment thereof comprising the complementary determining regions (CDRs) of one of the anti-PD-1 antibodies or antigen-binding fragments described above, such as the CDRs of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In some embodiments, the CDRs of the anti-PD-1 antibody are delineated using the Kabat numbering scheme (Kabat, E. A., et al. (1991) Sequences of Proteins of Immunological Interest, Fifth Edition, U.S. Department of Health and Human Services, NTH Publication No. 91-3242).

In certain embodiments, the PD-1 inhibitor is an anti-PD-1 antibody or antigen-binding fragment thereof comprising the heavy chain variable region and the light chain variable region of one of the anti-PD-1 antibodies or antigen-binding fragments described above, such as the heavy chain variable region and the light chain variable region of one anti-PD-1 antibody or antigen-binding fragment selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In certain embodiments, the PD-Iinhibitor is an anti-PD-1 antibody or antigen-binding fragment thereof selected from the group consisting of nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

The CDR sequences of pembrolizumab are identified herein by SEQ ID NOs: 59-61 (VH CDRs 1, 2 and 3, respectively) and by SEQ ID NOs: 62-64 (VL CDRs 1, 2 and 3, respectively. The VH and VL sequences are identified by SEQ ID NOs: 65 and 66, respectively and the heavy and light chain sequences are identified by SEQ ID NOs:67 and 68, respectively. Hence, in one embodiment the PD-1 inhibitor is an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63 and 64, respectively.

In a further embodiment the PD-1 inhibitor is an antibody comprising a heavy chain variable region (VH) comprising or consisting of or consisting essentially of the sequence set forth in SEQ ID NO: 65, and a light chain variable region (VL) comprising, consisting of or consisting essentially of the sequence set forth in SEQ ID NO: 66. The PD-1 inhibitor may in particular be an antibody comprising a heavy chain comprising, consisting of or consisting essentially of the amino acid sequence set forth in SEQ ID NO: 67, and a light chain comprising, consisting of or consisting essentially of the amino acid sequence set forth in SEQ ID NO: 68.

The CDR sequences of nivolumab are identified herein by SEQ ID NOs: 69-71 (VH CDRs 1, 2 and 3, respectively) and by SEQ ID NOs: 72-74 (VL CDRs 1, 2 and 3, respectively. The VH and VL sequences are identified by SEQ ID NOs: 75 and 76, respectively and the heavy and light chain sequences are identified by SEQ ID NOs: 77 and 78, respectively. Hence, in one embodiment the PD-1 inhibitor is an antibody comprising a heavy chain variable region (VH) comprising the CDR1, CDR2 and CDR3 sequences set forth in SEQ ID NO: 69, 70 and 71, respectively, and a light chain variable region (VL) comprising the CDR1, CDR2, and CDR3 sequences set forth in SEQ ID NO: 72, 73 and 74, respectively.

In a further embodiment the PD-1 inhibitor is an antibody comprising a heavy chain variable region (VH) comprising or consisting of or consisting essentially of the sequence set forth in SEQ ID NO: 75, and a light chain variable region (VL) comprising, consisting of or consisting essentially of the sequence set forth in SEQ ID NO: 76. The PD-1 inhibitor may in particular be an antibody comprising a heavy chain comprising, consisting of or consisting essentially of the amino acid sequence set forth in SEQ ID NO: 77, and a light chain comprising, consisting of or consisting essentially of the amino acid sequence set forth in SEQ ID NO: 78.

Anti-PD-1 antibodies of the disclosure are preferably monoclonal, and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein binds specifically to PD-1 (e.g., human PD-1). The immunoglobulin molecules of the disclosure can be of any isotype (e.g., IgG, IgE, IgM, IgD, IgA and IgY), class (e.g., IgG1, IgG2, IgG3, IgG4, IgA1 and IgA2) or subclass of immunoglobulin molecule. In certain embodiments of the disclosure, the anti-PD-1 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken.

The anti-PD-1 antibodies disclosed herein may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

The anti-PD-1 antibodies disclosed herein may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003; 27(1):55-77 (“IMGT” numbering scheme); Honegger A and Ph0ckthun A, “Yet another numbering scheme for immunoglobulin variable domains: an automatic modeling and analysis tool,” J Mol Biol, 2001; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a CDR or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given VH or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

In some embodiments, numbering of amino acid residues in CDR sequences of anti-PD-1 antibodies or antigen-binding fragments thereof provided herein are according to the IMGT numbering scheme as described in Lefranc, M. P. et al., Dev. Comp. Immunol., 2003, 27, 55-77.

In some embodiments, the anti-PD-1 antibodies disclosed herein comprise the CDRs of the antibody nivolumab. See WO 2006/121168. In some embodiments, the CDRs of the antibody nivolumab are delineated using the Kabat numbering scheme (Kabat, E. A., et al. (1991) Sequences of Proteins of Immunological Interest, Fifth Edition, U.S. Department of Health and Human Services, NTH Publication No. 91-3242). The present disclosure encompasses an anti-PD-1 antibody or derivative thereof comprising a heavy or light chain variable domain, said variable domain comprising (a) a set of three CDRs, in which said set of CDRs are from the monoclonal antibody nivolumab, and (b) a set of four framework regions, in which said set of framework regions differs from the set of framework regions in the monoclonal antibody nivolumab, and in which said anti-PD-1 antibody or derivative thereof binds to PD-1. In certain embodiments, the anti-PD-1 antibody is nivolumab.

Anti-PD-1 antibodies disclosed herein may also be described or specified in terms of their binding affinity to PD-1 (e.g., human PD-1). Preferred binding affinities include those with a dissociation constant or Kd less than 5×10⁻² M, 10^{×2} M, 5×10⁻³ M, 10⁻³ M, 5×10⁻⁴ M, 10⁻⁴ M, 5×10⁻⁵ M, 10^{×5} M, 5×10^{×6} M, 10⁻⁶ M, 5×10⁻⁷ M, 10^{×7} M, 5×10⁻⁸ M, 10⁻⁸M, 5×10^{×9} M, 10^{×9} M, 5×10⁻¹⁰ M, 10^{×10} M, 5×10⁻¹¹ M, 10⁻¹¹ M, 5×10⁻¹² M, 10^{×12} M, 5×10^{×13} M, 10^{×13} M, 5×10^{×14} M, 10^{×14} M, 5×10⁻⁵ M, or 10⁻¹⁵ M.

The anti-PD-1 antibodies also include derivatives and constructs that are modified, i.e., by the covalent attachment of any type of molecule to the antibody such that covalent attachment does not prevent the antibody from binding to PD-1. For example, but not by way of limitation, the anti-PD-1 antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative or construct may contain one or more non-classical amino acids.

In a preferred embodiment, the PD-1 inhibitor is an antibody, in particular an antagonistic or blocking antibody, which disrupts or inhibits the PD-1 pathway (interaction of PD-1 with one or more of its ligands (such as PD-L1 and/or PD-L2). In one preferred embodiment, the PD-1 inhibitor is an antibody, in particular an antagonistic or blocking antibody, which disrupts or inhibits the interaction between PD-1 and PD-L1.

PD-1 inhibitors may be administered in the form of nucleic acid, such DNA or RNA molecules, encoding a PD-1 inhibitor, e.g., an inhibitory nucleic acid molecule or an antibody or fragment thereof.

For example, antibodies can be delivered encoded in expression vectors, as described herein. Nucleic acid molecules can be delivered as such, e.g., in the form of a plasmid or mRNA molecule, or complexed with a delivery vehicle, e.g., a liposome, lipoplex or nucleic-acid lipid particles. PD-1t inhibitors may also be administered via an oncolytic virus comprising an expression cassette encoding the PD-1 inhibitor. PD-1 may also be administered by administration of endogeneic or allogeneic cells able to express a PD-1 inhibitor, e.g., in the form of a cell-based therapy.

Preferably, the PD-1 inhibitor is administered in a suitable amount. The amount of PD-1 inhibitor administered in each dose and/or treatment cycle may in particular be in a range, wherein more than 5%, preferably more than 10%, more preferably more than 15%, even more preferably more than 20%, even more preferably more than 25%, even more preferably more than 30%, even more preferably more than 35%, even more preferably more than 40%, even more preferably more than 45%, most preferably more than 50% of said PD-1 inhibitors bind to PD-1.

In certain embodiments, the PD-1 inhibitor is pembrolizumab or a biosimilar thereof and the amount of PD-1 inhibitor administered, e.g., in each dose and/or in each treatment cycle, is about 10-about 1000 mg in total such as about 100-about 600 mg in total, e.g., about 150-about 600 mg in total, about 150-about 500 mg in total, about 175-about 500 mg in total, about 175-about 450 mg in total, about 200-about 450 mg in total or such as about 200-about 400 mg in total.

In certain embodiments, the PD-1 inhibitor is pembrolizumab or a biosimilar thereof and the amount of PD-1 inhibitor administered, e.g., in each dose and/or in each treatment cycle, is 10-1000 mg in total such as 100-600 mg in total, e.g., 150-600 mg in total, 150-500 mg in total, 175-500 mg in total, 175-450 mg in total, 200-450 mg in total or such as 200-400 mg in total.

In certain embodiments, the PD-1 inhibitor is pembrolizumab or a biosimilar thereof and the amount of PD-1 inhibitor administered, e.g., in each dose and/or in each treatment cycle, is about 100-600 mg in total; and/or about 6.84×10⁻⁷-4.11×10⁻⁷ mol in total.

In certain embodiments, the PD-1 inhibitor is pembrolizumab or a biosimilar thereof and the amount of PD-1 inhibitor administered, e.g., in each dose and/or in each treatment cycle, is about 100-400 mg in total; and/or about 6.84×10⁻⁷-2.73×10⁻⁶ mol in total, such as 100-400 mg in total; and/or 6.84×10⁻⁷-2.73×10⁻⁶ mol in total.

In certain embodiments, the PD-1 inhibitor is pembrolizumab or a biosimilar thereof and the amount of PD-1 inhibitor administered, e.g., in each dose and/or in each treatment cycle, is about 200-400 mg in total; and/or about 6.84×10⁻⁷-2.73×10⁻⁶ mol in total, such as 200-400 mg in total; and/or 6.84×10⁻⁷-2.73×10⁻⁶ mol in total.

In certain embodiments, the amount of PD-1 inhibitor administered, e.g., in each dose and/or in each treatment cycle, is about 200 mg or about 1.37×10⁻⁶ mol in total, such as 200 mg or 1.37×10⁻⁶ mol in total.

In certain embodiments, the PD-1 inhibitor is pembrolizumab or a biosimilar thereof and the amount of PD-1 inhibitor administered, e.g., in each dose and/or in each treatment cycle, is about 200 mg or about 1.37×10⁻⁶ mol in total, such as 200 mg or 1.37×10⁻⁶ mol in total.

In certain embodiments, the amount of PD-1 inhibitor administered, e.g., in each dose and/or in each treatment cycle, is about 400 mg in total or about 2.73×10⁻⁶ in total, such as 400 mg in total or 2.73×10⁻⁶ in total.

In certain embodiments, the PD-1 inhibitor is pembrolizumab or a biosimilar thereof and the amount of PD-1 inhibitor administered, e.g., in each dose and/or in each treatment cycle, is about 400 mg in total or about 2.73×10⁻⁶ in total, such as 400 mg in total or 2.73×10⁻⁶ in total.

PD-1 inhibitors may be administered in any manner and by any route known in the art. The mode and route of administration will depend on the type of PD-1 inhibitor to be used. In a preferred embodiment, the PD-1 inhibitor is administered systemically, such as parenterally, in particular intravenously.

PD-1 inhibitors may be administered in the form of any suitable pharmaceutical composition as described herein. In a preferred embodiment, the PD-1 inhibitor is administered in the form of an infusion, such as an intravenous infusion.

The antibody binding to PD-1 may comprise a heavy chain variable region (VH) comprising a HCDR1, HCDR2, and HCDR3 sequence and a light chain variable region (VL) comprising a LCDR1, LCDR2, and LCDR3 sequence, wherein the HCDR1, HCDR2 and HCDR3 sequence comprises or has the sequence as set forth in SEQ ID NO: 104, SEQ ID NO: 101, and SEQ ID NO: 100, respectively, and the LCDR1, LCDR2 and LCDR3 sequence comprises or has the sequence as set forth in SEQ ID NO: 107, QAS, and SEQ ID NO: 105, respectively. A specific, but not limiting example of such an antibody is MAB-19-0202.

The terms “a heavy chain variable region” (also referred to as “VH”) and “a light chain variable region” (also referred to as “VL”) are used here in their most general meaning and comprise any sequences that are able to comprise complementarity determining regions (CDR), interspersed with other regions, which also termed framework regions (FR). The framework reagions inter alia space the CDRs so that they are able to form the antigen-binding site, in particular after folding and pairing of VH and VL. Preferably each VH and VL is composed of three CDRs and four FRs, arranged from amino-terminus to carboxy-terminus in the following order: FR1, CDR1, FR2, CDR2, FR3, CDR3, FR4. That is, the terms “a heavy chain variable region” and “a light chain variable region” are not to be construed to be limited to such sequences as they can be found in a native antibody or in the VH and VL sequences as exemplified herein (SEQ ID NOs: 109 to 112 of the sequence listing). These terms include any sequences capable of comprising and adequately positioning CDRs, for example such sequences as derived from VL and VH regions of native antibodies or as derived from the sequences as set forth in SEQ ID NOs: 109 to 112 of the sequence listing. It will be appreciated by those skilled in the art that in particular the sequences of the framework regions can be modified (includings both variants with regard to amino acid substitutions and variants with regard to the sequence length, i.e., insertion or deletion variants) without losing the charactistics of the VH and VL, respectively. In a preferred embodiment any modification is limited to the framework regions. But, a person skilled in the art is also well aware of the fact that also CDR, hypervariable and variable regions can be modified without losing the ability to bind PD-1. For example, CDR regions will be either identical or highly homologous to the regions specified herein. By “highly homologous” it is contemplated that from 1 to 5, preferably from 1 to 4, such as 1 to 3 or 1 or 2 substitutions may be made in the CDRs. In addition, the hypervariable and variable regions may be modified so that they show substantial homology with the regions specifically disclosed herein.

In the antibody binding to PD-1, the CDRs as specified herein have been identified by using two different CDR identification methods. The first numbering scheme used herein is according to Kabat (Wu and Kabat, 1970; Kabat et al., 1991), the second scheme is the IMGT numbering (Lefranc, 1997; Lefranc et al., 2005). In a third approach, the intersection of both identification schemes has been used.

The antibody binding to PD-1 may comprise one or more CDRs, a set of CDRs or a combination of sets of CDRs as described herein comprises said CDRs together with their intervening framework regions (also referred to as framing region or FR herein) or with portions of said framework regions. Preferably, the portion will include at least about 50% of either or both of the first and fourth framework regions, the 50% being the C-terminal 50% of the first framework region and the N-terminal 50% of the fourth framework region. Construction of antibodies made by recombinant DNA techniques may result in the introduction of residues N- or C-terminal to the variable regions encoded by linkers introduced to facilitate cloning or other manipulation steps, including the introduction of linkers to join variable regions of the disclosure to further protein sequences including immunoglobulin heavy chains, other variable domains (for example in the production of diabodies) or protein labels.

The antibody binding to PD-1 may comprise a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in any one of SEQ ID NO: 111. In one embodiment, the antibody comprises a heavy chain variable region (VH), wherein the VH comprises the sequence as set forth in any one of SEQ ID NO: 111. In one embodiment, the antibody comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in any one of SEQ ID NO: 112. In one embodiment, the antibody comprises a light chain variable region (VL), wherein the VL comprises the sequence as set forth in any one of SEQ ID NO: 112.

The antibody binding to PD-1 may comprise a heavy chain variable region (VH) and a light chain variable region (VL), wherein the VH comprises or has the sequence as set forth in SEQ ID NO: 111 and the VL comprises or has the sequence as set forth in SEQ ID NO: 112, or respective variants of these sequences. Another example of an antibody binding to PD-1 may comprise a VH comprising or having the sequence as set forth in SEQ ID NO: 111, or a variant thereof, and a VL comprising or having the sequence as set forth in SEQ ID NO: 112, or a variant thereof. A specific, but not limiting example of such an antibody is MAB-19-0618. The antibody MAB-19-0618 has been derived from MAB-19-0202. Also encompassed by the present disclosure are variants of the said heavy chain variable regions (VH) and the said light chain variable regions (VL) and the respective combinations of these variant VHs and VLs.

The antibody binding to PD-1 may comprises a heavy chain and a light chain, which heavy chain comprises a heavy chain constant region comprising or having the sequence as set forth in SEQ ID NO: 93 or 90 and a heavy chain variable region (VH) comprising or having the sequence as set forth in SEQ ID NO: 111, and which light chain comprises a light chain constant region comprising or having the sequence as set forth in SEQ ID NO: 97 and a light chain variable region (VL) comprising or having the sequence as set forth in SEQ ID NO: 112.

The antibody binding to PD-1 may comprises a heavy chain and a light chain, which heavy chain comprises a heavy chain constant region comprising or having the sequence as set forth in SEQ ID NO: 93 or 90 and a heavy chain variable region (VH) comprising the CDR1, CDR2 and CDR3 sequences of the sequence as set forth in SEQ ID NO: 111, and which light chain comprises a light chain constant region comprising or having the sequence as set forth in SEQ ID NO: 97 and a light chain variable region comprising the CDR1, CDR2 and CDR3 sequences of the sequence as set forth in SEQ ID NO: 112. For example, the CDR1, CDR2 and CDR3 sequences are as specified herein.

The antibody binding to PD-1 may be a monoclonal, chimeric or a monoclonal, humanized antibody or a fragment of such an antibody. The antibodies can be whole antibodies or antigen-binding fragments thereof including, for example, bispecific antibodies.

In the antibody binding to PD-lone or more, preferably both heavy chain constant regions may have been modified so that binding of C1q to said antibody is reduced compared to a wild-type antibody, preferably reduced by at least 70%, at least 80%, at least 90%, at least 95%, at least 97%, or 100%. In one embodiment, the C1q binding can be determined by ELISA.

By “wild type” or “WT” or “native” herein is meant an amino acid sequence that is found in nature, including allelic variations. A wild type amino acid sequence, peptide or protein has an amino acid sequence that has not been intentionally modified.

In the antibody binding to PD-1, one or more, preferably both heavy chain constant regions may have been modified so that binding to one or more of the IgG Fc-gamma receptors to the antibody is reduced compared to a wild-type antibody, preferably by at least 70%, at least 80%, at least 90%, at least 95%, at least 97% or 100%. In one embodiment, the one or more IgG Fc-gamma receptors are selected from at least one of Fc-gamma RI, Fc-gamma RII, and Fc-gamma RIII. In one embodiment, the IgG Fc-gamma receptor is Fc-gamma RI.

In one embodiment, the antibody binding to PD-1 is not capable of inducing Fc-gamma RI-mediated effector functions or wherein the induced Fc-gamma RI-mediated effector functions are reduced compared to a wild-type antibody, preferably by at least 70%, at least 80%, at least 90%, at least 95%, at least 97% or 100%.

In one embodiment, the antibody binding to PD-1 is not capable of inducing at least one of complement dependent cytotoxicity (CDC) mediated lysis, antibody dependent cellular cytotoxicity (ADCC) mediated lysis, apoptosis, homotypic adhesion and/or phagocytosis or wherein at least one of complement dependent cytotoxicity (CDC) mediated lysis, antibody dependent cellular cytotoxicity (ADCC) mediated lysis, apoptosis, homotypic adhesion and/or phagocytosis is induced in a reduced extent, preferably reduced by at least 70%, at least 80%, at least 90%, at least 95%, at least 97% or 100%.

Antibody-dependent cell-mediated cytotoxicity is also referred to as “ADCC” herein. ADCC describes the cell-killing ability of effector cells as described herein, in particular lymphocytes, which preferably requires the target cell being marked by an antibody.

ADCC preferably occurs when antibodies bind to antigens on tumor cells and the antibody Fe domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that leads to antigen presentation and the induction of tumor-directed T-cell responses. Preferably, in vivo induction of ADCC will lead to tumor-directed T-cell responses and host-derived antibody responses.

Complement-dependent cytotoxicity is also referred to as “CDC” herein. CDC is another cell-killing method that can be directed by antibodies. IgM is the most effective isotype for complement activation.

IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement C1).

Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a.

Preferably, the complement cascade ends in the formation of a membrane attack complex, which creates pores in the cell membrane that facilitate free passage of water and solutes into and out of the cell and may lead to apoptosis.

In one embodiment, the antibody binding to PD-1 has reduced or depleted effector functions. In one embodiment, the antibody does not mediate ADCC or CDC or both.

In one embodiment, one or more, preferably both heavy chain constant regions of the antibody binding to PD-1 have been modified so that binding of neonatal Fc receptor (FcRn) to the antibody is unaffected, as compared to a wild-type antibody.

In one embodiment, the PD-1 to which the antibody is able to bind is human PD-1. In one embodiment, the PD-1 has or comprises the amino acid sequence as set forth in SEQ ID NO: 113 or SEQ ID NO: 114, or the amino acid sequence of PD-1 has at least 70%, at least 75%, at least 80%, at least 85%, at least 90%, at least 95%, at least 97%, at least 99%, or 100% identity to the amino acid sequence as set forth in SEQ ID NO: 113 or SEQ ID NO: 114, or is an immunogenic fragment thereof. In one embodiment, the antibody has the ability to bind to a native epitope of PD-1 present on the surface of living cells.

In one embodiment, the antibody binding to PD-1 comprises a heavy chain constant region, wherein the heavy chain constant region comprises an aromatic or non-polar amino acid at the position corresponding to position 234 in a human IgG1 heavy chain according to EU numbering and an amino acid other than glycine at the position corresponding to position 236 in a human IgG1 heavy chain according to EU numbering.

The term “amino acid corresponding to position . . . ” and similar expressions as used herein refer to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present disclosure.

With reference to, e.g., the amino acid sequence according to SEQ ID NO. 93 of the sequence listing of the present disclosure the amino acid positions corresponding to positions 234 to 236 in a human IgG1 heavy chain according to EU numbering are the amino acid positions 117 to 119 of SEQ ID NO. 93, with F being positioned at position 117 (corresponding to positions 234 in a human IgG1 heavy chain according to EU numbering), E being positioned at position 118 (corresponding to positions 235 in a human IgG1 heavy chain according to EU numbering) and R being positioned at position 119 (corresponding to positions 236 in a human IgG1 heavy chain according to EU numbering). In the sequence as shown below, the FER amino acid sequence is underlined and shown in bold letters.

ASTKGPSVFPLAPSSKSTSGGTAALGCLVKDYFPEPVTVSWNSGALTSGVHTFPAVLQSS 60
GLYSLSSVVTVPSSSLGTQTYICNVNHKPSNTKVDKRVEPKSCDKTHTCPPCPAPEFERG 120
PSVFLFPPKPKDTLMISRTPEVTCVVVDVSHEDPEVKENWYVDGVEVHNAKTKPREEQYN 180
STYRVVSVLTVLHQDWLNGKEYKCKVSNKALPAPIEKTISKAKGQPREPQVYTLPPSREE 240
MTKNQVSLTCLVKGFYPSDIAVEWESNGQPENNYKTTPPVLDSDGSFFLYSKLTVDKSRW 300
QQGNVFSCSVMHEALHNHYTQKSLSLSPG 329

Unless otherwise indicated herein or otherwise clearly contradicted by the context, all references to amino acid positions in antibody heavy chain constant regions throughout this disclosure refer to the positions corresponding to the respective positions in a human IgG1 heavy chain according to EU numbering as set forth in Kabat (described in Kabat, E. A. et al., Sequences of proteins of immunological interest. 5th Edition—US Department of Health and Human Services, NIH publication No. 91-3242, pp 662,680,689 (1991))..

In one embodiment, the antibody binding to PD-1 comprises a heavy chain constant region which has a reduced or depleted Fc-mediated effector function or which induces Fc-mediated effector function to a lesser extent compared to another antibody comprising the same antigen binding regions and heavy chain constant regions (CHs) comprising human IgG1 hinge, CH2 and CH3 regions.

In one particular embodiment, said heavy chain constant region (CHs) in the antibody binding to PD-1 are modified so that the antibody induces Fc-mediated effector function to a lesser extent compared to an antibody which is identical except for comprising non-modified heavy chain constant regions (CHs).

The term “Fc-mediated effector function” as used herein refers to such functions in particular being selected from the list of IgG Fc receptor (FcgammaR, FcγR) binding, C1q binding, ADCC, CDC and any combinations thereof.

In the context of the present disclosure, the term “has a reduced or depleted Fc-mediated effector function” used in relation to an antibody, including a multispecific antibody, means that the antibody cause an overall decrease of Fc-mediated effector functions, such function in particular being selected from the list of IgG Fc receptor (FcgammaR, FcγR) binding, C1q binding, ADCC or CDC, preferably of 5% or greater, 10% or greater, 20% or greater, more preferably of 50% or greater, and most preferably of 75% or greater, in the level compared to a human IgG1 antibody comprising (i) the same CDR sequences, in particular comprising the same first and second antigen-binding regions, as said antibody and (ii) two heavy chains comprising human IgG1 hinge, CH2 and CH3 regions. A “depleted Fc-mediated effector function” or similar phrases includes a complete or essentially complete inhibition, i.e., a reduction to zero or essentially to zero.

In the context of the present disclosure, the term “induce Fc-mediated effector function to a lesser extent” used in relation to an antibody, including a multispecific antibody, means that the antibody induces Fc-mediated effector functions, such function in particular being selected from the list of IgG Fc receptor (FcgammaR, FcγR) binding, C1q binding, ADCC or CDC, to a lesser extent compared to a human IgG1 antibody comprising (i) the same CDR sequences, in particular comprising the same first and second antigen-binding regions, as said antibody and (ii) two heavy chains comprising human IgG1 hinge, CH2 and CH3 regions.

The Fc-mediated effector function may be determined by measuring binding of the binding agent to Fcγ receptors, binding to C1q, or induction of Fc-mediated cross-linking of Fcγ receptors. In particular, the Fc-mediated effector function may be determined by measuring binding of the binding agent to C1q and/or IgG FC-gamma RI.

In one embodiment relating to use of the antibody binding to PD-1, the amino acid at the position corresponding to position 236 in a human IgG1 heavy chain according to EU numbering is a basic amino acid.

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present disclosure, amino acids may be classified based on structure and chemical characteristics.

In the present disclosure, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala: A: alanine; Asp: D: aspartic acid; Glu: E: glutamic acid; Phe: F: phenylalanine; Gly: G: glycine; His: H: histidine; Ile: I: isoleucine; Lys: K: lysine; Leu: L: leucine; Met: M methionine; Asn: N: asparagine; Pro: P: proline; Gln: Q: glutamine; Arg: R: arginine; Ser: S serine; Thr: T: threonine; Val: V: valine; Trp: W: tryptophan; Tyr: Y: tyrosine; Cys: C: cysteine.

Naturally occurring amino acids may also be generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

In one embodiment relating to use of an antibody binding to PD-1, the basic amino acid at the position corresponding to position 236 in a human IgG1 heavy chain according to EU numbering is selected from the group consisting of lysine, arginine and histidine. In one embodiment, the basic amino acid at the position corresponding to position 236 in a human IgG1 heavy chain according to EU numbering is arginine (G236R). Such an amino acid subsitition is also referred to herein as G236R. The term “G236R” indicates that at position 236 in a human IgG1 heavy chain according to EU numbering the amino acid glycine (G) is substituted by arginine (R). Within the present disclosure similar terms are used for other amino acid positions and amino acids. Unless indicated to the contrary the referenced amino acid position in these terms is the amino acid position in a human IgG1 heavy chain according to EU numbering.

In one embodiment relating to use of an antibody binding to PD-1, the amino acid at the position corresponding to position 234 in a human IgG1 heavy chain according to EU numbering is an aromatic amino acid. In one embodiment, the aromatic amino acid at this position is selected from the group consisting of phenylalanine, tryptophan and tyrosine.

In one embodiment relating to use of an antibody binding to PD-1, the amino acid at the position corresponding to position 234 in a human IgG1 heavy chain according to EU numbering is a non-polar amino acid. In one embodiment, the non-polar amino acid at this position is selected from the group consisting of alanine, valine, leucine, isoleucine, proline, phenylalanine, methionine and tryptophan. In one embodiment, the non-polar amino acid at this position is selected from the group consisting of isoleucine, proline, phenylalanine, methionine and tryptophan.

In one embodiment relating to use of an antibody binding to PD-1, the amino acid at the position corresponding to position 234 in a human IgG1 heavy chain according to EU numbering is phenylalanine (L234F).

Exemplary combinations of possible amino acids at the positions corresponding to positions 234 and 236 in a human IgG1 heavy chain according to EU numbering are set forth in the table below:

TABLE 5

| Amino acid position 234 | Amino acid position 236 |
|---|---|
| Phenylalanine (F) | Arginine (R) |
| Tryptophan (W) | Arginine (R) |
| Tyrosine (Y) | Arginine (R) |
| Alanine (A) | Arginine (R) |
| Valine (V) | Arginine (R) |
| Leucine (L) | Arginine (R) |
| Isoleucine (I) | Arginine (R) |
| Proline (P) | Arginine (R) |
| Methionine (M) | Arginine (R) |
| Phenylalanine (F) | Lysine (K) |
| Tryptophan (W) | Lysine (K) |
| Tyrosine (Y) | Lysine (K) |
| Alanine (A) | Lysine (K) |
| Valine (V) | Lysine (K) |
| Leucine (L) | Lysine (K) |
| Isoleucine (I) | Lysine (K) |
| Proline (P) | Lysine (K) |
| Methionine (M) | Lysine (K) |
| Phenylalanine (F) | Histidine (H) |
| Tryptophan (W) | Histidine (H) |
| Tyrosine (Y) | Histidine (H) |
| Alanine (A) | Histidine (H) |
| Valine (V) | Histidine (H) |
| Leucine (L) | Histidine (H) |
| Isoleucine (I) | Histidine (H) |
| Proline (P) | Histidine (H) |
| Methionine (M) | Histidine (H) |

For example, at the positions corresponding to the positions 234 and 236 in a human IgG1 heavy chain according to EU numbering, in particular the following amino acids may be present in the heavy chain constant region of the antibody binding to PD-1:234F/236R, 234W/236R, 234Y/236R, 234A/236R, 234L/236R, 234F/236K, 234W/236K, 234Y/236K, 234A/236K, 234L/236K, 234F/236H, 234W/236H, 234Y/236H, 234A/236H, or 234L/236H.

The aforementioned amino acids or amino acids substitutions at positions 234 and 236 may be present only in one heavy chain of the antibody binding to PD-1 or in both heavy chains of the antibody binding to PD-1. The respective amino acids present in first and the second heavy chain of the antibody may be selected independently from each other.

For example, at least one heavy chain of the antibody binding to PD-1 can comprise the following sequence (SEQ ID NO: 93):

GLYSLSSVVTVPSSSLGTQTYICNVNHKPSNTKVDKRVEPKSCDKTHTCPPCPAPEFLRG 120
PSVFLFPPKPKDTLMISRTPEVTCVVVDVSHEDPEVKENWYVDGVEVHNAKTKPREEQYN 180
STYRVVSVLTVLHQDWLNGKEYKCKVSNKALPAPIEKTISKAKGQPREPQVYTLPPSREE 240
MTKNQVSLTCLVKGFYPSDIAVEWESNGQPENNYKTTPPVLDSDGSFFLYSKLTVDKSRW 300
QQGNVFSCSVMHEALHNHYTQKSLSLSPG 329

In one embodiment relating to the antibody binding to PD-1, the said heavy chain in which the amino acids at the position corresponding to positions 234 and 236 in a human IgG1 heavy chain according to EU numbering are as specified above, furthermore the amino acid at the position corresponding to position 235 in a human IgG1 heavy chain according to EU numbering is an acidic amino acid. In one embodiment, the acidic amino acid at this position is selected from aspartate or glutamate. In one embodiment, the amino acid at the position corresponding to position 235 in a human IgG1 heavy chain according to EU numbering is glutamate (L235E).

In one embodiment relating to the antibody binding to PD-i, in the heavy chain constant region the amino acids at the position corresponding to positions 234, 235 and 236 in a human IgG1 heavy chain according to EU numbering are a non-polar or aromatic amino acid at position 234, an acidic amino acid at position 235 and a basic amino acid at position 236.

Exemplary combinations of possible amino acids at the positions corresponding to positions 234, 235 and 236 in a human IgG1 heavy chain according to EU numbering are set forth in the table below:

TABLE 6

| Amino acid | | Amino acid |
|---|---|---|
| position 234 | Amino acid position 235 | position 236 |
| Phenylalanine (F) | Asparatate (D) or Glutamate (E) | Arginine (R) |
| Tryptophan (W) | Asparatate (D) or Glutamate (E) | Arginine (R) |
| Tyrosine (Y) | Asparatate (D) or Glutamate (E) | Arginine (R) |
| Alanine (A) | Asparatate (D) or Glutamate (E) | Arginine (R) |
| Valine (V) | Asparatate (D) or Glutamate (E) | Arginine (R) |
| Leucine (L) | Asparatate (D) or Glutamate (E) | Arginine (R) |
| Isoleucine (I) | Asparatate (D) or Glutamate (E) | Arginine (R) |
| Proline (P) | Asparatate (D) or Glutamate (E) | Arginine (R) |
| Methionine (M) | Asparatate (D) or Glutamate (E) | Arginine (R) |
| Phenylalanine (F) | Asparatate (D) or Glutamate (E) | Lysine (K) |
| Tryptophan (W) | Asparatate (D) or Glutamate (E) | Lysine (K) |
| Tyrosine (Y) | Asparatate (D) or Glutamate (E) | Lysine (K) |
| Alanine (A) | Asparatate (D) or Glutamate (E) | Lysine (K) |
| Valine (V) | Asparatate (D) or Glutamate (E) | Lysine (K) |
| Leucine (L) | Asparatate (D) or Glutamate (E) | Lysine (K) |
| Isoleucine (I) | Asparatate (D) or Glutamate (E) | Lysine (K) |
| Proline (P) | Asparatate (D) or Glutamate (E) | Lysine (K) |
| Methionine (M) | Asparatate (D) or Glutamate (E) | Lysine (K) |
| Phenylalanine (F) | Asparatate (D) or Glutamate (E) | Histidine (H) |
| Tryptophan (W) | Asparatate (D) or Glutamate (E) | Histidine (H) |
| Tyrosine (Y) | Asparatate (D) or Glutamate (E) | Histidine (H) |
| Alanine (A) | Asparatate (D) or Glutamate (E) | Histidine (H) |
| Valine (V) | Asparatate (D) or Glutamate (E) | Histidine (H) |
| Leucine (L) | Asparatate (D) or Glutamate (E) | Histidine (H) |
| Isoleucine (I) | Asparatate (D) or Glutamate (E) | Histidine (H) |
| Proline (P) | Asparatate (D) or Glutamate (E) | Histidine (H) |
| Methionine (M) | Asparatate (D) or Glutamate (E) | Histidine (H) |

For example, at the positions corresponding to the positions 234, 235 and 236 in a human IgG1 heavy chain according to EU numbering, in particular the following amino acids may be present in the heavy chain constant region of the antibody binding to PD-i: 234F/235E/236R, 234W/235E/236R, 234Y/235E/236R, 234A/235E/236R, 234L/235E/236R, 234F/235D/236R, 234W/235D/236R, 234Y/235D/236R, 234A/235D/236R, 234L/235D/236R, 234F/235L/236R, 234W/235L/236R, 234Y/235L/236R, 234A/235L/236R, 234L/235L/236R, 234F/235A/236R, 234W/235A/236R, 234Y/235A/236R, 234A/235A/236R, 234L/235A/236R, 234F/235E/236K, 234W/235E/236K, 234Y/235E/236K, 234A/235E/236K, 234L/235E/236K, 234F/235D/236K, 234W/235D/236K, 234Y/235D/236K, 234A/235D/236K, 234L/235D/236K, 234F/235L/236K, 234W/235L/236K, 234Y/235L/236K, 234A/235L/236K, 234L/235L/236K, 234F/235A/236K, 234W/235A/236K, 234Y/235A/236K, 234A/235A/236K, 234L/235A/236K, 234F/235E/236H, 234W/235E/236H, 234Y/235E/236H, 234A/235E/236H, 234L/235E/236H, 234F/235D/236H, 234W/235D/236H, 234Y/235D/236H, 234A/235D/236H, 234L/235D/236H, 234F/235L/236H, 234W/235L/236H, 234Y/235L/236H, 234A/235L/236H, 234L/235L/236H, 234F/235A/236H, 234W/235A/236H, 234Y/235A/236H, 234A/235A/236H, or 234L/235A/236H.

The aforementioned amino acids or amino acids substitutions at positions 234, 235 and 236 may be present only in one heavy chain of the antibody or in both heavy chains of the antibody. The respective amino acids present in first and the second heavy chain of the antibody may be selected independently from each other.

For example, at least one heavy chain of the antibody binding to PD-1 can comprise the following sequence (SEQ ID NO: 90 or 93):

ASTKGPSVFPLAPSSKSTSGGTAALGCLVKDYFPEPVTVSWNSGALTSGVHTFPAVLQSS 60
GLYSLSSVVTVPSSSLGTQTYICNVNHKPSNTKVDKRVEPKSCDKTHTCPPCPAPEFERG 120
PSVFLFPPKPKDTLMISRTPEVTCVVVDVSHEDPEVKENWYVDGVEVHNAKTKPREEQYN 180
STYRVVSVLTVLHQDWLNGKEYKCKVSNKALPAPIEKTISKAKGQPREPQVYTLPPSREE 240
MTKNQVSLTCLVKGFYPSDIAVEWESNGQPENNYKTTPPVLDSDGSFFLYSKLTVDKSRW 300
QQGNVFSCSVMHEALHNHYTQKSLSLSPG 329

Any permutations and combinations of all described amino acid substitutions at positions 234, 236 and 235, if applicable, in this application, e.g., as shown in Tables 5 and 6, should be considered disclosed by the description of the present application unless the context indicates otherwise. For example, in one embodiment of the antibody the first heavy chain comprises the amino acids FER at the position corresponding to positions 234 to 236 in a human IgG1 heavy chain according to EU numbering or the first heavy chain comprises or consists essentially of or consists of an amino acid sequence set forth in SEQ ID NO: 93, and the second heavy chain of said antibody comprises other amino acids, e.g., the amino acids AAG or LLG at the positions corresponding to positions 234 to 236 in a human IgG1 heavy chain according to EU numbering or comprises or the second heavy chain of said antibody comprises or consists essentially of or consists of an amino acid sequence set forth in SEQ ID NO: 92 or 98. In another embodiment of the antibody, the first and the second heavy chains comprise the same amino acids at the position corresponding to positions 234 to 236 in a human IgG1 heavy chain according to EU numbering, i.e., the same aromatic or non-polar amino acid at the position corresponding to position 234 in a human IgG1 heavy chain according to EU numbering, e.g. F, and the same amino acid other than glycine at the position corresponding to position 236 in a human IgG1 heavy chain according to EU numbering, e.g., R, such as the specific combination of FER or FLR.

In one embodiment, the antibody binding to PD-1 comprises at least one or two heavy chain constant regions,wherein the amino acid corresponding to position 234 is phenylalanine, the amino acid corresponding to position 235 is glutamate, and the amino acid corresponding to position 236 is arginine (L234F/L235E/G236R=FER).

In one embodiment, the antibody binding to PD-1 comprises one or more a heavy chain constant region (CH) comprising a sequence having at least 70%, at least 75%, at least 80%, at least 85%, at least 90%, at least 95%, at least 97%, at least 99%, or 100% identity to the amino acid sequence of the heavy chain constant region sequence as set forth in SEQ ID NO: 93.

In one embodiment, the antibody binding to PD-1 comprises one or more, e.g., two heavy chain constant region (CH), wherein the heavy chain constant region comprises the sequence as set forth in SEQ ID NO: 93.

The antibody is preferably of the IgG1 isotype.

As used herein, the term “isotype” refers to the immunoglobulin class that is encoded by heavy chain constant region genes. When the IgG1 isotype, is mentioned herein, the term is not limited to a specific isotype sequence, e.g., a particular IgG1 sequence, but is used to indicate that the antibody is closer in sequence to that isotype, e.g. IgG1, than to other isotypes. Thus, e.g., an IgG1 antibody disclosed herein may be a sequence variant of a naturally-occurring IgG1 antibody, including variations in the constant regions.

IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

For example or in an embodiment, an antibody, preferably a monoclonal antibody, used according to the present invention the present invention is a IgG1, κ isotype or λ isotype, preferably comprising human IgG1/c or human IgG1/5 constant parts, or the antibody, preferably the monoclonal antibody, is derived from a IgG1, λ (lambda) or IgG1, κ (kappa) antibody, preferably from a human IgG1, λ (lambda) or a human IgG1, κ (kappa) antibody.

In one embodiment, the antibody binding to PD-1 comprises a light chain having a light chain constant region (LC) comprising a sequence having at least 70%, at least 75%, at least 80%, at least 85%, at least 90%, at least 95%, at least 97%, at least 99%, or 100% identity to the amino acid sequence of the LC sequence as set forth in SEQ ID NO: 97. In one embodiment, the antibody comprises a light chain having a light chain constant region (LC) comprising the sequence as set forth in SEQ ID NO: 97.

In one embodiment of the invention, the antibody binding to PD-1 is a full-length IgG1 antibody, e.g., e.g., IgG1, x. In one embodiment of the invention, the binding agent is a full-length human IgG1 antibody, e.g., IgG1, x.

In one embodiment, the antibody binding to PD-1 can be derivatized, linked to or co-expressed to other binding specificities. In another embodiment, the antibody can be derivatized, linked to or co-expressed with another functional molecule, e.g., another peptide or protein (e.g., a Fab′ fragment). For example, the can be functionally linked (e.g., by chemical coupling, genetic fusion, noncovalent association or otherwise) to one or more other molecular entities, such as another antibody (e.g., to produce a bispecific or a multispecific antibody).

The antibody binding to PD-1 may be a human antibody. The term “human antibody”, as used herein, is intended to include antibodies having variable and constant regions derived from human germline immunoglobulin sequences. The human antibody binding to PD-1 may include amino acid residues not encoded by human germline immunoglobulin sequences (e.g., mutations introduced by random or site-specific mutagenesis in vitro or by somatic mutation in vivo).

The present disclosure includes the use of bispecific and multispecific molecules comprising at least one first binding specificity for PD-1 and a second binding specificity (or further binding specifities) for a second target epitope (or for further target epitopes).

In one embodiment the first antigen-binding region of the multispecific antibody binding to PD-1 comprises the heavy chain variable region (VH) and/or the light chain variable region (VL) as set forth herein.

In one embodiment relating to the use of a multispecific antibody binding to PD-1, the antibody comprises first and second binding arms derived from full-length antibodies, such as from full-length IgG1, λ (lambda) or IgG1, κ (kappa) antibodies as mentioned above. In one embodiment, the first and second binding arms are derived from monoclonal antibodies. For example or in a preferred embodiment, the first and/or second binding arm is derived from a IgG1, κ isotype or λ isotype, preferably comprising human IgG1/c or human IgG1/5 constant parts.

The said first antigen-binding region binding to PD-1 of the multispecific or bispecific antibody used according to the present invention may comprise heavy and light chain variable regions of an antibody which competes for PD-1 binding with PD-L1 and/or PD-L2. In one embodiment relating to the use of the multispecific or bispecific antibody, the first antigen-binding region binding to PD-1 comprises the heavy chain variable region (VH) and/or the light chain variable region (VL) as set forth herein.

As used herein, the term “effector cell” refers to an immune cell which is involved in the effector phase of an immune response, as opposed to the cognitive and activation phases of an immune response.

Exemplary immune cells include cells of myeloid or lymphoid origin, e.g, lymphocytes (e.g., B cells and T cells including cytolytic T cells (CTLs), killer cells, natural killer cells, macrophages, monocytes, eosinophils, neutrophils, polymorphonuclear cells, granulocytes, mast cells, and basophils.

“Target cell” shall mean any undesirable cell in a subject (e.g., a human or animal) that can be targeted by an antibody. In preferred embodiments, the target cell is a tumor cell.

### Subject and Tumor or Cancer to be Treated

The subject to be treated according to the present disclosure is preferably a human subject.

In one preferred embodiment, the tumor or cancer to be treated is a solid tumor or cancer. The tumor or cancer may be a metastatic tumor or cancer.

Preferably, the tumor or cancer may be selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, endometrial cancer, prostate cancer, penile cancer, cervical cancer, Hodgkin's lymphoma, non-Hodgkin's lymphoma, Merkel cell carcinoma and mesothelioma. More preferably, the tumor or cancer is selected from the group consisting of melanoma, lung cancer, colorectal cancer, pancreatic cancer, and head and neck cancer.

In particular embodiments, the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)), squamous cell carcinoma of the head and neck (SCCHN) (e.g. cancer of the oral cavity, pharynx or larynx) and cervical cancer.

Preferably, the tumor is a PD-L1 positive tumor. In certain embodiments, it is preferred that PD-L1 is expressed in >1% of the cancer cells or tumor cells. The expression of PD-L1 may be determined using techniques known to the person skilled in the art and may e.g. be assessed by immunohistochemistry (IHC).

The tumor or cancer may in particular be a lung cancer. The lung cancer may be a non-small cell lung cancer (NSCLC), such as a squamous or a non-squamous NSCLC. Lung cancer is the second most common malignancy with an estimated age-standardized incidence rate of 22.4 per 100,000 and a leading cause of cancer death for both men and women (Kantar, 2021). Worldwide, approximately 2,206,771 new cases of lung cancer and 1,796,144 deaths are estimated in 2020 (GLOBOCAN, 2020). Non-small-cell lung cancer (NSCLC) accounts for 85% to 90% of all cases, with a 5-year survival rate of approximately 18% across all stages of the disease, and only 3.5% for metastatic disease (Jemal et al., 2011) (Kantar, 2021; SEER, 2018). In the 1L setting, treatment typically consists of platinum-based chemotherapy in combination with immunotherapy, or a targeted therapy, depending on molecular and biomarker analysis and the histology of the tumor (NCCN, 2021d). More recently, the advent of PD-1 and programmed death ligand 1 (PD-L1) inhibitors have improved outcomes for patients without driver mutations (approximately 62% of the non-squamous population and 77% of the squamous population (Kantar, 2021)). More treatment alternatives are needed for patients whose tumors do not harbor certain oncogenic mutations or do not express the biomarker for checkpoint inhibitor (CPI) options. Novel combinations with complementary approaches to enhance response may further address the unmet need in this population. For patients in the 2L setting, SOC is limited to platinum-based chemotherapy, a CPI monotherapy or docetaxel with or without ramucirumab depending on the previous therapy received. For patients in the third-line (3L) setting, chemotherapy monotherapy is the standard. Novel therapies are needed to limit toxicity and potentially enhance efficacy in this population (NCCN, 2021d).

In one embodiment, wherein the tumor or cancer is lung cancer, this tumor or cancer is a non-small cell lung cancer (NSCLC), such as a squamous or non-squamous NSCLC. The tumor or cancer may in particular be a metastatic cancer, such as metastatic NSCLC.

In one embodiment, wherein the tumor or cancer is lung cancer, in particular NSCLC, the tumor or cancer does not have an epidermal growth factor (EGFR)-sensitizing mutation and/or anaplastic lymphoma (ALK) translocation/ROS1 rearrangement. EGFR-sensitizing mutations are those mutations that are amenable to treatment with an approved tyrosine kinase inhibitor (TKI).

In one embodiment, wherein the tumor or cancer is lung cancer, in particular NSCLC, the tumor or cancer comprises cancer cells and PD-L1 is expressed in >1% of the cancer cells. Such expression may be determined by any means and method known to the skilled person, such as by immunohistochemistry (IHC), such as determined by a local SOC testing (preferably an FDA-approved test) or at a central laboratory.

In one embodiment, the subject has not received prior systemic treatment of metastatic disease i.e., the subject has not received any systemic treatment of metastatic disease prior to receiving treatment according to the invention. According to this embodiment, the tumor or cancer is preferably a lung cancer, such as NSCLC.

In one embodiment, the subject has not received prior treatment with a checkpoint inhibitor/an immune checkpoint (ICP) inhibitor, i.e., before the treatment according to the first aspect, the subject has not received treatment with ICP inhibitor. In further embodiments, the subject has not received prior treatment with a PD-1 inhibitor or a PD-L1 inhibitor, such as anti-PD-1 antibody or an anti-PD-L1 antibody. In these embodiments the tumor or cancer is preferably a lung cancer, such as NSCLC.

In a further embodiment, the subject has not received prior treatment with a 4-1BB (CD137) targeted agent, with an antitumor vaccine, or with autologous cell immunotherapy. In one embodiment, the subject has not received prior treatment with an anti-4-1BB (CD137) antibody. In these embodiments the tumor or cancer is preferably a lung cancer, such as NSCLC.

In other embodiments the tumor or cancer has relapsed and/or is refractory after treatment, such as systemic treatment with a checkpoint inhibitor. The subject may have received at least one prior line of systemic therapy, such as systemic therapy comprising a PD-1 inhibitor or a PD-L1 inhibitor, such as an anti-PD-1 antibody or an anti-PD-L1 antibody. The cancer or tumor may in particular have relapsed and/or become refractory, or the subject may have progressed after treatment with a PD-1 inhibitor or a PD-L1 inhibitor, such as an anti PD-1 antibody or an anti-PD-L1 antibody, the PD-1 inhibitor or PD-L1 inhibitor being administered as monotherapy or as part of a combination therapy.

In particular embodiments the treatment according to the invention is provided to a subject having received prior treatment; e.g. as defined above, wherein the last prior treatment was with a PD1 inhibitor or PD-L1 inhibitor, such as an anti PD-1 antibody or an anti-PD-L1 antibody, the PD-1 inhibitor or PD-L1 inhibitor being administered as monotherapy or as part of a combination therapy. The last prior treatment may be with a PD1 inhibitor or PD-L1 inhibitor defined above.

Preferably, the therapy according to the invention is provided to a subject when the time from progression of that subject on last treatment with a PD1 inhibitor or PD-L1 inhibitor, such as an anti PD-1 antibody or an anti-PD-L1 antibody is 8 months or less, such as 7 months or less, 6 months or less, 5 months or less, 4 months or less, 3 months or less, 2 months or less, 1 month or less, 3 weeks or less or such as 2 weeks or less.

By analogy, it may be preferred to offer therapy according to the present invention to a subjects when the time from last dosing of a PD1 inhibitor or PD-L1 inhibitor, such as an anti PD-1 antibody or an anti-PD-L1 antibody as part of last prior treatment is 8 months or less, such as 7 months or less, 6 months or less, 5 months or less, 4 months or less, 3 months or less, 2 months or less, 1 month or less, 3 weeks or less or such as 2 weeks or less.

In further embodiments the cancer or tumor has relapsed and/or is refractory, or the subject has progressed during or after
i) platinum doublet chemotherapy following treatment with an anti-PD-1 antibody or an anti-PD-L1 antibody, or
ii) treatment with an anti-PD-1 antibody or an anti-PD-L1 antibody following platinum doublet chemotherapy.

Also, in these embodiments the tumor or cancer is preferably a lung cancer, such as NSCLC.

The subject receiving treatment according to the invention may in particular be a subject who has not received prior treatment with a taxane chemotherapeutic; e.g., docetaxel or paclitaxel, such as prior treatment of NSCLC with a taxane chemotherapeutic e.g., docetaxel.

### Treatment Regimen

The binding agent and the PD-1 inhibitor can be administered by any suitable way, such as intravenously, intraarterially, subcutaneously, intradermally, intramuscularly, intranodally, or intratumorally.

In one embodiment of the first aspect, the binding agent is administered to the subject by systemic administration. Preferably, the binding agent is administered to the subject by intravenous injection or infusion. In one embodiment, the binding agent is administered in at least one treatment cycle.

In one embodiment, the PD-1 inhibitor is in particular administered to the subject by systemic administration. Preferably, the PD-1 inhibitor is administered to the subject by intravenous injection or infusion. In one embodiment, the PD-1 inhibitor is administered in at least one treatment cycle.

In one embodiment, the binding agent and the PD-1 inhibitor are in particular administered to the subject by systemic administration. Preferably, the binding agent and the PD-1 inhibitor are administered to the subject by intravenous injection or infusion. In one embodiment, the binding agent and the PD-1 inhibitor are administered in at least one treatment cycle.

In one embodiment, each treatment cycle is about two weeks (14 days), three weeks (21 days) or four weeks (28 days), five weeks (35 days) or 6 weeks (48 days). In preferred embodiments each treatment cycle is three weeks (21 days). In other preferred embodiments, each treatment cycle is 6 weeks (48 days).

In particular embodiments, one dose of the binding agent and one dose of the PD-1 inhibitor are administered or infused every second week (1Q2W), every third week (1Q3W) or every fourth week (1Q4W), every fifth week (1Q5W), preferably every third week (1Q3W). In other embodiments, one dose of the binding agent and one dose of the PD-1 inhibitor are administered every six weeks (1Q6W). The amount of binding agent and the amount of PD-1 inhibitor is preferably as defined above.

In some embodiments, one dose or each dose is administered or infused on day 1 of each treatment 30 cycle. For example, one dose of the binding agent and one dose of the PD-1 inhibitor may be administered on day 1 of each treatment cycle.

In some embodiments a 100 mg dose of the binding agent and a 200 mg dose of the PD-1 inhibitor are administered every three weeks (1Q3W).

In other embodiments a 100 mg dose of the binding agent and a 400 mg dose of the PD-1 inhibitor are administered every six weeks (1Q6W).

In particular embodiments, 100 mg dose of the binding agent, which is acasunlimab or a biosimilar thereof and a 200 mg dose of the PD-1 inhibitor, which is nivolumab or a biosimilar thereof, are administered every three weeks (1Q3W), such as on day one of each three-week treatment cycle.

In particular embodiments, the tumor or cancer is NSCLC; and a 100 mg dose of the binding agent, which is acasunlimab or a biosimilar thereof and a 200 mg dose of the PD-1 inhibitor, which is nivolumab or a biosimilar thereof, are administered every three weeks (1Q3W), such as on day one of each three-week treatment cycle.

In other embodiments a 100 mg dose of the binding agent, which is acasunlimab or a biosimilar thereof and a 400 mg dose of the PD-1 inhibitor, which is nivolumab or a biosimilar thereof, are administered every six weeks (1Q6W), such as on day one of every six-week treatment cycle.

In still other embodiments, the tumor or cancer is NSCLC; and wherein a 100 mg dose of the binding agent, which is acasunlimab or a biosimilar thereof and a 400 mg dose of the PD-1 inhibitor, which is nivolumab, are administered every six weeks (1Q6W), such as on day one of every six-week treatment cycle.

The PD-1 inhibitor may be administered first, followed by the binding agent. Alternatively, the binding agent is administered first, followed by the PD-1 inhibitor.

Each dose may be administered or infused over a minimum of 30 minutes, such as over a minimum of 60 minutes, a minimum of 90 minutes, a minimum of 120 minutes or a minimum of 240 minutes.

The binding agent may in particular be administered by using intravenous (IV) infusion over 30 minutes, such as over a minimum of 40 minutes, a minimum of 50 minutes or such as over a minimum of 60 minutes.

The PD-1 inhibitor may in particular be administered as an intravenous infusion over 30 minutes, such as over a minimum of 40 minutes, a minimum of 50 minutes or such as over a minimum of 60 minutes.

The binding agent and the PD-1 inhibitor may be administered simultaneously. In an alternative preferred embodiment, the binding agent and the PD-1 inhibitor are administered separately.

The binding agent and the PD-1 inhibitor may be administered in any suitable form (e.g., naked as such).

However, it is preferred that the binding agent and the PD-1 inhibitor, are administered in the form of any suitable pharmaceutical composition as described herein. In one embodiment, at least the binding agent and the PD-1 inhibitor are administered in the form of separate pharmaceutical compositions (i.e., one pharmaceutical composition for the binding agent and one pharmaceutical composition for the PD-1 inhibitor), preferably the binding agent and the PD-1 inhibitor are administered in the form of separate pharmaceutical compositions (i.e., one pharmaceutical composition for the binding agent and one pharmaceutical composition for the PD-1 inhibitor.

A composition or pharmaceutical composition may be formulated with a carrier, excipient and/or diluent as well as any other components suitable for pharmaceutical compositions, including known adjuvants, in accordance with conventional techniques such as those disclosed in Remington: The Science and Practice of Pharmacy, 19^{th} Edition, Gennaro, Ed., Mack Publishing Co., Easton, PA, 1995. The pharmaceutically acceptable carriers or diluents as well as any known adjuvants and excipients should be suitable for the binding agent and/or the PD-1 inhibitor and the chosen mode of administration.

Suitability for carriers and other components of pharmaceutical compositions is determined based on the lack of significant negative impact on the desired biological properties of the chosen compound or pharmaceutical composition (e.g., less than a substantial impact [10% or less relative inhibition, 5% or less relative inhibition, etc.] upon antigen binding).

A composition, in particular the pharmaceutical composition of the binding agent and the pharmaceutical composition of the PD-1 inhibitor may include diluents, fillers, salts, buffers, detergents (e.g., a nonionic detergent, such as Tween-20 or Tween-80), stabilizers (e.g., sugars or protein-free amino acids), preservatives, solubilizers, and/or other materials suitable for inclusion in a pharmaceutical composition.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regards to the intended route of administration and standard pharmaceutical practice.

Pharmaceutically acceptable carriers include any and all suitable solvents, dispersion media, coatings, antibacterial and antifungal agents, isotonicity agents, antioxidants and absorption-delaying agents, and the like that are physiologically compatible with the active compound, in particular a binding agent and the PD-1 inhibitor.

Examples of suitable aqueous and non-aqueous carriers which may be employed in the (pharmaceutical) compositions include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the (pharmaceutical) compositions is contemplated.

The term “excipient” as used herein refers to a substance which may be present in a (pharmaceutical) composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water A (pharmaceutical) composition may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

A (pharmaceutical) composition may also comprise isotonicity agents, such as sugars, polyalcohols, such as mannitol, sorbitol, glycerol or sodium chloride in the composition.

A (pharmaceutical) composition may also contain one or more adjuvants appropriate for the chosen route of administration such as preservatives, wetting agents, emulsifying agents, dispersing agents, preservatives or buffers, which may enhance the shelf life or effectiveness of the composition. The composition as used herein may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

“Pharmaceutically acceptable salts” comprise, for example, acid addition salts which may, for example, be formed by using a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, suitable pharmaceutically acceptable salts may include alkali metal salts (e.g., sodium or potassium salts); alkaline earth metal salts (e.g., calcium or magnesium salts); ammonium (NH₄); and salts formed with suitable organic ligands (e.g., quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, arginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, galactate, galacturonate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isobutyrate, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, phthalate, picrate, pivalate, polygalacturonate, propionate, salicylate, stearate, sulfate, suberate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

In one embodiment, the binding agent, and the PD-1 inhibitor used herein may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except in so far as any conventional media or agent is incompatible with the active compound, use thereof in the compositions is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

In certain embodiments the binding agent for use according to the invention is formulated in a composition or formulation comprising histidine, sucrose and Polysorbate-80, and having a pH from about 5 to about 6, such as from 5 to 6. In particular, the binding agent for use according to the invention may be in a composition or formulation comprising about 20 mM histidine, about 250 mM Sucrose, about 0.02% Polysorbate-80, and having a pH of about 5.5, such as a composition or formulation comprising 20 mM histidine, 250 mM Sucrose, 0.02% Polysorbate-80, and having a pH of 5.5. The formulation may in particular embodiments comprise about 10 to about 30 mg binding agent/mL, such as 10^{×30} mg binding agent/mL, in particular about 20 mg binding agent/mL, such as 20 mg binding agent/mL.

The binding agent for use according to the invention may be provided in a composition as defined above and may then be diluted in 0.9% NaCl (saline) prior to administration.

In a second aspect, the present disclosure provides a kit comprising (i) a binding agent comprising a first binding region binding to CD137 and a second binding region binding to PD-L1, and (ii) a PD-1 inhibitor wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,

then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

The embodiments disclosed herein with respect to the first aspect (in particular regarding the binding agent, and the PD-1 inhibitor also apply to the kit of the second aspect. In one embodiment, the kit comprises at least two containers, wherein one thereof contains the binding agent (as such or in the form of a (pharmaceutical) composition) and the second container contains the PD-1 inhibitor (as such or in the form of a (pharmaceutical) composition).

In a third aspect, the present disclosure provides a kit of the second aspect for use in a method for reducing or preventing progression of a tumor or treating cancer in a subject. The embodiments disclosed herein with respect to the first aspect (in particular regarding the binding agent, the PD-1 inhibitor, the treatment regimen, the specific tumor/cancer, and the subject) and/or the second aspect also apply to the kit for use of the third aspect.

In a fourth aspect, the present disclosure provides a method for reducing or preventing progression of a tumor or treating cancer in a subject, said method comprising administering to said subject a binding agent prior to, simultaneously with, or after administration of a PD-1 inhibitor, wherein the binding agent comprises a first binding region binding to CD137 and a second binding region binding to PD-L1, and

wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,

then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

The embodiments disclosed herein with respect to the first aspect (in particular regarding the binding agent, the PD-1 inhibitor, the treatment regimen, the specific tumor/cancer, and the subject) also apply to the method of the fourth aspect.

In a further aspect, the present disclosure provides a PD-1 inhibitor for use in a method for reducing or preventing progression of a tumor or treating cancer in a subject, said method comprising administering to said subject the PD-1 inhibitor prior to, simultaneously with, or after administration of a binding agent, wherein the binding agent comprises a first binding region binding to CD137 and a second binding region binding to PD-L1, and

wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,

then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

The embodiments disclosed herein with respect to the first aspect (in particular regarding the binding agent, the PD-1 inhibitor, the treatment regimen, the specific tumor/cancer, and the subject) also apply to the PD-1 inhibitor for use of this further aspect.

A further aspect of the invention concerns a binding agent comprising a first binding region binding to CD137 and a second binding region binding to PD-L1 for use in reducing or preventing progression of a tumor or treating cancer in a subject, wherein last prior treatment received by the subject was with a PD1 inhibitor or PD-L1 inhibitor, such as an anti PD-1 antibody or an anti-PD-L1 antibody.

The time from progression on last treatment of the subject with a PD-1 inhibitor or PD-L1 inhibitor, such as an anti PD-1 antibody or an anti-PD-L1 antibody, is preferably 8 months or less, such as 7 months or less, 6 months or less, 5 months or less, 4 months or less, 3 months or less, 2 months or less, 1 month or less, 3 weeks or less or such as 2 weeks or less.

The time from last dosing of a PD-1 inhibitor or PD-L1 inhibitor, such as an anti PD-1 antibody or an anti-PD-L1 antibody, as part of last prior treatment is preferably 8 months or less, such as 7 months or less, 6 months or less, 5 months or less, 4 months or less, 3 months or less, 2 months or less, 1 month or less, 3 weeks or less or such as 2 weeks or less.

It will be understood that the binding agent may have any of the features as defined above in relation to the first aspect of the invention. Likewise, the tumor or cancer and or the subject to which the binding agent is administered may be as defined above. The route and frequency of administration and amounts of binding agent administered may be as defined in relation to the first aspect of the invention above.

Yet a further aspect of the invention provides a method of reducing or preventing progression of a tumor or treating cancer in a subject, comprising a step of administering to said subject a binding agent comprising a first binding region binding to CD137 and a second binding region binding to PD-L1, wherein last prior treatment received by the subject was with a PD1 inhibitor or PD-L1 inhibitor, such as an anti PD-1 antibody or an anti-PD-L1 antibody.

The time from progression on last treatment of the subject with a PD-1 inhibitor or PD-L1 inhibitor, such as an anti PD-1 antibody or an anti-PD-L1 antibody, is preferably 8 months or less, such as 7 months or less, 6 months or less, 5 months or less, 4 months or less, 3 months or less, 2 months or less, 1 month or less, 3 weeks or less or such as 2 weeks or less.

The time from last dosing of a PD-1 inhibitor or PD-L1 inhibitor, such as an anti PD-1 antibody or an anti-PD-L1 antibody, as part of last prior treatment is preferably 8 months or less, such as 7 months or less, 6 months or less, 5 months or less, 4 months or less, 3 months or less, 2 months or less, 1 month or less, 3 weeks or less or such as 2 weeks or less.

It will be understood that the binding agent may have any of the features as defined above in relation to the first aspect of the invention. Likewise, the tumor or cancer and or the subject to which the binding agent is administered may be as defined above. The route and frequency of administration and amounts of binding agent administered may be as defined in relation to the first aspect of the invention above.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Items of the Present Disclosure

1. A binding agent for use in a method for reducing or preventing progression of a tumor or treating cancer in a subject, said method comprising administering to said subject the binding agent prior to, simultaneously with, or after administration of a PD-1 inhibitor, wherein the binding agent comprises a first binding region binding to CD137 and a second binding region binding to PD-L1; and

Wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively, then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

2. The binding agent for use of item 1, wherein PD-L1 is human PD-L1, in particular human PD-L1 comprising the sequence set forth in SEQ ID NO: 40, and/or CD137 is human CD137, in particular human CD137 comprising the sequence set forth in SEQ ID NO: 38.

3. The binding agent for use of any one of items 1 to 3, wherein the PD-1 inhibitor is a PD-1 antibody.

4. The binding agent for use of any one of items 1 to 4, wherein the PD-1 inhibitor is a PD-1 blocking antibody.

5. The binding agent for use of any one of the preceding items, wherein the PD-1 inhibitor is pembrolizumab or a biosimilar thereof.

6. The binding agent for use of any one of the preceding items, wherein when the PD-1 inhibitor is nivolumab or a biosimilar thereof.

7. The binding agent for use of any one of the preceding items, wherein
a) the first binding region comprises a heavy chain variable region (VH) comprising the CDR1, CDR2, and CDR3 sequences of SEQ ID NO: 1 or 9, and a light chain variable region (VL) comprising the CDR1, CDR2, and CDR3 sequences of SEQ ID NO: 5 or 10;
and
b) the second antigen-binding region comprises a heavy chain variable region (VH) comprising the CDR1, CDR2, and CDR3 sequences of SEQ ID NO: 11, and a light chain variable region (VL) comprising the CDR1, CDR2, and CDR3 sequences of SEQ ID NO: 15.

8. The binding agent for use of any one of the preceding items, wherein
a) the first binding region comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively;
and
b) the second antigen-binding region comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively.

9. The binding agent for use of any one of the preceding items, wherein the first binding region comprises a heavy chain variable region (VH) comprising an amino acid sequence having at least 90%, at least 95%, at least 97%, at least 99%, or 100% sequence identity to SEQ ID NO: 1 or 9 and a light chain variable region (VL) region and comprising an amino acid sequence having at least 90%, at least 95%, at least 97%, at least 99%, or 100% sequence identity to SEQ ID NO: 5 or 10.

10. The binding agent for use of any one of the preceding items, wherein the second binding region comprises a heavy chain variable region (VH) comprising an amino acid sequence having at least 90%, at least 95%, at least 97%, at least 99%, or 25 100% sequence identity to SEQ ID NO: 11 and a light chain variable region (VL) region comprising an amino acid sequence having at least 90%, at least 95%, at least 97%, at least 99%, or 100% sequence identity to SEQ ID NO: 15.

11. The binding agent for use of any one of the preceding items, wherein the first binding region comprises a heavy chain variable region (VH) comprising the amino acid sequence set forth in SEQ ID NO: 1 or 9 and a light chain variable region (VL) region comprising the amino acid sequence set forth in SEQ ID NO: 5 or 10.

12. The binding agent for use of any one of the preceding items, wherein the second binding region comprises a heavy chain variable region (VH) comprising the amino acid sequence set forth in SEQ ID NO: 11 and a light chain variable region (VL) region comprising the amino acid sequence set forth in SEQ ID NO: 15.

13. The binding agent for use of any one of the preceding items, wherein
a) the first binding region comprises a heavy chain variable region (VH) comprising the amino acid sequence set forth in SEQ ID NO: 1 and a light chain variable region (VL) region comprising the amino acid sequence set forth in SEQ ID NO: 5;
and
b) the second binding region comprises a heavy chain variable region (VH) comprising the amino acid sequence set forth in SEQ ID NO: 11 and a light chain variable region (VL) region comprising the amino acid sequence set forth in SEQ ID NO: 15.

14. The binding agent for use of any one of the preceding items, wherein the binding agent is a multispecific antibody, such as a bispecific antibody.

15. The binding agent for use of any one of the preceding items, wherein the binding agent is in the format of a full-length antibody or an antibody fragment.

16. The binding agent for use of any one of items 6-12, wherein each variable region comprises three complementarity determining regions (CDR1, CDR2, and CDR3) and four framework regions (FR1, FR2, FR3, and FR4).

17. The binding agent for use of item 13, wherein said complementarity determining regions and said framework regions are arranged from amino-terminus to carboxy-terminus in the following order: FR1, CDR1, FR2, CDR2, FR3, CDR3, FR4.

18. The binding agent for use of any one of items 7-17, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

19. The binding agent for use of any one of items 7-18, which comprises
i) a polypeptide comprising said first light chain variable region (VL) and further comprising a first light chain constant region (CL), and
ii) a polypeptide comprising said second light chain variable region (VL) and further comprising a second light chain constant region (CL).

20. The binding agent for use of any one of items 7-19, wherein the binding agent is an antibody comprising a first binding arm and a second binding arm, wherein the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

21. The binding agent for use of any one of the preceding items, which comprises
i) a first heavy chain and light chain comprising said antigen-binding region capable of binding to CD137, and
ii) a second heavy chain and light chain comprising said antigen-binding region capable of binding PD-L1.

22. The binding agent for use of any one of the preceding items, wherein said binding agent comprises
i) a first heavy chain and light chain comprising said antigen-binding region capable of binding to CD137, the first heavy chain comprising a first heavy chain constant region and the first light chain comprising a first light chain constant region; and
ii) a second heavy chain and light chain comprising said antigen-binding region capable of binding PD-L1, the second heavy chain comprising a second heavy chain constant region and the second light chain comprising a second light chain constant region.

23. The binding agent for use of any one of items 18-22, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

24. The binding agent for use of any one of items 18-23, wherein each of the first and second heavy chain constant regions (CHs) comprises a CH3 region and wherein the two CH3 regions comprise asymmetrical mutations.

25. The binding agent for use of any one of items 18-23, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

26. The binding agent for use of item 25, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

27. The binding agent for use of any of the preceding items, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.

28. The binding agent for use of item 27, wherein said first and second heavy chain constant regions (CHs) are modified so that the antibody induces Fc-mediated effector function to a lesser extent compared to an antibody which is identical except for comprising non-modified first and second heavy chain constant regions (CHs).

29. The binding agent for use of item 28, wherein each of said non-modified first and second heavy chain constant regions (CHs) comprises the amino acid sequence set forth in SEQ ID NO: 19 or 25.

30. The binding agent for use of item 28 or 29, wherein said Fc-mediated effector function is measured by binding to Fcγ receptors, binding to C1q, or induction of Fe-mediated crosslinking of Fcγ receptors.

31. The binding agent for use of item 30, wherein said Fc-mediated effector function is measured by binding to C1q.

32. The binding agent for use of any one of items 27-31, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

33. The binding agent for use of any one of items 18-32, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

34. The binding agent for use of item 33, wherein the positions corresponding to positions L234 and L235 in a human IgG1 heavy chain according to EU numbering are F and E, respectively, in said first and second heavy chains.

35. The binding agent for use of item 33 or 34, wherein the positions corresponding to positions L234, L235, and D265 in a human IgG1 heavy chain according to EU numbering are F, E, and A, respectively, in said first and second heavy chain constant regions.

36. The binding agent for use of any one of items 33-35, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

37. The binding agent for use of any one of items 33-36, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

38. The binding agent for use of any one of items 18-37, wherein the constant region of said first and/or second heavy chain comprises or consists essentially of or consists of an amino acid sequence selected from the group consisting of
a) the sequence set forth in SEQ ID NO: 19 or 25 [IgG1-FC];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4, at most 3, at most 2 substitutions or at most 1 substitution, compared to the amino acid sequence defined in a) or b).

39. The binding agent for use of any one of items 18-38, wherein the constant region of said first or second heavy chain, such as the second heavy chain, comprises or consists essentially of or consists of an amino acid sequence selected from the group consisting of
a) the sequence set forth in SEQ ID NO: 20 or 26 [IgG1-F405L];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 9 substitutions, such as at most 8, at most 7, at most 6, at most 5, at most 4, at most 3, at most 2 substitutions or at most 1 substitution, compared to the amino acid sequence defined in a) or b).

40. The binding agent for use of any one of items 18-38, wherein the constant region of said first or second heavy chain, such as the first heavy chain comprises or consists essentially of or consists of an amino acid sequence selected from the group consisting of
a) the sequence set forth in SEQ ID NO: 21 or 27 [IgG1-K409R];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

41. The binding agent for use of any one of items 18-37, wherein the constant region of said first and/or second heavy chain comprises or consists essentially of or consists of an amino acid sequence selected from the group consisting of
a) the sequence set forth in SEQ ID NO: 22 or 28 [IgG1-Fc_FEA];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 7 substitutions, such as at most 6 substitutions, at most 5, at most 4, at most 3, at most 2 substitutions or at most 1 substitution, compared to the amino acid sequence defined in a) or b).

42. The binding agent for use of any one of items 18-41, wherein the constant region of said first and/or second heavy chain, such as the second heavy chain, comprises or consists essentially of or consists of an amino acid sequence selected from the group consisting of
a) the sequence set forth in SEQ ID NO: 24 or 30[IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).

43. The binding agent for use of any one of items 18-42, wherein the constant region of said first and/or second heavy chain, such as the first heavy chain, comprises or consists essentially of or consists of an amino acid sequence selected from the group consisting of
a) the sequence set forth in SEQ ID NO: 23 or 29 [IgG1-Fc_FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).

44. The binding agent for use of any one of the preceding items, wherein said binding agent comprises a kappa (x) light chain constant region.

45. The binding agent for use of any one of the preceding items, wherein said binding agent comprises a lambda (a) light chain constant region.

46. The binding agent for use of any one of the preceding items, wherein said first light chain constant region is a kappa (x) light chain constant region or a lambda (Q) light chain constant region.

47. The binding agent for use of any one of the preceding items, wherein said second light chain constant region is a lambda (Q) light chain constant region or a kappa (x) light chain constant region.

48. The binding agent for use of any one of the preceding items, wherein said first light chain constant region is a kappa (x) light chain constant region and said second light chain constant region is a lambda (Q) light chain constant region or said first light chain constant region is a lambda (Q) light chain constant region and said second light chain constant region is a kappa (x) light chain constant region.

49. The binding agent for use of any one of items 44-48, wherein the kappa (x) light chain comprises an amino acid sequence selected from the group consisting of
a) the sequence set forth in SEQ ID NO:35,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

50. The binding agent for use of any one of items 45-49, wherein the lambda (Q) light chain comprises an amino acid sequence selected from the group consisting of
a) the sequence set forth in SEQ ID NO: 36,
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

51. The binding agent for use of any one of the preceding items, wherein the binding agent is of an isotype selected from the group consisting of IgG1, IgG2, IgG3, and IgG4.

52. The binding agent for use of any one of the preceding items, wherein the binding agent is a full-length IgG1 antibody.

53. The binding agent for use of any one of the preceding items, wherein the binding agent is an antibody of the IgG1m(f) allotype.

54. The binding agent for use of any one of the preceding items, wherein the binding agent comprises
i) a first heavy chain and light chain comprising said antigen-binding region capable of binding to CD137, wherein the first heavy chain comprising the sequence set forth in SEQ ID NO: 31, and the first light chain comprising the sequence set forth in SEQ ID NO: 32;
ii) a second heavy chain and light chain comprising said antigen-binding region capable of binding PD-L1, wherein the second heavy chain comprising the sequence set forth in SEQ ID NO: 33, and the second light chain comprising the sequence set forth in SEQ ID NO: 34.

55. The binding agent for use according to any one of the preceding items, wherein the binding agent is acasunlimab or a biosimilar thereof.

56. The binding agent for use according to any one of the preceding items, wherein the binding agent is in a composition or formulation comprising histidine, sucrose and Polysorbate-80, and has a pH from 5 to 6.

57. The binding agent for use according to any one of the preceding items, wherein the binding agent is in a composition or formulation comprising about 20 mM histidine, about 250 mM Sucrose, about 0.02% Polysorbate-80, and having a pH of about 5.5.

58. The binding agent for use according to any one of the preceding items, wherein the binding agent is in a composition or formulation comprising 10^{×30} mg binding agent/mL, such as 20 mg binding agent/mL.

59. The binding agent for use according to any one of the preceding items, wherein the binding agent is in a composition as defined in any one of items 56 to 58 and is diluted in 0.9% NaCl (saline) prior to administration.

60. The binding agent for use according to any one of the preceding items, the PD-1 inhibitor is an antibody binding to PD-1, wherein the antibody binding to PD-1 comprises a VH region CDR1, CDR2, and CDR3 comprising the sequences as set forth in SEQ ID NOs: 104, 101, and 100, respectively, and a VL region CDR1, CDR2, and CDR3 comprising the sequences as set forth in SEQ ID NO: 107, QAS and SEQ ID NO: 105, respectively.

61. The binding agent for use according to item 60, wherein the antibody binding to PD-1 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 111.

62. The binding agent for use according to item 60 or 61, wherein the antibody binding to PD-1 comprises a heavy chain variable region (VH), wherein the VH comprises the sequence as set forth in SEQ ID NO: 111.

63. The binding agent for use according to any one of items 60-62, wherein the antibody binding to PD-1 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 112.

64. The binding agent for use according to item 63, wherein the antibody binding to PD-1 comprises a light chain variable region (VL), wherein the VL comprises the sequence as set forth in SEQ ID NO: 112.

65. The binding agent for use according to any one of items 60-64, wherein the antibody binding to PD-1 comprises a heavy chain variable region (VH) and a light chain variable region (VL), wherein the VH comprises or has the sequence as set forth in SEQ ID NO: 11I and the VL comprises or has the sequence as set forth in SEQ ID NO: 112.

66. The binding agent for use according to any one of items 60-65, wherein the antibody binding to PD-1 comprises a heavy chain constant region, wherein the heavy chain constant region comprises an aromatic or non-polar amino acid at the position corresponding to position 234 in a human IgG1 heavy chain according to EU numbering and an amino acid other than glycine at the position corresponding to position 236 in a human IgG1 heavy chain according to EU numbering.

67. The binding agent for use according to item 66, wherein the amino acid at the position corresponding to position 236 is a basic amino acid.

68. The binding agent for use according to item 67, wherein the basic amino acid is selected from the group consisting of lysine, arginine and histidine.

69. The binding agent for use according to item 67 or 68, wherein the basic amino acid is arginine (G236R).

70. The binding agent for use according to any one of items 66-69, wherein the amino acid at the position corresponding to position 234 is an aromatic amino acid.

71. The binding agent for use according to item 70, wherein the aromatic amino acid is selected from the group consisting of phenylalanine, tryptophan and tyrosine.

72. The binding agent for use according to any one of items 66-69, wherein the amino acid at the position corresponding to position 234 is a non-polar amino acid.

73. The binding agent for use according to item 72, wherein the non-polar amino acid is selected from the group consisting of alanine, valine, leucine, isoleucine, proline, phenylalanine, methionine and tryptophan.

74. The binding agent for use according to item 72 or 73, wherein the non-polar amino acid is selected from the group consisting of isoleucine, proline, phenylalanine, methionine and tryptophan.

75. The binding agent for use according to any one of items 66-74, wherein the amino acid at the corresponding to position 234 is phenylalanine (L234F).

76. The binding agent for use according to any one of items 66-75, wherein the amino acid at the position corresponding to position 235 in a human IgG1 heavy chain according to EU numbering in said heavy chain constant region of the antibody binding to PD-1 is an acidic amino acid.

77. The binding agent for use according to item 76, wherein the acidic amino acid is aspartate or glutamate.

78. The binding agent for use according to any one of items 66-77, wherein the amino acid at the position corresponding to position 235 in a human IgG1 heavy chain according to EU numbering in said heavy chain constant region of the antibody binding to PD-1 is glutamate (L235E).

79. The binding agent for use according to any one of items 66-78, wherein the amino acids at the position corresponding to positions 234, 235 and 236 in said heavy chain constant region of the antibody binding to PD-1 are a non-polar or an aromatic amino acid at position 234, an acidic amino acid at position 235 and a basic amino acid at position 236.

80. The binding agent for use according to any one of items 66-79, wherein the amino acid corresponding to position 234 is phenylalanine, the amino acid corresponding to position 235 is glutamate, and the amino acid corresponding to position 236 is arginine in said heavy chain constant region of the antibody binding to PD-1 (L234F/L235E/G236R).

81. The binding agent for use according to any one of items 60-80, wherein the heavy chain constant region of the antibody binding to PD-1 comprises a sequence having at least 70%, at least 75%, at least 80%, at least 85%, at least 90%, at least 95%, at least 97%, at least 99%, or 100% identity to the amino acid sequence of the heavy chain constant region sequence as set forth in SEQ ID NO: 93.

82. The binding agent for use according to any one of items 60-81, wherein the heavy chain constant region of the antibody binding to PD-1 comprises the sequence as set forth in SEQ ID NO: 93.

83. The binding agent for use according to any one of items 60-82, wherein the isotype of the heavy chain constant region of the antibody binding to PD-1 is IgG1.

84. The binding agent for use according to any one of items 60-83, wherein the antibody binding to PD-1 is a monoclonal, chimeric or humanized antibody or a fragment of such an antibody.

85. The binding agent for use according to any one of items 60-84, wherein the antibody binding to PD-1 has a reduced or depleted Fc-mediated effector function.

86. The binding agent for use according to any one of items 60-85, wherein binding of complement protein C1q to the constant region of the antibody binding to PD-1 is reduced compared to a wild-type antibody, preferably by at least 70%, at least 80%, at least 90%, at least 95%, at least 97% or 100%.

87. The binding agent for use according to any one of items 60-86, wherein binding to one or more of the IgG Fc-gamma receptors to the antibody binding to PD-1 is reduced compared to a wild-type antibody, preferably by at least 70%, at least 80%, at least 90%, at least 95%, at least 97% or 100%.

88. The binding agent for use according to item 87, wherein the one or more IgG Fc-gamma receptors are selected from at least one of Fc-gamma RI, Fc-gamma RII and Fc-gamma RIII.

89. The binding agent for use according to item 87 or 88, wherein the IgG Fc-gamma receptor is Fc-gamma RI.

90. The binding agent for use according to any one of items 60-89, wherein the antibody binding to PD-1 is not capable of inducing Fc-gamma RI-mediated effector functions or wherein the induced Fc-gamma RI-mediated effector functions are reduced compared to a wild-type antibody, preferably by at least 70%, at least 80%, at least 90%, at least 95%, at least 97% or 100%,

91. The binding agent for use according to any one of items 60-90, wherein the antibody binding to PD-1 is not capable of inducing at least one of complement dependent cytotoxicity (CDC) mediated lysis, antibody dependent cellular cytotoxicity (ADCC) mediated lysis, apoptosis, homotypic adhesion and/or phagocytosis or wherein at least one of complement dependent cytotoxicity (CDC) mediated lysis, antibody dependent cellular cytotoxicity (ADCC) mediated lysis, apoptosis, homotypic adhesion and/or phagocytosis is induced in a reduced extent, preferably reduced by at least 70%, at least 80%, at least 90%, at least 95%, at least 97% or 100%.

92. The binding agent for use according to any one of items 60-91, wherein binding of neonatal Fc receptor (FcRn) to the antibody binding to PD-1 is unaffected, as compared to a wild-type antibody.

93. The binding agent for use according to any one of items 60-92, wherein PD-1 is human PD-1.

94. The binding agent for use according to item 93, wherein the PD-1 has or comprises the amino acid sequence as set forth in SEQ ID NO: 113 or SEQ ID NO: 114, or the amino acid sequence of PD-1 has at least 70%, at least 75%, at least 80%, at least 85%, at least 90%, at least 95%, at least 97%, at least 99%, or 100% identity to the amino acid sequence as set forth in SEQ ID NO: 113 or SEQ ID NO: 114, or is an immunogenic fragment thereof.

95. The binding agent for use according to any one of items 60-94, the antibody binding to PD-1 binds to a native epitope of PD-1 present on the surface of living cells.

96. The binding agent for use according to any one of items 60-95, wherein the antibody binding to PD-1 is a multispecific antibody comprising a first antigen-binding region binding to PD-1 and at least one further antigen-binding region binding to another antigen.

97. The binding agent for use according to item 96, wherein the antibody binding to PD-1 is a bispecific antibody comprising a first antigen-binding region binding to PD-1 and a second antigen-binding region binding to another antigen.

98. The binding agent for use according to item 96 or 97, wherein the first antigen-binding region binding to PD-1 comprises the heavy chain variable region (VH) and/or the light chain variable region (VL) as set forth in any one of items 61 to 65.

99. The binding agent for use of any one of the preceding items, wherein the subject is a human subject.

100. The binding agent for use of any one of the preceding items, wherein the tumor or cancer is a solid tumor or cancer.

101. The binding agent for use according to any one of the preceding items, wherein said tumor is a PD-L1 positive tumor.

102. The binding agent for use of any one of the preceding items, wherein the tumor or cancer is selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, endometrial cancer, prostate cancer, penile cancer, cervical cancer, Hodgkin's lymphoma, non-Hodgkin's lymphoma, Merkel cell carcinoma and mesothelioma.

103. The binding agent for use according to any one of the preceding items, wherein the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)) and squamous cell carcinoma of the head and neck (SCCHN) (e.g. cancer of the oral cavity, pharynx or larynx).

104. The binding agent for use of item 102 or 103, wherein the tumor or cancer is lung cancer, in particular a non-small cell lung cancer (NSCLC), such as a squamous or non-squamous NSCLC.

105. The binding agent for use of any one of items 100 to 104, wherein the tumor or cancer is metastatic, such as metastatic NSCLC.

106. The binding agent for use of item 104 or 105, wherein the lung cancer, in particular NSCLC, does not have an epidermal growth factor (EGFR)-sensitizing mutation and/or anaplastic lymphoma (ALK) translocation/ROS1 rearrangement.

107. The binding agent for use of any one of items 104 to 106, wherein the lung cancer, in particular NSCLC, comprises cancer cells and PD-L1 is expressed in >1% of the cancer cells or tumor cells e.g. as assessed by immunohistochemistry (IHC).

108. The binding agent for use of the preceding items, wherein the subject has not received prior systemic treatment of metastatic disease.

109. The binding agent for use of any one of the preceding items, wherein the subject has not received prior treatment with a checkpoint inhibitor; e.g., a PD-1 inhibitor or a PD-L1 inhibitor, such as anti-PD-1 antibody or an anti-PD-L1 antibody.

110. The binding agent for use of any one of the preceding items, wherein the subject has not received prior treatment with a 4-1BB (CD137) targeted agent, such as an anti-4-1BB (CD137) antibody, with an antitumor vaccine, or with autologous cell immunotherapy

111. The binding agent for use of any one of items 1 to 107, wherein the tumor or cancer has relapsed and/or is refractory after treatment, such as systemic treatment with a checkpoint inhibitor.

112. The binding agent for use of any one of items 1 to 107 and 111, wherein the subject has received at least 1 prior line of systemic therapy, such as systemic therapy comprising a PD-1 inhibitor or a PD-L1 inhibitor, such as an anti-PD-1 antibody or an anti-PD-L1 antibody.

113. The binding agent for use of any one of items 1 to 107, 111 and 112, wherein the cancer or tumor has relapsed and/or is refractory, or the subject has progressed after treatment with a PD-1 inhibitor or a PD-L1 inhibitor, such as an anti PD-1 antibody or an anti-PD-L1 antibody, the PD-1 inhibitor or PD-L1 inhibitor being administered as monotherapy or as part of a combination therapy.

114. The binding agent for use of any one of items 1 to 107 and 111 to 113, wherein last prior treatment was with a PD1 inhibitor or PD-L1 inhibitor, such as an anti PD-1 antibody or an anti-PD-L1 antibody, the PD-1 inhibitor or PD-L1 inhibitor being administered as monotherapy or as part of a combination therapy.

115. The binding agent for use of any one of items 1 to 107 and 111 to 114, wherein the time from progression on last treatment with a PD1 inhibitor or PD-L1 inhibitor, such as an anti PD-1 antibody or an anti-PD-L1 antibody is 8 months or less, such as 7 months or less, 6 months or less, 5 months or less, 4 months or less, 3 months or less, 2 months or less, 1 month or less, 3 weeks or less or such as 2 weeks or less.

116. The binding agent for use of any one of items 1 to 107 and 111 to 115, wherein the time from last dosing of a PD1 inhibitor or PD-L1 inhibitor, such as an anti PD-1 antibody or an anti-PD-L1 antibody as part of last prior treatment is 8 months or less, such as 7 months or less, 6 months or less, 5 months or less, 4 months or less, 3 months or less, 2 months or less, 1 month or less, 3 weeks or less or such as 2 weeks or less.

117. The binding agent for use of any one of items 1 to 107 and 111 to 116, wherein the cancer or tumor has relapsed and/or is refractory, or the subject has progressed during or after
i) platinum doublet chemotherapy following treatment with an anti-PD-1 antibody or an anti-PD-L1 antibody, or
ii) treatment with an anti-PD-1 antibody or an anti-PD-L1 antibody following platinum doublet chemotherapy.

118. The binding agent for use of any one of the preceding items, wherein the subject has not received prior treatment with a taxane chemotherapeutic agent e.g., docetaxel, such as prior treatment of NSCLC with a taxane chemotherapeutic agent e.g., docetaxel.

119. The binding agent for use of any one of the preceding items, wherein the binding agent and the PD-1 inhibitor are administered in at least one treatment cycle, each treatment cycle being two weeks (14 days), three weeks (21 days), four weeks (28 days), 5 weeks (35 days) or six weeks (42 days).

120. The binding agent for use of any one of the preceding items, wherein one dose of the binding agent and one dose of the PD-1 inhibitor are administered every second week (1Q2W) every third week (1Q3W), every fourth week (1Q4W), every fifth week (1Q5W) or every sixth week (1Q6W).

121. The binding agent for use of any one of the preceding items, wherein one dose of the binding agent and one dose of the PD-1 inhibitor are administered every six weeks (1Q6W).

122. The binding agent for use of any one of the preceding items, wherein one dose of the binding agent and one dose of the PD-1 inhibitor are administered on day 1 of each treatment cycle.

123. The binding agent for use of any one of the preceding items, wherein the amount of said binding agent administered in each dose and/or in each treatment cycle is 100 mg.

124. The binding agent for use of any one of the preceding items, wherein the amount of said PD-1 inhibitor administered in each dose and/or in each treatment cycle is 200 mg.

125. The binding agent for use of any one of the preceding items, wherein the amount of said PD-1 inhibitor administered in each dose and/or in each treatment cycle is 400 mg.

126. The binding agent for use of any one of the preceding items, wherein a 100 mg dose of the binding agent and a 200 mg dose of the PD-1 inhibitor are administered every three weeks (1Q3W).

127. The binding agent for use of any one of the preceding items, wherein a 100 mg dose of the binding agent and a 400 mg dose of the PD-1 inhibitor are administered every six weeks (1Q6W).

128. The binding agent for use of any one of the preceding items, wherein the tumor or cancer is NSCLC; and wherein a 100 mg dose of the binding agent, which is acasunlimab or a biosimilar thereof and a 200 mg dose of the PD-1 inhibitor, which is nivolumab, are administered every three weeks (1Q3W), such as on day one of each three-week treatment cycle.

129. The binding agent for use of any one of the preceding items, wherein the PD-1 inhibitor is administered first, followed by the binding agent.

130. The binding agent for use of any one of the preceding items, wherein the binding agent is administered by using intravenous (IV) infusion over a minimum of 30 minutes, such as over a minimum of 60 minutes.

131. The binding agent for use of any one of the preceding items, wherein the binding agent is administered by using intravenous (IV) infusion over 30 minutes.

132. The binding agent for use of any one of the preceding items, wherein the PD-1 inhibitor is administered as an intravenous infusion over 30 minutes.

133. A kit comprising (i) a binding agent comprising a first binding region binding to CD137 and a second binding region binding to PD-L1, and (ii) a PD-1 inhibitor;
wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively, then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

134. The kit according to item 133, wherein the binding agent is as defined in any one of items 1, 2 and 7-58 and/or the PD-1 inhibitor is as defined in any one of items 3 to 6, and 59-97.

135. The kit according to item 133 or 134, wherein the binding agent, the PD-1 inhibitor, and, if present, the one or more additional therapeutic agents are for systemic administration, in particular for injection or infusion, such as intravenous injection or infusion.

136. The kit according to any one of items 133-135 for use in a method for reducing or preventing progression of a tumor or treating cancer in a subject.

137. The kit for use according to item 136, wherein the tumor or cancer and/or the subject and/or the method is/are as defined in any one of items 1-132.

138. A method for reducing or preventing progression of a tumor or treating cancer in a subject, said method comprising administering to said subject a binding agent prior to, simultaneously with, or after administration of a PD-1 inhibitor, wherein the binding agent comprises a first binding region binding to CD137 and a second binding region binding to PD-L1, and wherein
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,
then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

139. The method of item 138, wherein the tumor or cancer and/or the subject and/or the method and/or the binding agent and/or the PD-1 inhibitor is/are as defined in any one of items 1-132.

Further aspects of the present disclosure are disclosed herein.

### EXAMPLES

### Example 1: MC38 Mouse Colon Cancer Tumor Outgrowth

### Methods

MC38 mouse colon cancer cells were cultured in Dulbecco's Modified Eagle Medium supplemented with 10% heat-inactivated fetal bovine serum at 37° C., 5% CO₂. MC38 cells were harvested from a cell culture growing in log-phase and quantified.

MC38 cells (1×10⁶ tumor cells in 100 μL PBS) were injected subcutaneously in the right lower flank of female C57BL/6 mice (obtained from Vital River Laboratories Research Models and Services; age 6-8 weeks at start of experiment).

Tumor growth was evaluated three times per week using a caliper. Tumor volumes (mm³) were calculated from caliper measurements as ([length]×[width]²)/2, where the length is the longest tumor dimension and the width is the longest tumor dimension perpendicular to the length.

Treatment was initiated when tumors had reached a median volume of 64 mm³. Mice were randomized into groups (n=10/group) with equal average tumor volume prior to treatment (64 mm³). On treatment days, the mice were injected intraperitoneally with mbsIgG2a-PD-Llx4-1BB (5 mg/kg; injection volume of 10 μL/g body weight; two doses weekly for three weeks [2QWX3]), an anti-mouse PD-1 antibody (anti-mPD-1; 10 mg/kg; injection volume of 10 μL/g body weight; 2QWX3; clone RMP1-14; Leinco Technologies, cat. no. P372), a combination of mbsIgG2a-PD-Llx4-1BB (5 mg/kg) with anti-mPD-1 (10 mg/kg; in two separate injections [mbsIgG2a-PD-Llx4-1BB followed by anti-mPD-1 after 20 min] with an injection volume of 10 μL/g body weight; 2QWX3), or PBS with an injection volume of 10 μL/g body weight (Table 7).

The mice were monitored daily for clinical signs of illness. Body weight measurements were performed three times a week after randomization. The experiment ended for the individual mice when the tumor volume exceeded 1500 mm³ or when the animals reached humane endpoints (e.g. when mice showed body weight loss >20%, when tumors showed ulceration [>75%], when serious clinical signs were observed and/or when the tumor growth blocked the physical activity of the mouse).

TABLE 7
Treatment groups and dosing regimen

| Treatment | N per | | | Dosing | Dosing | Seq ids/ |
|---|---|---|---|---|---|---|
| group | group | Treatment | Dose^{a} | route | regimen | Supplier, cat. no. |
| 1 | 10 | PBS | N/A | IP | 2QW × 3^{a} | N/A |
| 2 | 10 | Anti-mPD-1 | 10 mg/kg | IP | 2QW × 3^{a} | Leinco Technologies, |
| | | | | | | cat. no. P372 |
| 3 | 10 | mbsIgG2a-PD-L1 × | 5 mg/kg | IP | 2QW × 3^{a} | Seq ids: 86, 87, 81, |
| | | 4-1BB | | | | 82, 83, 84, 85 |
| 4 | 10 | mbsIgG2a-PD-L1 × | 5 mg/kg + | IP | 2QW × 3^{a} | Seq ids: 86, 87, 81, |
| | | 4-1BB + Anti-mPD-1 | 10 mg/kg | | | 82, 83, 84, 85 |
| | | | | | | Leinco Technologies, |
| | | | | | | cat. no. P372 |

^{a}2QW × 3: two doses weekly for three weeks

### Results

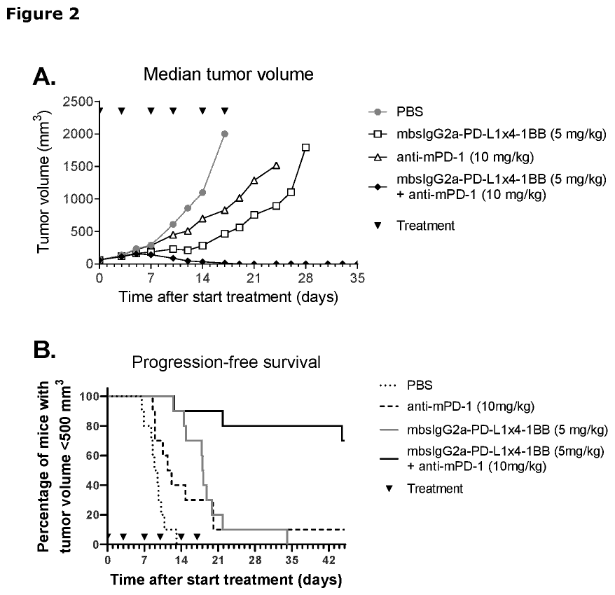

Rapid tumor outgrowth was observed in MC38-bearing mice treated with PBS (FIG. 2A). In mice treated with anti-mPD-1 (10 mg/kg) or mbslgG2a-PD-Lx4-1BB (5 mg/kg) delayed tumor outgrowth was observed, with a more pronounced delay in tumor outgrowth induced by mbs.gG2a-PD-L1×4-1BB (FIG. 2A). In mice treated with mbsbgG2a-PD-L1×4-1BB (5 mg/kg) combined with anti-mPD-1 (10 mg/kg; both 2QWx3) complete tumor regressions were observed in 6/10 mice at day 21 post-treatment initiation compared to no complete tumor regressions observed for either agent alone in this model (FIG. 2A). Kaplan-Meier analysis showed that treatment with the combination of mbsIgG2a-PD-L1×4-1BB and anti-mPD-1 induced a significant increase in progression-free survival, defined as the percentage of mice with tumor volume smaller than 500 mm³, when compared to the PBS-treated group (p<0.001) and compared to either antibody alone (p<0.001; Mantel-Cox; FIG. 2B3, Table 8). Hence, therapeutic synergy was observed with this combination, defined as superior (p<0.05) antitumnor efficacy relative to the activity shown by each agent as monotherapy.

These results provide rationale for evaluating the combination of GEN1046 with an anti-PD-i antibody to further amplify the anti-tumor immune response in cancer patients to produce durable and deep clinical responses and enhance survival.

TABLE 8
Mantel-Cox analysis of the progression-free survival induced by mbsIgG2a-PD-L1x4-1BB,
anti-mPD-1 (either alone or in combination) in the MC38 model in C57BL/6 mice

| | | | Progression-free survival¹ |
|---|---|---|---|
| Treatment groups compared | | | Mantel-Cox P value |
| PBS | VS | Anti-mPD-1 | 0.012 |
| PBS | VS | mbslgG2a-PD-L1x4-1BB | <0.001 |
| PBS | VS | mbslgG2a-PD-L1x4-1BB + anti-mPD-1 | <0.001 |
| Anti-mPD-1 | VS | mbslgG2a-PD-L1x4-1BB | 0.515 |
| Anti-mPD-1 | VS | mbslgG2a-PD-L1x4-1BB + anti-mPD-1 | 0.001 |
| mbsIgG2a-PD-L1x4-1BB (5 mg/kg) | VS | mbslgG2a-PD-L1x4-1BB + anti-mPD-1 | <0.001 |

¹Tumor volume <500 mm³ was used as the cut-off for progression-free survival. Mantel-Cox analysis was performed at Day 45.

### Example 2: Antigen-Specific CD8⁺ T Cell Proliferation Assay to Determine the Proliferation Dose-Response of GEN1046 and Anti-PD-1 Antibody Nivolumab in an Antigen-Specific T Cell Assay with Active PD1/PD-L1 Axis

To measure induction of T cell proliferation by GEN1046 or Nivolumab, an antigen-specific T cell proliferation assay with active PD1/PD-L1 axis was performed.

| Test | Supplier, | Comprising |
|---|---|---|
| compound | cat. no. | SEQ ID NOS |
| GEN1046 | N/A | CD137 binding arm: |
| | | SEQ ID NOS: 1, 5, 35, 29 |
| | | PD-L1 binding arm: |
| | | SEQ ID NOS: 11, 15, 36, 30 |

HLA-A2+peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were frozen for future T-cell isolation. For differentiation into immature DCs (iDCs), 1×10⁻⁶ monocytes/ml were cultured for five days in RPMI GlutaMAX (Life technologies GmbH, cat. no. 61870-044) containing 5% human AB serum (Sigma-Aldrich Chemie GmbH, cat. no. H4522-100ML), sodium pyruvate (Life technologies GmbH, cat. no. 11360-039), non-essential amino acids (Life technologies GmbH, cat. no. 11140-035), 100 IU/mL penicillin-streptomycin (Life technologies GmbH, cat. no.15140-122), 1000 IU/mL granulocyte-macrophage colony-stimulating factor (GM-CSF; Miltenyi, cat. no. 130-093-868) and 1000 IU/mL interleukin-4 (IL-4; Miltenyi, cat. no. 130-093-924). Once during these five days, half of the medium was replaced with fresh medium. iDCs were harvested by collecting non-adherent cells and adherent cells were detached by incubation with PBS containing 2 mM EDTA for 10 min at 37°. After washing iDCs were frozen in RPMI GlutaMAX containing 10% v/v DMSO (AppliChem GmbH, cat. no A3672,0050)±50% v/v human AB serum for future antigen-specific T cell assays.

One day prior to the start of an antigen-specific CD8⁺ T cell proliferation assay, frozen PBLs and iDCs, from the same donor, were thawed. CD8⁺ T cells were isolated from PBLs by MACS technology using anti-CD8 MicroBeads (Miltenyi, cat. no. 130-045-201), according to the manufacturer's instructions. About 10-15×10⁶ CD8+ T cells were electroporated with 10 μg of in vitro translated (IVT)-RNA encoding the alpha-chain plus 10 μg of IVT-RNA encoding the beta-chain of a claudin-6-specific murine TCR (HLA-A2-restricted; described in WO 2015150327 Al) plus 10 μg IVT-RNA encoding PD-1 in 250 μL X-Vivo l5 (Biozym Scientific GmbH, cat. no.881026) in a 4-mm electroporation cuvette (VWR International GmbH, cat. no. 732-0023) using the BTX ECM® 830 Electroporation System device (BTX; 500 V, 1×3 ms pulse). Immediately after electroporation, cells were transferred into fresh IMDM medium (Life Technologies GmbH, cat. no. 12440-061) supplemented with 5% human AB serum and rested at 37° C., 5% CO₂ for at least 1 hour. T cells were labeled using 1.6 μM carboxyfluorescein succinimidyl ester (CFSE; Invitrogen, cat. no. C34564) in PBS according to the manufacturer's instructions, and incubated in IMDM medium supplemented with 5% human AB serum, O/N.

Up to 5×10⁶ thawed iDCs were electroporated with either 1 μg (GEN1046 dose-response) or 3 μg (Pembrolizumab dose-response) IVT-RNA encoding full length claudin-6, in 250 μL X-Vivoi5 medium, using the electroporation system as described above (300 V, 1×12 ms pulse) and incubated in IMDM medium supplemented with 5% human AB serum, O/N.

The next day, cells were harvested. Cell surface expression of claudin-6 and PD-L1 on DCs and TCR and PD-1 on T cells was checked by flow cytometry. DCs were stained with an Alexa647-conjugated CLDN6-specific antibody (non-commercially available; in-house production) and with anti-human CD274 antibody (PD-L1, eBioscienes, cat. no.12-5983) and T cells were stained with an anti-Mouse TCR B Chain antibody (Becton Dickinson GmbH, cat. no. 553174) and with anti-human CD279 antibody (PD-1, eBioscience, cat. no. 17-2799). Electroporated DCs were incubated with electroporated, CFSE-labeled T cells in a ratio of 1:10 in the presence of GEN1046 (at 3-fold serial dilutions from 1 to 0.00015 μg/mL) or clinical-grade Nivolumab (at 4-fold serial dilutions from 0.8 to 0.00005 μg/mL; Opdivo, Phoenix Apotheke, PZN 11024601) in IMDM GlutaMAX supplemented with 5% human AB serum in a 96-well round-bottom plate. Flow cytometric analysis of T cell proliferation based on CFSE-dilution was performed after 5 days on a BD FACSCanto™ II or BD FACSCelesta™ flow cytometer (Becton Dickinson GmbH). Acquired data was analyzed using FlowJo software version 10.7.1. The expansion index values (determines the fold-expansion of the overall culture) per treatment condition were calculated and plotted as a function of the GEN1046 or Nivolumab concentration. Dose-response curves were generated and EC₂₀, EC₅₀, EC₉₀ and Hill-Slope values were calculated in GraphPad Prism version 9 (GraphPad Software, Inc.) using a 4-parameter logarithmic fit.

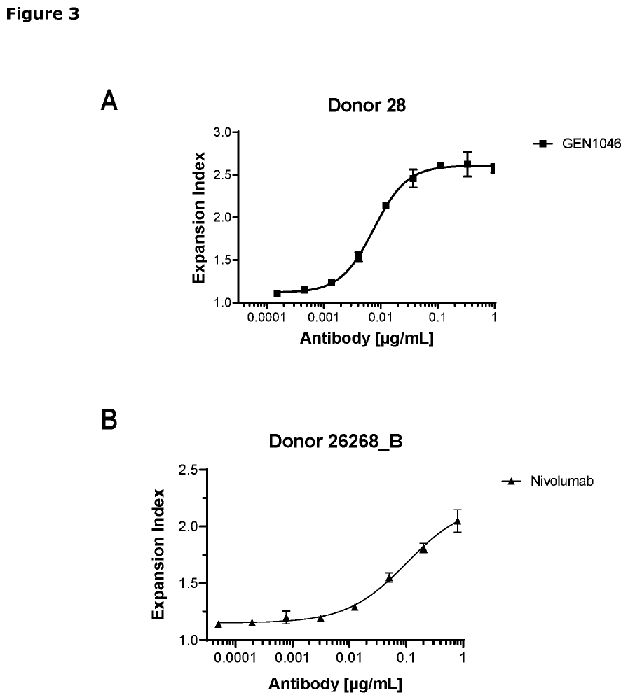

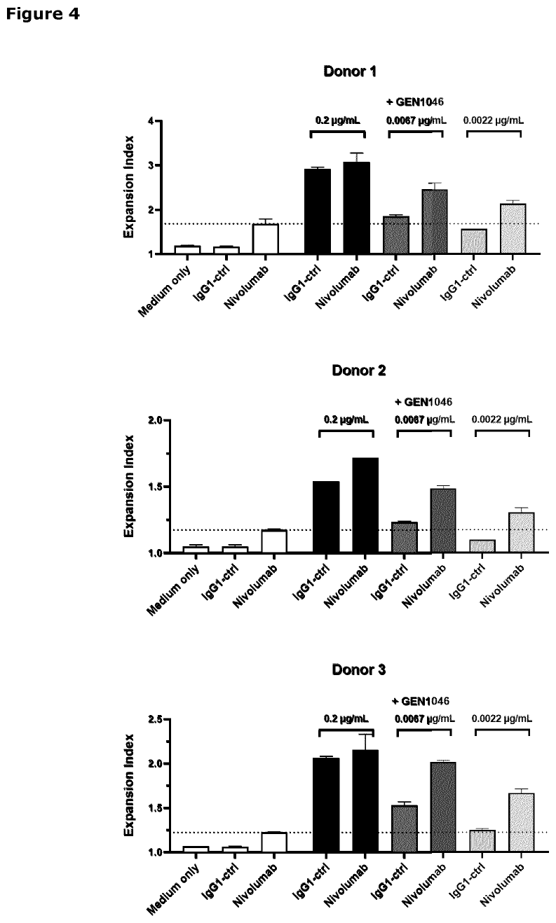

The GEN1046 dose response was analyzed at 3-fold serial dilutions from 1 to 0.00015 μg/mL (FIG. 3A) with EC₂₀, EC₅₀, EC₉₀ and Hill-Slope values given in Table 9. A strong proliferation induction effect was seen with a mean EC₅₀ of 0.0064 μg/mL across four donors tested.

The Nivolumab dose response was analyzed at 4-fold serial dilutions from 0.8 to 0.00005 μg/mL (FIG. 3B) with EC₅₀, EC₉₀ and Hill-Slope values given in Table 10. A strong proliferation induction effect was seen with a mean EC₅₀ of 0.0784 μg/mL across four donors tested.

TABLE 9
Determination of EC₂₀, EC₅₀ and EC₉₀-values of GEN1046 based on CD8⁺ T-cell expansion
data as measured by an antigen-specific T-cell proliferation assay. Data shown are the values
calculated based on the four parameter logarithmic fits.

| | EC₅₀ value | | Calc. EC₂₀ | Calc. EC₉₀ |
|---|---|---|---|---|
| Donor | [μg/ml] | Hill-Slope | [μg/ml] | [μg/ml] |
| 28 | 0.00754 | 1.485 | 0.00296 | 0.03311 |
| 89 | 0.00776 | 1.469 | 0.00302 | 0.03464 |
| 02 | 0.00523 | 1.910 | 0.00253 | 0.01651 |
| 72 | 0.00506 | 1.334 | 0.00179 | 0.02626 |
| Mean | 0.0064 | 1.549 | 0.0026 | 0.0276 |

TABLE 10
Determination of EC₅₀ and EC₉₀-values of approved
anti-PD-1 antibody Nivolumab based on
CD8⁺ T-cell expansion data as measured by an antigen-specific
T-cell proliferation assay. Data shown
are the values calculated based on the four parameter
logarithmic fits. Mean is the arithmetic mean.

| | EC₅₀ value | | Calc. EC₉₀ |
|---|---|---|---|
| Donor | [μg/ml] | Hill-Slope | [μg/ml] |
| 26268 B | 0.1011 | 0.8314 | 1.4207 |
| 26685 A | 0.0759 | 0.8351 | 1.0542 |
| 26395 B | 0.0583 | 0.7417 | 1.1278 |
| Mean | 0.0784 | 0.8027 | 1.201 |

### Example 3: Release of the PD-1/PD-L1-Mediated T Cell Inhibition and Additional Co-Stimulation of CD8⁺ T Cell Proliferation by GEN1046 in the Presence or Absence of Anti-PD-1 Antibody Nivolumab

To measure induction of T cell proliferation by DuoBody-PD-Llx4-1BB in combination with anti-PD-1 antibody Nivolumab or IgG1-ctrl antibody, an antigen-specific T cell proliferation assay with active PD1/PD-L1 axis was performed (general assay set-up analogous to example 2). In short, claudin-6-IVT-RNA electroporated DCs were incubated with claudin-6-specific TCR- and PD1-IVT-RNA electroporated, CFSE-labeled T cells (ratio of 1:10) in the presence of GEN1046 in combination with a fixed concentration of Nivolumab or IgG1-ctrl control antibody in IMDM GlutaMAX supplemented with 5% human AB serum in a 96-well round-bottom plate. Three different concentrations of GEN1046 were tested, representing optimal, half-maximal and sub-optimal effective concentrations (0.2 μg/mL >EC90; 0.0067 μg/mL ˜EC50; 0.0022 μg/mL ˜EC20, see Example 2, Table 9). Nivolumab and the IgG1-ctrl control antibody were tested at a concentration of 1.6 μg/mL and 0.8 μg/mL, respectively, a concentration well above the EC₉₀ value for Nivolumab (see Example 2, Table 10). Medium and 0.8 μg/mL IgG1-ctrl only were used to determine baseline proliferation. Nivolumab (1.6 μg/mL) was used as additional checkpoint inhibition control. Flow cytometric analysis of T cell proliferation based on CFSE-dilution was performed after 5 days on a BD FACSCanto™ II or BD FACSCelesta™ flow cytometer (Becton Dickinson GmbH). Acquired data was analyzed using FlowJo software version 10.7.1. The expansion index values per treatment condition were calculated and plotted using GraphPad Prism version 9 (GraphPad Software, Inc.).

Incubation of PD-1 and claudin-6-specific TCR expressing CD8⁺ T cells with DCs expressing PD-L1 and cognate antigen resulted in a minimal proliferation induction with expansion index values slightly above 1 in the medium only and IgG1-ctrl treated cultures for all three donors tested (see FIG. 4). Releasing the PD-1:PD-L1 mediated inhibition by adding Nivolumab to the co-culture setting resulted in a modest increase of the expansion index, indicated by the dashed line in the graph. A more pronounced as well as dose-dependent increase in T cell proliferation was observed after addition of GEN1046, with the highest concentration tested resulting in the highest proliferation induction compared to the medium and low concentration single compound treatment conditions. Of note, the lowest concentration of 0.0022 μg/mL GEN1046 (w/o Nivolumab combination) resulted in expansion index values which were on par or even below those values recorded for the Nivolumab only control, being indicative of a sub-optimal PD-1:PD-L1 checkpoint blockade. In striking contrast, independent of the GEN1046 concentration tested, T cell proliferation induction for the GEN1046 with Nivolumab combination was always superior to the GEN1046 without Nivolumab condition. The difference in expansion indices in between the w/and w/o Nivolumab condition was particularly strong for the medium and low GEN1046 concentrations. Especially, in case of the sub-optimal GEN1046 condition (0.0022 μg/mL ˜EC20), addition of Nivolumab rescued the CD8⁺ T cell proliferation with considerably higher expansion indices compared to those observed for the Nivolumab only control.

### Example 4: First-In-Human, Open-Label, Dose-Escalation Trial with Expansion Cohorts to Evaluate Safety of GEN1046 in Subjects with Malignant Solid Tumors

The study is an open-label, multi-center, phase 1/2a safety trial of GEN1046 (DuoBody®-PD-L1×4-1BB). The trial consists of 2 parts; a first-in-human (FIH) dose escalation (phase 1) and an expansion (phase 2a). The dose escalation evaluated GEN1046 in subjects with solid malignant tumors to determine the maximum tolerated dose (MTD) or maximum administered dose and/or the recommended phase 2 dose (RP2D).

The expansion further evaluates the safety, tolerability, PK, and anti-tumor activity of the selected dose(s) in select solid tumors expansion cohorts for non-small cell lung cancer (NSCLC) (PD-1/L1 pre-treated and PD-1/L1 naive), urothelial cancer (UC), endometrial cancer (EC), triple negative breast cancer (TNBC) (in subjects who have received prior treatment with a PD-1/L1 inhibitor and in subjects who have not received such treatment); and squamous cell carcinoma of the head and neck (SCCHN).

TABLE 11
Expansion cohorts

| Cohort | | | Sub- | Prior | |
|---|---|---|---|---|---|
| No. | n | Cancer Type | cohort | Treatment | Trial Treatment |
| EC1 | 140 | NSCLC | | Prior CPI | GEN1046 100 mg |
| | | | | treatment | 1Q3W |
| EC2 | 40 | NSCLC | | PD-1/L1 naive | GEN1046 100 mg |
| | | | | | 1Q3W |
| EC3 | 40 | UC | | Prior CPI | GEN1046 100 mg |
| | | | | treatment | 1Q3W |
| EC4 | 40 | Endometrial | | PD-1/L1 naive | GEN1046 100 mg |
| | | cancer | | | 1Q.3W |
| EC5 | 40 | TNBC | 5a | Prior CPI | GEN1046 100 mg |
| | | | | treatment | 1Q3W |
| | | | 5b | PD-1/L1 naive | GEN1046 100 mg |
| | | | | | 1Q3W |
| EC6 | 40 | SCCHN | 6a | Prior CPI | GEN1046 100 mg |
| | | | | treatment | 1Q3W |
| | | | 6b | PD-1/L1 naive | GEN1046 100 mg |
| | | | | | 1Q3W |

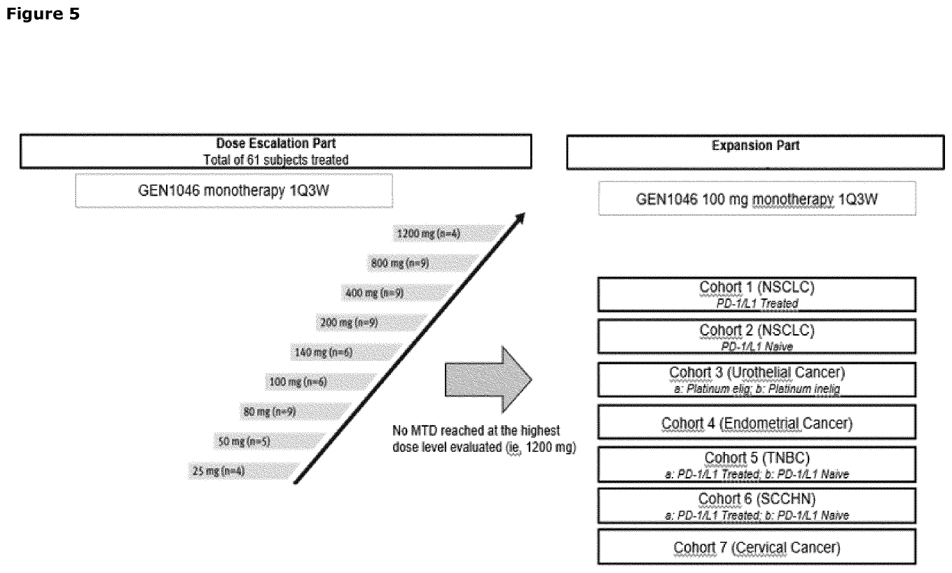

A diagram of the trial design is provided in FIG. 5. Further disclosure of the dose escalation and the expansion cohorts, as well as preliminary results from dose escalation are provided in International Patent Application WO 2021/156326.

### Preliminary Results and Conclusions

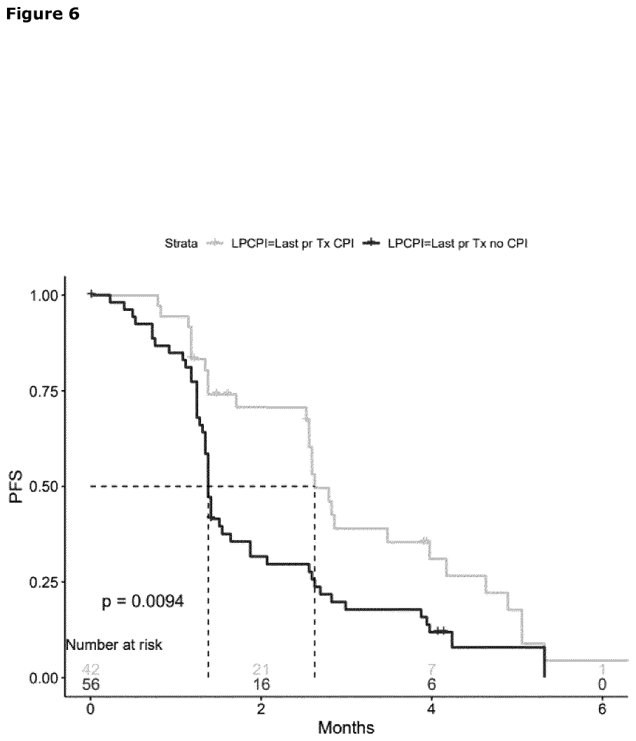

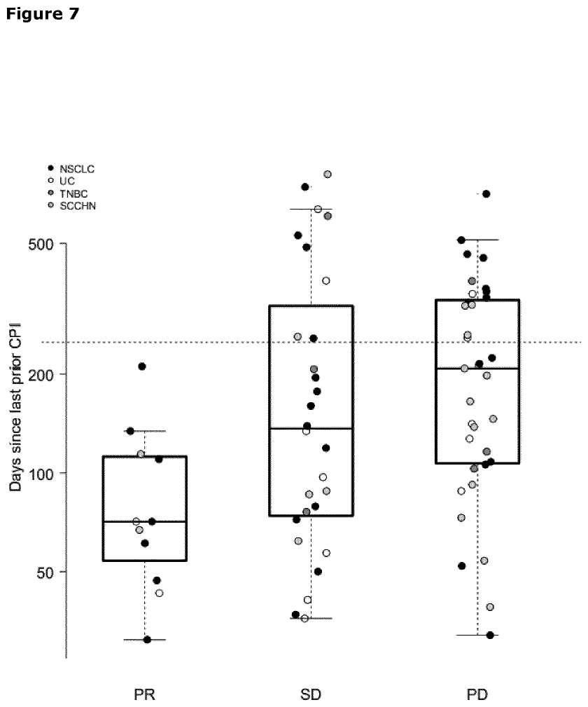

Doses of 25 to 1200 mg Q3W that were evaluated in the escalation phase of the FIH trial were safe and generally well tolerated. The MTD was not reached.
Preliminary evaluation of safety data showed no dose dependency, indicating there is no dose response with respect to frequency of AEs.
Responses according to RECIST v1.1 were observed at GEN1046 doses of 80 to 200 mg Q3W in the dose-escalation phase of the FIH trial. Additionally, responses were also observed in expansion with a dose of 100 mg Q3W.
Consistent modulation of pharmacodynamic markers (proliferating [Ki67+] effector memory CD8⁺ T cells and total CD8⁺ T cells and increased levels of IFNγ and IP-10) was observed in peripheral blood at dose levels <200 mg. Reduced modulation of these endpoints was observed at higher dose levels (>400 mg).
The semi-mechanistic PK/pharmacodynamic model (see example 13 in WO 2021/156326) predicted a bell-shaped response for trimer formation, which peaked around 100 mg Q3W. To balance the trimer levels and target engagement with respect to PD-L1 RO, a dose of 100 mg Q3W was chosen that may provide optimum initial response to GEN1046.
Progression-free survival (PFS) was longer in subjects having received prior treatment with checkpoint inhibitor (FIG. 6).
Clinical response to GEN1046 monitherapy in checkpoint inhibitor pre-treated NSCLC subjects associates with time from last prior anti-PD-1 therapy (FIG. 7).
   NSCLC subjects with benefit on GEN1046 monotherapy showed a trend for more recent treatment with last anti-PD-1 agent Shorter time since anti-PD-1 agent containing therapy may suggest residual anti-PD-1 activity is facilitating response to GEN1046. Supportive of this, patients treated with anti-PD-1 agents in the clinic exhibit long-term PD-1 receptor occupancy by the therapeutic antibody which can last for more than 200 days (Brahmer et al., JCO 2010; 28(19): 3167-3175). Having therapeutic a-PD-1 agent still bound to the PD-1 receptors may in turn lead to a larger number of free PD-L1 molecules being available for binding to GEN1046.
   Presence of residual a-PD-1 activity may also allow for more complete blockade of the PD-1 pathway (blocking interaction of PD-1 with both PD-L1 and PD-L2), which may be important for the biological activity of GEN1046 in the post-CPI setting.
   More recent anti-PD-1 treatment may have direct impact on the tumor microenvironment, for example by initiating an anti-tumor immune response which can be enhanced by GEN1046 if it is given immediately or soon after progression on the anti-PD-1 containing therapy.
   Responders presented with “low” PD-1+CD8 T cell frequency, which may reflect receptor occupancy (RO) by prior a-PD-1 treatment
   Conversely, non-responders presented with generally high PD-1+CD8 T cell frequency which may indicate a more exhausted phenotype

### Example 5: Generation of IgG1-PD1 and Screening Materials

The techniques and methods used herein are described herein or carried out in a manner known per se and as described, for example, in Sambrook et al., Molecular Cloning: A Laboratory Manual, 2^{nd} Edition (1989) Cold Spring Harbor Laboratory Press, Cold Spring Harbor, N.Y. All methods including the use of kits and reagents are carried out according to the 30 manufacturers' information unless specifically indicated.

### PD-1 and FcγR Constructs

Plasmids encoding various full-length PD-1 variants were generated: human (Homo sapiens; 35 UniProtKB ID: Q15116), cynomolgus monkey (Macaca fascicularis; UniProtKB ID: BOLAJ3), dog (Canis familiaris; UniProtKB ID: E2RPS2), rabbit (Oryctolagus cuniculus; UniProtKB ID: GISUFO), pig (Sus scrofa; UniProtKB ID: A0A287A1C3), rat (Rattus norvegicus; UniProtKB ID: D3ZIN8), and mouse (Mus musculus; UniProtKB ID: Q02242), as well as a plasmid encoding human FeyRIa (UniProt KB ID: P12314).

### Generation of CHO-S Cell Lines Transiently Expressing Full-Length PD-1 or FcγR Variants

CHO-S cells (a subclone of CHO cells adapted to suspension growth; ThermoFisher Scientific, cat. no. R800-07) were transfected with PD-1 or FcγR plasmids using FreeStyle™ MAX Reagent (ThermoFisher Scientific, cat. no. 16447100) and OptiPRO™ serum-free medium (ThermoFisher Scientific, cat. no. 12309019), according to the manufacturer's instructions.

### Production of Antibody Variants

### IgG1-PD1

Three New Zealand White rabbits were immunized with recombinant human His-tagged PD-1 protein (R&D Systems, cat. no. 8986-PD). Single B cells from blood were sorted and supernatants screened for production of PD-1 specific antibodies by human PD-1 enzyme-linked immunosorbent assay (ELISA), cellular human PD-1 binding assay and by human PD-1/PD-L1 blockade bioassay. From screening-positive B cells, RNA was extracted, and sequencing was performed. The variable regions of heavy and light chain were gene synthesized and cloned N-terminal of human immunoglobulin constant parts (IgG1/x) containing mutations L234A and L235A (LALA; Labrijn et al., Sci Rep 2017, 7:2476) wherein the amino acid position number is according to Eu numbering (SEQ ID NO: 98) to minimize interactions with Fcγ receptors.

Transient transfections of HEK293-FreeStyle cells using 293-free transfection reagent (Novagen/Merck) were executed by Tecan Freedom Evo device. Produced chimeric antibodies were purified from cell supernatant using protein-A affinity chromatography on a Dionex Ultimate 3000 HPLC with plate autosampler. Purified antibodies were used for further analysis in particular retesting by human PD-1 ELISA, cellular human PD-1 binding assay, human PD-1/PD-L1 blockade bioassay, and T-cell proliferation assay. The chimeric rabbit antibody MAB-19-0202 (SEQ ID NO: 109 and 110) was identified as best performing clone and subsequently humanized.

The variable region sequences of the chimeric PD-1 antibody MAB-19-0202 are shown in the following tables. Table 12 shows the variable regions of the heavy chain, while table 13 shows the variable regions of the light chain. In both cases the framing regions (FRs) as well as the complementarity determining regions (CDRs) according to Kabat numbering are defined. The underlined amino acids indicate the CDRs according to the IMGT numbering. The bold letters indicate the intersection of Kabat and IMGT numbering.

TABLE 12
HEAVY CHAIN

| Sequence | | | | | | | |
|---|---|---|---|---|---|---|---|
| ID | FR1 | CDR1 | FR2 | CDR2 | FR3 | CDR3 | FR4 |
| MAB-19- | QSVEE | SYN | WVR | IISGG | RFTI | AFYDD | WGPGTL |
| 0202-HC | SGGRL | MG | QAP | TIGH | SKTS | YDYN | VTVSS |
| SEQ ID | VTPGT | | GKG | YASW | STTV | V | |
| NO: 109 | PLTLT | | LEYI | AKG | DLKM | | |
| | CTVSG | | G | | TSLT | | |
| | FSLY | | | | TEDT | | |
| | | | | | ATYF | | |
| | | | | | CAR | | |

TABLE 13
LIGHT CHAIN

| Sequence | | | | | | | |
|---|---|---|---|---|---|---|---|
| ID | FR1 | CDR1 | FR2 | CDR2 | FR3 | CDR3 | FR4 |
| MAB-19- | AAVL | QSSQ | WYQ | QAS | GVPSR | AGG | FGGGT |
| 0202-LC | TQTP | SVYG | QKPG | KLE | FKGSG | YSSS | EVVVK |
| SEQ ID | SPVS | NNQL | QPPK | T | LSGTQ | SDTT | |
| NO: 110 | AAVG | S | LLIY | | FTTIS | | |
| | GTVT | | | | DLESD | | |
| | ISC | | | | DAATY | | |
| | | | | | YC | | |

Humanized heavy and light chain variable region antibody sequences were generated by structural modelling-assisted CDR grafting, gene synthesized and cloned N-terminal of human immunoglobulin constant parts (IgG1/x with LALA mutations). Humanized antibodies were used for further analysis in particular retesting by human PD-1 ELISA, cellular human PD-1 binding assay, human PD-1/PD-L1 blockade bioassay, and the T-cell proliferation assay. The humanized antibody MAB-19-0618 (SEQ ID NO: III and 112) was identified as best performing clone.

The allocation of the humanized light and heavy chains to antibody ID of the recombinant humanized sequences are listed in Table 14. The variable region sequences of the humanized light and heavy chains are shown in Table 15 and 16. Table 15 shows the variable regions of the heavy chain, while table 16 shows the variable regions of the light chain. In both cases the framing regions (FRs) as well as the complementarity determining regions (CDRs) according to Kabat numbering are defined. The underlined amino acids indicate the CDRs according to the IMGT numbering.

TABLE 14

| | light chain | | heavy chain | |
|---|---|---|---|---|
| | humanized | Light chain | humanized | Heavy chain |
| antibody ID | variant | SEQ ID NO: | variant | SEQ ID NO: |
| MAB-19- | MAB-19- | 112 | MAB-19-0202-H5 | 111 |
| 0618 | 0202-L4 | | | |

TABLE 15
HEAVY CHAIN

| Sequence | | | | | | | |
|---|---|---|---|---|---|---|---|
| ID | FR1 | CDR1 | FR2 | CDR2 | FR3 | CDR3 | FR4 |
| MAB-19- | QVQLV | SYN | WV | IISG | RFTIS | AFY | WG |
| 0202-H5 | ESGGG | MG | RQ | GTI | RDTSK | DDY | PG |
| SEQ ID | LVQPG | | AP | GHY | TTLYL | DYN | TL |
| NO: 111 | TSLRL | | GK | ASW | QMNSL | V | VT |
| | SCSVS | | GL | AKG | TTEDT | | VS |
| | GFSLY | | EY | | ATYFC | | S |
| | | | IG | | AR | | |

TABLE 16
LIGHT CHAIN

| Sequence | | | | | | | |
|---|---|---|---|---|---|---|---|
| ID | FR1 | CDR1 | FR2 | CDR2 | FR3 | CDR3 | FR4 |
| MAB-19- | AIQLT | QSS | WY | QAS | GVPSR | AGG | FG |
| 0202-L4 | QSPSS | QSV | QQ | KLE | FRGSG | YSS | GG |
| SEQ ID | LSASV | YGN | KP | T | SGTQF | SSD | TE |
| NO: 112 | GGTVT | NQL | GQ | | TLTIS | TT | VV |
| | ITC | S | PP | | SLQSE | | VK |
| | | | KL | | DFATY | | |
| | | | LI | | YC | | |
| | | | Y | | | | |

The sequences of the variable regions of the heavy and light chains of MAB-19-0618 were gene synthesized and cloned by ligation-independent cloning (LIC) into expression vectors with codon-optimized sequences encoding the human IgG1m(f) heavy chain constant domain containing the Fc-silencing mutations L234F, L235E and G236R (FER) wherein the amino acid position number is according to Eu numbering (SEQ ID NO: 93) and the human kappa light chain constant domain (SEQ ID NO: 97). The resulting antibody was designated IgG1-PD1.

The GS Xceed® Expression System (Lonza) was used to generate a stable cell line expressing IgG1-PD1. The sequences encoding the heavy and light chain of IgG1-PD1 were cloned into the expression vectors pXC-18.4 and pXC-Kappa (containing the glutamine synthetase [GS]gene), respectively, by Lonza Biologics plc. Next, a double gene vector (DGV) encoding both the heavy and light chain of IgG1-PD1 was constructed by ligating the complete expression cassette from the heavy chain vector into the light chain vector. The DNA of this DGV was linearized with the restriction enzyme PvuI-HF (New England Biolabs, R3150L) and used for stable transfection of CHOK1SV® GS-KO® cells. IgG1-PD1 was purified for functional characterization.

### IgG1-CD52-E430G

A human IgG1 antibody with an E430G hexamerization-enhancing mutation (WO2013/004842 A2) in the Fc domain (SEQ ID NO: 95) and antigen-binding domains identical to CAMPATH-1H, a CD52-specific antibody, was used as a positive control in C1q binding experiments (Crowe et al., 1992 Clin Exp Immunol. 87(1):105-110) (SEQ ID NO. 116 and 120).

### Control Antibodies

Human IgG1 antibodies with antigen-binding domains identical to b12, an HIV1 gp120-specific antibody, were used as negative controls in several experiments (Barbas et al., J Mol Biol. 1993 Apr. 5; 230(3):812-2). VH and VL domains of b12 (SEQ ID NO. 123 and 127) were prepared by de novo gene synthesis (GeneArt Gene Synthesis; ThermoFisher Scientific, Germany) and cloned into expression vectors containing a human IgG1 heavy chain constant region (i.e. CH1, hinge, CH2 and CH3 region) of the human IgG1m(f) allotype (SEQ ID NO: 92) or a variant thereof (containing the L234F/L235E/G236R mutations and an additional, in the context of this study functionally irrelevant, K409R mutation in the Fc domain, abbreviated as the FERR mutations) (SEQ ID NO: 94) or containing a human IgG4 heavy chain constant region (SEQ ID NO: 96); or the constant region of the human kappa light chain (LC) (SEQ ID NO: 97), as appropriate for the selected binding domains. Antibodies were obtained by transfection of heavy and light chain expression vectors in production cell lines and purified for functional characterization.

### Example 6: Binding of IgG1-PD1 to PD-1 from Various Species

Binding of IgG1-PD1 to PD-1 of species commonly used for nonclinical toxicology studies was assessed by flow cytometry using CHO-S cells transiently expressing PD-1 from different animal species.

CHO-S cells (5×10⁴ cells/well) were seeded in round-bottom 96-well plates. Antibody dilutions (1.7×10⁻⁴-30 μg/mL or 5.6×10⁻⁵-10 μg/mL, 3fold dilutions) of IgG1-PD1, IgG1-ctrl-FERR, and pembrolizumab were prepared in Genmab (GMB) fluorescence-activated cell sorting (FACS) buffer (phosphate-buffered saline [PBS; Lonza, cat. no. BE17-517Q, diluted to 1× PBS in distilled water] supplemented with 0.1% [w/v] bovine serum albumin [BSA; Roche, cat. no. 10735086001] and 0.02% [w/v] sodium azide [NaN3; bioWORLD, cat. no. 41920044-3]). An IgG4 isotype control (BioLegend, cat. no. 403702) for pembrolizumab was included only at the highest concentration tested (30 μg/mL or 10 μg/mL). Cells were centrifuged, supernatant was removed, and cells were incubated in 50 μL of the antibody dilutions for 30 min at 4° C. Cells were washed twice with GMB FACS buffer and incubated with 50 μL secondary antibody R-phycoerythrin (PE)-conjugated goat-anti-human IgG F(ab′)2 (Jackson ImmunoResearch, cat. no. 109-116-098; diluted 1:500 in GMB FACS buffer) for 30 min at 4° C., protected from light. Cells were washed twice with GMB FACS buffer, resuspended in GMB FACS buffer supplemented with 2 mM ethylenediaminetetraacetic acid (EDTA; Sigma-Aldrich, cat. no. 03690) and 4′,6-diamidino-2-phenylindole (DAPI) viability marker (1:5,000; BD Pharmingen, cat. no. 564907). Antibody binding to viable cells (as identified by DAPI exclusion) was analyzed by flow cytometry on an Intellicyt® iQue PLUS Screener (Intellicyt Corporation) using FlowJo software. Binding curves were analyzed using non-linear regression analysis (four-parameter dose-response curve fits) in GraphPad Prism.

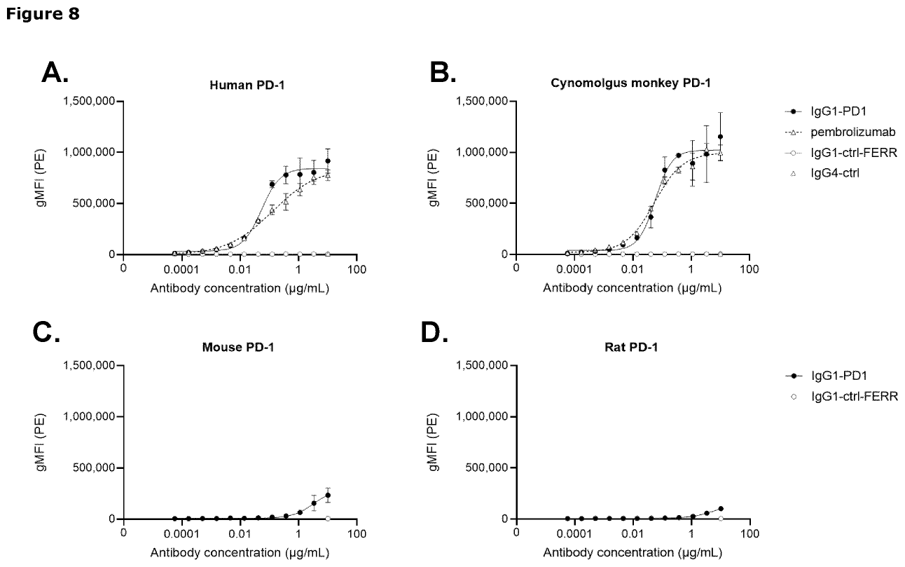

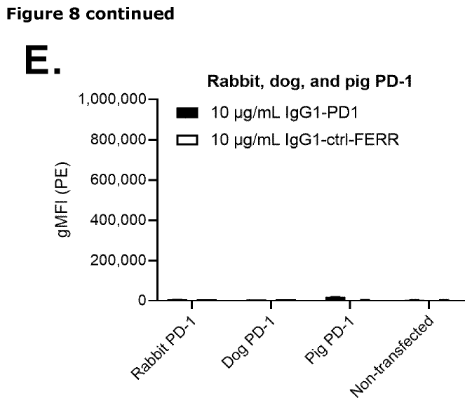

Binding of IgG1-PD1 to PD-1 of different species was evaluated by flow cytometry using CHO-S cells transiently transfected to express human, cynomolgus monkey, dog, rabbit, pig, rat, or mouse PD-1 protein on the cell surface. Dose-dependent binding of IgG1-PD1 was observed for human and cynomolgus monkey PD-1 (FIG. 8A-B). Pembrolizumab demonstrated comparable binding. Substantially reduced cross-reactivity of IgG1-PD1, and only at the highest concentrations, was observed to rodent PD-1 (mouse, rat; FIG. 8C-D) and no binding was observed to PD-1 of other species frequently used in toxicology studies (rabbit, dog, pig; FIG. 8E). No IgG1-PD1 binding was observed to non-transfected control cells (FIG. 8E), nor was binding of IgG1-ctrl-FERR, included as a negative control, observed to PD-1 of any of the tested species (FIG. 8).

In conclusion, IgG1-PD1 showed comparable binding to membrane-expressed human and cynomolgus monkey PD-1 and significantly lower or no binding to mouse, rat, rabbit, dog, and pig PD-1.

### Example 7: Binding to Human and Cynomolgus Monkey PD-1 Determined by Surface Plasmon Resonance

Binding of immobilized IgG1-PD1, pembrolizumab, and nivolumab to human and cynomolgus monkey PD-1 was analyzed by surface plasmon resonance (SPR) using a Biacore 8K SPR system. Recombinant human and cynomolgus monkey PD-1 extracellular domain (ECD) with a C-terminal His-tag were obtained from Sino Biological (cat. no. HPLC-10377-H08H and 90311-C08H, respectively).

Biacore Series S Sensor Chips CM5 (Cytiva, cat. no. 29149603) were covalently coated with anti-Fc antibody using amine coupling and the Human Antibody Capture Kit, Type 2 (Cytiva, cat. no. BRI00050 and BR100839) according to the manufacturer's instructions.

Subsequently, IgG1-PD1 (2 nM), nivolumab (Bristol-Myers Squibb, lot no. ABP6534; 1.25 nM), and pembrolizumab (Merck Sharp & Dohme, lot. no. T019263; 1.25 nM), diluted in HBS-EP+buffer (Cytiva, cat. no. BR100669; diluted to 1× in distilled water [B Braun, cat. no. 00182479E]), were captured onto the surface at 25° C., with a flow rate of 10 μL/min and a contact time of 60 seconds. This resulted in captured levels of approximately 50 resonance units (RU).

After three start-up cycles of HBS-EP+buffer, human or cynomolgus monkey PD-1 ECD samples (0.19-200 nM; 2-fold dilution in HBS-EP+buffer; 12 cycles) were injected to generate binding curves. Each sample that was analyzed on an antibody coated surface (active surface) was also analyzed on a parallel flow cell without antibody (reference surface), which was used for background correction.

At the end of each cycle, the surface was regenerated using 10 mM Glycine-HCl pH 1.5 (Cytiva, cat. no. BR100354). The data were analyzed using the predefined “Multi-cycle kinetics using capture” evaluation method in the Biacore Insight Evaluation software (Cytiva). The sample with the highest concentration of human or cynomolgus monkey PD-1 (200 nM) was omitted from analysis to allow better curve fits of the data.

Immobilized IgG1-PD1 bound to human PD-1 ECD with a binding affinity (K_{D}) of 1.45±0.05 nM (Table 17). Nivolumab and pembrolizumab bound human PD-1 ECD with a binding affinity comparable to the K_{D} of IgG1-PD1, i.e., with K_{D} values in the low nanomolar range (4.43±0.08 nM and 3.59±0.10 nM, respectively) (Table 17).

Immobilized IgG1-PD1 bound to cynomolgus monkey PD-1 ECD with a K_{D} of 2.74 i 0.58 nM (Table 18), comparable to the affinity of IgG1-PD1 for human PD-1. Nivolumab and pembrolizumab bound cynomolgus monkey PD-1 ECD with a binding affinity comparable to the K_{D} of IgG1-PD1 for cynomolgus monkey PD-1 ECD and comparable to the K_{D} of nivolumab and pembrolizumab for human PD-1 ECD, i.e., with K_{D} values in the low nanomolar range (2.93±0.58 nM and 0.90±0.06 nM, respectively) (Table 18).

TABLE 17
Binding affinities of PD-1 antibodies to the extracellular domain of human PD-1 as determined
by surface plasmon resonance. The association rate constant kₐ (1/Ms), dissociation rate constant
k_{d} (1/s) and equilibrium dissociation constant K_{D} (M) of IgG1-PD1, nivolumab, and pembrolizumab
for the ECD of human PD-1 were determined by SPR.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1^{a} | 1.45 × 10⁻⁹ | 4.51 × 10⁻¹¹ | 5.17 × 10⁵ | 2.03 × 10⁴ | 7.51 × 10⁻⁴ | 9.61 × 10⁻⁶ |
| Nivolumab^{b} | 4.43 × 10⁻⁹ | 8.49 × 10⁻¹¹ | 4.06 × 10⁵ | 3.54 × 10³ | 1.80 × 10⁻³ | 2.12 × 10⁻⁵ |
| Pembrolizumab^{b} | 3.59 × 10⁻⁹ | 9.90 × 10⁻¹¹ | 2.12 × 10⁶ | 1.36 × 10⁶ | 7.57 × 10⁻³ | 4.71 × 10⁻³ |

^{a}Average and SD from three independent experiments.
^{b}Average and SD from two independent experiments.
Abbreviations: K_{D} = equilibrium dissociation constant; kₐ = association rate constant; k_{d} = dissociation rate constant or off-rate; SD = standard deviation.

TABLE 18
Binding affinities of PD-1 antibodies to the extracellular domain of cynomolgus monkey PD-1
as determined by surface plasmon resonance. The association rate constant kₐ (1/Ms), dissociation rate
constant k_{d} (1/s) and equilibrium dissociation constant K_{D} (M) of IgG1-PD1, nivolumab, and
pembrolizumab for the ECD of cynomolgus monkey PD-1 were determined by SPR.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1^{a} | 2.74 × 10⁻⁹ | 5.79 × 10⁻¹⁰ | 5.34 × 10⁵ | 9.39 x 10⁴ | 1.43 × 10⁻³ | 7.23 × 10⁻⁵ |
| Nivolumab^{a} | 2.93 × 10⁻⁹ | 5.81 × 10⁻¹⁰ | 3.28 × 10⁵ | 5.07 × 10⁴ | 9.43 × 10⁻⁴ | 9.07 × 10⁻⁵ |
| Pembrolizumab^{b} | 8.99 × 10⁻¹⁰ | 5.73 × 10⁻¹¹ | 7.14 × 10⁵ | 3.04 × 10⁴ | 6.42 × 10⁻⁴ | 6.86 × 10⁻⁵ |

^{a}Average and SD from three independent experiments.
^{b}Average and SD from two independent experiments.
Abbreviations: K_{D} = equilibrium dissociation constant; kₐ = association rate constant; k_{d} = dissociation rate constant or off-rate; SD = standard deviation.

### Example 8: Effect of IgG1-PD1 on PD-1 Ligand Binding and PD-1/PD-L1 Signaling

To confirm that IgG1-PD1 functions as a classical immune checkpoint inhibitor, the capacity of IgG1-PD1 to disrupt PD-1 ligand binding and PD-1 checkpoint function was assessed in vitro.

Competitive binding of IgG1-PD1 with recombinant human PD-L1 and PD-L2 to membrane-expressed human PD-1 was assessed by flow cytometry. CHO-S cells transiently transfected with human PD-1 (see Example 5; 5×10⁴ cells/well) were added to the wells of a round-bottom 96-well plate (Greiner, cat. no. 650180), pelleted, and placed on ice. Biotinylated recombinant human PD-L1 (R&D Systems, cat. no. AVI156) or PD-L2 (R&D Systems, cat. no. AVIi224), diluted in PBS (Cytiva, cat. no. SH3A3830.03), was added to the cells (final concentration: 1 μg/mL), immediately after which a concentration range of IgG1-PD1, pembrolizumab (MSD, lot no. T019263 and T036998), or IgG1-ctrl-FERR, diluted in PBS, was added (final concentrations: 30 μg/mL −0.5 ng/mL in three-fold dilution steps). Cells were then incubated for 45 min at RT. Cells were washed twice with PBS and incubated with 50 μL streptavidin-allophycocyanin (R&D Systems, cat. no. F0050; diluted 1:20 in PBS) for 30 min at 4° C., protected from light. Cells were washed twice with PBS and resuspended in 20 μL GMB FACS buffer. Streptavidin-allophycocyanin binding was analyzed by flow cytometry on an Intellicyt® iQue Screener PLUS (Sartorius) using FlowJo software.

The effect of IgG1-PD1 on the functional interaction of PD-1 and PD-L1 was determined using a bioluminescent cell-based PD-1/PD-L1 blockade reporter assay (Promega, cat. no. J1255), essentially as described by the manufacturer. Briefly, cocultures of PD-L1 aAPC/CHO-Kl Cells and PD-1 Effector Cells were incubated with serially diluted IgG1-PD1, pembrolizumab (MSD, lot no. 10749880 or T019263), nivolumab (Bristol-Myers Squibb, lot no. 11024601), or IgG1-ctrl-FERR (final assay concentrations: 15-0.0008 μg/mL in 3-fold dilutions or 10-0.0032 μg/mL in 5-fold dilutions) for 6 h at 37° C., 5% CO₂. Cells were then incubated at RT with reconstituted Bio-Glo™ for 5-30 min, after which luminescence (in relative light units [RLU]) was measured using an Infinite® F200 PRO Reader (Tecan) or an EnVision Multilabel Plate Reader (PerkinElmer).

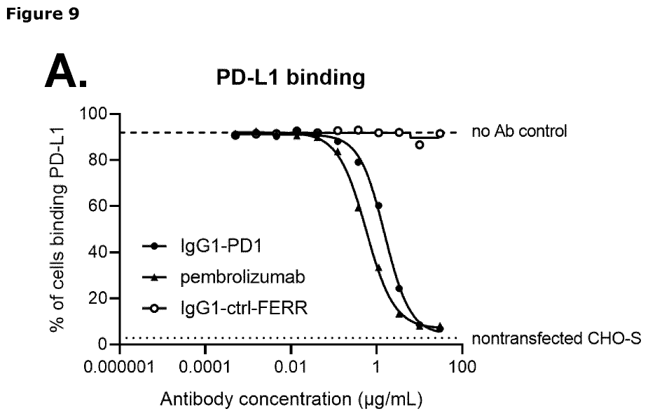

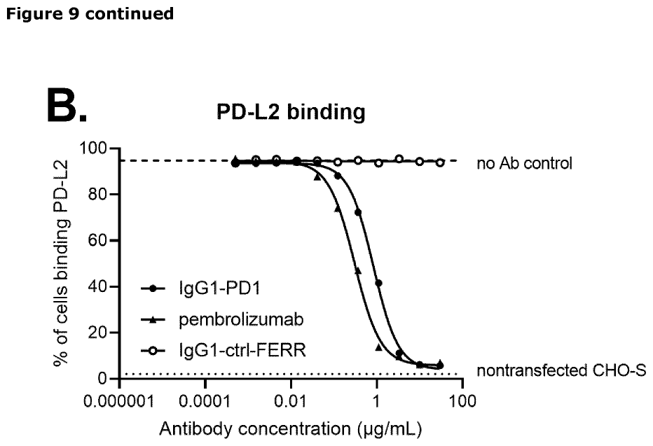

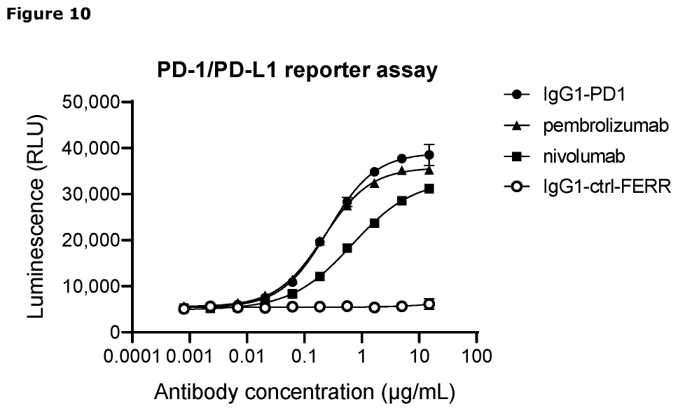

Dose-response curves were analyzed by non-linear regression analysis (four-parameter dose-response curve fits) using GraphPad Prism software, and the concentrations at which 50% of the maximal (inhibitory) effect was observed (EC₅₀/IC₅₀) were derived from the fitted curves. IgG1-PD1 disrupted binding of human PD-L1 and PD-L2 to membrane-expressed human PD-1 in a dose-dependent manner (FIG. 9), with IC₅₀ values of 2.059+0.653 μg/mL (13.9+4.4 nM) for PD-L1 binding inhibition and 1.659+0.721 μg/mL (11.2+4.9 nM) for PD-L2 binding inhibition, i.e., in the nanomolar range (Table 19). Pembrolizumab showed PD-L1 and PD-L2 binding inhibition with comparable potency, i.e., with IC₅₀ values in the nanomolar range.

Functional blockade of the PD-1/PD-L1 axis was tested using a cell-based bioluminescent PD-1/PD-L1 blockade reporter assay. Cocultures of reporter Jurkat T cells expressing human PD-1 and harboring an NFAT-RE-driven luciferase, and PD-L1 aAPC/CHOKI cells expressing human PD-L1 and an antigen-independent TCR activator, were incubated in absence and presence of concentration dilution series of IgG1-PD1, pembrolizumab, or nivolumab. IgG1-ctrl-FERR was included as a negative control. Blockade of the PD-1/PD-L1 interaction results in the release of the PD1/PDL1 mediated inhibitory signal, leading to TCR activation and NFAT-RE-mediated luciferase expression (luminescence measured). IgG1-PD1 induced a dose-dependent increase of TCR signaling in PD-1′ reporter T cells (FIG. 10). The EC₅₀ was 0.165+0.056 μg/mL (1.12+0.38 nM; Table 20). Pembrolizumab similarly alleviated PD-1 mediated inhibition of TCR signaling, with an EC₅₀ of 0.129+0.051 μg/mL (0.86+0.34 nM), ie, with comparable potency. Nivolumab alleviated the inhibition of TCR signaling with an EC₅₀ of 0.479+0.198 μg/mL (3.28+1.36 nM), i.e., with slightly lower potency.

In summary, IgG1-PD1 acts as a classical immune checkpoint inhibitor in vitro, by blocking PD-1 ligand binding and disrupting PD-1 immune checkpoint function.

TABLE 19
IC₅₀ values of IgG1-PD1-mediated inhibition of PD-1 ligand binding
IC₅₀ values were calculated from the competition binding curves.

| Competitive binding | | | | Competitive binding | | | |
|---|---|---|---|---|---|---|---|
| with human PD-L1 | | | | with human PD-L2 | | | |
| (average IC50 [ + SD]) | | | | (average IC50 [ + SD]) | | | |
| IgG1-PD1 | | pembrolizumab | | IgG1-PD1 | | pembrolizumab | |
| μg/mL | nM | μg/ml | nM | μg/mL | nM | μg/mL | nM |
| 2.059 | 13.9 | 1.134 | 7.6 | 1.659 | 11.2 | 1.186 | 8.0 |
| [±0.653] | [±4.4] | [±0.493] | [±3.3] | [±0.721] | [±4.9] | [±0.770] | [±5.2] |

Abbreviations: IC₅₀ = concentration at which 50% of the inhibitory effect was observed; PD-1 = programmed cell death protein 1; PD-L1 = programmed cell death 1 ligand 1; PD-L2 = programmed cell death 1 ligand 2; SD = standard deviation.

TABLE 20
EC₅₀ of PD-1/PD-L1 checkpoint blockade
Cocultures of PD-1⁺ reporter T cells and PD-L1 aAPC/CHO-K
cells were incubated with concentration series of IgG1-PD1,
pembrolizumab, or nivolumab in PD-1/PD-L1 blockade
reporter assays. Inhibition of PD-1/PD-L1 checkpoint
function, resulting in downstream TCR signaling and luciferase
expression in the reporter T cells, was determined by
measuring luminescence. From the resulting dose-response
curves, EC₅₀ values were calculated.
Average EC₅₀ [± SD]

| IgG1-PD1 | | Pembrolizumab | | Nivolumab | |
|---|---|---|---|---|---|
| μg/mL | nM | μg/mL | nM | μg/mL | nM |
| 0.165 | 1.12 | 0.129 | 0.86 | 0.479 | 3.28 |
| [±0.056] | [±0.38] | [±0.051] | [±0.34] | [±0.198] | [±1.36] |

Abbreviations: aAPC = artificial antigen-presenting cell; CHO = Chinese hamster ovary; EC₅₀ = concentration at which 50% of the maximal effect is observed; PD-1 = programmed cell death protein 1; PD-L1 = programmed cell death 1 ligand 1; SD = standard deviation; TCR = T-cell receptor.

### Example 9: Antigen-Specific Proliferation Assay to Determine the Capacity of IgG1-PD1 to Enhance Proliferation of Activated T Cells

To determine the capacity of IgG1-PD1 to enhance T-cell proliferation, an antigen-specific proliferation assay was conducted using PD-1-overexpressing human CD8⁺ T cells.

HLA-A*02′ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved in RPMI 1640 containing 10% DMSO (AppliChem GmbH, cat. no A3672,0050) and 10% human albumin (CSL Behring, PZN 00504775) for T-cell isolation. For differentiation into immature DCs (iDCs), 1×10⁶ monocytes/mL were cultured for five days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 200 ng/mL granulocyte-macrophage colony-stimulating factor (GM-CSF; Miltenyi, cat. no. 130-093-868) and 200 ng/mL interleukin-4 (IL-4; Miltenyi, cat. no. 130-093-924). After three days in culture, half of the medium was replaced with fresh medium. On day 5, iDCs were harvested by collecting non-adherent cells and adherent cells were detached by incubation with Dulbecco's phosphate-buffered saline (DPBS) containing 2 mM EDTA for 10 min at 37°. After washing with DPBS iDCs were cryopreserved in fetal bovine serum (FBS; Sigma-Aldrich, cat. no. F7524) containing 10% DMSO for future use in antigen-specific T cell assays.

One day prior to the start of an antigen-specific CD8⁺ T cell proliferation assay, frozen PBLs and iDCs from the same donor were thawed. CD8⁺ T cells were isolated from PBLs by MACS technology using anti-CD8 MicroBeads (Miltenyi, cat. no. 130-045-201), according to the manufacturer's instructions. About 10×10⁶ to 15×10⁶ CD8⁺ T cells were electroporated with each 10 μg of in vitro translated (IVT)-RNA encoding the alpha and beta chains of a murine TCR specific for human claudin-6 (CLDN6; HLA-A*02-restricted; described in WO 2015150327 Al) plus 10 μg IVT-RNA encoding PD-1 (UniProt Q15116) in 250 μL X-Vivol5 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 1×3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 1.6 μM carboxyfluorescein succinimidyl ester (CFSE; Life Technologies GmbH, cat. No V12883) in PBS according to the manufacturer's instructions and incubated in IMDM medium supplemented with 5% pooled human serum overnight.

Up to 5×10⁶ thawed iDCs were electroporated with 2 μg IVT-RNA encoding full-length human CLDN6 (WO 2015150327 Al), in 250 μL X-Vivol5 medium, using the electroporation system as described above (300 V, 1×12 ms pulse) and incubated in IMDM medium supplemented with 5% pooled human serum overnight.

The next day, cells were harvested. Cell-surface expression of CLDN6 on iDCs, as well as cell-surface expression of the CLDN6-specific TCR and PD-1 on T cells was confirmed by flow cytometry. To this end, iDCs were stained with a DyLight650-conjugated CLDN6-specific antibody (non-commercially available; in-house production). T cells were stained with a brilliant violet (BV)421-conjugated anti-mouse TCR-P chain antibody (Becton Dickinson GmbH, cat. no. 562839) and an allophycocyanin (APC)-conjugated anti-human PD-1 antibody (Thermo Fisher Scientific, cat. no. 17-2799-42).

Electroporated iDCs were incubated with electroporated, CFSE-labeled T cells at a ratio of 1:10 in the presence of IgG1-PD1, pembrolizumab (Keytruda®, MSD Sharp & Dohme GmbH, PZN 10749897), or nivolumab (Opdivo®, Bristol-Myers Squibb, PZN 11024601) at 4-fold serial dilutions (range 0.00005 to 0.8 μg/mL) in IMDM medium containing 5% pooled human serum in a 96-well round-bottom plate. The negative control antibody IgG1-ctrl-FERR was used at a single concentration of 0.8 μg/mL. After 4 d of culture, the cells were stained with an APC-conjugated anti-human CD8 antibody. T-cell proliferation was evaluated by flow cytometry analysis of CFSE dilution in CD8⁺ T cells using a BD FACSCelesta™ flow cytometer (Becton Dickinson GmbH).

Flow cytometry data was analyzed using FlowJo software version 10.7.1. CFSE label dilution of CD8⁺ T cells was assessed using the proliferation modeling tool in FlowJo, and expansion indices calculated using the integrated formula. Dose-response curves were generated in GraphPad Prism version 9 (GraphPad Software, Inc.) using a 4-parameter logarithmic fit. Statistical significance was determined by Friedman's test and Dunn's multiple comparisons test using GraphPad Prism version 9.

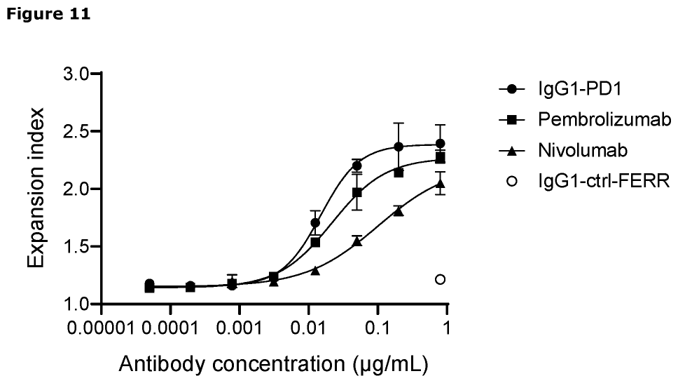

Antigen-specific proliferation of CD8⁺ T cells was enhanced by IgG1-PD1 in a dose-dependent manner (FIG. 11), with EC₅₀ values in the picomolar range (Table 21). Treatment with pembrolizumab or nivolumab also enhanced T-cell proliferation in a dose-dependent manner. The average EC₅₀ of pembrolizumab was comparable to IgG1-PD1, whereas the EC₅₀ of nivolumab was significantly (P=0.0267) higher than that of IgG1-PD1.

TABLE 21
EC₅₀ values in the antigen-specific proliferation assay
EC₅₀ values of IgG1-PD1, pembrolizumab, and nivolumab
were determined using the CD8⁺ T-cell expansion indices as
measured by an antigen-specific T-cell proliferation assay. Data
shown are the values calculated based on the 4-parameter logarithmic fit.
Average EC₅₀ [± SD]

| IgG1-PD1 | | Pembrolizumab | | Nivolumab | |
|---|---|---|---|---|---|
| μg/mL | nM | μg/mL | nM | μg/mL | nM |
| 0.0124 | 0.0837 | 0.0152 | 0.1018 | 0.0701 | 0.4802 |
| [±0.0018] | [±0.0123] | [±0.0049] | [±0.0333] | [±0.0238] | [±0.1632] |

Abbreviations: EC₅₀ = half-maximal effective concentration; FERR = L234F/L235E/G236R-K409R; PD1 = programmed cell death protein 1; SD = standard deviation.

### Example 10: Effect of IgG1-PD1 on Cytokine Secretion in an Allogeneic MLR Assay

To investigate the capacity of IgG1-PD1 to enhance cytokine secretion in a mixed lymphocyte reaction (MLR) assay, three unique, allogeneic pairs of human mature dendritic cells (mDCs) and CD8⁺ T cells were cocultured in the presence of IgG1-PD1. The levels of IFNγ were measured using an IFNγ-specific immunoassay, while the levels of monocyte chemoattractant protein-1 (MCP-1), GM-CSF, interleukin (IL)-1l, IL-2, IL-4, IL-5, IL-6, IL-8, IL-10, IL12-p40, IL-15, IL-17u, and tumor necrosis factor (TNFu) were determined using a customized Luminex multiplex immunoassay.

Human CD14⁺ monocytes were obtained from healthy donors (BioIVT). For differentiation into immature dendritic cells (iDCs), monocytes were cultured for 6 d in RPMI-1640 complete medium (ATCC modification formula; Thermo Fisher, cat. no. A1049101) supplemented with 10% heat-inactivated fetal bovine serum (FBS; Gibco, cat. no. 16140071), 100 ng/mL GM-CSF and 300 ng/mL IL-4 (BioLegend, cat. no. 766206) at 37° C. On day 4, the medium was replaced with fresh medium with supplements. To mature the iDCs, the cells were incubated in RPMI-1640 complete medium supplemented with 10% FBS, 100 ng/mL GM-CSF, 300 ng/mL IL-4, and 5 μg/mL lipopolysaccharide (LPS; Thermo Fisher Scientific, cat. no. 00 4976 93) at 37° C. for 24 h prior to start of the MLR assay. In parallel, purified CD8⁺ T cells obtained from allogeneic healthy donors (BioIVT) were thawed and incubated in RPMI-1640 complete medium supplemented with 10% FBS and 10 ng/mL IL-2 (BioLegend, cat. no. 589106) at 37° C. O/N.

The next day, the LPS-matured dendritic cells (mDCs) and allogeneic CD8⁺ T cells were harvested and resuspended in prewarmed AIM-V medium (Thermo Fisher Scientific, cat. no. 12055091) at 4×10⁻⁵ cells/mL and 4×10⁶ cells/mL, respectively. The mDCs (20,000 cells/well) were incubated with allogeneic naive CD8⁺ T cells (200,000 cells/well) in the presence of an antibody concentration range (0.001-30 μg/mL) of IgG1-PD1, IgG1-ctrl-FERR, or pembrolizumab (MSD, cat. no. T019263) or in the presence of 30 μg/mL IgG4 isotype control (BioLegend, cat. no. 403702) in AIM-V medium in a 96-well round-bottom plate at 37° C.

After 5 d, cell-free supernatant was transferred from each well to a new 96-well plate and stored at −80° C. until further analysis of cytokine concentrations.

The IFNγ levels were determined using an IFNγ-specific immunoassay (Alpha Lisa IFNγ kit; Perkin Elmer, cat. no. AL217) on an Envision instrument, according to the manufacturer's instructions.

The levels of MCP-1, GM-CSF, IL-10, IL-2, IL-4, IL-5, IL-6, IL-8, IL-10, IL12-p40, IL-15, IL-17u and TNFu were determined using a customized Luminex® multiplex immunoassay (Millipore, order no. SPR1526) based on the Human TH17 Magnetic Bead Panel (MILLIPLEX®). Briefly, cell-free supernatants were thawed and 10 μL of each sample was added to 10 μL Assay Buffer in wells of a 384-well plate (Greiner Bio-One, cat. no. 781096) prewashed with 1× Wash Buffer. In parallel, 10 μL of Standard or Control in Assay Buffer was added to the wells, after which 10 μL of assay medium was added. Magnetic beads against the different cytokines were mixed and diluted to 1× concentrations in Bead Diluent, after which 10 μL of the mixed beads was added to each well. The plate was sealed and incubated at 4° C., shaking, O/N. Wells were washed three times with 60 μL 1× Wash Buffer. Subsequently, 10 μL of Custom Detection Antibodies was added to each well, and the plate was sealed and incubated at RT, shaking, for 1 h. Next, 10 μL of streptavidin-PE was added to each well, and the plate was sealed and incubated at RT, shaking, for 30 min. Wells were washed three times with 60 μL 1× Wash Buffer as described above, after which beads were resuspended in 75 μL Luminex Sheath Fluid by shaking at RT for 5 min. Samples were run on a Luminex FlexMap 3D system.

At the start and at the end of the MLR assay, expression of PD-1 on the CD8⁺ T cells and expression of PD-L1 on the mDCs was confirmed by flow cytometry using PE-Cy7-conjugated 15 anti-PD-1 (BioLegend, cat. no. 329918; 1:20), allophycocyanin-conjugated anti-PD-L1 (BioLegend, cat. no. 329708; 1:80), BUV496-conjugated anti-CD3 (BD Biosciences, cat. no. 612940; 1:20), and BUV395-con^{j}ugated anti-CD8 (BD Biosciences, cat. no. 563795; 1:20).

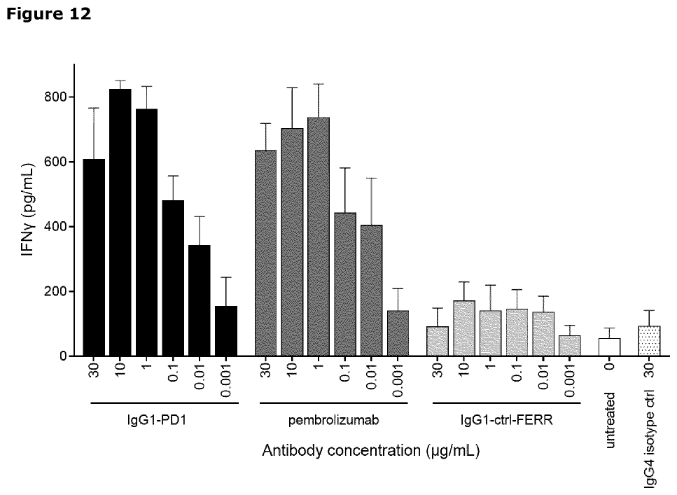

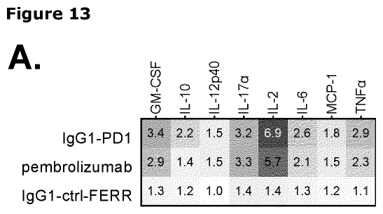

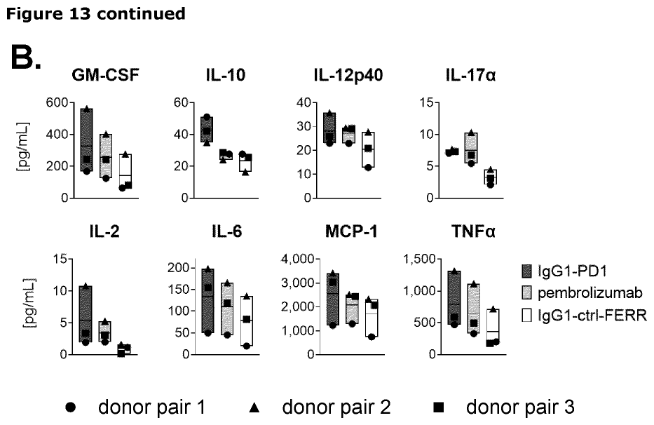

IgG1-PD1 consistently enhanced secretion of IFNγ (FIG. 12) in a dose-dependent manner. IgG1-PD1 also enhanced secretion of MCP-1, GM-CSF, IL-2, IL-6, IL-12p40, IL-17a, IL-10, and TNFα (FIG. 13). Pembrolizumab had a comparable effect on cytokine secretion.

### Example 11: Evaluation of C1q Binding to IgG1-PD1

Binding of complement protein C1q to IgG1-PD1 harboring the FER Fc-silencing mutations in the constant heavy chain region was assessed using activated human CD8⁺ T cells. As a positive control, IgG1-CD52-E430G was included, which has VH and VL domains based on the CD52 antibody CAMPATH-1H and which has an Fc-enhanced backbone that is known to efficiently bind C1q when bound to the cell surface. As non-binding negative control antibodies, IgG1-ctrl-FERR and IgG1-ctrl were included.

Human CD8⁺ T cells were purified (enriched) from buffy coats obtained from healthy volunteers (Sanquin) by negative selection using the RosetteSep™ Human CD8⁺ T Cell Enrichment Cocktail (Stemcell Technologies, cat. no. 15023C.2) or by positive selection via magnetic activated cell sorting (MACS), using CD8 MicroBeads (Miltenyi Biotec, cat. no. 130-045-201) and LS columns (Miltenyi Biotec, cat. no. 130-042-401), all according to the manufacturer's instructions. Purified T cells were resuspended in T-cell medium (Roswell Park Memorial Institute [RPMI]-1640 medium with 25 mM HEPES and L-glutamine [Lonza, cat. no. BE12-115F], supplemented with 10% heat-inactivated donor bovine serum with iron [DBSI; Gibco, cat. no. 20731-030] and penicillin/streptomycin [pen/strep; Lonza, cat. no. DE17-603E]).

Anti-CD3/CD28 beads (Dynabeads™ Human T-Activator CD3/CD28; ThermoFisher Scientific, cat. no. 11132D) were washed with PBS and resuspended in T-cell medium. The beads were added to the enriched human CD8⁺ T cells at a 1:1 ratio and incubated at 37° C., 5% CO₂ for 48 h. Next, the beads were removed using a magnet, and the cells were washed twice in PBS and counted again.

PD-1 expression on the activated CD8⁺ T cells was confirmed by flow cytometry, using IgG1-PD1 (30 μg/mL) and R-phycoerythrin (PE)-conjugated goat-anti-human IgG F(ab′)2 (diluted 1:200 in GMB FACS buffer; Jackson ImmunoResearch, cat. no. 109-116-098), or a commercial PE-conjugated PD-1 antibody (BioLegend, cat. no. 329906; diluted 1:50).

Activated CD8⁺ T cells were seeded in a round-bottom 96-well plate (30,000 or 50,000 cells/well), pelleted, and resuspended in 30 μL assay medium (RPMI-1640 with 25 mM HEPES and L-glutamine, supplemented with 0.1% [w/v] bovine serum albumin fraction V [BSA; Roche, cat. no. 10735086001] and penicillin/streptomycin). Subsequently, 50 μL of IgG1-PD1, IgG1-ctrl-FERR, IgG1-CD52-E430G, or IgG1-ctrl (final concentrations of 1.7×10⁻⁴-30 μg/mL in 3-fold dilution steps in assay medium) was added to each of the wells and incubated at 37° C. for 15 min to allow the antibodies to bind to the cells.

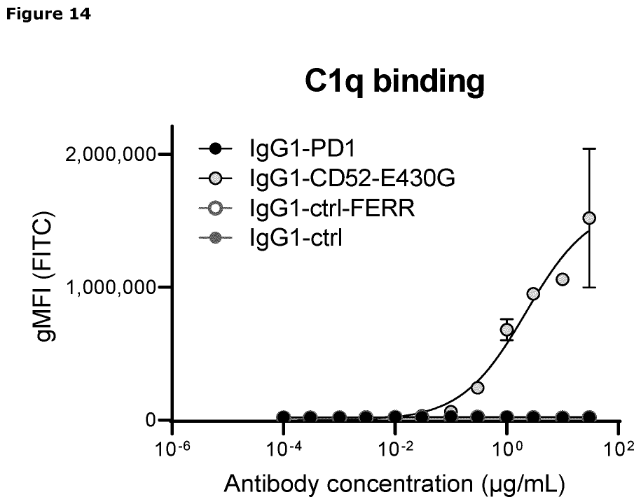

Human serum (20 μL/well; Sanquin, lot 20L15-02), as a source of C1q, was added to a final concentration of 20%. Cells were incubated on ice for 45 min, followed by two washes with cold GMB FACS buffer and incubation with 50 μL fluorescein isothiocyanate (FITC)-conjugated rabbit anti-human C1q (final concentration of 20 μg/mL [DAKO, cat no. F0254]; diluted 1:75 in GMB FACS buffer) in the presence or absence of allophycocyanin-conjugated mouse-anti-CD8 (BD Biosciences, cat. no. 555369; diluted 1:50 in GMB FACS buffer) in the dark at 4° C. for 30 min. Cells were washed twice with cold GMB FACS buffer, resuspended in 20 μL of GMB FACS buffer supplemented with 2 mM ethylenediaminetetraacetic acid (EDTA; Sigma-Aldrich, cat. no. 03690) and 4′,6-diamidino-2-phenylindole (DAPI) viability dye (1:5,000; BD Pharmingen, cat. no. 564907). C1q binding to viable cells (as identified by DAPI exclusion) was analyzed by flow cytometry on an IntelliCyt® iQue Screener PLUS (Sartorius) or iQue3 (Sartorius). Binding curves were analyzed using non-linear regression analysis (sigmoidal dose-response with variable slope) using GraphPad Prism software.

Whereas dose-dependent C1q binding was observed to membrane-bound IgG1-CD52-E430G, no C1q binding was observed to membrane-bound IgG1-PD1 or to the non-binding control antibodies (FIG. 14).

These results indicate that the functionally inert backbone of IgG1-PD1 does not bind C1q.

### Example 12: Binding of IgG1-PD1 to Fcγ Receptors as Determined by SPR

The binding of IgG1-PD1 to immobilized FcγRs (FcγRIa, FcγRIIa, FcγRIIb and FcγRIIIa) was assessed in vitro by SPR. Both polymorphic variants were included for FcγRIIa (H131 and R131) and FcγRIIIa (V158 and F158). As a positive control for FcγR binding, IgG1-ctrl with a wild-type Fc region was included.

In a first experiment, binding of IgG1-PD1, or IgG1-ctrl to immobilized human recombinant FcγR variants (FcγRIa, FcγRIIa, FcγRIIb, and FcγRIIIa) was analyzed using a Biacore 8K SPR system. In a second set of experiments, using the same method, binding of IgG1-PD1, nivolumab (Bristol-Meyers Squibb, lot no. ABP6534), pembrolizumab (Merck Sharp & Dohme, lot no. U013442), dostarlimab (GlaxoSmithKline, lot. no. 1822049), cemiplimab (Regeneron, lot no. 1F006A), IgG1-ctrl, or IgG4-ctrl was analyzed.

Biacore Series S Sensor Chips CM5 (Cytiva, cat. no. 29104988) were covalently coated with anti-Histidine (His) antibody using amine-coupling and His capture kits (Cytiva, cat. no. BR100050 and cat. no. 29234602) according to the manufacturer's instructions. FcγRIa, FcγRIIa (H131 and R131), FcγRIIb and FcγRIIIa (V158 and F158) (SinoBiological, cat. no. 10256-H08S-B, 10374-H08H1, 10374-H27H, 10259-H27H, 10389-H27H1, and 10389-H27H, respectively) diluted in HBS-EP+(Cytiva, cat. no. BR100669) were captured onto the surface of the anti-His coated sensor chip with a flow rate of 10 μL/min and a contact time of 60 seconds toresult in captured levels of approximately 350-600 resonance units (RU).

After three start-up cycles of HBS-EP+buffer, test antibodies (IgG1-PD1, nivolumab, pembrolizumab, dostarlimab, cemiplimab, IgG1-ctrl, or IgG4-ctrl) were injected to generate binding curves, using antibody ranges as indicated in Table 22. Each sample that was analyzed on a surface with captured FcγRs (active surface) was also analyzed on a parallel flow cell without captured FcγRs (reference surface), which was used for background correction. The third start-up cycle containing HBS-EP+as a (mock) analyte was subtracted from other sensorgrams to yield double-referenced data.

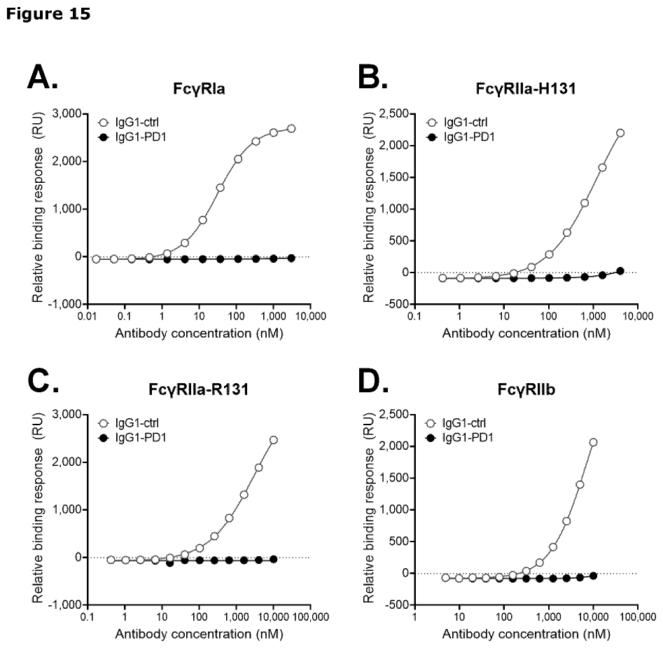

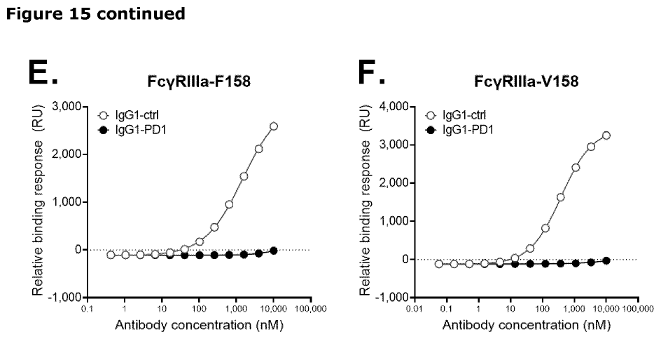

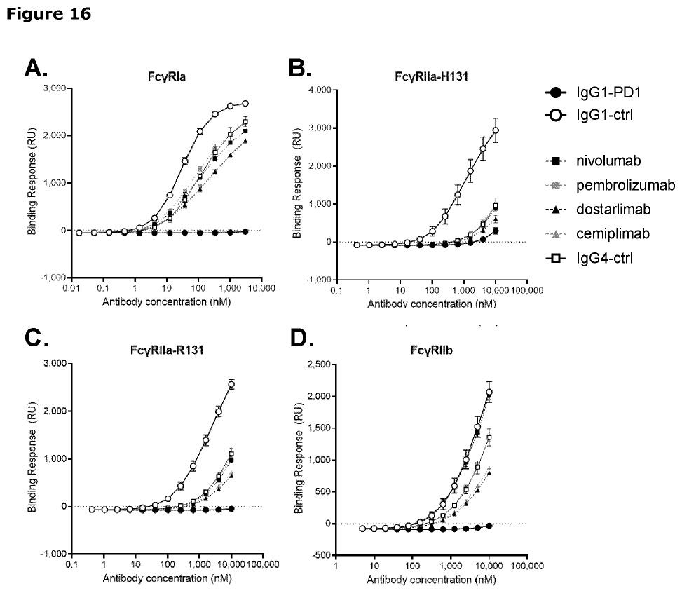

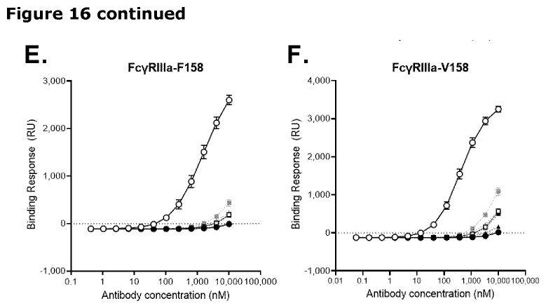

At the end of each cycle, the surface was regenerated using 10 mM Glycine-HCl pH 1.5 (Cytiva, cat. no. BR100354). Sensorgrams were generated using Biacore Insight Evaluation software (Cytiva) and a four-parameter logistic fit was applied on end-point measurements (binding plateau versus post-capture baseline). Data of the first experiment (n=1; qualified SPR assay) is shown in FIG. 15; data of the second set of experiments (n=3) is shown in FIG. 16.

TABLE 22
Test conditions for binding to individual FcγRs

| | Anti-PD-1 antibody concentration range tested | | |
|---|---|---|---|
| | Start concentration | Fold | Lowest concentration |
| FcγR | (nM) | dilution | (nM) |
| FcγRIa | 3,000 | 1:3 | 0.02 |
| FcγRIIa-H131 | 10,000 | 1:2.5 | 0.42 |
| FcγRIIa-R131 | 10,000 | 1:2.5 | 0.42 |
| FcγRIIb | 10,000 | 1:2 | 4.9 |
| FcγRIIIa-V158 | 10,000 | 1:3 | 0.06 |
| FcγRIIIa-F158 | 10,000 | 1:2.5 | 0.42 |

Results from the first experiment showed binding oflgG1-ctrl to all FcγRs, while no binding was observed for IgG1-PD1 to FcγRIa, FcγRIIa (H131 and R131), FcγRIIb, and FcγRIIIa (V158 and F158) (FIG. 15).

Results from the second set of experiments confirmed lack of FcγR binding for IgG1-PD1 (FIG. 16). IgG4-ctrl and the other anti-PD-1 antibodies tested (nivolumab, pembrolizumab, dostarlimab, and cemiplimab; all of the IgG4 subclass) demonstrated clear binding to FcγRIa, FcγRIIa-H131, FcγRIIa-R131, and FcγRIIb, and minimal to very minimal binding to FcγRIIIa-F158 and FcγRIIIa-V158.

These data confirm lack of FcγR binding for the Fc domain of IgG1-PD1 and demonstrate FcγR binding to nivolumab, pembrolizumab, dostarlimab, and cemiplimab. Taken together, these data suggest that the Fc domain of IgG1-PD1 is unable to induce FcγR-mediated effector functions (ADCC, ADCP).

### Example 13: Binding of IgG1-PD1 to Cell Surface Expressed FcγRIa as Determined by Flow Cytometry

Binding of IgG1-PD1, nivolumab, pembrolizumab, dostarlimab, and cemiplimab to human cell surface expressed FcγRIa was analyzed using flow cytometry.

FcγRIa was expressed on transiently transfected CHO-S cells, and cell surface expression was confirmed by flow cytometry using FITC-conjugated anti-FcγRI antibody (BioLegend, cat. no. 305006; 1:25). Binding of anti-PD-1 antibodies to transfected CHO-S cells was assessed as described in Example 6. Briefly, antibody dilutions (final concentrations: 1.69×10⁻⁴-10 μg/mL, 3-fold dilutions) of IgG1-PD1, nivolumab (Bristol-Meyers Squibb, lot no. ABP6534), pembrolizumab (Merck Sharp & Dohme, lot no. U013442), dostarlimab (GlaxoSmithKline, lot. no. 1822049), cemiplimab (Regeneron, lot no. 1F006A), IgG1-ctrl, and IgG1-ctrl-FERR were prepared in GMB FACS buffer. Cells were centrifuged, supernatant was removed, and cells (30,000 cells in 50 μL) were incubated with 50 μL of the antibody dilutions for 30 min at 4° C. Cells were washed twice with GMB FACS buffer and incubated with 50 μL secondary antibody (PE-conjugated goat-anti-human IgG F(ab′)2; 1:500) for 30 min at 4° C., protected from light. Cells were washed twice with GMB FACS buffer and resuspended in GMB FACS buffer supplemented with 2 mM EDTA and DAPI viability marker (1:5,000). Antibody binding to viable cells was analyzed by flow cytometry on an Intellicyt iQue PLUS Screener (Intellicyt Corporation) using FlowJo software by gating on PE-positive, DAPI-negative cells. Binding curves were analyzed using non-linear regression analysis (four-parameter dose-response curve fits) in GraphPad Prism.

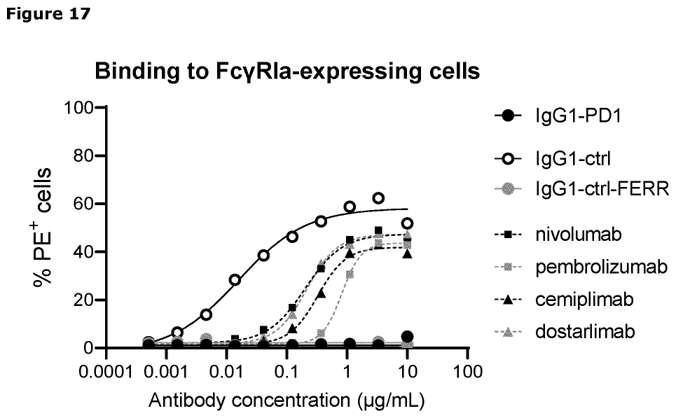

In the flow cytometry binding assays, the positive control antibody IgG1-ctrl (with a wild-type Fc region) showed binding to cells transiently expressing FcγRIa, while no binding was observed for the negative control antibody IgG1-ctrl-FERR (with an Fc region containing the FER inertness mutations and an additional, in the context of this study functionally irrelevant, K409R mutation) (FIG. 17). No binding was observed for IgG1-PD1, while concentration-dependent binding was observed for pembrolizumab, nivolumab, cemiplimab, and dostarlimab.

These data confirm lack of FcγRIa binding for the Fc domain of IgG1-PD1 and demonstrate FcγRIa binding to nivolumab, pembrolizumab, dostarlimab, and cemiplimab. Taken together, these data suggest that the Fc domain of IgG1-PD1 is unable to induce FcγRIa-mediated effector functions.

### Example 14: Binding to Neonatal Fc Receptor by IgG1-PD1

The neonatal Fc receptor (FcRn) is responsible for the long plasma half-life of IgG by protecting IgG from degradation. IgG binds to FcRn in an acidic (pH 6.0) endosomal environment but dissociates from FcRn at neutral pH (pH 7.4). This pH-dependent binding of antibodies to FcRn causes recycling of the antibody together with FcRn, preventing intracellular antibody degradation, and therefore is an indicator for the in vivo pharmacokinetics of that antibody. The binding of IgG1-PD1 to immobilized FcRn was assessed in vitro at pH 6.0 and pH 7.4 by means of surface plasmon resonance (SPR).

Binding of IgG1-PD1 to immobilized human FcRn was analyzed using a Biacore 8K SPR system. Biacore Series S Sensor Chips CM5 (Cytiva, cat. no. 29104988) were covalently coated with anti-histidine (His) antibody using amine coupling and His capture kits (Cytiva, cat. no. BR100050 and cat. no. 29234602) according to the manufacturer's instructions. FcRn (SinoBiological, cat. no. CT071-H27H-B) diluted to a 5 nM coating concentration in PBS-P+buffer pH 7.4 (Cytiva, cat. no. 28995084) or in PBS-P+buffer with the pH adjusted to 6.0 (by addition of hydrochloric acid [Sigma-Aldrich, cat. no. 07102]) was captured onto the surface of the anti-His coated sensor chip with a flow rate of 10 μL/min and a contact time of 60 seconds. This resulted in captured levels of approximately 50 RU. After three start-up cycles of pH 6.0 or pH 7.4 PBS-P+buffer, test antibodies (6.25-100 nM two-fold dilution series of IgG1-PD1, pembrolizumab (MSD, lot. no. T019263), or nivolumab (Bristol-Myers Squibb, lot. no. ABP6534) in pH 6.0 or pH 7.4 PBS-P+buffer) were injected to generate binding curves. Each sample that was analyzed on a surface with captured FcRn (active surface) was also analyzed on a parallel flow cell without captured FcRn (reference surface), which was used for background correction. The third start-up cycle containing HBS-EP+as a (mock) analyte was subtracted from other sensorgrams to yield double-referenced data. At the end of each cycle, the surface was regenerated using 10 mM Glycine HCl pH 1.5 (Cytiva, cat. no. BR100354). The data were analyzed using the predefined “Multi-cycle kinetics using capture” evaluation method in the Biacore Insight Evaluation software (Cytiva). Data is based on three separate experiments with technical duplicates.

At pH 6.0, IgG1-PD1 bound FcRn with an average affinity (K_{D}) of 50 nM (Table 23), which is comparable to an IgG1-ctrl antibody with a wild-type Fc region (a broad range of affinities is reported for wild-type IgG1 molecules in literature; in previous in-house experiments with the same assay set-up, an average K_{D} of 34 nM was measured for IgG1-ctrl across 12 data points). The affinity of pembrolizumab and nivolumab was approximately two-fold lower (K_{D} of 116 nM and 133 nM, respectively). No FcRn binding was observed at pH 7.4 (not shown). Taken together, these results demonstrate that the FER inertness mutations in the IgG1-PD1 Fc region do not affect FcRn binding and suggest that IgG1-PD1 will retain typical IgG pharmacokinetic properties in vivo.

TABLE 23
Affinity for FcRn as determined by SPR Binding of IgG1-PD1, pembrolizumab, and nivolumab
to sensor chips coated with human FcRn was analyzed by SPR. The average affinity
and SD are based on three independent measurements with technical duplicates.

| | K_{D} (M) | | kₐ (1/M × s) | | k_{d} (1/s) | |
|---|---|---|---|---|---|---|
| Antibody | Average | SD | Average | SD | Average | SD |
| IgG1-PD1 | 4.99 × 10⁻⁸ | 6.85 × 10⁻⁹ | 8.65 × 10⁵ | 6.79 × 10⁴ | 4.29 × 10⁻² | 4.88 × 10⁻³ |
| Nivolumab | 1.33 × 10⁻⁷ | 2.01 × 10⁻⁸ | 5.18 × 10⁵ | 4.70 × 10⁴ | 6.89 × 10⁻² | 1.19 × 10⁻² |
| Pembrolizumab | 1.16 × 10⁻⁷ | 1.69 × 10⁻⁸ | 5.46 × 10⁵ | 4.42 × 10⁴ | 6.36 × 10⁻² | 1.15 × 10⁻² |

Abbreviations: K_{D} = equilibrium dissociation constant; kₐ = association rate constant; k_{d} = dissociation rate constant or off-rate; SD = standard deviation.

### Example 15: Pharmacokinetic Analysis of IgG1-PD1 in Absence of Target Binding

The pharmacokinetic properties of IgG1-PD1 were analyzed in mice. PD-1 is expressed mainly on activated B and T cells, and as such, its expression is expected to be limited in non-tumor bearing SCID mice, which lack mature B and T cells. Furthermore, IgG1-PD1 shows substantially reduced cross-reactivity to cells transiently overexpressing mouse PD-1 (Example 6). Therefore, the pharmacokinetic (PK) properties of IgG1-PD1 in non-tumor bearing SCID mice are expected to reflect the PK properties of IgG1-PD1 in absence of target binding. The mice in this study were housed in the Central Laboratory Animal Facility (Utrecht, the Netherlands). All mice were kept in individually ventilated cages with food and water provided ad libitum. All experiments were in compliance with the Dutch animal protection law (WoD) translated from the directives (2010/63/EU) and were approved by the Dutch Central Commission for animal experiments and by the local Ethical committee). SCID mice (C.B-17/IcrHan*Hsd-Prkdc^{s}id, Envigo) were injected intravenously with 1 or 10 mg/kg IgG1-PD1, using 3 mice per group. Blood samples (40 μL) were collected from the saphenous vein or the cheek veins at 10 min, 4 h, 1 day, 2 days, 8 days, 14 days, and 21 days after antibody administration. Blood was collected into vials containing K2-ethylenediaminetetraacetic acid and stored at −65° C. until determination of antibody concentrations.

By a total human IgG (hIgG) electrochemiluminescence immunoassay (ECLIA), specific hIgG concentrations were determined. Meso Scale Discovery (MSD) standard plates (96-well MULTI-ARRAY plate, cat. no. L15XA-3) were coated with mouse anti-hIgG capture antibody (IgG2amm-1015-6A05) diluted in PBS (Lonza, cat. no. BE17-156Q) for 16-24 h at 2-8° C. After washing the plate with PBS-Tween (PBS-T; PBS supplemented with 0.05% (w/v) Tween-20 [Sigma, cat. no. P1379]) to remove non-bound antibody, the unoccupied surfaces were blocked for 60+5 min at RT (PBS-T supplemented with 3% (w/v) Blocker-A [MSD, cat. no. R93AA-1]) followed by washing with PBS-T. Mouse plasma samples were initially diluted 50-fold (2% mouse plasma) in assay buffer (PBS-T supplemented with 1% (w/v) Blocker-A). To create a reference curve, IgG1-PD1 (same batch as the material used for injection) was diluted (measuring range: 0.156-20.0 μg/mL; anchor points: 0.0781 and 40.0 μg/mL) in Calibrator Diluent (2% mouse plasma [K2EDTA, pooled plasma, BIOIVT, cat. no. MSEOOPLK2PNN] in assay buffer). To accommodate for the expected wide range of antibody concentrations present in the samples, samples were additionally diluted 1:10 or 1:50 in Sample Diluent (2% mouse plasma in assay buffer). The coated and blocked plates were incubated with 50 μL diluted mouse samples, the reference curve, and appropriate quality control samples (pooled mouse plasma spiked with IgG1-PD1, covering the range of the reference curve) at RT for 90+5 min. After washing with PBS-T, the plates were incubated with SULFO-TAG-conjugated mouse anti-hIgG detection antibody IgG2amm-1015-4A01 at RT for 90+5 min. After washing with PBS-T, immobilized antibodies were visualized by adding Read Buffer (MSD GOLD Read Buffer, cat. no. R92TG-2) and measuring light emission at −620 nm using an MSD Sector S600 plate reader. Processing of analytical data was performed using SoftMax Pro GxP Software v7.1. Extrapolation below the run lower limit of quantitation (LLOQ) or above the upper limit of quantitation (ULOQ) was not allowed.

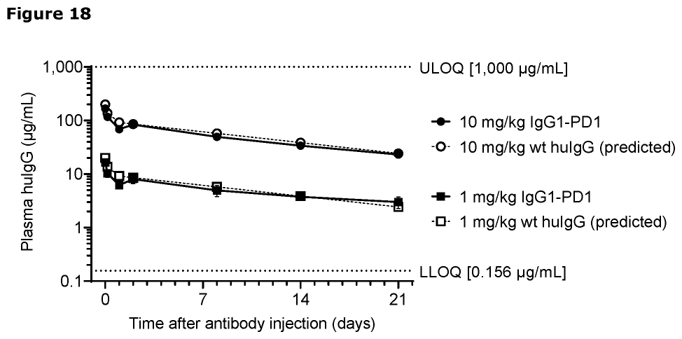

The plasma clearance profile of IgG1-PD1 in absence of target binding was comparable to the clearance profile of a wild-type human IgG1 antibody in SCID mice predicted by a two-compartment model based on IgG1 clearance in humans (Bleeker et al., 2001, Blood. 98(10):3136-42) (FIG. 18). No clinical observations were noted, and no body weight loss was observed.

In conclusion, these data indicate that the PK properties of IgG1-PD1 are comparable to those of normal human IgG antibodies in absence of target binding.

### Example 16: Antitumor Activity of IgG1-PD1 in Human PD-1 Knock-In Mice

IgG1-PD1 shows only limited binding to cells transiently overexpressing mouse PD-1 (Example 6). Therefore, to assess antitumor activity of IgG1-PD1 in vivo, C57BL/6 mice engineered to express the human PD-1 extracellular domain (ECD) in the mouse PD-1 gene locus (hPD-1 knock-in [KI] mice) were used.

All animal experiments were performed at Crown Bioscience Inc. and approved by their Institutional Animal Care and Use Committee (IACUC) prior to execution. Animals were housed and handled in accordance with good animal practice as defined by the regulations of the Association for Assessment and Accreditation of Laboratory Animal Care (AAALAC). Female homozygous human PD-1 knock-in mice on a C57BL/6 background (hPD-1 KI mice; Beijing Biocytogen Co., Ltd; C57BL/6-Pdcd1^{tm1(PDCD1)}/Bcgen, stock no. 110003), 7-9 weeks old, were injected subcutaneously (SC) with syngeneic MC38 colon cancer cells (1×10⁶ cells) in the right lower flank. Tumor growth was evaluated using a caliper (three times per week after randomization), and tumor volumes (mm³) were calculated from caliper measurements as: tumor volume=0.5× (length×width²), where the length is the longest tumor dimension, and the width is the longest tumor dimension perpendicular to the length. Mice were randomized (9 mice per group) based on tumor volume and body weight when tumors had reached an average volume of approximately 60 mm³ (denoted as day 0). At the start of treatment, mice were injected intravenously (IV; dosing volume 10 mL/kg in PBS) with 0.5, 2, or 10 mg/kg IgG1-PD1 or pembrolizumab (obtained from Merck by Crown Bioscience Inc., lot no. T042260), or with 10 mg/kg isotype control antibody IgG1-ctrl-FERR. Subsequent doses were administered intraperitoneally (IP). A dosing regimen of two doses weekly for three weeks (2QWx3) was used. Animals were monitored daily for morbidity and mortality and monitored routinely for other clinical observations. The experiment ended for individual mice when the tumor volume exceeded 1,500 mm³ or when the animals reached other humane endpoints.

To compare progression-free survival between the groups, curve fits were applied to the individual tumor growth graphs to establish the day of progression beyond a tumor volume of 500 mm³ for each mouse. These day values were plotted in a Kaplan-Meier survival curve and used to perform a Mantel-Cox analysis between individual curves using SPSS software. The difference in tumor volumes between the groups was compared using a nonparametric Mann-Whitney analysis (in GraphPad Prism) on the last day that all groups were still intact (ie, until the first tumor-related death in the study, i.e., day 11). P-values are presented accompanied by median values (per group) including the 95% confidence interval of the difference in median (Hodges Lehmann).

The mice showed no signs of illness, but two mice were found dead (one in the 2 mg/kg IgG1-PD1 group and one in the 2 mg/kg pembrolizumab treatment group). The cause of these deaths was undetermined.

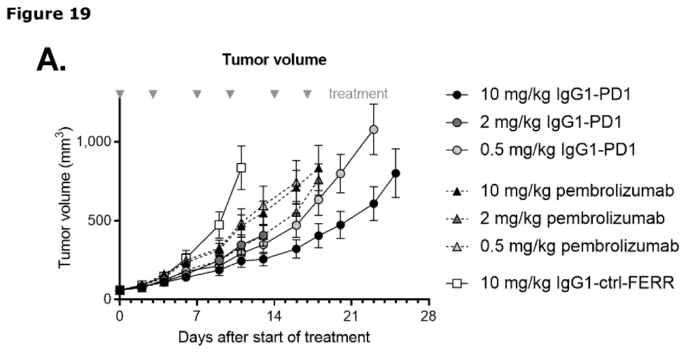

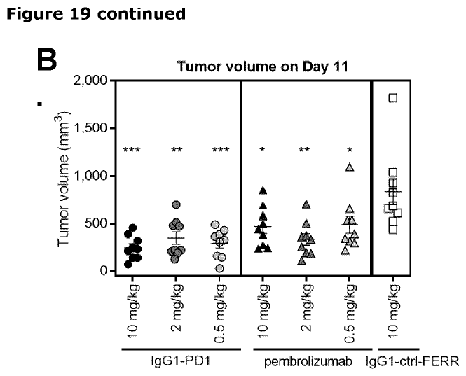

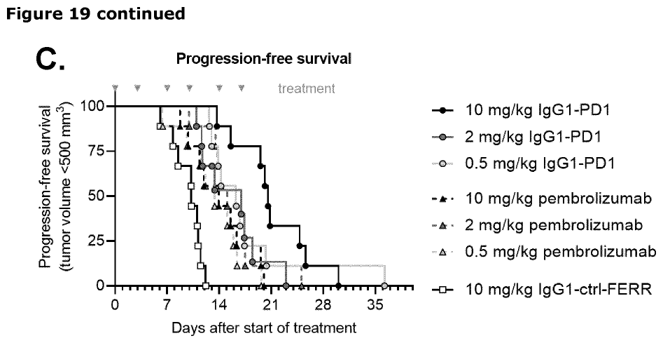

Treatment with IgG1-PD1 and pembrolizumab inhibited tumor growth at all doses tested (FIG. 19A). On Day 11, the last day that all treatment groups were complete, tumors in mice treated with IgG1-PD1 or pembrolizumab were significantly smaller at all doses tested than tumors in mice treated with 10 mg/kg IgG1-ctrl-FERR (FIG. 19B). In addition, at 10 mg/kg, tumor volumes in mice treated with IgG1-PD1 were significantly smaller than in mice treated with an equivalent dose of pembrolizumab (Mann-Whitney test, p=0.0188).

Treatment with IgG1-PD1 or pembrolizumab significantly increased progression-free survival (PFS) at all doses tested compared to mice treated with 10 mg/kg IgG1-ctrl-FERR (FIG. 19C). At 10 mg/kg, progression-free survival in mice treated with IgG1-PD1 was significantly extended as compared to mice treated with pembrolizumab (median PFS 10 mg/kg IgG1-PD1: 20.56 days, median PFS 10 mg/kg pembrolizumab: 13.94 days; P-value=0.0021).

In conclusion, IgG1-PD1 exhibited potent antitumor activity in MC38 tumor-bearing hPD-1 KI mice.

### Example 17: Effect of GEN1046 in Combination with IgG1-PD1 on IL-2 Secretion in an Allogeneic MLR Assay

To analyze if the combination of GEN1046 with IgG1-PD1 could result in potentiation of cytokine production in a mixed lymphocyte reaction (MLR) assay over single agent activity, two unique, allogeneic pairs of human mature dendritic cells (mDCs) and CD8⁺ T cells were co-cultured in the presence of GEN1046 alone, IgG1-PD1 alone or a combination of both antibodies. Interleukin (IL)-2 secretion was assessed in the supernatants of the co-cultures using an IL-2-specific immunoassay.

Methods Monocytes and T Cells from Healthy Donors

CD14⁺ monocytes and purified CD8⁺ T cells were obtained from BioIVT. Two unique allogeneic donor pairs were used for the MLR assay.

### Differentiation of Monocytes to Immature Dendritic Cells

Human CD14+monocytes were obtained from healthy donors. For differentiation to immature dendritic cells (iDCs), 1-1.5×10⁶ monocytes/mL were cultured for six days in Roswell Park Memorial Institute (RPMI) 1640 complete medium (ATCC modification formula; ThermoFisher, cat. no. A1049101) supplemented with 10% heat-inactivated Fetal Bovine Serum (FBS; Gibco, cat. no. 16140071), 100 ng/mL granulocyte-macrophage colony-stimulating factor (GM-CSF; BioLegend, cat. no. 766106) and 300 ng/mL interleukin-4 (IL-4; BioLegend, cat. no. 766206) in T25 culture flasks (Falcon, cat. no. 353108) at 37° C. After four days, the medium was replaced with fresh medium and supplements.

Maturation of iDCs to mDCs

Prior to start of the MLR assay, iDCs were harvested by collecting non-adherent cells and differentiated to mature DCs (mDCs) by incubating at 1-1.5×10⁶ cells/mL in RPMI 1640 complete medium supplemented with 10% FBS, 100 ng/mL GM-CSF, 300 ng/mL IL-4 and 5 μg/mL lipopolysaccharide (LPS; ThermoFisher, cat. no. 00-4976-93) for 24 h at 37° C.

### Mixed Lymphocyte Reaction (MLR)

One day prior to the start of an MLR assay, purified CD8⁺ T cells obtained from allogeneic healthy donors were thawed, resuspended at 1×10⁶ cells/mL in RPMI 1640 complete medium supplemented with 10% FBS and 10 ng/mL IL-2 (BioLegend, cat. no. 589106) and incubated O/N at 37° C.

The next day, the LPS-matured dendritic cells (mDCs, see Maturation of iDCs) and allogeneic purified CD8⁺ T cells were harvested and resuspended in AIM-V medium (ThermoFisher, cat. no. 12055091) at 4×10⁵ cells/mL and 4×10⁶ cells/mL, respectively.

Co-cultures were seeded at a DC:T cell ratio of 1:10, corresponding to 20,000 mDCs incubated with 200,000 allogeneic purified CD8⁺ T cells, and cultured in the presence of IgG1-PD1 (1 μg/mL) as single agent, research-grade pembrolizumab (1 μg/mL, Seleckchem, cat. no. A2005 (non-clinical/research-grade version of the clinical product pembrolizumab), GEN1046 (0.001 to 30 μg/mL) as single agent, or both agents combined in AIM-V medium in a 96-well round-bottom plate (Falcon, cat. no. 353227) at 37° C. for 5 days. Co-cultures treated with bsIgG1-PD-L1xctrl (30 μg/mL), bsIgG1-ctrlx4-1BB (30 μg/mL), IgG4 (Biolegend, cat. no. 403702), IgG1-ctrl-FERR (100 μg/mL) or IgG1-ctrl-FEAL (30 μg/mL) were included as controls. After 5 days, the plates were centrifuged at 500×g for 5 min and the supernatant was carefully transferred from each well to a new 96-well round bottom plate.

The collected supernatants from the MLR assay were analyzed for IL-2 levels as part of the Milliplex MAP-Human cytokine/chemokine Magnetic bead panel (Millipore Sigma, cat. no. HCYTOMAG-60K-08) on a Luminex FLEXMAP 3D instrument.

TABLE 24

| Test compound | Supplier, cat. no. | Comprising SEQ ID NOS |
|---|---|---|
| GEN1046 | N/A | CD137 binding arm: |
| | | SEQ ID NOs: 1, |
| | | 5, 35, 29 |
| | | PD-L1 binding arm: |
| | | SEQ ID NOs: 11, |
| | | 15, 36, 30 |
| bsIgG1-PD-L1xctrl | N/A | SEQ ID NO: 11, 15, 79, |
| | | 80, 35, 36, 29, 30 |
| bsIgG1-ctrlx4-1BB | N/A | SEQ ID NO: 35, 36, 1, |
| | | 5, 79, 80, 29, 30 |
| IgG1-PD1 | N/A | SEQ ID NO: 88, 89, 90, 35 |
| IgG1-ctrl-FEAL¹ | N/A | SEQ ID NO: 79, 80, 30, 35 |
| IgG1-ctrl-FERR¹ | N/A | SEQ ID NO: 79, 80, 91, 35 |

¹Control binding moiety based on anti-HIV gp120 antibody IgG1-b12 (Barbas et al., 1993, J Mol Biol 230: 812-823)

### Results

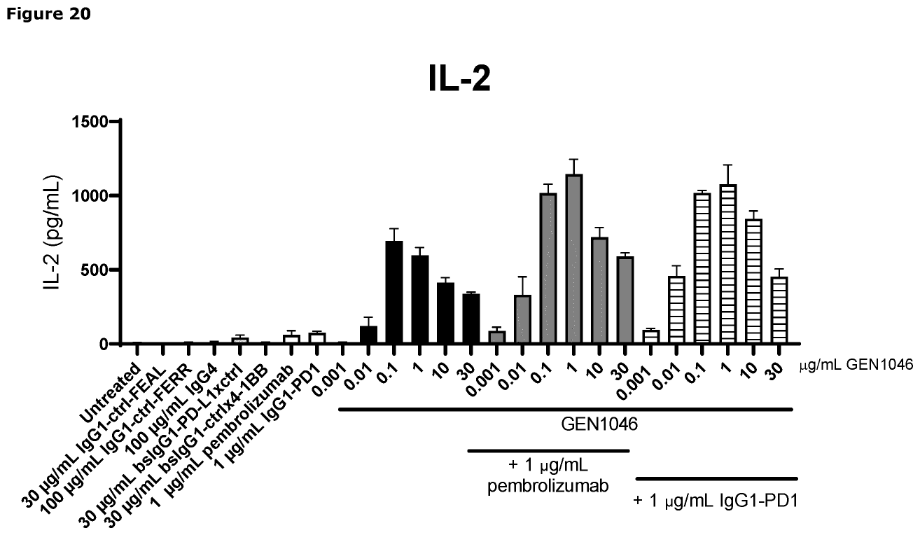

Treatment with either GEN1046, pembrolizumab or IgG1-PD1 alone enhanced the secretion of IL-2 compared to non-binding control antibodies. The combination of GEN1046 with 1 μg/mL IgG1-PD1 further potentiated secretion of IL-2 compared to either GEN1046 or IgG1-PD1 alone (FIG. 20). As single agent, GEN1046 showed a concentration dependent response, whit peak induction of IL-2 at 0.1-1 μg/mL. Potentiation of IL-2 production by 1 μg/mL IgG1-PD-1 or 1 μg/mL pembrolizumab was observed across all concentrations of GEN1046.

### Conclusion

These results indicate that combining GEN1046 and IgG1-PD1 potentiates IL-2 secretion relative to single agent activity in an mDC/CD8⁺ T cell MLR assay.

### Example 18: Antigen-Specific Stimulation Assay to Determine the Capacity of GEN1046 in Combination with IgG1-PD1 to Enhance T-Cell Proliferation and Cytokine Secretion

To determine the capacity of GEN1046 in combination with IgG1-PD1 to enhance T-cell proliferation, an antigen-specific stimulation assay was conducted using co-cultures of PD1-overexpressing human CD8⁺ T cells and cognate antigen-expressing immature dendritic cells (iDCs). Cytokine concentrations were assessed in supernatants of the co-cultures.

### Methods

### Isolation of Cells and Differentiation of Monocytes to Immature Dendritic Cells

HLA-A*02⁺ peripheral blood mononuclear cells (PBMCs) were obtained from healthy donors (Transfusionszentrale, University Hospital, Mainz, Germany). Monocytes were isolated from PBMCs by magnetic-activated cell sorting (MACS) technology using anti-CD14 MicroBeads (Miltenyi; cat. no. 130-050-201), according to the manufacturer's instructions. The peripheral blood lymphocytes (PBLs, CD14-negative fraction) were cryopreserved for CD8⁺ T-cell isolation. For differentiation into iDCs, 1×10⁶ monocytes/mL were cultured for 5 days in RPMI 1640 (Life Technologies GmbH, cat. no. 61870-010) containing 5% pooled human serum (One Lambda Inc., cat. no. A25761), 1 mM sodium pyruvate (Life technologies GmbH, cat. no. 11360-039), 1× non-essential amino acids (Life Technologies GmbH, cat. no. 11140-035), 200 ng/mL granulocyte-macrophage colony-stimulating factor (GM-CSF; Miltenyi, cat. no. 130-093-868) and 200 ng/mL interleukin-4 (IL-4; Miltenyi, cat. no. 130-093-924). On day 3, half of the medium was replaced with fresh medium containing supplements. iDCs were harvested by collecting non-adherent cells and adherent cells were detached by incubation with Dulbecco's phosphate-buffered saline (DPBS) containing 2 mM EDTA for 10 min at 37°. After washing with DPBS iDCs were cryopreserved in FBS (Sigma-Aldrich, cat. no. F7524) containing 10% DMSO (AppliChem GmbH, cat. no A3672,0050) for future use in antigen-specific T-cell assays.

Electroporation of iDCs and CD8⁺ T cells and CFSE-labeling

One day prior to the start of an antigen-specific CD8⁺ T cell stimulation assay, frozen PBLs and iDCs from the same donor were thawed. CD8⁺ T cells were isolated from PBLs by MACS technology using anti-CD8 MicroBeads (Miltenyi, cat. no. 130-045-201), according to the manufacturer's instructions. About 10×10⁶ to 15×10⁶ CD8⁺ T cells were electroporated with each 10 μg of in vitro transcribed (IVT)-RNA encoding the alpha and beta chains of a murine TCR specific for human claudin-6 (CLDN6; HLA-A*02-restricted; described in WO 2015150327 Al) plus 10 μg IVT-RNA encoding human PD1 (UniProt Q15116) in 250 μL X-Vivol5 medium (Lonza, cat. no. BE02-060Q). The cells were transferred to a 4-mm electroporation cuvette (VWR International GmbH, cat. no. 732-0023) and electroporated using the BTX ECM® 830 Electroporation System (BTX; 500 V, 3 ms pulse). Immediately after electroporation, cells were transferred into fresh IMDM GlutaMAX medium (Life Technologies GmbH, cat. no. 319800-030) containing 5% pooled human serum and rested at 37° C., 5% CO₂ for at least 1 hour. T cells were labeled using 0.8 μM carboxyfluorescein succinimidyl ester (CFSE; Life Technologies GmbH, cat. No V12883) in PBS according to the manufacturer's instructions and incubated in IMDM medium supplemented with 5% human AB serum overnight.

Up to 5×10⁶ thawed iDCs were electroporated with 2 μg IVT-RNA encoding full-length human CLDN6 (WO 2015150327 Al), in 250 μL X-Vivol5 medium, using the electroporation system as described above (300 V, 12 ms pulse) and incubated in IMDM medium supplemented with 5% pooled human serum overnight.

The next day, cells were harvested. Cell-surface expression of CLDN6 on iDCs, as well as cell-surface expression of the CLDN6-specific TCR and PD1 on T cells was confirmed by flow cytometry. To this end, iDCs were stained with a fluorescently labeled CLDN6-specific antibody (non-commercially available; in-house production). T cells were stained with a brilliant violet (BV)421-conjugated anti-mouse TCR-P chain antibody (Becton Dickinson GmbH, cat. no. 562839) and an allophycocyanin (APC)-conjugated anti-human PD1 antibody (Thermo Fisher Scientific, cat. no. 17-2799-42).

### Antigen-Specific In Vitro T-Cell Stimulation Assay

Electroporated iDCs were incubated with electroporated, CFSE-labeled CD8⁺ T cells at a ratio of 1:10 in the presence of IgG1-PD1 (0.8 μg/mL), clinical grade pembrolizumab (Keytruda®, Merck Sharp & Dohme GmbH, PZN 10749897) (0.8 μg/mL), or the negative control antibody IgG1-ctrl-FERR (0.8 μg/mL), either alone or in combination with GEN1046 (0.0022, 0.0067, or 0.2 μg/mL), in IMDM medium containing 5% pooled human serum in a 96-well round-bottom plate. After 4 days of culture, the cells were stained with an APC-conjugated anti-human CD8 antibody. T-cell proliferation was evaluated by flow cytometry analysis of CFSE dilution in CD8⁺ T cells using a BD FACSCelesta™ flow cytometer (Becton Dickinson GmbH). Flow cytometry data was analyzed using FlowJo software version 10.7.1. CFSE label dilution of CD8⁺ T cells was assessed using the proliferation modeling tool in FlowJo, and expansion indices calculated using the integrated formula.

### Determination of Cytokine Concentrations

Cytokine concentrations in supernatants that had been collected from T cell/iDC co-cultures after 4 days were determined by multiplexed electrochemiluminescence immunoassay using a custom-made U-Plex biomarker group 1 (human) assay for the detection of panel of 10 human cytokines (GM-CSF, IL-2, IL-8, IL-10, IL-12p70, IL-13, interferon [IFN]-γ, IFN-γ-inducible protein [IP]-10 [also known as C—X-C motif chemokine ligand 10], macrophage chemoattractant protein [MCP]-1, and tumor necrosis factor [TNF]-α; Meso Scale Discovery, cat. No. K15067L-2) following the manufacturer's protocol.

TABLE 25

| Test compound | Supplier, cat. no. | Comprising SEQ ID NOS |
|---|---|---|
| GEN1046 | N/A | CD137 binding arm: |
| | | SEQ ID NOs: 1, |
| | | 5, 35, 29 |
| | | PD-L1 binding arm: |
| | | SEQ ID NOs: 11, |
| | | 15, 36, 30 |
| IgG1-PD1 | N/A | SEQ ID NO: 88, 89, 90, 35 |
| IgG1-ctrl-FERR¹ | N/A | SEQ ID NO: 79, 80, 91, 35 |
| Pembrolizumab, | Keytruda ®, | N/A |
| clinical grade | Merck Sharp & | |
| | Dohme GmbH, | |
| | PZN 10749897 | |

### Results

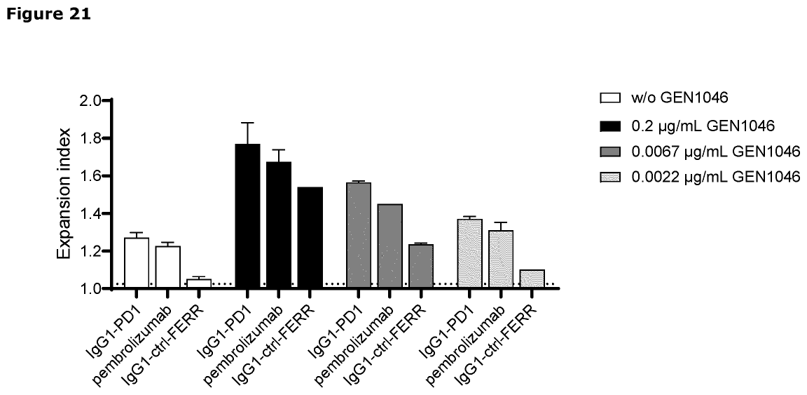

Combination treatment with GEN1046 and IgG1-PD1 potentiated CD8⁺ T-cell proliferation, compared to GEN1046 combined with IgG1-ctrl-FERR and compared to IgG1-PD1 as single treatment (FIG. 21). Increased proliferation was seen at all concentrations of GEN1046 in combination with IgG1-PD1, compared to GEN1046 alone. Combination treatment with pembrolizumab and GEN1046 also enhanced proliferation compared to both compounds as single agents.

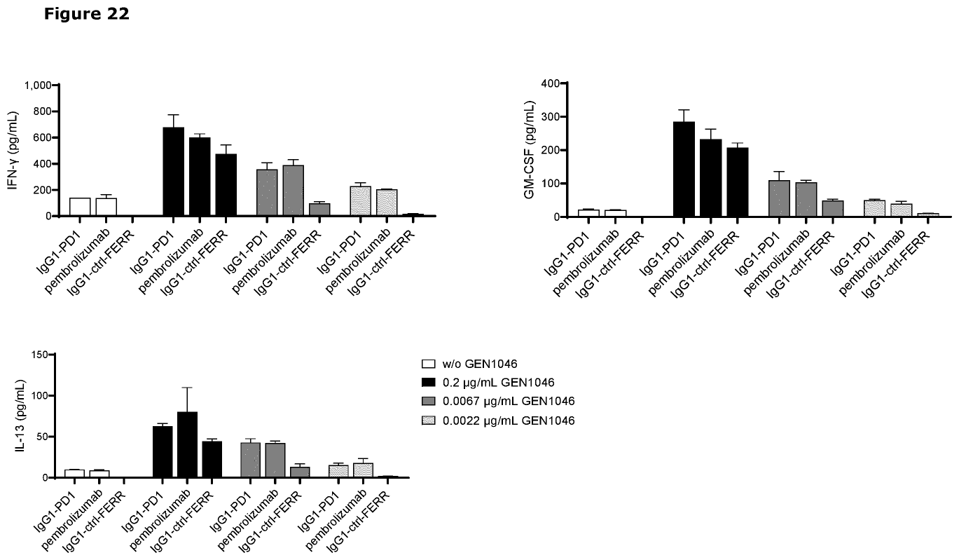

Combination treatment with GEN1046 and IgG1-PD1 potentiated the secretion of the proinflammatory cytokines GM-CSF, IFN-γ, and IL-13, compared to GEN1046 combined with IgG1-ctrl-FERR and compared to IgG1-PD1 as single treatment (FIG. 22). Increased cytokine secretion was seen at all concentrations of GEN1046 in combination with IgG1-PD1, compared to GEN1046 alone. Substantial potentiation of GEN1046 single-agent activity was detected when intermediate (0.0067 μg/mL) or low (0.0022 μg/mL) concentrations of GEN1046 were combined with IgG1-PD1. Potentiation of IgG1-PD1 single-agent activity was increasingly pronounced in combination with increasing GEN1046 concentrations. Combination treatment with pembrolizumab and GEN1046 also enhanced cytokine secretion compared to both compounds as single agents. Secretion of other cytokines tested were detected at low absolute concentrations, not consistently enhanced, or not enhanced, by the combination compared to single-agent treatments.

### Example 19: Anti-Tumor Activity in MC38 Mouse Colon Cancer Tumor Outgrowth Upon Treatment with a Combination of mbsIgG2a-PD-L1×4-1BB with Anti-mPD-1

Objective: To investigate the anti-tumor activity of mbsIgG2a-PD-L1×4-1BB antibody either alone or in combination with an anti-mPD-1 antibody in the MC38 colon cancer model in C57BL/6 mice.

### Methods

MC38 mouse colon cancer cells were cultured in Dulbecco's Modified Eagle Medium supplemented with 10% heat-inactivated fetal bovine serum at 37° C., 5% CO₂. MC38 cells were harvested from a cell culture growing in log-phase and quantified.

MC38 cells (1×10⁶ tumor cells in 100 μL PBS) were injected subcutaneously in the right lower flank of female C57BL/6 mice (obtained from Shanghai Lingchang Biotechnology Co., Ltd and Services; age 6-8 weeks at start of experiment).

Tumor growth was evaluated three times per week using a caliper. Tumor volumes (mm³) were calculated from caliper measurements as ([length]×[width]²)/2, where the length is the longest tumor dimension and the width is the longest tumor dimension perpendicular to the length.

Treatment was initiated when tumors had reached a mean volume of 60 mm³. Mice were randomized into groups (n=10/group) with equal mean tumor volume prior to treatment. On treatment days (two doses weekly for three weeks [2QWX3]), the mice were injected intraperitoneally with the antibodies indicated in Table 26 in an injection volume of 10 μL/g body weight. For combination treatments, antibodies were injected in two separate injections with 20 min in between (Table 26). Dose levels were based on previous experience with these antibodies in the MC38 mouse model.

The mice were monitored daily for clinical signs of illness. Body weight measurements were performed three times a week after randomization. The antibodies and combinations thereof were well tolerated, as mice showed minimal body weight loss (<20%) upon treatment, rather an increase in body weight. The experiment ended for the individual mice when the tumor volume exceeded 1500 mm³ or when the animals reached humane endpoints (e.g. when mice showed body weight loss >20%, when tumors showed ulceration [>75%], when serious clinical signs were observed and/or when the tumor growth blocked the physical activity of the mouse).

Mice that showed complete regression of tumors after antibody treatment were rechallenged with MC38 tumor cells 121 days after treatment initiation. Mice were inoculated with 1×10⁶ fresh MC38 tumor cells on the opposite flank of the original tumor cell inoculation. As control treatment of tumor outgrowth, a group of age matched naive C57BL/6 mice (n=6) was inoculated with MC38 tumor cells from the same cell culture.

TABLE 26
Treatment groups and dosing regimen

| Treatment | N per | | | Dosing | Seq ids/ |
|---|---|---|---|---|---|
| group | group | Treatment | Dose | regimen | Supplier, cat. no. |
| 1 | 10 | mIgG2a-ctrl- | 5 mg/kg | 2QW × 3 | Seq ids: 79, |
| | | AAKR | | | 80, 84, 85 |
| 2 | 10 | Anti-mPD-1 | 10 mg/kg | 2QW × 3 | clone RMP1-14, |
| | | | | | Leinco |
| | | | | | Technologies, |
| | | | | | cat. no. P372 |
| 3 | 10 | mbsIgG2a- | 5 mg/kg | 2QW × 3 | Seq ids: 86, 87, |
| | | PD-L1 × | | | 81, 82, 83, 84, 85 |
| | | 4-1BB | | | |
| 4 | 10 | mbsIgG2a- | 5 mg/kg | 2QW × 3 | See above: |
| | | PD-L1 × | + | | group 2 and 3 |
| | | 4-1BB^{a} + | 10 mg/kg | | |
| | | Anti-mPD-1 | | | |

^{a}mbsIgG2a-PD-L1 × 4-1BB was injected first and the second antibody was injected after 20 min

### Results

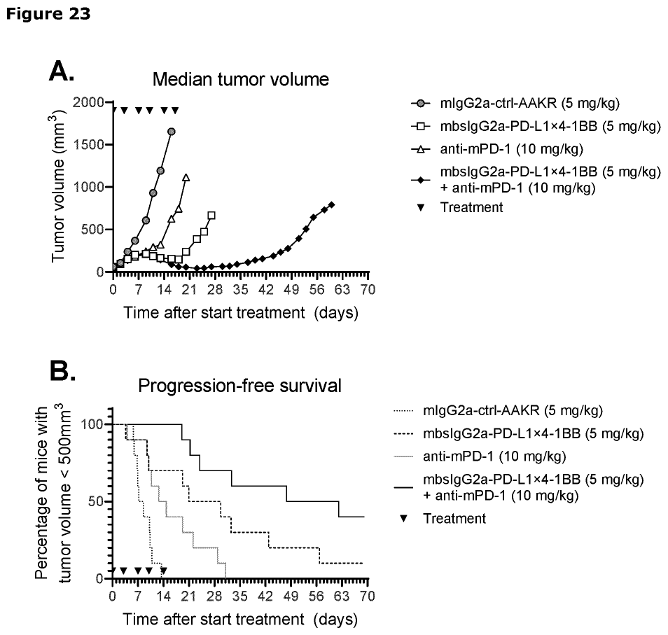

Rapid tumor outgrowth was observed in MC38-bearing mice treated with nonbinding control antibody mIgG2a-ctrl-AAKR (5 mg/kg; FIG. 23A).

In mice treated with anti-mouse PD-1 antibody (anti-mPD-1; 10 mg/kg) or mbsIgG2a-PD-L1×4-1BB (5 mg/kg; FIG. 23A) as single agents, delayed tumor outgrowth was observed, with a more pronounced delay in tumor outgrowth induced by mbsIgG2a-PD-L1×4-1BB. In mice treated with mbsIgG2a-PD-Llx4-1BB (5 mg/kg) combined with anti-mPD-1 (10 mg/kg; both 2QWx3) tumor outgrowth was further delayed compared to each agent alone (FIG. 23A) and complete tumor regressions were observed in 4/10 mice at day 23 post-treatment initiation (compared to complete tumor regressions in 1/10 and 0/10 mice observed for mbsIgG2a-PD-L1×4-1BB and anti-mPD-1 alone, respectively; Table 28), suggestive of synergistic activity of the combination. Kaplan-Meier analysis showed that treatment with the combination of mbsIgG2a-PD-L1×4-1BB and anti-mPD-1 led to a significant increase in progression-free survival, defined as the percentage of mice with tumor volume smaller than 500 mm³, when compared to the control antibody-treated group (p<0.001) and compared to either antibody alone (p<0.05; Mantel-Cox; FIG. 23B, Table 27). Hence, therapeutic synergy was observed with this combination, defined as superior (p<0.05) antitumor efficacy relative to the activity shown by each agent as monotherapy.

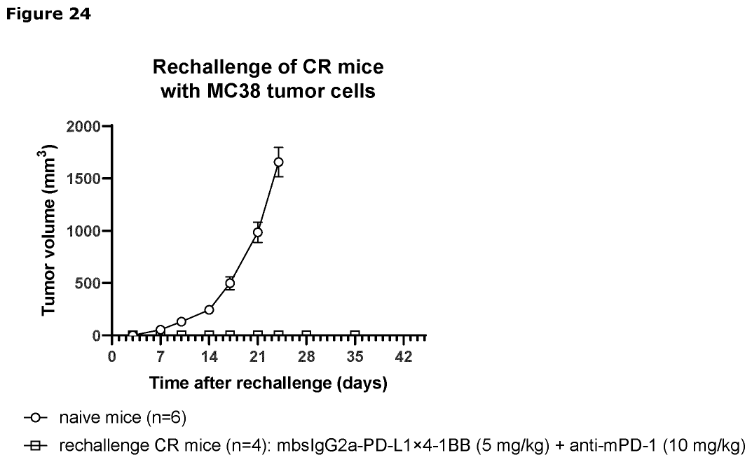

Mice with complete tumor regression, eg, where the tumors disappeared completely for the duration of the observation period (Table 28), were (re)challenged with MC38 tumor cells that were SC injected on Day 121 after the treatment with antibodies was initiated. A control group of six age-matched tumor-naive mice was SC injected with MC38 tumor cells at the same time. In all naive mice, the MC38 tumor grew out to 1,500 mm³ at Day 24 after tumor inoculation, whereas there was no tumor outgrowth observed in the rechallenged mice during the entire follow-up period of 35 days after the rechallenge (156 days after the original inoculation with MC38 tumor cells), consistent with the development of immune memory (FIG. 24).

These results provide rationale for evaluating the combination of GEN1046 with an anti-PD-1 antibody to further amplify the anti-tumor immune response in cancer patients to produce durable and deep clinical responses and enhance survival.

TABLE 27
Mantel-Cox analysis of the progression-free survival induced
by mbsIgG2a-PD-L1 × 4-1BB, anti-mPD-1, or a combination
thereof in the MC38 model in C57BL/6 mice

| | | | Progression-free |
|---|---|---|---|
| | | | survival¹ |
| | | | Mantel-Cox |
| Treatment groups compared | | | P value |
| mIgG2a-ctrl-AAKR | vs | Anti-mPD-1 | 0.008 |
| mIgG2a-ctrl-AAKR | vs | mbsIgG2a-PD-L1 × 4-1BB | 0.002 |
| mIgG2a-ctrl-AAKR | vs | mbsIgG2a-PD- | <0.001 |
| | | L1 × 4-1BB + anti-mPD-1 | |
| Anti-mPD-1 | vs | mbsIgG2a-PD-L1 × 4-1BB | 0.070 |
| Anti-mPD-1 | vs | mbsIgG2a-PD- | <0.001 |
| | | L1 × 4-1BB + anti-mPD-1 | |
| mbsIgG2a-PD- | vs | mbsIgG2a-PD- | 0.043 |
| L1 × 4-1BB | | L1 × 4-1BB + anti-mPD-1 | |

¹Tumor volume <500 mm³ was used as the cut-off for progression-free survival. Mantel-Cox analysis was performed at Day 69.
²A p-value <0.05 was considered significant

TABLE 28
Complete tumor regressions upon treatment of MC38-tumor bearing mice.

| | | | Complete |
|---|---|---|---|
| | | | tumor |
| | | | regressions |
| | | | (no. of |
| | | | mice |
| | | | with CR/ |
| | | | total no. |
| Treat- | | | of mice |
| ment | tumor regressions | | per |
| group | Treatment | Dose | group) |
| 1 | mIgG2a-ctrl-AAKR | 5 mg/kg | 0/10 |
| 2 | Anti-mPD-1 | 10 mg/kg | 0/10 |
| 3 | mbsIgG2a-PD-L1 × 4-1BB | 5 mg/kg | 1/10 |
| 4 | mbsIgG2a-PD-L1 × | 5 mg/kg + | 4/10 |
| | 4-1BB + Anti-mPD-1 | 10 mg/kg | |

Example 20: Cytokine Analysis in Peripheral Blood of MC38-Tumor Bearing Mice Treated with Combinations of mbsIgG2a-PD-L1×4-1BB with an Anti-mPD-1 Antibody

Objective: To investigate cytokine levels in peripheral blood of MC38-tumor bearing C57BL/6 mice treated with mbsIgG2a-PD-L1x4-1BB either alone or in combination with an anti-mPD-1 antibody.

### Methods

In the experiment described in Example 19, blood samples were collected from the MC38-tumor bearing C57BL/6 mice at the following time points: Day −1 (baseline; one day before treatment with the first dose), Day 2 (2 days after first dose) and Day 5 (2 days after second dose) after initiation of treatment.

Cytokines were analyzed in plasma samples by electrochemiluminescence (ECLIA) using the V-PLEX Proinflammatory Panel 1 mouse Kit (MSD LLC, cat. no. K15048D-2) and the V-PLEX Cytokine Panel 1 mouse Kit (MSD LLC, cat. no. K15245D-2) on a MESO QuickPlex SQ 120 instrument (MSD, LLC. R31QQ-3), according to the manufacturer's instructions.

### Results

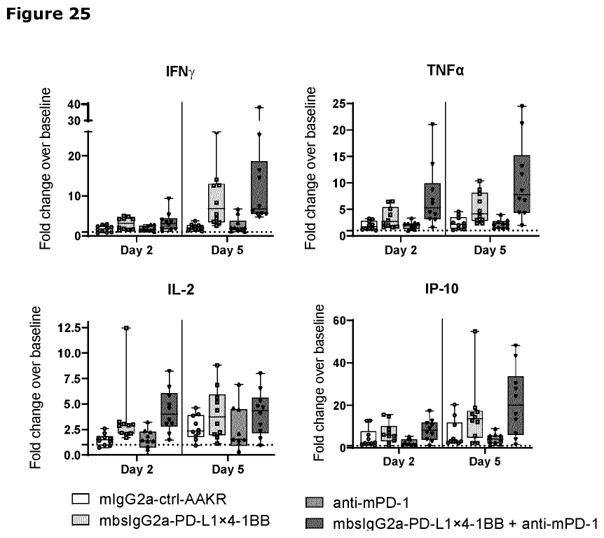

In mice treated with mIgG2a-ctrl-AAKR (5 mg/kg) or anti-mouse PD-1 antibody (anti-mPD-1; 10 mg/kg) as single agent, no or minor changes in the levels of IFNγ, TNFα, IL-2 and IP-10 were observed on Day 2 or Day 5 compared to Day −1 (FIG. 25). In mice treated with mbsIgG2a-PD-L1x4-1BB (5 mg/kg), plasma levels of IFNγ, TNFα, IL-2 and IP-10 were increased at Day 2 and further enhanced at Day 5. In mice treated with the combination of mbsIgG2a-PD-L1x4-1BB (5 mg/kg) and anti-mPD-1 (10 mg/kg), the increase in the levels of IFNγ, TNFα, IL-2 and IP-10 was potentiated on Day 2 and/or Day 5 relative to each single agent (FIG. 25). On Day 5 levels of IFNγ, TNFα and IP-10 were >3-fold higher in mice treated with the combination of mbsIgG2a-PD-L1x4-1BB and anti-mPD-1 compared to both mIgG2a-ctrl-AAKR and the anti-PD-1 treated groups, and levels of TNFα and IP-10 were >1.48-fold higher compared to the mbsIgG2-PD-L1x4-1BB treated groups (Table 29).

These results provide rationale for evaluating the combination of GEN1046 with an anti-PD-1 antibody to further amplify the anti-tumor immune response in cancer patients.

TABLE 29
Fold change in cytokine levels in response to the
combination of mbsIgG2a-PD-L1 × 4-1BB
with anti-mPD-1 compared to single agents

| | | | | Ratio of | |
|---|---|---|---|---|---|
| | | | | median | |
| | | | | fold | |
| | Treatment groups | | | changes | |
| Cytokine | compared | | | Day 2 | Day 5 |
| IFNγ | mbsIgG2a-PD- | vs | mIgG2a-ctrl- | 1.77 | 3.39 |
| | L1 × 4-1BB + anti-mPD-1 | | AAKR | | |
| IFNγ | mbsIgG2a-PD- | vs | Anti-mPD-1 | 1.93 | 3.42 |
| | L1 × 4-1BB + anti-mPD-1 | | | | |
| IFNγ | mbsIgG2a-PD- | vs | mbsIgG2a- | 0.98 | 0.99 |
| | L1 × 4-1BB + anti-mPD-1 | | PD-L1 × 4-1BB | | |
| TNFα | mbsIgG2a-PD- | vs | mIgG2a- | 3.07 | 3.56 |
| | L1 × 4-1BB + anti-mPD-1 | | ctrl-AAKR | | |
| TNFα | mbsIgG2a-PD- | vs | Anti-mPD-1 | 2.59 | 3.44 |
| | L1 × 4-1BB + anti-mPD-1 | | | | |
| TNFα | mbsIgG2a-PD- | vs | mbsIgG2a- | 1.97 | 1.87 |
| | L1 × 4-1BB + anti-mPD-1 | | PD-L1 × 4-1BB | | |
| IL-2 | mbsIgG2a-PD- | vs | mIgG2a- | 2.66 | 1.85 |
| | L1 × 4-1BB + anti-mPD-1 | | ctrl-AAKR | | |
| IL-2 | mbsIgG2a-PD- | vs | Anti-mPD-1 | 2.87 | 2.87 |
| | L1 × 4-1BB + anti-mPD-1 | | | | |
| IL-2 | mbsIgG2a-PD- | vs | mbsIgG2a- | 1.39 | 1.17 |
| | L1 × 4-1BB + anti-mPD-1 | | PD-L1 × 4-1BB | | |
| IP-10 | mbsIgG2a-PD- | vs | mIgG2a- | 3.54 | 6.41 |
| | L1 × 4-1BB + anti-mPD-1 | | ctrl-AAKR | | |
| IP-10 | mbsIgG2a-PD- | vs | Anti-mPD-1 | 4.70 | 4.94 |
| | L1 × 4-1BB + anti-mPD-1 | | | | |
| IP-10 | mbsIgG2a-PD- | vs | mbsIgG2a- | 1.41 | 1.48 |
| | L1 × 4-1BB + anti-mPD-1 | | PD-L1 × 4-1BB | | |

Example 21: The Combination of mbsIgG2a-PD-Llx4-1BB and Anti-mPD-1 Potentiates Anti-Tumor Immunity in the MC38 Mouse Colon Cancer Tumor Model Via Distinct and Complementary Immune Modulatory Effects

Objective: As described in Example 19, mbsIgG2a-PD-L1×4-1BB combined with anti-mPD-1 showed potent anti-tumor activity with a durable response in the MC38 colon cancer model in C57BL/6 mice. Therefore, this model was used to further study the mechanism of action of the combination of mbsIgG2a-PD-L1×4-1BB and anti-mPD-1 in vivo. MC38-bearing mice were treated with mbsIgG2a-PD-L1×4-1BB, anti-mPD-1 or the combination thereof.

### Methods

### MC38 Colon Cancer Model

MC38 mouse colon carcinoma tumors from two independent studies were collected for immunohistochemistry and flow cytometry assessments to characterize the in vivo activity of mbsIgG2a-PD-L1×4-1BB and anti-mPD-1 as monotherapy and in combination.

The MC38 tumor model was established as described in Example 19. Treatment of mice bearing MC38 subcutaneous tumors was initiated when tumors had reached a tumor volume of 50-70 mm³. Mice were randomized into groups with equal mean tumor volume prior to treatment. On treatment days (two doses weekly for two weeks [2QWx2]), the mice were injected intraperitoneally with the antibodies indicated in Table 30 in an injection volume of 10 μL/g body weight. For combination treatments, antibodies were injected in two separate injections with 20 min in between (Table 30).

The mice were monitored daily for clinical signs of illness. Body weight measurements were performed three times a week after randomization. On Day 7 or 14 after initiation of treatment, mice (n=5 per group) were euthanized for resection of the tumors.

TABLE 30
Treatment groups and dosing regimen

| Treat- | | | | |
|---|---|---|---|---|
| ment | | | Dosing | Seq ids/ |
| group | Treatment | Dose | regimen | Supplier, cat. no. |
| 1 | PBS | N/a | 2QW × 2 | 1/a |
| 2 | Anti-mPD-1 | 10 mg/kg | 2QW × 2 | clone RMP1-14, |
| | | | | Leinco |
| | | | | Technologies, |
| | | | | cat. no. P372 |
| 3 | mbsIgG2a-PD-L1 × | 5 mg/kg | 2QW × 2 | Seq ids: 86, 87, |
| | 4-1BB | | | 81, 82, 83, 84, 85 |
| 4 | mbsIgG2a-PD-L1 × | 5 mg/kg | 2QW × 2 | See above: |
| | 4-1BB^{a} + | + | | group 2 and 3 |
| | Anti-mPD-1 | 10 mg/kg | | |

^{a}mbsIgG2a-PD-L1 × 4-1BB was injected first and the second antibody was injected after 20 min

### Immunohistochemistry and In Situ Hybridization of Tumor Tissue

Tumors were dissected, fixed in formalin, paraffin embedded and sectioned (4 pm). For histologic assessment, tumor sections were deparaffinized and stained with the Tissue-Tek Prisma H&E Stain Kit (Sakura [Torrance, CA], 6190) using the Tissue-Tek Prisma Plus Automated Slide Stainer (Sakura). For evaluation of CD3, CD4 and CD8+cells within the tumor, sections were deparaffinized and antigens were retrieved using CC1 buffer (Roche, 950-124), followed by quenching of endogenous peroxidase (Dako Agilent, S2003) and blocking of aspecific binding sites with blocking buffer (Roche, 05268869001) using the Roche Ventana Discovery (DISC) autostainer platform. Sections were incubated with primary antibodies (listed in Table 31), which were detected using anti-rabbit immunohistochemistry detection kits: for CD3 and CD4 with only anti-rabbit DISC, Omnimap (Roche, 05269679001) for CD8 sequentially with DISC anti-rabbit HQ (Roche, 07017812001) and DISC, and amplification for anti-HQ HRP Multimer (Roche, 06442544001). HRP was visualized using 3,3′-diaminobenzidine (ChromoMap DAB; Roche, 05266645001) according to manufacturer instructions. For evaluation of PD-L1+cells within the tumor, sections were deparaffinized and antigens were retrieved using ER2 buffer (Leica Biosystems, AR9640), followed by quenching of endogenous peroxidase (Dako Agilent, S2003) and blocking aspecific binding sites with blocking buffer (Leica Biosystems, DS9800) using the Leica Bond Rx autostainer platform. Sections were incubated with the primary antibody (listed in Table 31) which were detected using anti-rabbit immunohistochemistry detection kit (Leica Biosystems, DS9800) according to manufacturer instructions. For evaluation of 4-1BB+ and PD-L2+cells within the tumor, RNAscope assays have been performed on Leica Bond Rx with corresponding RNAscope probes (ACDBio, 493658 and 447788, respectively) and RNAscope detection kits (ACDBio, 322150) for detection of gene-specific mRNA molecules. In all assays, nuclei were counterstained by incubation with Mayer hematoxylin. Staining specificity was controlled by incorporating isotype, positive and negative control staining on consecutive tissue sections. Stained slides were subjected to whole slide imaging (Zeiss, Axioscan) and whole slide images were uploaded to and analyzed with Halo software (Indica Labs, Albuquerque, NM) using preprogrammed software analysis tools to determine CD3+, CD4+, CD8+ and PD-L1+cells (CytoNuclear v2.0.9) and to determine 4-1BB+ and PD-L2+cells (ISH v4.1.3). Quantitative data on CD3+, CD4+, CD8+, and PD-L1+cells were subsequently expressed as percentage of marker-positive cells in relation to total cell numbers. Quantitative data on 4-1BB+ and PD-L2+cells were expressed as RNAscope H-scores by creating four RNAscope intensity buckets and calculating H-scores with the formula: H-score=[(0× % cells with 0 dots/cell)+(1× % cells with 1-3 dots/cell)+(2× % cells with 4-9 dots/cell)+(3× % cells with 10^{×15} dots/cell)+(4× % cells with >15 dots/cell)].

TABLE 31
Antibodies used for immunohistochemistry

| Target | Label | Clone | Supplier | Catalog no. |
|---|---|---|---|---|
| CD3 | unconjμgated | 2GV6 | Ventana | 790-4341 |
| CD4 | unconjμgated | EPR19514 | Abcam | Ab183685 |
| CD8α | unconjμgated | D4W2Z | Cell Signaling Technology | 98941 |
| PD-L1 | unconjμgated | D5V3B | Cell Signalling Technology | 64988 |

### Flow Cytometry of Tumor Tissue

Dissociated tumor cells were blocked with 1 μg/mL Mouse BD Fc Block™ (Fc blocking buffer; BD, cat. no. 553141) at 4° C. in the dark for 10 min. For staining of cell surface markers, the fluorescently-labeled antibody mixture described in Table 32 (except Ki67 and GzmB) diluted in Fc blocking buffer were added to the cells, and incubated at 4° C. for 30 min, protected from light. For intracellular staining (Ki67 and GzmB), the cells were permeabilized by incubation with 200 μL Fix/Perm concentrate (eBioscience, cat. no. 00-5123) diluted in Fix/Perm dilution buffer (1:4; eBioscience, cat. no. 00-5223) at RT for 30 min, protected from light. After washing twice in Permeabilization buffer (eBioscience, cat. no. 00-8333), cells were incubated with Ki67 and GzmB antibodies (Table 32) diluted in Permabilization buffer at RT for 30 min, protected from light. Finally, cells were resuspended in 250 μL FACS buffer (PBS supplemented with 10% FBS [Gibco, cat, no. 10099-141] and 40 mM EDTA [Boston BioProducts, cat no. BM-711-K]) and measured at the BD LSRFortessa™ X20 cell analyzer (BD Biosciences, San Jose, CA, USA). Data were analyzed using Kaluza Analysis Software.

TABLE 32
Antibodies used for flow cytometry

| Target | Label¹ | Clone | Supplier | Cat. no. |
|---|---|---|---|---|
| CD45 | BV785 | 30-F11 | Biolegend | 103149 |
| CD3 | BUV395 | 17A2 | BD | 740268 |
| CD4 | BV510 | GK1.5 | Biolegend | 100449 |
| CD8 | PE-eFluor610 | 53-6.7 | eBiosciences | 61-0081-82 |
| Ki67 | PerCP/Cy5.5 | SolA15 | eBioscience | 46-5698-82 |
| GzmB | AF700 | QA16A02 | Biolegend | 372222 |
| Live/dead | eFluor780 | N/A | eBioscience | 65-0865 |

### Results & Conclusion

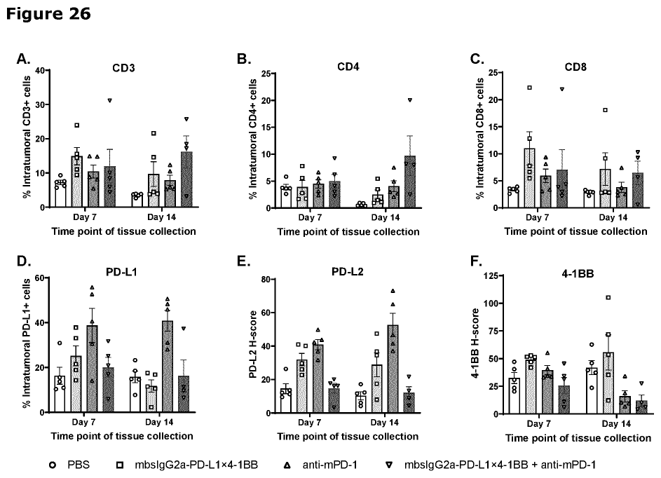

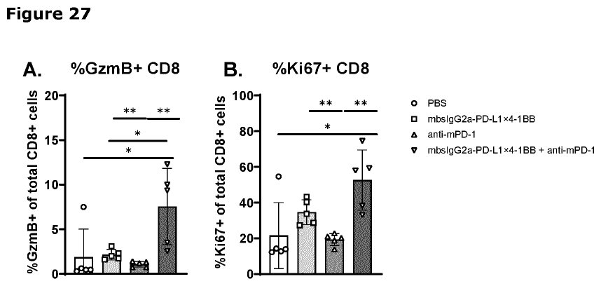

Tumor tissue sections were evaluated for T cell subsets and target expression by immunohistochemistry (IHC) and in situ hybridization (ISH) on day 7 and day 14 following treatment initiation (FIG. 26) and dissociated tumor tissues were evaluated for Ki67T proliferating and GzmB+cytotoxic intratumoral CD8⁺ T cells by flow cytometry on day 7 post treatment initiation (FIG. 27).

Treatment with mbsIgG2a-PD-Llx4-1BB and anti-mPD-1 as single agents enhanced the percentage of CD3+cells within the tumor on Day 7 and Day 14 post-treatment. The combination of mbsIgG2a-PD-L1×4-1BB with anti-mPD-1 further increased the percentage of CD3Y cells on Day 14 (FIG. 26A). No differences in the percentage of CD4+cells were observed between treatment groups on Day 7. In contrast, the percentage of CD4+cells were increased by treatment with mbsIgG2a-PD-Llx4-1BB and anti-mPD-1 as single agents compared to the PBS-treated group on Day 14 and even further enhanced by the combination of mbsIgG2a-PD-L1×4-1BB with anti-mPD-1 (FIG. 26B).

The percentage of CD8+cells was increased by mbsIgG2a-PD-L1×4-1BB compared to the PBS group on both Day 7 and Day 14, but not by anti-mPD-1. The combination mbsIgG2a-PD-Llx4-1BB with anti-mPD-1 showed similar levels of CD8+cells compared to mbsIgG2a-PD-Llx4-1BB alone, suggesting that the increase in CD8+cells was driven by mbsIgG2a-PD-L1×4-1BB (FIG. 26C).

On Day 7 and/or Day 14, intratumoral PD-L1 and PD-L2 expression was increased by mbsIgG2a-PD-L1×4-1BB and anti-mPD-1 as single agents compared to the PBS-treated mice. By contrast, the combination of mbsIgG2a-PD-L1×4-1BB with anti-mPD-1 did not show such an increase, as the levels of intratumoral PD-L1 and PD-L2 were comparable to the levels in PBS-treated mice (FIG. 26D-E). Finally, tumoral expression of 4-1BB was increased by mbsIgG2a-PD-L1×4-1BB on Day 7. By contrast, expression of 4-1BB was decreased by anti-mPD-1 as single agent and by the combination ofmbsIgG2a-PD-L1×4-1BB with anti-mPD-1 on Day 14 (FIG. 26F)

In dissociated tumor tissues, it was found that the percentage of GzmB+within the total intratumoral CD8⁺ T cell population was significantly enhanced by the combination of mbsIgG2a-PD-L1×4-1BB and anti-mPD-1 compared to each single agent (FIG. 27A), suggesting increased CD8 T-cell cytotoxicity. Similarly, the percentage of Ki67T within the total tumor-infiltrating CD8⁺ T cell population was enhanced by the combination of mbsIgG2a-PD-L1×4-1BB and anti-mPD-1 compared to each single agent alone, suggesting increased CD8 T-cell proliferation (FIG. 27B).

Together, these results suggest that the combination of mbsIgG2a-PD-L1×4-1BB and anti-mPD-1 leads to distinct and complementary modulation of the tumor immune contexture compared to treatment with mbsIgG2a-PD-L1x4-1BB or anti-mPD-1 as single agents. In particular, the greater frequency of proliferating and cytotoxic CD8⁺ TILs in the mbsIgG2a-PD-L1x4-1BB with anti-PD1 combination treated group indicates enhanced functional and effector functions of TILs likely associated with improved antitumor activity.

1. A binding agent for use in a method for reducing or preventing progression of a tumor or treating cancer in a subject, said method comprising administering to said subject the binding agent prior to, simultaneously with, or after administration of a PD-1 inhibitor, wherein the binding agent comprises a first binding region binding to CD137 and a second binding region binding to PD-L1; and
Wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,
then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

2. The binding agent for use of claim 1, wherein PD-L1 is human PD-L1, in particular human PD-L1 comprising the sequence set forth in SEQ ID NO: 40, and/or CD137 is human CD137, in particular human CD137 comprising the sequence set forth in SEQ ID NO: 38.

3. The binding agent for use of any one of claims 1 to 3, wherein the PD-1 inhibitor is a PD-1 antibody.

4. The binding agent for use of any one of claims 1 to 4, wherein the PD-1 inhibitor is a PD-1 blocking antibody.

5. The binding agent for use of any one of the preceding claims, wherein the PD-1 inhibitor is pembrolizumab or a biosimilar thereof.

6. The binding agent for use of any one of the preceding claims, wherein when the PD-1 inhibitor is nivolumab or a biosimilar thereof.

7. The binding agent for use of any one of the preceding claims, wherein
a) the first binding region comprises a heavy chain variable region (VH) comprising the CDR1, CDR2, and CDR3 sequences of SEQ ID NO: 1 or 9, and a light chain variable region (VL) comprising the CDR1, CDR2, and CDR3 sequences of SEQ ID NO: 5 or 10;
and
b) the second antigen-binding region comprises a heavy chain variable region (VH) comprising the CDR1, CDR2, and CDR3 sequences of SEQ ID NO: 11, and a light chain variable region (VL) comprising the CDR1, CDR2, and CDR3 sequences of SEQ ID NO: 15.

8. The binding agent for use of any one of the preceding claims, wherein
a) the first binding region comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively;
and
b) the second antigen-binding region comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively.

9. The binding agent for use of any one of the preceding claims, wherein
the first binding region comprises a heavy chain variable region (VH) comprising an amino acid sequence having at least 90%, at least 95%, at least 97%, at least 99%, or 100% sequence identity to SEQ ID NO: 1 or 9 and a light chain variable region (VL) region and comprising an amino acid sequence having at least 90%, at least 95%, at least 97%, at least 99%, or 100% sequence identity to SEQ ID NO: 5 or 10.

10. The binding agent for use of any one of the preceding claims, wherein
the second binding region comprises a heavy chain variable region (VH) comprising an amino acid sequence having at least 90%, at least 95%, at least 97%, at least 99%, or 25 100% sequence identity to SEQ ID NO: 11 and a light chain variable region (VL) region comprising an amino acid sequence having at least 90%, at least 95%, at least 97%, at least 99%, or 100% sequence identity to SEQ ID NO: 15.

11. The binding agent for use of any one of the preceding claims, wherein
the first binding region comprises a heavy chain variable region (VH) comprising the amino acid sequence set forth in SEQ ID NO: 1 or 9 and a light chain variable region (VL) region comprising the amino acid sequence set forth in SEQ ID NO: 5 or 10.

12. The binding agent for use of any one of the preceding claims, wherein the second binding region comprises a heavy chain variable region (VH) comprising the amino acid sequence set forth in SEQ ID NO: 11 and a light chain variable region (VL) region comprising the amino acid sequence set forth in SEQ ID NO: 15.

13. The binding agent for use of any one of the preceding claims, wherein
a) the first binding region comprises a heavy chain variable region (VH) comprising the amino acid sequence set forth in SEQ ID NO: 1 and a light chain variable region (VL) region comprising the amino acid sequence set forth in SEQ ID NO: 5;
and
b) the second binding region comprises a heavy chain variable region (VH) comprising the amino acid sequence set forth in SEQ ID NO: 11 and a light chain variable region (VL) region comprising the amino acid sequence set forth in SEQ ID NO: 15.

14. The binding agent for use of any one of the preceding claims, wherein the binding agent is a multispecific antibody, such as a bispecific antibody.

15. The binding agent for use of any one of the preceding claims, wherein the binding agent is in the format of a full-length antibody or an antibody fragment.

16. The binding agent for use of any one of claims 6-12, wherein each variable region comprises three complementarity determining regions (CDR1, CDR2, and CDR3) and four framework regions (FR1, FR2, FR3, and FR4).

17. The binding agent for use of claim 13, wherein said complementarity determining regions and said framework regions are arranged from amino-terminus to carboxy-terminus in the following order: FR1, CDR1, FR2, CDR2, FR3, CDR3, FR4.

18. The binding agent for use of any one of claims 7-17, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

19. The binding agent for use of any one of claims 7-18, which comprises
i) a polypeptide comprising said first light chain variable region (VL) and further comprising a first light chain constant region (CL), and
ii) a polypeptide comprising said second light chain variable region (VL) and further comprising a second light chain constant region (CL).

20. The binding agent for use of any one of claims 7-19, wherein the binding agent is an antibody comprising a first binding arm and a second binding arm, wherein
the first binding arm comprises
i) a polypeptide comprising said first heavy chain variable region (VH) and a first heavy chain constant region (CH), and
ii) a polypeptide comprising said first light chain variable region (VL) and a first light chain constant region (CL);
and the second binding arm comprises
iii) a polypeptide comprising said second heavy chain variable region (VH) and a second heavy chain constant region (CH), and
iv) a polypeptide comprising said second light chain variable region (VL) and a second light chain constant region (CL).

21. The binding agent for use of any one of the preceding claims, which comprises
i) a first heavy chain and light chain comprising said antigen-binding region capable of binding to CD137, and
ii) a second heavy chain and light chain comprising said antigen-binding region capable of binding PD-L1.

22. The binding agent for use of any one of the preceding claims, wherein said binding agent comprises
i) a first heavy chain and light chain comprising said antigen-binding region capable of binding to CD137, the first heavy chain comprising a first heavy chain constant region and the first light chain comprising a first light chain constant region; and
ii) a second heavy chain and light chain comprising said antigen-binding region capable of binding PD-L1, the second heavy chain comprising a second heavy chain constant region and the second light chain comprising a second light chain constant region.

23. The binding agent for use of any one of claims 18-22, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

24. The binding agent for use of any one of claims 18-23, wherein each of the first and second heavy chain constant regions (CHs) comprises a CH3 region and wherein the two CH3 regions comprise asymmetrical mutations.

25. The binding agent for use of any one of claims 18-23, wherein in said first heavy chain constant region (CH) at least one of the amino acids in a position corresponding to a position selected from the group consisting ofT366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

26. The binding agent for use of claim 25, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

27. The binding agent for use of any of the preceding claims, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.

28. The binding agent for use of claim 27, wherein said first and second heavy chain constant regions (CHs) are modified so that the antibody induces Fc-mediated effector function to a lesser extent compared to an antibody which is identical except for comprising non-modified first and second heavy chain constant regions (CHs).

29. The binding agent for use of claim 28, wherein each of said non-modified first and second heavy chain constant regions (CHs) comprises the amino acid sequence set forth in SEQ ID NO: 19 or 25.

30. The binding agent for use of claim 28 or 29, wherein said Fc-mediated effector function is measured by binding to Fcγ receptors, binding to C1q, or induction of Fe-mediated crosslinking of Fcγ receptors.

31. The binding agent for use of claim 30, wherein said Fc-mediated effector function is measured by binding to C1q.

32. The binding agent for use of any one of claims 27-31, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

33. The binding agent for use of any one of claims 18-32, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

34. The binding agent for use of claim 33, wherein the positions corresponding to positions L234 and L235 in a human IgG1 heavy chain according to EU numbering are F and E, respectively, in said first and second heavy chains.

35. The binding agent for use of claim 33 or 34, wherein the positions corresponding to positions L234, L235, and D265 in a human IgG1 heavy chain according to EU numbering are F, E, and A, respectively, in said first and second heavy chain constant regions.

36. The binding agent for use of any one of claims 33-35, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

37. The binding agent for use of any one of claims 33-36, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

38. The binding agent for use of any one of claims 18-37, wherein the constant region of said first and/or second heavy chain comprises or consists essentially of or consists of an amino acid sequence selected from the group consisting of
a) the sequence set forth in SEQ ID NO: 19 or 25 [IgG1-FC];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4, at most 3, at most 2 substitutions or at most 1 substitution, compared to the amino acid sequence defined in a) or b).

39. The binding agent for use of any one of claims 18-38, wherein the constant region of said first or second heavy chain, such as the second heavy chain, comprises or consists essentially of or consists of an amino acid sequence selected from the group consisting of
a) the sequence set forth in SEQ ID NO: 20 or 26 [IgG1-F405L];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 9 substitutions, such as at most 8, at most 7, at most 6, at most 5, at most 4, at most 3, at most 2 substitutions or at most 1 substitution, compared to the amino acid sequence defined in a) or b).

40. The binding agent for use of any one of claims 18-38, wherein the constant region of said first or second heavy chain, such as the first heavy chain comprises or consists essentially of or consists of an amino acid sequence selected from the group consisting of
a) the sequence set forth in SEQ ID NO: 21 or 27 [IgG1-K409R];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

41. The binding agent for use of any one of claims 18-37, wherein the constant region of said first and/or second heavy chain comprises or consists essentially of or consists of an amino acid sequence selected from the group consisting of
a) the sequence set forth in SEQ ID NO: 22 or 28 [IgG1-Fc_FEA];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 7 substitutions, such as at most 6 substitutions, at most 5, at most 4, at most 3, at most 2 substitutions or at most 1 substitution, compared to the amino acid sequence defined in a) or b).

42. The binding agent for use of any one of claims 18-41, wherein the constant region of said first and/or second heavy chain, such as the second heavy chain, comprises or consists essentially of or consists of an amino acid sequence selected from the group consisting of
a) the sequence set forth in SEQ ID NO: 24 or 30[IgG1-Fc_FEAL];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4 substitutions, at most 3, at most 2 substitutions or at most 1 substitution, compared to the amino acid sequence defined in a) or b).

43. The binding agent for use of any one of claims 18-42, wherein the constant region of said first and/or second heavy chain, such as the first heavy chain, comprises or consists essentially of or consists of an amino acid sequence selected from the group consisting of
a) the sequence set forth in SEQ ID NO: 23 or 29 [IgG1-Fc FEAR];
b) a subsequence of the sequence in a), such as a subsequence, wherein 1, 2, 3, 4, 5, 6, 7, 8, 9 or 10 consecutive amino acids has/have been deleted, starting from the N-terminus or C-terminus of the sequence defined in a); and
c) a sequence having at most 6 substitutions, such as at most 5 substitutions, at most 4, at most 3, at most 2 substitutions or at most 1 substitution, compared to the amino acid sequence defined in a) or b).

44. The binding agent for use of any one of the preceding claims, wherein said binding agent comprises a kappa (x) light chain constant region.

45. The binding agent for use of any one of the preceding claims, wherein said binding agent comprises a lambda (Q) light chain constant region.

46. The binding agent for use of any one of the preceding claims, wherein said first light chain constant region is a kappa (x) light chain constant region or a lambda (Q) light chain constant region.

47. The binding agent for use of any one of the preceding claims, wherein said second light chain constant region is a lambda (k) light chain constant region or a kappa (x) light chain constant region.

48. The binding agent for use of any one of the preceding claims, wherein said first light chain constant region is a kappa (x) light chain constant region and said second light chain constant region is a lambda (k) light chain constant region or said first light chain constant region is a lambda (k) light chain constant region and said second light chain constant region is a kappa (x) light chain constant region.

49. The binding agent for use of any one of claims 44-48, wherein the kappa (x) light chain comprises an amino acid sequence selected from the group consisting of
a) the sequence set forth in SEQ ID NO:35,
b) a subsequence of the sequence in a), such as a subsequence, wherein 1, 2, 3, 4, 5, 6, 7, 20 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

50. The binding agent for use of any one of claims 45-49, wherein the lambda (k) light chain comprises an amino acid sequence selected from the group consisting of
a) the sequence set forth in SEQ ID NO: 36,
b) a subsequence of the sequence in a), such as a subsequence, wherein 1, 2, 3, 4, 5, 6, 7, 30 8, 9 or 10 consecutive amino acids has/have been deleted, starting from the N-terminus or C-terminus of the sequence defined in a); and
c) a sequence having at most 10 substitutions, such as at most 9 substitutions, at most 8, at most 7, at most 6, at most 5, at most 4 substitutions, at most 3, at most 2 substitutions or at most 1 substitution, compared to the amino acid sequence defined in a) or b).

51. The binding agent for use of any one of the preceding claims, wherein the binding agent is of an isotype selected from the group consisting of IgG1, IgG2, IgG3, and IgG4.

52. The binding agent for use of any one of the preceding claims, wherein the binding agent is a full-length IgG1 antibody.

53. The binding agent for use of any one of the preceding claims, wherein the binding agent is an antibody of the IgG1m(f) allotype.

54. The binding agent for use of any one of the preceding claims, wherein the binding agent comprises
i) a first heavy chain and light chain comprising said antigen-binding region capable of binding to CD137, wherein the first heavy chain comprising the sequence set forth in SEQ ID NO: 31, and the first light chain comprising the sequence set forth in SEQ ID NO: 32;
ii) a second heavy chain and light chain comprising said antigen-binding region capable of binding PD-L1, wherein the second heavy chain comprising the sequence set forth in SEQ ID NO: 33, and the second light chain comprising the sequence set forth in SEQ ID NO: 34.

55. The binding agent for use according to any one of the preceding claims, wherein the binding agent is acasunlimab or a biosimilar thereof.

56. The binding agent for use according to any one of the preceding claims, wherein the binding agent is in a composition or formulation comprising histidine, sucrose and Polysorbate-80, and has a pH from 5 to 6.

57. The binding agent for use according to any one of the preceding claims, wherein the binding agent is in a composition or formulation comprising about 20 mM histidine, about 250 mM Sucrose, about 0.02% Polysorbate-80, and having a pH of about 5.5.

58. The binding agent for use according to any one of the preceding claims, wherein the binding agent is in a composition or formulation comprising 10^{×30} mg binding agent/mL, such as 20 mg binding agent/mL.

59. The binding agent for use according to any one of the preceding claims, wherein the binding agent is in a composition as defined in any one of claims 56 to 58 and is diluted in 0.9% NaCl (saline) prior to administration.

60. The binding agent for use according to any one of the preceding claims, the PD-1 inhibitor is an antibody binding to PD-1, wherein the antibody binding to PD-1 comprises a VH region CDR1, CDR2, and CDR3 comprising the sequences as set forth in SEQ ID NOs: 104, 101, and 100, respectively, and a VL region CDR1, CDR2, and CDR3 comprising the sequences as set forth in SEQ ID NO: 107, QAS and SEQ ID NO: 105, respectively.

61. The binding agent for use according to claim 60, wherein the antibody binding to PD-1 comprises a heavy chain variable region (VH) comprising a sequence having at least 70%, at least 75%, at least 80%, at least 85%, at least 90%, at least 95%, at least 97%, at least 99%, or 100% identity to the amino acid sequence of the VH sequence as set forth in SEQ ID NO: 111.

62. The binding agent for use according to claim 60 or 61, wherein the antibody binding to PD-1 comprises a heavy chain variable region (VH), wherein the VH comprises the sequence as set forth in SEQ ID NO: 111.

63. The binding agent for use according to any one of claims 60-62, wherein the antibody binding to PD-1 comprises a light chain variable region (VL) comprising a sequence having at least 70%, at least 75%, at least 80%, at least 85%, at least 90%, at least 95%, at least 97%, at least 99%, or 100% identity to the amino acid sequence of the VL sequence as set forth in SEQ ID NO: 112.

64. The binding agent for use according to claim 63, wherein the antibody binding to PD-1 comprises a light chain variable region (VL), wherein the VL comprises the sequence as set forth in SEQ ID NO: 112.

65. The binding agent for use according to any one of claims 60-64, wherein the antibody binding to PD-1 comprises a heavy chain variable region (VH) and a light chain variable region (VL), wherein the VH comprises or has the sequence as set forth in SEQ ID NO: 111 and the VL comprises or has the sequence as set forth in SEQ ID NO: 112.

66. The binding agent for use according to any one of claims 60-65, wherein the antibody binding to PD-1 comprises a heavy chain constant region, wherein the heavy chain constant region comprises an aromatic or non-polar amino acid at the position corresponding to position 234 in a human IgG1 heavy chain according to EU numbering and an amino acid other than glycine at the position corresponding to position 236 in a human IgG1 heavy chain according to EU numbering.

67. The binding agent for use according to claim 66, wherein the amino acid at the position corresponding to position 236 is a basic amino acid.

68. The binding agent for use according to claim 67, wherein the basic amino acid is selected from the group consisting of lysine, arginine and histidine.

69. The binding agent for use according to claim 67 or 68, wherein the basic amino acid is arginine (G236R).

70. The binding agent for use according to any one of claims 66-69, wherein the amino acid at the position corresponding to position 234 is an aromatic amino acid.

71. The binding agent for use according to claim 70, wherein the aromatic amino acid is selected from the group consisting of phenylalanine, tryptophan and tyrosine.

72. The binding agent for use according to any one of claims 66-69, wherein the amino acid at the position corresponding to position 234 is a non-polar amino acid.

73. The binding agent for use according to claim 72, wherein the non-polar amino acid is selected from the group consisting of alanine, valine, leucine, isoleucine, proline, phenylalanine, methionine and tryptophan.

74. The binding agent for use according to claim 72 or 73, wherein the non-polar amino acid is selected from the group consisting of isoleucine, proline, phenylalanine, methionine and tryptophan.

75. The binding agent for use according to any one of claims 66-74, wherein the amino acid at the corresponding to position 234 is phenylalanine (L234F).

76. The binding agent for use according to any one of claims 66-75, wherein the amino acid at the position corresponding to position 235 in a human IgG1 heavy chain according to EU numbering in said heavy chain constant region of the antibody binding to PD-1 is an acidic amino acid.

77. The binding agent for use according to claim 76, wherein the acidic amino acid is aspartate or glutamate.

78. The binding agent for use according to any one of claims 66-77, wherein the amino acid at the position corresponding to position 235 in a human IgG1 heavy chain according to EU numbering in said heavy chain constant region of the antibody binding to PD-1 is glutamate (L235E).

79. The binding agent for use according to any one of claims 66-78, wherein the amino acids at the position corresponding to positions 234, 235 and 236 in said heavy chain constant region of the antibody binding to PD-1 are a non-polar or an aromatic amino acid at position 234, an acidic amino acid at position 235 and a basic amino acid at position 236.

80. The binding agent for use according to any one of claims 66-79, wherein the amino acid corresponding to position 234 is phenylalanine, the amino acid corresponding to position 235 is glutamate, and the amino acid corresponding to position 236 is arginine in said heavy chain constant region of the antibody binding to PD-1 (L234F/L235E/G236R).

81. The binding agent for use according to any one of claims 60-80, wherein the heavy chain constant region of the antibody binding to PD-1 comprises a sequence having at least 70%, at least 75%, at least 80%, at least 85%, at least 90%, at least 95%, at least 97%, at least 99%, or 100% identity to the amino acid sequence of the heavy chain constant region sequence as set forth in SEQ ID NO: 93.

82. The binding agent for use according to any one of claims 60-81, wherein the heavy chain constant region of the antibody binding to PD-1 comprises the sequence as set forth in SEQ ID NO: 93.

83. The binding agent for use according to any one of claims 60-82, wherein the isotype of the heavy chain constant region of the antibody binding to PD-1 is IgG1.

84. The binding agent for use according to any one of claims 60-83, wherein the antibody binding to PD-1 is a monoclonal, chimeric or humanized antibody or a fragment of such an antibody.

85. The binding agent for use according to any one of claims 60-84, wherein the antibody binding to PD-1 has a reduced or depleted Fc-mediated effector function.

86. The binding agent for use according to any one of claims 60-85, wherein binding of complement protein C1q to the constant region of the antibody binding to PD-1 is reduced compared to a wild-type antibody, preferably by at least 70%, at least 80%, at least 90%, at least 95%, at least 97% or 100%.

87. The binding agent for use according to any one of claims 60-86, wherein binding to one or more of the IgG Fc-gamma receptors to the antibody binding to PD-1 is reduced compared to a wild-type antibody, preferably by at least 70%, at least 80%, at least 90%, at least 95%, at least 97% or 100%.

88. The binding agent for use according to claim 87, wherein the one or more IgG Fc-gamma receptors are selected from at least one of Fc-gamma RI, Fc-gamma RII and Fc-gamma RIII.

89. The binding agent for use according to claim 87 or 88, wherein the IgG Fc-gamma receptor is Fc-gamma RI.

90. The binding agent for use according to any one of claims 60-89, wherein the antibody binding to PD-1 is not capable of inducing Fc-gamma RI-mediated effector functions or wherein the induced Fc-gamma RI-mediated effector functions are reduced compared to a wild-type antibody, preferably by at least 70%, at least 80%, at least 90%, at least 95%, at least 97% or 100%.

91. The binding agent for use according to any one of claims 60-90, wherein the antibody binding to PD-1 is not capable of inducing at least one of complement dependent cytotoxicity (CDC) mediated lysis, antibody dependent cellular cytotoxicity (ADCC) mediated lysis, apoptosis, homotypic adhesion and/or phagocytosis or wherein at least one of complement dependent cytotoxicity (CDC) mediated lysis, antibody dependent cellular cytotoxicity (ADCC) mediated lysis, apoptosis, homotypic adhesion and/or phagocytosis is induced in a reduced extent, preferably reduced by at least 70%, at least 80%, at least 90%, at least 95%, at least 97% or 100%.

92. The binding agent for use according to any one of claims 60-91, wherein binding of neonatal Fc receptor (FcRn) to the antibody binding to PD-1 is unaffected, as compared to a wild-type antibody.

93. The binding agent for use according to any one of claims 60-92, wherein PD-1 is human PD-1.

94. The binding agent for use according to claim 93, wherein the PD-1 has or comprises the amino acid sequence as set forth in SEQ ID NO: 113 or SEQ ID NO: 114, or the amino acid sequence of PD-1 has at least 70%, at least 75%, at least 80%, at least 85%, at least 90%, at least 95%, at least 97%, at least 99%, or 100% identity to the amino acid sequence as set forth in SEQ ID NO: 113 or SEQ ID NO: 114, or is an immunogenic fragment thereof.

95. The binding agent for use according to any one of claims 60-94, the antibody binding to PD-1 binds to a native epitope of PD-1 present on the surface of living cells.

96. The binding agent for use according to any one of claims 60-95, wherein the antibody binding to PD-1 is a multispecific antibody comprising a first antigen-binding region binding to PD-1 and at least one further antigen-binding region binding to another antigen.

97. The binding agent for use according to claim 96, wherein the antibody binding to PD-1 is a bispecific antibody comprising a first antigen-binding region binding to PD-1 and a second antigen-binding region binding to another antigen.

98. The binding agent for use according to claim 96 or 97, wherein the first antigen-binding region binding to PD-1 comprises the heavy chain variable region (VH) and/or the light chain variable region (VL) as set forth in any one of claims 61 to 65.

99. The binding agent for use of any one of the preceding claims, wherein the subject is a human subject.

100. The binding agent for use of any one of the preceding claims, wherein the tumor or cancer is a solid tumor or cancer.

101. The binding agent for use according to any one of the preceding claims, wherein said tumor is a PD-L1 positive tumor.

102. The binding agent for use of any one of the preceding claims, wherein the tumor or cancer is selected from the group consisting of melanoma, ovarian cancer, lung cancer (e.g., non-small cell lung cancer (NSCLC)), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, endometrial cancer, prostate cancer, penile cancer, cervical cancer, Hodgkin's lymphoma, non-Hodgkin's lymphoma, Merkel cell carcinoma and mesothelioma.

103. The binding agent for use according to any one of the preceding claims, wherein the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)) and squamous cell carcinoma of the head and neck (SCCHN) (e.g. cancer of the oral cavity, pharynx or larynx).

104. The binding agent for use of claim 102 or 103, wherein the tumor or cancer is lung cancer, in particular a non-small cell lung cancer (NSCLC), such as a squamous or non-squamous NSCLC.

105. The binding agent for use of any one of claims 100 to 104, wherein the tumor or cancer is metastatic, such as metastatic NSCLC.

106. The binding agent for use of claim 104 or 105, wherein the lung cancer, in particular NSCLC, does not have an epidermal growth factor (EGFR)-sensitizing mutation and/or anaplastic lymphoma (ALK) translocation/ROS1 rearrangement.

107. The binding agent for use of any one of claims 104 to 106, wherein the lung cancer, in particular NSCLC, comprises cancer cells and PD-L1 is expressed in >1% of the cancer cells or tumor cells e.g. as assessed by immunohistochemistry (IHC).

108. The binding agent for use of the preceding claims, wherein the subject has not received prior systemic treatment of metastatic disease.

109. The binding agent for use of any one of the preceding claims, wherein the subject has not received prior treatment with a checkpoint inhibitor; e.g., a PD-1 inhibitor or a PD-L1 inhibitor, such as anti-PD-1 antibody or an anti-PD-L1 antibody.

110. The binding agent for use of any one of the preceding claims, wherein the subject has not received prior treatment with a 4-1BB (CD137) targeted agent, such as an anti-4-1BB (CD137) antibody, with an antitumor vaccine, or with autologous cell immunotherapy.

111. The binding agent for use of any one of claims 1 to 107, wherein the tumor or cancer has relapsed and/or is refractory after treatment, such as systemic treatment with a checkpoint inhibitor.

112. The binding agent for use of any one of claims 1 to 107 and 111, wherein the subject has received at least 1 prior line of systemic therapy, such as systemic therapy comprising a PD-1 inhibitor or a PD-L1 inhibitor, such as an anti-PD-1 antibody or an anti-PD-L1 antibody.

113. The binding agent for use of any one of claims 1 to 107, 111 and 112, wherein the cancer or tumor has relapsed and/or is refractory, or the subject has progressed after treatment with a PD-1 inhibitor or a PD-L1 inhibitor, such as an anti PD-1 antibody or an anti-PD-L1 antibody, the PD-1 inhibitor or PD-L1 inhibitor being administered as monotherapy or as part of a combination therapy.

114. The binding agent for use of any one of claims 1 to 107 and 111 to 113, wherein last prior treatment was with a PD1 inhibitor or PD-L1 inhibitor, such as an anti PD-1 antibody or an anti-PD-L1 antibody, the PD-1 inhibitor or PD-L1 inhibitor being administered as monotherapy or as part of a combination therapy.

115. The binding agent for use of any one of claims 1 to 107 and 111 to 114, wherein the time from progression on last treatment with a PD1 inhibitor or PD-L1 inhibitor, such as an anti PD-1 antibody or an anti-PD-L1 antibody is 8 months or less, such as 7 months or less, 6 months or less, 5 months or less, 4 months or less, 3 months or less, 2 months or less, 1 month or less, 3 weeks or less or such as 2 weeks or less.

116. The binding agent for use of any one of claims 1 to 107 and 111 to 115, wherein the time from last dosing of a PD1 inhibitor or PD-L1 inhibitor, such as an anti PD-1 antibody or an anti-PD-L1 antibody as part of last prior treatment is 8 months or less, such as 7 months or less, 6 months or less, 5 months or less, 4 months or less, 3 months or less, 2 months or less, 1 month or less, 3 weeks or less or such as 2 weeks or less.

117. The binding agent for use of any one of claims 1 to 107 and 111 to 116, wherein the cancer or tumor has relapsed and/or is refractory, or the subject has progressed during or after
i) platinum doublet chemotherapy following treatment with an anti-PD-1 antibody or an anti-PD-L1 antibody, or
ii) treatment with an anti-PD-1 antibody or an anti-PD-L1 antibody following platinum doublet chemotherapy.

118. The binding agent for use of any one of the preceding claims, wherein the subject has not received prior treatment with a taxane chemotherapeutic agent e.g., docetaxel, such as prior treatment of NSCLC with a taxane chemotherapeutic agent e.g., docetaxel.

119. The binding agent for use of any one of the preceding claims, wherein the binding agent and the PD-1 inhibitor are administered in at least one treatment cycle, each treatment cycle being two weeks (14 days), three weeks (21 days), four weeks (28 days), 5 weeks (35 days) or six weeks (42 days).

120. The binding agent for use of any one of the preceding claims, wherein one dose of the binding agent and one dose of the PD-1 inhibitor are administered every second week (1Q2W) every third week (1Q3W), every fourth week (1Q4W), every fifth week (1Q5W) or every sixth week (1Q6W).

121. The binding agent for use of any one of the preceding claims, wherein one dose of the binding agent and one dose of the PD-1 inhibitor are administered every six weeks (1Q6W).

122. The binding agent for use of any one of the preceding claims, wherein one dose of the 15 binding agent and one dose of the PD-1 inhibitor are administered on day 1 of each treatment cycle.

123. The binding agent for use of any one of the preceding claims, wherein the amount of said binding agent administered in each dose and/or in each treatment cycle is 100 mg.

124. The binding agent for use of any one of the preceding claims, wherein the amount of said PD-1 inhibitor administered in each dose and/or in each treatment cycle is 200 mg.

125. The binding agent for use of any one of the preceding claims, wherein the amount of said PD-1 inhibitor administered in each dose and/or in each treatment cycle is 400 mg.

126. The binding agent for use of any one of the preceding claims, wherein a 100 mg dose of the binding agent and a 200 mg dose of the PD-1 inhibitor are administered every three weeks (1Q3W).

127. The binding agent for use of any one of the preceding claims, wherein a 100 mg dose of the binding agent and a 400 mg dose of the PD-1 inhibitor are administered every six weeks (1Q6W).

128. The binding agent for use of any one of the preceding claims, wherein the tumor or cancer is NSCLC; and wherein a 100 mg dose of the binding agent, which is acasunlimab or a biosimilar thereof and a 200 mg dose of the PD-1 inhibitor, which is nivolumab, are administered every three weeks (1Q3W), such as on day one of each three-week treatment cycle.

129. The binding agent for use of any one of the preceding claims, wherein the PD-1 inhibitor is administered first, followed by the binding agent.

130. The binding agent for use of any one of the preceding claims, wherein the binding agent is administered by using intravenous (IV) infusion over a minimum of 30 minutes, such as over a minimum of 60 minutes.

131. The binding agent for use of any one of the preceding claims, wherein the binding agent is administered by using intravenous (IV) infusion over 30 minutes.

132. The binding agent for use of any one of the preceding claims, wherein the PD-1 inhibitor is administered as an intravenous infusion over 30 minutes.

133. A kit comprising (i) a binding agent comprising a first binding region binding to CD137 and a second binding region binding to PD-L1, and (ii) a PD-1 inhibitor;
wherein when
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,
then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

134. The kit according to claim 133, wherein the binding agent is as defined in any one of claims 1, 2 and 7-58 and/or the PD-1 inhibitor is as defined in any one of claims 3 to 6, and 59-97.

135. The kit according to claim 133 or 134, wherein the binding agent, the PD-1 inhibitor, and, if present, the one or more additional therapeutic agents are for systemic administration, in particular for injection or infusion, such as intravenous injection or infusion.

136. The kit according to any one of claims 133-135 for use in a method for reducing or preventing progression of a tumor or treating cancer in a subject.

137. The kit for use according to claim 136, wherein the tumor or cancer and/or the subject and/or the method is/are as defined in any one of claims 1-132.

138. A method for reducing or preventing progression of a tumor or treating cancer in a subject, said method comprising administering to said subject a binding agent prior to, simultaneously with, or after administration of a PD-1 inhibitor, wherein the binding agent comprises a first binding region binding to CD137 and a second binding region binding to PD-L1, and
wherein
a) the first binding region binding to CD137 comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, 7, and 8, respectively; and
b) the second binding region binding to PD-L1 comprises a heavy chain variable region (VH) comprising the CDR1, CDR2, and CDR3 sequences set forth in: SEQ ID NO: 12, 13, and 14, respectively, and a light chain variable region (VL) comprising the CDR1, CDR2, and CDR3 sequences set forth in: SEQ ID NO: 16, 17, and 18, respectively,
then the PD-1 inhibitor is not an antibody comprising a heavy chain variable region (VH) comprising the CDR1, CDR2 and CDR3 sequences set forth in SEQ ID NO: 59, 60 and 61, respectively, and a light chain variable region (VL) comprising the CDR1, CDR2, and CDR3 sequences set forth in SEQ ID NO: 62, 63, and 64, respectively, or an antigen-binding fragment thereof.

139. The method of claim 138, wherein the tumor or cancer and/or the subject and/or the method and/or the binding agent and/or the PD-1 inhibitor is/are as defined in any one of claims 1-132.
